SEQUENCE LISTING (83 sequences)

SEQ ID NO: 1 - protein, 15 aa, synthetic construct
  REGION 1..15: note = Description of sequence: Linker 1
GGGGSGGGGSGGGGS

SEQ ID NO: 2 - protein, 19 aa, synthetic construct
  REGION 1..19: note = Description of sequence: Linker 2
GGGGSGGGGSGGGSGGGGS

SEQ ID NO: 3 - protein, 24 aa, synthetic construct
  REGION 1..24: note = Description of sequence: Linker 3
GGGGSGGGGSGGGGSGGGSGGGGS

SEQ ID NO: 4 - protein, 29 aa, synthetic construct
  REGION 1..29: note = Description of sequence: Linker 4
GGGGSGGGGSGGGGSGGGGSGGGSGGGGS

SEQ ID NO: 5 - protein, 5 aa, synthetic construct
  REGION 1..5: note = Description of sequence: Linker 5
GGGGS

SEQ ID NO: 6 - protein, 10 aa, synthetic construct
  REGION 1..10: note = Description of sequence: Linker 6
GGGGSGGGGS

SEQ ID NO: 7 - protein, 220 aa, Homo sapiens
  REGION 1..220: note = Description of sequence: Human CD28
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 8 - protein, 164 aa, Homo sapiens
  REGION 1..164: note = Description of sequence: Human CD3zeta
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 22 aa, Homo sapiens
  REGION 1..22: note = Description of sequence: Native ITAM1
QNQLYNELNLGRREEYDVLDKR

SEQ ID NO: 10 - DNA, 66 bp, Homo sapiens
  misc_feature 1..66: note = Description of sequence: Nucleotide sequence encoding Native ITAM1
  source 1..66: mol_type = genomic DNA
cagaaccagctctataacgagctcaatctaggacgaagagaggagtacgatgttttggacaagaga

SEQ ID NO: 11 - protein, 22 aa, synthetic construct
  REGION 1..22: note = Description of sequence: Mutant ITAM1
QNQLFNELNLGRREEFDVLDKR

SEQ ID NO: 12 - DNA, 66 bp, synthetic construct
  misc_feature 1..66: note = Description of sequence: Nucleotide sequence encoding Mutant ITAM1
  source 1..66: mol_type = other DNA
cagaaccagctctttaacgagctcaatctaggacgaagagaggagttcgatgttttggacaagaga

SEQ ID NO: 13 - protein, 23 aa, Homo sapiens
  REGION 1..23: note = Description of sequence: Native ITAM2
QEGLYNELQKDKMAEAYSEIGMK

SEQ ID NO: 14 - DNA, 69 bp, Homo sapiens
  misc_feature 1..69: note = Description of sequence: Nucleotide sequence encoding Native ITAM2
  source 1..69: mol_type = other DNA
caggaaggcctgtacaatgaactgcagaaagataagatggcggaggcctacagtgagattgggatgaaa

SEQ ID NO: 15 - protein, 23 aa, synthetic construct
  REGION 1..23: note = Description of sequence: Mutant ITAM2
QEGLFNELQKDKMAEAFSEIGMK

SEQ ID NO: 16 - DNA, 69 bp, synthetic construct
  misc_feature 1..69: note = Description of sequence: Nucleotide sequence encoding Mutant ITAM2
  source 1..69: mol_type = other DNA
caggaaggcctgttcaatgaactgcagaaagataagatggcggaggccttcagtgagattgggatgaaa

SEQ ID NO: 17 - protein, 22 aa, Homo sapiens
  REGION 1..22: note = Description of sequence: Native ITAM3
HDGLYQGLSTATKDTYDALHMQ

SEQ ID NO: 18 - DNA, 66 bp, Homo sapiens
  misc_feature 1..66: note = Description of sequence: Nucleotide sequence encoding Native ITAM3
  source 1..66: mol_type = other DNA
cacgatggcctttaccagggtctcagtacagccaccaaggacacctacgacgcccttcacatgcag

SEQ ID NO: 19 - protein, 22 aa, synthetic construct
  REGION 1..22: note = Description of sequence: Mutant ITAM3
HDGLFQGLSTATKDTFDALHMQ

SEQ ID NO: 20 - DNA, 66 bp, synthetic construct
  misc_feature 1..66: note = Description of sequence: Nucleotide sequence encoding Mutant ITAM3
  source 1..66: mol_type = other DNA
cacgatggccttttccaggggctcagtacagccaccaaggacaccttcgacgcccttcacatgcag

SEQ ID NO: 21 - protein, 112 aa, synthetic construct
  REGION 1..112: note = Description of sequence: 1XX
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 22 - DNA, 336 bp, synthetic construct
  misc_feature 1..336: note = Description of sequence: Nucleotide sequence encoding 1XX
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 23 - DNA, 123 bp, synthetic construct
  misc_feature 1..123: note = Description of sequence: Nucleotide of 180-220 of CD28
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 24 - protein, 218 aa, Mus musculus
  REGION 1..218: note = Description of sequence: Mouse CD28
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 25 - protein, 255 aa, Homo sapiens
  REGION 1..255: note = Description of sequence: Human 4-1BB
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 26 - protein, 20 aa, Homo sapiens
  REGION 1..20: note = Description of sequence: Human IL2 signal peptide
MYRMQLLSCIALSLALVTNS

SEQ ID NO: 27 - protein, 20 aa, Mus musculus
  REGION 1..20: note = Description of sequence: Mouse IL2 signal peptide
MYSMQLASCVTLTLVLLVNS

SEQ ID NO: 28 - protein, 20 aa, Homo sapiens
  REGION 1..20: note = Description of sequence: Human kappa signal peptide
METPAQLLFLLLLWLPDTTG

SEQ ID NO: 29 - protein, 20 aa, Mus musculus
  REGION 1..20: note = Description of sequence: Mouse kappa signal peptide
METDTLLLWVLLLWVPGSTG

SEQ ID NO: 30 - protein, 21 aa, Homo sapiens
  REGION 1..21: note = Description of sequence: Human CD8 signal peptide
MALPVTALLLPLALLLHAARP

SEQ ID NO: 31 - protein, 18 aa, Mus musculus
  REGION 1..18: note = Description of sequence: truncated human CD8 signal peptide
MALPVTALLLPLALLLHA

SEQ ID NO: 32 - protein, 16 aa, Homo sapiens
  REGION 1..16: note = Description of sequence: Human albumin signal peptide
MKWVTFISLLFSSAYS

SEQ ID NO: 33 - protein, 30 aa, Homo sapiens
  REGION 1..30: note = Description of sequence: Human prolactin signal peptide
MDSKGSSQKGSRLLLLLVVSNLLLCQGVVS

SEQ ID NO: 34 - protein, 4627 aa, synthetic construct
  REGION 1..4627: note = Description of sequence: TRAC genome
ATATCCAGAACCCTGACCCTGCCGTGTACCAGCTGAGAGACTCTAAATCCAGTGACAAGTCTGTCTGCCTATTCACCGATTTTGATTCTCAAACAAATGTGTCACAAAGTAAGGATTCTGATGTGTATATCACAGACAAAACTGTGCTAGACATGAGGTCTATGGACTTCAAGAGCAACAGTGCTGTGGCCTGGAGCAACAAATCTGACTTTGCATGTGCAAACGCCTTCAACAACAGCATTATTCCAGAAGACACCTTCTTCCCCAGCCCAGGTAAGGGCAGCTTTGGTGCCTTCGCAGGCTGTTTCCTTGCTTCAGGAATGGCCAGGTTCTGCCCAGAGCTCTGGTCAATGATGTCTAAAACTCCTCTGATTGGTGGTCTCGGCCTTATCCATTGCCACCAAAACCCTCTTTTTACTAAGAAACAGTGAGCCTTGTTCTGGCAGTCCAGAGAATGACACGGGAAAAAAGCAGATGAAGAGAAGGTGGCAGGAGAGGGCACGTGGCCCAGCCTCAGTCTCTCCAACTGAGTTCCTGCCTGCCTGCCTTTGCTCAGACTGTTTGCCCCTTACTGCTCTTCTAGGCCTCATTCTAAGCCCCTTCTCCAAGTTGCCTCTCCTTATTTCTCCCTGTCTGCCAAAAAATCTTTCCCAGCTCACTAAGTCAGTCTCACGCAGTCACTCATTAACCCACCAATCACTGATTGTGCCGGCACATGAATGCACCAGGTGTTGAAGTGGAGGAATTAAAAAGTCAGATGAGGGGTGTGCCCAGAGGAAGCACCATTCTAGTTGGGGGAGCCCATCTGTCAGCTGGGAAAAGTCCAAATAACTTCAGATTGGAATGTGTTTTAACTCAGGGTTGAGAAAACAGCTACCTTCAGGACAAAAGTCAGGGAAGGGCTCTCTGAAGAAATGCTACTTGAAGATACCAGCCCTACCAAGGGCAGGGAGAGGACCCTATAGAGGCCTGGGACAGGAGCTCAATGAGAAAGGAGAAGAGCAGCAGGCATGAGTTGAATGAAGGAGGCAGGGCCGGGTCACAGGGCCTTCTAGGCCATGAGAGGGTAGACAGTATTCTAAGGACGCCAGAAAGCTGTTGATCGGCTTCAAGCAGGGGAGGGACACCTAATTTGCTTTTCTTTTTTTTTTTTTTTTTTTTTTTTTTTTTTGAGATGGAGTTTTGCTCTTGTTGCCCAGGCTGGAGTGCAATGGTGCATCTTGGCTCACTGCAACCTCCGCCTCCCAGGTTCAAGTGATTCTCCTGCCTCAGCCTCCCGAGTAGCTGAGATTACAGGCACCCGCCACCATGCCTGGCTAATTTTTTGTATTTTTAGTAGAGACAGGGTTTCACTATGTTGGCCAGGCTGGTCTCGAACTCCTGACCTCAGGTGATCCACCCGCTTCAGCCTCCCAAAGTGCTGGGATTACAGGCGTGAGCCACCACACCCGGCCTGCTTTTCTTAAAGATCAATCTGAGTGCTGTACGGAGAGTGGGTTGTAAGCCAAGAGTAGAAGCAGAAAGGGAGCAGTTGCAGCAGAGAGATGATGGAGGCCTGGGCAGGGTGGTGGCAGGGAGGTAACCAACACCATTCAGGTTTCAAAGGTAGAACCATGCAGGGATGAGAAAGCAAAGAGGGGATCAAGGAAGGCAGCTGGATTTTGGCCTGAGCAGCTGAGTCAATGATAGTGCCGTTTACTAAGAAGAAACCAAGGAAAAAATTTGGGGTGCAGGGATCAAAACTTTTTGGAACATATGAAAGTACGTGTTTATACTCTTTATGGCCCTTGTCACTATGTATGCCTCGCTGCCTCCATTGGACTCTAGAATGAAGCCAGGCAAGAGCAGGGTCTATGTGTGATGGCACATGTGGCCAGGGTCATGCAACATGTACTTTGTACAAACAGTGTATATTGAGTAAATAGAAATGGTGTCCAGGAGCCGAGGTATCGGTCCTGCCAGGGCCAGGGGCTCTCCCTAGCAGGTGCTCATATGCTGTAAGTTCCCTCCAGATCTCTCCACAAGGAGGCATGGAAAGGCTGTAGTTGTTCACCTGCCCAAGAACTAGGAGGTCTGGGGTGGGAGAGTCAGCCTGCTCTGGATGCTGAAAGAATGTCTGTTTTTCCTTTTAGAAAGTTCCTGTGATGTCAAGCTGGTCGAGAAAAGCTTTGAAACAGGTAAGACAGGGGTCTAGCCTGGGTTTGCACAGGATTGCGGAAGTGATGAACCCGCAATAACCCTGCCTGGATGAGGGAGTGGGAAGAAATTAGTAGATGTGGGAATGAATGATGAGGAATGGAAACAGCGGTTCAAGACCTGCCCAGAGCTGGGTGGGGTCTCTCCTGAATCCCTCTCACCATCTCTGACTTTCCATTCTAAGCACTTTGAGGATGAGTTTCTAGCTTCAATAGACCAAGGACTCTCTCCTAGGCCTCTGTATTCCTTTCAACAGCTCCACTGTCAAGAGAGCCAGAGAGAGCTTCTGGGTGGCCCAGCTGTGAAATTTCTGAGTCCCTTAGGGATAGCCCTAAACGAACCAGATCATCCTGAGGACAGCCAAGAGGTTTTGCCTTCTTTCAAGACAAGCAACAGTACTCACATAGGCTGTGGGCAATGGTCCTGTCTCTCAAGAATCCCCTGCCACTCCTCACACCCACCCTGGGCCCATATTCATTTCCATTTGAGTTGTTCTTATTGAGTCATCCTTCCTGTGGTAGCGGAACTCACTAAGGGGCCCATCTGGACCCGAGGTATTGTGATGATAAATTCTGAGCACCTACCCCATCCCCAGAAGGGCTCAGAAATAAAATAAGAGCCAAGTCTAGTCGGTGTTTCCTGTCTTGAAACACAATACTGTTGGCCCTGGAAGAATGCACAGAATCTGTTTGTAAGGGGATATGCACAGAAGCTGCAAGGGACAGGAGGTGCAGGAGCTGCAGGCCTCCCCCACCCAGCCTGCTCTGCCTTGGGGAAAACCGTGGGTGTGTCCTGCAGGCCATGCAGGCCTGGGACATGCAAGCCCATAACCGCTGTGGCCTCTTGGTTTTACAGATACGAACCTAAACTTTCAAAACCTGTCAGTGATTGGGTTCCGAATCCTCCTCCTGAAAGTGGCCGGGTTTAATCTGCTCATGACGCTGCGGCTGTGGTCCAGCTGAGGTGAGGGGCCTTGAAGCTGGGAGTGGGGTTTAGGGACGCGGGTCTCTGGGTGCATCCTAAGCTCTGAGAGCAAACCTCCCTGCAGGGTCTTGCTTTTAAGTCCAAAGCCTGAGCCCACCAAACTCTCCTACTTCTTCCTGTTACAAATTCCTCTTGTGCAATAATAATGGCCTGAAACGCTGTAAAATATCCTCATTTCAGCCGCCTCAGTTGCACTTCTCCCCTATGAGGTAGGAAGAACAGTTGTTTAGAAACGAAGAAACTGAGGCCCCACAGCTAATGAGTGGAGGAAGAGAGACACTTGTGTACACCACATGCCTTGTGTTGTACTTCTCTCACCGTGTAACCTCCTCATGTCCTCTCTCCCCAGTACGGCTCTCTTAGCTCAGTAGAAAGAAGACATTACACTCATATTACACCCCAATCCTGGCTAGAGTCTCCGCACCCTCCTCCCCCAGGGTCCCCAGTCGTCTTGCTGACAACTGCATCCTGTTCCATCACCATCAAAAAAAAACTCCAGGCTGGGTGCGGGGGCTCACACCTGTAATCCCAGCACTTTGGGAGGCAGAGGCAGGAGGAGCACAGGAGCTGGAGACCAGCCTGGGCAACACAGGGAGACCCCGCCTCTACAAAAAGTGAAAAAATTAACCAGGTGTGGTGCTGCACACCTGTAGTCCCAGCTACTTAAGAGGCTGAGATGGGAGGATCGCTTGAGCCCTGGAATGTTGAGGCTACAATGAGCTGTGATTGCGTCACTGCACTCCAGCCTGGAAGACAAAGCAAGATCCTGTCTCAAATAATAAAAAAAATAAGAACTCCAGGGTACATTTGCTCCTAGAACTCTACCACATAGCCCCAAACAGAGCCATCACCATCACATCCCTAACAGTCCTGGGTCTTCCTCAGTGTCCAGCCTGACTTCTGTTCTTCCTCATTCCAGATCTGCAAGATTGTAAGACAGCCTGTGCTCCCTCGCTCCTTCCTCTGCATTGCCCCTCTTCTCCCTCTCCAAACAGAGGGAACTCTCCTACCCCCAAGGAGGTGAAAGCTGCTACCACCTCTGTGCCCCCCCGGCAATGCCACCAACTGGATCCTACCCGAATTTATGATTAAGATTGCTGAAGAGCTGCCAAACACTGCTGCCACCCCCTCTGTTCCCTTATTGCTGCTTGTCACTGCCTGACATTCACGGCAGAGGCAAGGCTGCTGCAGCCTCCCCTGGCTGTGCACATTCCCTCCTGCTCCCCAGAGACTGCCTCCGCCATCCCACAGATGATGGATCTTCAGTGGGTTCTCTTGGGCTCTAGGTCCTGCAGAATGTTGTGAGGGGTTTATTTTTTTTTAATAGTGTTCATAAAGAAATACATAGTATTCTTCTTCTCAAGACGTGGGGGGAAATTATCTCATTATCGAGGCCCTGCTATGCTGTGTATCTGGGCGTGTTGTATGTCCTGCTGCCGATGCCTTC

SEQ ID NO: 35 - protein, 178 aa, synthetic construct
  REGION 1..178: note = Description of sequence: TRBC2 polypeptide
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 36 - DNA, 534 bp, synthetic construct
  misc_feature 1..534: note = Description of sequence: Nucleotide of 35
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 37 - protein, 175 aa, synthetic construct
  REGION 1..175: note = Description of sequence: TRBC1 polypeptide
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 38 - protein, 176 aa, synthetic construct
  REGION 1..176: note = Description of sequence: Variant TRBC1 polypeptide
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 39 - DNA, 528 bp, synthetic construct
  misc_feature 1..528: note = Description of sequence: Nucleotide of 38
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 40 - DNA, 1448 bp, synthetic construct
  misc_feature 1..1448: note = Description of sequence: Genome TRBC1
  source 1..1448: mol_type = other DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 41 - DNA, 1489 bp, synthetic construct
  misc_feature 1..1489: note = Description of sequence: Genome TRBC2
  source 1..1489: mol_type = other DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 42 - protein, 10 aa, synthetic construct
  REGION 1..10: note = Description of sequence: hinge/spacer region
IPNIQNPDPA

SEQ ID NO: 43 - DNA, 30 bp, synthetic construct
  misc_feature 1..30: note = Description of sequence: Nucleotide coding sequence of SEQ ID NO: 42
  source 1..30: mol_type = other DNA
attcccaatatccagaaccctgaccctgcc

SEQ ID NO: 44 - protein, 11 aa, synthetic construct
  REGION 1..11: note = Description of sequence: TCRb joining region
LEDLKNVFPPE

SEQ ID NO: 45 - protein, 33 aa, synthetic construct
  REGION 1..33: note = Description of sequence: Nucleotide sequence encoding TCRb joining region
CTGGAGGATCTGAAAAACGTGTTCCCTCCTGAA

SEQ ID NO: 46 - protein, 182 aa, Homo sapiens
  REGION 1..182: note = Description of sequence: CD3gamma human
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 47 - protein, 171 aa, synthetic construct
  REGION 1..171: note = Description of sequence: CD3delta 1
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 48 - protein, 127 aa, synthetic construct
  REGION 1..127: note = Description of sequence: CD3delta 2
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 49 - protein, 207 aa, synthetic construct
  REGION 1..207: note = Description of sequence: CD3 epsilon
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 50 - protein, 685 aa, Homo sapiens
  REGION 1..685: note = Description of sequence: DLL-4 human
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 51 - protein, 1218 aa, Homo sapiens
  REGION 1..1218: note = Description of sequence: JAG-1 human
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 52 - protein, 254 aa, synthetic construct
  REGION 1..254: note = Description of sequence: 4-1BBL
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 53 - DNA, 762 bp, synthetic construct
  misc_feature 1..762: note = Description of sequence: Nucleotide sequence encoding 4-1BBL
  source 1..762: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 54 - protein, 448 aa, synthetic construct
  REGION 1..448: note = Description of sequence: Urelumab heavy chain
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 216 aa, synthetic construct
  REGION 1..216: note = Description of sequence: Urelumab light chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 288 aa, synthetic construct
  REGION 1..288: note = Description of sequence: CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 57 - DNA, 864 bp, synthetic construct
  misc_feature 1..864: note = Description of sequence: Nucleotide sequence encoding CD80
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 58 - protein, 309 aa, synthetic construct
  REGION 1..309: note = Description of sequence: CD80/4-1BB sequence
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 59 - protein, 349 aa, synthetic construct
  REGION 1..349: note = Description of sequence: Fusion protein CD40/4-1BB/CD28
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

SEQ ID NO: 60 - protein, 8 aa, synthetic construct
  REGION 1..8: note = Description of sequence: 19E3 VH CDR1
GFTFSDFD

SEQ ID NO: 61 - protein, 8 aa, synthetic construct
  REGION 1..8: note = Description of sequence: 19E3 VH CDR2
INGGSGVI

SEQ ID NO: 62 - protein, 13 aa, synthetic construct
  REGION 1..13: note = Description of sequence: 19E3 VH CDR3
AREELGRRYYFDY

SEQ ID NO: 63 - protein, 7 aa, synthetic construct
  REGION 1..7: note = Description of sequence: 19E3 VL CDR1
SEHSSYN

SEQ ID NO: 64 - protein, 7 aa, synthetic construct
  REGION 1..7: note = Description of sequence: 19E3 VL CDR2
LKSDGSH

SEQ ID NO: 65 - protein, 13 aa, synthetic construct
  REGION 1..13: note = Description of sequence: 19E3 VL CDR3
GAGYTISGQYGYV

SEQ ID NO: 66 - protein, 216 aa, synthetic construct
  REGION 1..216: note = Description of sequence: 19E3 Full VH
MNSGLKLVFFVLILKGVQCEVQLVESGGGLVQTGKSLKLSCEASGFTFSDFDMNWVRQAPGKGLEWVAYINGGSGVIFYADAVKGRFTISRDNAKNLLFLQMNNLKSEDSAMYYCAREELGRRYYFDYWGQGTMVTVSSATTTAPSVYPLAPACDSTTSTTNTVTLGCLVKGYFPEPVTVSWNSGALTSGVHTFPSVLHSGLYSLSSSVTVPSSTW

SEQ ID NO: 67 - protein, 214 aa, synthetic construct
  REGION 1..214: note = Description of sequence: 19E3 Full VL
MAWIPLLFFLLHCTGSFSQPVLTQSPSASASLSGSVKLTCTLSSEHSSYNIAWYQQHPDKAPKYVMYLKSDGSHFKGDGIPDRFSGSSSGAHRYLSISNVQSEDDATYFCGAGYTISGQYGYVFDSGTQLTVLGGPKSSPKVTVFPPSPEELRTNKATLVCLVNDFYPGSATVTWKANGATINDGVKTTKPSKQGQNYMTSSYLSLTADQWRSH

SEQ ID NO: 68 - DNA, 648 bp, synthetic construct
  misc_feature 1..648: note = Description of sequence: 19E3 DNA Full VH
  source 1..648: mol_type = other DNA
atgaactcaggactcaaattggttttctttgtccttattctgaaaggtgtccagtgtgaggtgcagttggtggagtctgggggaggcttagtacagactggaaagtccctgaaactctcatgtgaggcctctggattcaccttcagtgactttgacatgaactgggtccgccaggctccagggaaggggctggagtgggtcgcatacattaatggtggtagtggtgttatcttttatgctgacgctgtgaagggccgattcaccatctccagagacaacgccaagaacttactgttcctacagatgaacaatctcaagtctgaggactcagccatgtattactgtgcaagagaggaactgggacggaggtactactttgattactggggccaaggaaccatggtcaccgtctcctcagccacaacaacagccccatctgtctatcccttggcccctgcctgtgacagcacaaccagcaccacgaacacggtgaccctgggatgcctggtcaagggctatttccctgagccggtgaccgtaagctggaactctggagccctgaccagcggcgtgcacaccttcccatctgtcctgcattctgggctctactccctcagcagctcagtgactgtaccttccagcacctgg

SEQ ID NO: 69 - DNA, 642 bp, synthetic construct
  misc_feature 1..642: note = Description of sequence: 19E3 DNA Full VL
  source 1..642: mol_type = other DNA
atggcctggattcctctcctcttcttcctccttcattgcacagggtctttctctcaacctgtgttgactcagtcaccctctgcctctgcctccctgagtggctcagtcaaactcacctgcaccctgagtagtgagcacagctcctacaacatagcatggtaccagcaacatccagacaaggctcccaagtatgtgatgtaccttaagagtgatggaagccacttcaagggagatgggatccctgatcgcttctctggctccagctctggggctcatcgctacttaagcatctccaatgtccagtctgaagatgatgctacctatttctgtggtgcaggttataccatttctggacaatatgggtatgtttttgacagcggaacccagctcaccgtcctaggtggacccaagtcttctcccaaagtcacagtgtttccaccttcacctgaggagctccggacaaacaaagccacactggtgtgtctggttaatgacttctacccgggttctgcaacagtgacctggaaggcaaatggagcaactatcaatgatggggtgaagactacaaagccttccaaacagggccaaaactacatgaccagcagctacctaagtttgacagcagaccagtggagatcccac

SEQ ID NO: 70 - protein, 8 aa, synthetic construct
  REGION 1..8: note = Description of sequence: 12D11 VH CDR1
GFDLSDYF

SEQ ID NO: 71 - protein, 10 aa, synthetic construct
  REGION 1..10: note = Description of sequence: 12D11 VH CDR2
IYPQSYDYAT

SEQ ID NO: 72 - protein, 8 aa, synthetic construct
  REGION 1..8: note = Description of sequence: 12D11 VH CDR3
TKDGDVGF

SEQ ID NO: 73 - protein, 6 aa, synthetic construct
  REGION 1..6: note = Description of sequence: 12D11 VL CDR1
QDISNE

SEQ ID NO: 74 - skipped

SEQ ID NO: 75 - protein, 9 aa, synthetic construct
  REGION 1..9: note = Description of sequence: 12D11 VL CDR3
LQHYSFPLT

SEQ ID NO: 76 - protein, 213 aa, synthetic construct
  REGION 1..213: note = Description of sequence: 12D11 Full VH
MGLGLQWVFFVALLKGVHCAVRLLESGGGLVKPEGALKLSCVASGFDLSDYFMGWVRQAPGKGLEWVAHIYPQSYDYATYYSGSVQGRFTISRDDSRSMVYLQMNNLRTEDTATYYCTKDGDVGFWGQGTMVTVSSATTTAPSVYPLAPACDSTTSTTNTVTLGCLVKGYFPEPVTVSWNSGALTSGVHTFPSVLHSGLYSLSSSVTVPSSTW

SEQ ID NO: 77 - protein, 196 aa, synthetic construct
  REGION 1..196: note = Description of sequence: 12D11 Full VL
MANKSPAQALAILLLWLSGVRCDIQVTQSPTLLSASLGDKVTINCLASQDISNELNWYQQKSGQSPTLLIYNGKNLQSGVPSRFSGQYSGRSFTISINNVEPEDVATYFCLQHYSFPLTFGDGSKLEMKRADAKPTVSIFPPSSEQLGTGSATLVCFVNNFYPKDINVKWKVDGSEKRDGVLQSVTDQDSKDSTYS

SEQ ID NO: 78 - DNA, 639 bp, synthetic construct
  misc_feature 1..639: note = Description of sequence: 12D11 DNA Full VH
  source 1..639: mol_type = other DNA
atgggattgggactgcagtgggttttctttgttgctcttttaaaaggtgtccactgtgcggtgcggcttctggagtcgggtggaggattagtgaagcctgagggggcactgaaactctcctgtgtggcctctggattcgacttaagtgactatttcatgggctgggtccgccaggctccagggaaggggctggagtgggttgctcacatataccctcaaagttatgattatgcaacctattactcgggttcggtgcaaggcagattcaccatctccagagatgattcccgaagcatggtctacctgcaaatgaacaacctgagaactgaggacacggccacttattactgtacaaaagacggggacgtgggtttctggggccaaggaaccatggtcaccgtctcctcagccacaacaacagccccatctgtctatcccttggcccctgcctgtgacagcacaaccagcaccacgaacacggtgaccctgggatgcctggtcaagggctatttccctgagccggtgaccgtaagctggaactctggagccctgaccagcggcgtgcacaccttcccatctgtcctgcattctgggctctactccctcagcagctcagtgactgtaccttccagcacctgg

SEQ ID NO: 79 - DNA, 588 bp, synthetic construct
  misc_feature 1..588: note = Description of sequence: 12D11 DNA Full VL
  source 1..588: mol_type = other DNA
atggccaacaagtctcctgctcaggcactggcaattttgttactgtggctgtcaggtgtcagatgtgacattcaagtgacacaatctcccaccctcctgtcagcatctctaggagacaaagtgaccatcaattgcctggcaagtcaggacattagcaatgagttaaactggtaccagcagaagtcaggacaatctcctacactgttgatttataatggaaaaaatttgcagtctggtgtcccgtcaaggttcagtggccagtattcagggagaagtttcactatcagcatcaacaatgtggaacctgaagatgttgcaacttatttttgtcttcagcattacagttttccgctcacgttcggtgatggctccaagctggagatgaaacgggctgatgctaagccaaccgtctccatcttcccaccatccagtgagcagttgggcactggaagcgccacacttgtgtgcttcgtgaacaacttctaccccaaagacatcaatgtcaagtggaaagtagatggcagtgaaaaacgagatggcgtcctgcagagtgtcactgatcaggacagcaaagacagcacctacagc

SEQ ID NO: 80 - protein, 140 aa, synthetic construct
  REGION 1..140: note = Description of sequence: TRAC polypeptide
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 81 - DNA, 420 bp, synthetic construct
  misc_feature 1..420: note = Description of sequence: Nucleotide sequence encoding TRAC polypeptide
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 82 - protein, 143 aa, synthetic construct
  REGION 1..143: note = Description of sequence: TRAC polypeptide v2
IPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 83 - DNA, 429 bp, synthetic construct
  misc_feature 1..429: note = Description of sequence: Nucleotide sequence encoding TRAC polypeptide v2
  source 1..429: mol_type = other DNA
attcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

(19) United States
(12) Patent Application Publication
(10) Pub. No.: US 2025/0207095 A1
(43) Pub. Date: Jun. 26, 2025

(54) MANUFACTURING PROCESSES FOR ADOPTIVE CELL THERAPIES

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Sjoukje VAN DER STEGEN, New York, NY (US); Isabelle RIVIERE, New York, NY (US); Michel SADELAIN, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/030,414

(22) Filed: Jan. 17, 2025

Related U.S. Application Data
(63) Continuation of application No. PCT/US2023/028571, filed on Jul. 25, 2023.
(60) Provisional application No. 63/510,269, filed on Jun. 26, 2023.
(60) Provisional application No. 63/395,544, filed on Aug. 5, 2022.
(60) Provisional application No. 63/392,099, filed on Jul. 25, 2022.

(51) Int. Cl. C12N 5/0783; A61K 40/11; A61K 40/31; A61K 40/42; A61P 35/00; C07K 16/28; C07K 16/30

(52) U.S. Cl. CPC C12N 5/0636; A61K 40/11; A61K 40/31; A61K 40/4211; A61K 40/4276; A61P 35/00; C07K 16/2803; C07K 16/2878; C07K 16/3069; C07K 2317/622; C07K 2317/75; C12N 2501/115; C12N 2501/125; C12N 2501/155; C12N 2501/165; C12N 2501/2303; C12N 2501/2306; C12N 2501/2307; C12N 2501/2311; C12N 2501/26; C12N 2506/45; C12N 2510/00

(57) ABSTRACT

The presently disclosed subject matter provides methods for improving the production of cells comprising an antigen-recognizing receptor (e.g., a chimeric antigen receptor (CAR) or a TCR like fusion molecule). The methods disclosed herein can improve the activity and/or efficiency of the cells.

### CROSS-REFERENCE TO RELATED APPLICATIONS

This application is a continuation application of International Patent Application No. PCT/US23/28571, filed Jul. 25, 2023, which claims priority to U.S. Provisional Application No. 63/392,099, filed Jul. 25, 2022, to U.S. Provisional Application No. 63/395,544, filed Aug. 5, 2022, and to U.S. Provisional Application No. 63/510,269, filed Jun. 26, 2023, the contents of each of which are incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter encoded as XML in UTF-8 text. The electronic document, created on Jan. 17, 2025, is entitled “0727341717.xml”, and is 127,190 bytes in size.

### 1. INTRODUCTION

The presently disclosed subject matter provides methods and compositions for the production of cells comprising an antigen-recognizing receptor (e.g., a chimeric antigen receptor (CAR) or a TCR-like fusion molecule).

### 2. BACKGROUND OF THE INVENTION

Autologous chimeric antigen receptor (CAR) T cell therapy can provide substantial clinical benefit to patients with refractory hematological malignancies. This approach is however challenged by costly and sometimes delayed or unsuccessful cell manufacturing. Readily available CAR T cells that can be produced on a large scale are direly needed. Genetically engineered, T cell-derived induced pluripotent stem cells (TiPS) are a promising source for “off-the-shelf” immunotherapeutic CAR T cells. However, in vitro TiPS differentiation often yields apTCR-T cells with innate features.

Therefore, there remains a need in the art for additional manufacturing processes.

### 3. SUMMARY OF THE INVENTION

In certain non-limiting embodiments, the presently disclosed subject matter provides a method of expanding a population of induced T cells. In certain embodiments, the method comprises: (a) contacting an induced T cell comprising an antigen-recognizing receptor with a polypeptide that engages the antigen-recognizing receptor and an agonist of 4-1BB, and (b) culturing the induced T cell to thereby produce an expanded population of induced T cells; wherein the antigen-recognizing receptor is a chimeric antigen receptor (CAR) or a TCR like fusion protein (HIT).

In certain embodiments, the polypeptide that engages the antigen-recognizing receptor is an antibody or antigen-binding fragment thereof. In certain embodiments, the antibody or antigen-binding fragment thereof binds to a scFv of the CAR. In certain embodiments, the antibody or antigen-binding fragment thereof binds to an idiotypic variable domain of a scFv of the CAR. In certain embodiments, wherein the antibody or antigen-binding fragment thereof binds to an antigen-binding chain of the HIT. In certain embodiments, the antibody or antigen-binding fragment thereof binds to an idiotypic variable domain of an antigen-binding chain of the HIT.

In certain embodiments, the antibody or antigen-binding fragment thereof comprises:
(a) a heavy variable region comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62; and a light variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; or
(b) a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

In certain embodiments, the antigen-recognizing receptor binds to CD19 and the antibody or antigen-binding fragment thereof comprises a heavy variable region comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62; and a light variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65.

In certain embodiments, the antigen-recognizing receptor binds to CD19 and the antibody or antigen-binding fragment thereof comprises a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

In certain embodiments, the antigen-recognizing receptor binds to PSMA and the antibody or antigen-binding fragment thereof comprises a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

In certain embodiments, the polypeptide that engages the antigen-recognizing receptor is an antigen-containing polypeptide. In certain embodiments, the antigen-containing polypeptide is an antigen or a fragment thereof. In certain embodiments, the antigen-containing polypeptide is an Fc-fusion protein.

In certain embodiments, the agonist of 4-1BB is an antibody or antigen-binding fragment thereof that binds 4-1BB. In certain embodiments, the antibody or antigen-binding fragment thereof that binds 4-1BB is urelumab. In certain embodiments, the antibody or antigen-binding fragment thereof that binds 4-1BB comprises a heavy chain comprising an amino acid sequence that at least about 80%, at least about 85%, at least about 90%, at least about 95%, at least about 96%, at least about 97%, at least about 98%, or at least about 99%, at least about 100% identical to the amino sequence set forth in SEQ ID NO: 54, and a light chain comprising an amino acid sequence that at least about 80%, at least about 85%, at least about 90%, at least about 95%, at least about 96%, at least about 97%, at least about 98%, or at least about 99%, at least about 100% identical to the amino sequence set forth in SEQ ID NO: 55. In certain embodiments, the antibody or antigen-binding fragment thereof binds 4-1BB comprises a heavy chain comprising the amino sequence set forth in SEQ ID NO: 54, and a light chain comprising the amino sequence set forth in SEQ ID NO: 55.

In certain embodiments, the antigen-recognizing receptor binds to a first antigen that is a tumor antigen or a pathogen antigen. In certain embodiments, the first antigen is a tumor antigen or a pathogen antigen. In certain embodiments, the first antigen is selected from the group consisting of CD19, CD70, IL1RAP, ABCG2, AChR, ACKR6, ADAMTS13, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the HIT comprises an extracellular antigen-binding domain that binds to the first antigen and is capable of delivering an activation signal to the cell. In certain embodiments, the CAR comprises an extracellular antigen-binding domain that binds to the first antigen and an intracellular signaling domain that is capable of delivering an activation signal to the cell.

In certain embodiments, the intracellular signaling domain comprises a native CD3ζ polypeptide or a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the modified CD3ζ polypeptide comprises or consists of the amino acid sequence set forth in SEQ ID NO: 21.

In certain embodiments, the chimeric receptor is encoded by a polynucleotide integrated at a locus within the genome of the induced T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus.

In certain embodiments, culturing comprises contacting the induced T cell comprising a chimeric receptor with IL-7, IL-21, or a combination thereof.

In certain embodiments, the induced T cell is a cytotoxic T lymphocyte (CTL), a regulatory T cell, or a Natural Killer T (NKT) cell. In certain embodiments, the induced T cell is (a) CD3⁺, TCR⁻; (b) CD4⁺, CD3⁺, and TCR⁻; or (c) CD8⁺, CD3⁺, and TCR⁻. In certain embodiments, the induced T cell is: (a) CD3⁺, TCR⁻, CD25⁺, CD28⁺, CD69⁺, CD56⁺, CD45RA⁺; (b) CD3⁺, TCR⁻, CD4⁻, CD8αα⁻; (c) CD3⁺, TCR⁻, CD4⁻, CD8αβ⁻; (d) CD3⁺, TCR⁻, CD4⁻, CD8αα⁺; (e) CD3⁺, TCR⁻, CD4⁻, CD8αβ⁺; (f) CD3⁺, TCR⁻, CD4⁺, CD8αα⁻; (h) CD3⁺, TCR⁻, CD4⁺, CD8αβ⁻; (i) CD3⁺, TCR⁻, CD4⁺, CD8αα⁺; or (j) CD3⁺, TCR⁻, CD4⁺, CD8αβ⁺.

In certain embodiments, the induced T cell further comprises a gene disruption at a second locus selected from the group consisting of a CD52 locus, a CD70 locus, a PD1 locus, a CD38 locus, a PLZF locus, a SOX13 locus, and a combination thereof.

In certain embodiments, the induced T cell further comprises a second antigen-recognizing receptor that targets a second antigen. In certain embodiments, the second antigen-recognizing receptor is a chimeric antigen receptor (CAR), a chimeric costimulatory receptor (CCR), a T cell receptor (TCR), or a TCR like fusion molecule.

In certain embodiments, the second antigen is a tumor antigen or a pathogen antigen. In certain embodiments, the second antigen is independently selected from the group consisting of CD19, CD70, IL1RAP, ABCG2, AChR, ACKR6, ADAMTS13, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the intracellular signaling domain of the CAR further comprises at least one costimulatory signaling region. In certain embodiments, the at least one costimulatory signaling region comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain non-limiting embodiments, the presently disclosed subject matter provides methods of obtaining and expanding a population of induced T cells. In certain embodiments, the methods comprise:
(a) introducing into a pluripotent stem cell a polynucleotide encoding an antigen-recognizing receptor, wherein the antigen-recognizing receptor is a chimeric antigen receptor (CAR) or a TCR like fusion protein (HIT);
(b) contacting the pluripotent stem cell with a first cell culture medium comprising an activator of the bone morphogenic protein pathway to differentiate the pluripotent stem cell into a hematopoietic precursor;
(c) contacting the pluripotent stem cell with a second cell culture medium comprising a Notch ligand to differentiate the hematopoietic precursor into induced T cell; and
(d) expanding the induced T cell with the methods disclosed herein.

In certain embodiments, the pluripotent stem cell is an induced pluripotent stem cell. In certain embodiments, the pluripotent stem cell is a T cell-derived induced pluripotent stem cell. In certain embodiments, the activator of the bone morphogenic protein pathway is a BMP-4 polypeptide (BMP-4). In certain embodiments, the first cell culture medium further comprises a fibroblast growth factor.

In certain embodiments, the fibroblast growth factor is a basic fibroblast growth factor (bFGF). In certain embodiments, the pluripotent stem cell is in contact with the first cell culture medium for up to about 4 days. In certain embodiments, the first cell culture medium further comprises VEGF, SCF, FLT3L, IL3, IL-6, IL-11, TPO, IGF-1, EPO, SHH, angiotensin II, AGTR1, or a combination thereof.

In certain embodiments, the pluripotent stem cell is in contact with the first cell culture medium for up to about 10 days.

In certain embodiments, the Notch ligand is a DLL-1 polypeptide, a DLL-4 polypeptide, a JAG-1 polypeptide, a JAG-2 polypeptide, or a combination thereof. In certain embodiments, the Notch ligand is expressed by a feeder cell. In certain embodiments, the second cell culture medium further comprises SCF, FLT3L, IL-3, IL-7, IL-6, IL-11, TPO, IGF-1, EPO, SHH, angiotensin II, AGTR1, or a combination thereof. In certain embodiments, the hematopoietic precursor is in contact with the second cell culture medium for up to about 25 days.

In certain non-limiting embodiments, the presently disclosed subject matter provides induced T cells obtained by the method disclosed herein. In certain non-limiting embodiments, the presently disclosed subject matter provides compositions comprising the induced T cell obtained by the methods disclosed herein. In certain embodiments, the composition is a pharmaceutical composition further comprising a pharmaceutically acceptable excipient.

In certain non-limiting embodiments, the presently disclosed subject matter provides methods of reducing tumor burden in a subject. In certain embodiments, the methods comprise administering to the subject an effective amount of the induced T cell produced by the methods disclosed herein or the compositions disclosed herein. In certain embodiments, the methods reduce the number of tumor cells, reduces tumor size, and/or eradicates the tumor in the subject. In certain non-limiting embodiments, the presently disclosed subject matter provide methods of preventing and/or treating a neoplasm or a tumor in the subject. In certain embodiments, the methods comprise administering to the subject an effective amount of the induced T cell produced by the methods disclosed herein or the compositions disclosed herein.

In certain embodiments, the neoplasm or tumor is cancer. In certain embodiments, the neoplasm or tumor is a solid tumor. In certain embodiments, the neoplasm or tumor is a blood cancer.

In certain embodiments, the blood cancer is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain non-limiting embodiments, the presently disclosed subject matter provide methods of preventing and/or treating a pathogen infection in a subject. In certain embodiments, the methods comprise administering to the subject an effective amount of the induced T cell produced by the methods disclosed herein or the compositions disclosed herein.

In certain non-limiting embodiments, the presently disclosed subject matter provide methods of preventing and/or treating an autoimmune disease in a subject. In certain embodiments, the methods comprise administering to the subject an effective amount of the induced T cell produced by the methods disclosed herein or the compositions disclosed herein.

In certain non-limiting embodiments, the presently disclosed subject matter provide methods of preventing and/or treating an infectious disease in a subject. In certain embodiments, the methods comprise administering to the subject an effective amount of the induced T cell produced by the methods disclosed herein or the compositions disclosed herein.

In certain non-limiting embodiments, the presently disclosed subject matter provide kits comprising the induced T cell produced by the methods disclosed herein or the compositions disclosed herein. In certain embodiments, the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

In certain non-limiting embodiments, the presently disclosed subject matter provide compositions or kits disclosed herein for use in reducing tumor burden, treating and/or preventing a neoplasm or a tumor, preventing and/or treating a pathogen infection, preventing and/or treating an autoimmune disease, and/or preventing and/or treating an infectious disease, in a subject.

### 4. BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1G illustrate that DLL4 supports in vitro apTCR-T cell development of WT-TiPS but not CAR-TiPS. FIG. 1A shows a schematic representation of in vitro T cell differentiation protocol. Microscope images are at 4× magnification. FIG. 1B shows flow cytometric analysis of T lineage commitment of H1, FiPS, and WT-TiPS on OP9-mDLL1, gated on live CD45⁺CD7⁺ cells at day 40 (D40) in the differentiation. FIG. 1C shows flow cytometric analysis of T lineage commitment of WT-TiPS and TRAC^{-/-}-TiPS on OP9-mDLL1, gated on live CD45⁺ cells at D40 in the differentiation. FIGS. 1D and 1F show representative flow cytometric analysis of T lineage commitment of WT-TiPS and CAR-TiPS on D35 in differentiation on OP9 expressing the indicated human Notch ligand, gated on live CD45⁺CD7⁺ cells. FIGS. 1E and 1G show phenotype distribution of WT-TiPS (e, n=6 biological replicates) or CAR-TiPS (g, n=6 biological replicates) on D35 of differentiation on OP9-DLL4, gated on live CD45+CD7+ cells. All data are means±s.d.

FIGS. 2A-2E illustrate that TRAC-controlled 1928z-1XX CAR expression facilitates DP T cell development. FIG. 2A shows induction of αβTCR (upper panel) and CAR (lower panel) expression in WT-TiPS, CAR-TiPS, and TRAC-CAR-TiPS throughout T lymphoid development on OP9-DLL4 at the indicated timepoints. Gated on live CD45+CD7+ cells. FIGS. 2B and 2D show representative flow cytometric analysis of T lineage commitment markers of TRAC-1928z-TiPS (FIG. 2B) and TRAC-1XX-TiPS (FIG. 2D) gated on live CD45+ cells at D35 in differentiation on OP9-DLL4. FIGS. 2C and 2E show D35 phenotype distribution of TRAC-1928z-TiPS (Fig, n=3 biological replicates) and TRAC-1XX-TiPS (FIG. 2E, n=11 biological replicates) at D35 gated on live CD45+CD7+ cells. All data are means±s.d.

FIGS. 3A-3D illustrate that CAR regulation influences Notch and TCR target gene induction. FIG. 3A shows a schematic representation of Notch and (pre)TCR signaling interactions as reported in the literature. FIG. 3B shows ddPCR analysis of Notch and (pre)TCR target genes at D24, 27, 31, and 35 of T cell differentiation (normalized to RPL13A) (n=3 technical replicates).

FIG. 3C shows tSNE analysis of cell surface expression of CD4, CD8a, CD8b, and pTa on D35 TRAC-1XX-TiPS iT cells. Color scale represent level of marker expression. FIG. 3D shows distribution of pTa expression on D35 TRAC-1XX-TiPS (n=3 biological replicates). All data are means±s.d.

FIGS. 4A-4P illustrate that 4-1BBL costimulation enhances CD8αp TRAC-1XX-iT proliferation and function. FIG. 4A shows representative phenotype of TRAC-1XX-iT cells matured on 3T3-CD19 for 7 days (D35-D42), gated on live CD45⁺CD7+(left and right) and CD45⁺CD7+CD8α⁺ (middle). FIG. 4B shows distribution of CD8αα and CD8αβ phenotype in the CD8α⁺ compartment (n=3 biological replicates). FIG. 4C shows expansion of TRAC-1XX-iT cells from D35-D42 after maturation on 3T3-CD19 (n=6 biological replicates). FIG. 4D shows 4-1BB cell-surface expression on TRAC-1XX-iT cells on D35 8 h after exposure to parental 3T3 (black), 3T3-CD19 (red) or left unstimulated (grey). FIG. 4E shows representative phenotype of TRAC-1XX-iT cells matured on 3T3-CD19-41BBL, gated on live CD45⁺CD7+(left and right) and CD45⁺CD7+CD8α⁺ (middle). FIG. 4F shows distribution of CD8αα and CD8αβ phenotype in the CD8α⁺ compartment (n=6 biological replicates). FIG. 4G shows expansion of TRAC-1XX-iT cells from D35-D42 after maturation on 3T3-CD19-41BBL (n=6 biological replicates). FIG. 4H shows total cell expansion from DO-D42 in iT differentiation with maturation on 3T3-CD19±41BBL (n=6 biological replicates for each group). FIG. 4I shows cytotoxic activity measured in an 18 h bioluminescence assay, using firefly luciferase (FFLuc)-expressing NALM6 at the indicated effector-to-target (E:T) ratios (n=3; technical replicates). FIG. 4J shows 4 h intracellular cytokine detection of 3T3-CD19±41BBL matured cells in response to NALM6 (n=3 technical replicates). FIG. 4K shows representative expansion of D42 cells matured on 3T3-CD19±41BBL upon repeated weekly antigen exposure on 3T3-CD19. FIG. 4L shows schematic representation of NALM6 in vivo tumor model. FIG. 4M shows tumor burden (total flux in photons per second) of NALM6-bearing mice treated with 2×10⁶ D42 TRAC-1XX-iT cells (n=5, line=one mouse). FIG. 4N shows Kaplan-Meier analysis of mouse survival. FIG. 4O shows flow cytometric quantification of CAR T cells (left panel) and tumor cells (right panel) in bone marrow 6 days after T cell infusion (n=3). FIG. 4P shows cytotoxic activity measured in a 6 h flow cytometry assay, using primary CD19T CLL cells at the indicated E:Ts with 3T3-CD19-41BBL-matured TRAC-1XX-iT cells (n=3 technical replicates) * P<0.05, ** P<0.001, *** P<0.001, Welch's 2-sample t test (h, o), log-rank Mantel-Cox test (n). All data are means±s.d.

FIGS. 5A-5C illustrate that CD8αp TRAC-1XX-iT cells resemble peripheral-blood derived CD8αp T cells. FIG. 5A shows phenotype analysis of 3T3-CD19-41BBL matured D42 CD8αp TRAC-1XX-iT cells for TCR-T cell markers (left and middle panel) and NK-cell markers (right panel). Data is representative of four independent experiments, gated on live CD45+CD7+CD8ab+ cells. FIG. 5B shows dendrogram of hierarchical clustering analysis based on Euclidian distance matrix comparing the transcriptome of TRAC-1XX CD8αp apTCR-T cells (CD8, blue, n=4 biological replicates), TRAC-1XX CD4 apTCR-T cells (CD4, orange, n=3 biological replicates), γRV-1XX γδTCR-T cells (γδ, green, n=4 biological replicates), γRV-1XX NK cells (NK, purple, n=4 biological replicates) and CD8αβ+ TRAC-1XX-iT cells (iT CD8αβ, red, n=4 biological replicates). FIG. 5C shows correlation matrix using Pearson's statistics comparing same groups as in FIG. 5B.

FIGS. 6A-6I illustrate that TRAC-1XX-iT cells cure systemic NALM6 tumor model. Functional comparison of healthy-donor peripheral blood TRAC-1XX CD8αβ αβTCR-T cells (CD8 TRAC-1XX), CAR-iT, and TRAC-1XX-iT cells (matured on 3T3-CD19-41BBL). CD8 TRAC-1XX doses reflect number of CART T cells utilized in the assay. FIG. 6A shows cytotoxic activity using a 18 h Incucyte assay, using NLR-expressing NALM6 as target cells (n=3 technical replicates). FIG. 6B shows four h intracellular cytokine detection using NALM6 as target cells at a 11 E:T ratio (n=3 technical replicates). FIG. 6C shows NALM6 rechallenge assay. NLRT NALM6 and T cells were co-cultured at a 1:1 E:T. Every 72 h T cells were rechallenged with 1×NLRTNALM6 and cytokines. NALM6 clearance was measured in NLRT surface area reduction compared to the timepoint of rechallenge (n=3 technical replicates). FIG. 6D shows twenty-four h cytokine secretion using NALM6 as target cells at a 1:1 E:T ratio (CD8 TRAC-1XX n=15, TRAC-1XX-iT n=18, CAR-iT n=11 biological replicates). FIG. 6E shows schematic representation of systemic NALM6 tumor model. FIG. 6F shows tumor burden (total flux in photons per second) of NALM6-bearing untreated mice, or mice treated with 4×106 CD8 TRAC-1XX or TRAC-1XX-iT cells (n=7, line=one mouse). FIG. 6G shows Kaplan-Meier analysis of tumor-free survival. FIG. 6H shows flow cytometric quantification of tumor cells (left) and T cells (right) in bone marrow 12 days after T cell infusion (n=2-3 mice). FIG. 6I shows phenotype of persisting iT cells prior to infusion (day 0, n=1) and of cells derived from the bone marrow on day 6 and 12 days after iT cell infusion (n=3 mice). * P<0.05, ** P<0.001, *** P<0.001, Chi-Square test (FIG. 6B), Welch's 2-sample t test (FIGS. 6D and 6H), log-rank Mantel-Cox test (FIG. 6G). All data are means±s.d FIGS. 7A-7D illustrate T lymphoid commitment of hES, FiPS and TiPS on OP9-mDLL1.

FIG. 7A shows flow cytometric analysis of pluripotency marker expression on H1, FiPS and WT-TiPS. FIG. 7B shows flow cytometric analysis of T lymphoid markers of H1 during differentiation on OP9-mDLL1 at indicated timepoints. FIG. 7C shows flow cytometric analysis of T lymphoid markers of FiPS during differentiation on OP9-mDLL1 at indicated timepoints. FIG. 7D shows flow cytometric analysis of T lymphoid markers of WT-TiPS during differentiation on OP9-mDLL1 at indicated timepoints. Plots depicting CD7/CD5 are gated on live CD45⁺ cells, plots depicting CD3/TCRαβ, CD4/CD8a and CD8αβ/CD80 are gated on live CD45⁺CD7⁺ cells. CD3/TCRαβ and CD4/CD8α at D40 are as presented in FIG. 1B.

FIGS. 8A-8D illustrate generation, validation, and differentiation of TRAC^{-/-}-TiPS. FIG. 8A shows CRISPR/Cas9-targeted integration of EF1a-GFP-P2A-Puromycing-bGHpA (G2AP) expression unit into the TRAC locus. Top, TRAC locus; middle, plasmid containing the G2AP expression unit flanked by homology arms; bottom, edited TRAC locus. ‘FWD’ and ‘REV’ indicate the location of the forward and reverse primers used in FIG. 8B. FIG. 8B shows PCR validation of G2AP integration into the TRAC locus of TiPS clones. FIG. 8C shows flow cytometric analysis of pluripotency marker expression on TRAC^{-/-}-TiPS. Gated on live cells. FIG. 8D shows T lymphoid makers of WT-TiPS and TRAC^{-/-}-TiPS during differentiation on OP9-mDLL1 at the indicated timepoints. Gated on live CD45⁺ cells. D40 is as presented in FIG. 1C.

FIGS. 9A-9H illustrate early T lymphoid commitment of WT-TiPS and CAR-TiPS on human Notch ligands. FIG. 9A shows SFG γRV plasmid design to transduce human Notch ligands (DLL1, DLL4, JAG1 or JAG2) into parental OP9 cells. FIG. 9B shows Notch ligand expression on engineered OP9 lines. Filled grey histogram are stained parental OP9 cells, open black histogram are transduced OP9 cells. FIG. 9C shows DTX1 induction in WT-TiPS by OP9 expressing indicated Notch ligand. D20 differentiating WT-TiPS cells were co-cultured with indicated OP9. DTX1 induction was measured by ddPCR, relative to endogenous RPL13A. The fold change was calculated relative to 0 h. Data shown are average of n=2 technical replicates. FIGS. 9D and 9G show flow cytometric analysis of T lymphoid commitment marker expression (CD7, CD5, TCRαβ and CD56) of WT-TiPS (FIG. 9D) and CAR-TiPS (FIG. 9G) differentiated on OP9 expressing indicated human Notch ligands. Gated on live CD45⁺ cells. FIG. 9E shows flow cytometric analysis of pluripotency marker expression on CAR-TiPS. Gated on live cells. FIG. 9F shows phosphorylated-ERK1/2 levels in WT-TiPS (blue) and CAR-TiPS (red) on D35 (n=3 technical replicates). FIG. 9H shows the phenotype (left panels) and apoptosis levels (right panels) of WT-TiPS (top) and CAR-TiPS (bottom) from D27-D35 of differentiation on OP9-DLL4. Percentage of apoptotic cells in each T lineage developmental stage was based on percentage of live Annexin-V+ cells. * P<0.05, ** P<0.001, *** P<0.001, Welch's 2-sample t-test, data are means±s.d (FIG. 9F)

FIGS. 10A-10G illustrate CD8αβ single positive CAR+iT cell development. WT-TiPS were differentiated on OP9-DLL4 and transduced to express the 1928z CAR at D35 utilizing γRV SFG-1928z-P2A-LNGFR. Cells were expanded for 7 days in expansion media supplemented with IL-2. FIG. 10A shows CD4/CD8αβ expression prior to transduction (D35) and on D42 in LNGFRT cells, LNGFR-cells and untransduced control cells which remained in differentiation on OP9-DLL4. Gated on live CD45T cells. FIG. 10B shows cytotoxic activity of CART iT cells in a 18 h bioluminescence assay, using FFLuc-NALM6 as target cells (n=3 technical replicates, data are mean±s.d). FIG. 10C shows CRISPR/Cas9-targeted integration of CAR transgene into the TRAC locus. Top, TRAC locus; middle, plasmid containing the CAR transgene cassette flanked by homology arms; bottom, edited TRAC locus. FIGS. 10D and 10F show PCR validation of CAR integration into the TRAC locus of TRAC-1928z-TiPS (FIG. 10D) and TRAC-1XX-TiPS (FIG. 10F) clones. FIGS. 10E and 10G show pluripotency marker expression on TRAC-1928z-TiPS (FIG. 10E) and TRAC-1XX-TiPS (FIG. 10G), gated on live cells.

FIGS. 11A-11C illustrate T lineage commitment of TRAC-CAR-TiPS. FIG. 11A shows T lineage commitment marker expression (CD7/CD5, CD4/CD8α, CD8α/CD8b) of WT-TiPS (left), TRAC-1928z-TiPS (middle) and TRAC-1XX-TiPS on OP9-DLL4 at the indicated timepoints. CD7/CD5 is gated on live CD45⁺ cells, others are gated on live CD45⁺CD7⁺ cells. FIG. 11B shows flow cytometric analysis of T cell phenotype markers of D35 DP TRAC-1XX-iT cells. Gated on live CD45⁺CD7+CD4⁺CD8αβ+ cells. FIG. 11C shows intracellular and cell-surface expression of CD3 and TCRαβ on D35 TRAC-1XX-iT cells.

FIGS. 12A-12C illustrate tonic ITAM phosphorylation in CAR⁺ T cells. FIG. 12A shows representative flow cytometry plot of CAR expression and pITAM1 (top panel) or pITAM3 (bottom panel) in PBMC-derived T cells expressing γRV-1928z, TRAC-1928z or TRAC-1XX (gated on live CAR+), or in control TRAC^{-/-} cells (gated on live CAR-). FIG. 12B shows percentage of pITAM1⁺ in the populations shown in FIG. 12A (n=4-5 biological replicates, data are means±s.d.). FIG. 12C shows percentage of pITAM3⁺ in the populations shown in FIG. 12A (n=4-5 biological replicates, data are means±s.d.).

FIGS. 13A-13F illustrate DP TRAC-1XX-iT cell matures to CD8αβ SP iT cells on 3T3-CD19-41BBL. FIGS. 13A and 13C show flow cytometric analysis of D42 cells matured on 3T3-CD19 (FIG. 13A) or 3T3-CD19-41BBL (FIG. 13C). Gated on live CD45⁺CD7⁺ cells. FIG. 13B shows flow cytometric analysis of D35 and D42 phenotypes of stimulated DP TRAC-1XX-iT cells. D35 TRAC-1XX-iT cells were sorted for a CD4⁺CD8αβ⁺ DP phenotype, stimulated on 3T3-CD19-41BBL, and expanded for seven days. Gated on live CD45⁺CD7⁺ cells. FIG. 13D shows fold Expansion and T cell phenotype marker expression of TRAC-1XX-iT cells matured on 3T3-CD19-41BBL (3T3) or recombinant CD19-Fc. FIG. 13E shows a 4 h cytotoxicity assay of 3T3-CD19-41BBL stimulated TRAC-1XX-iT cells in response to NALM6-CD19+a NALM6-CD19-target cells (n=3 technical replicates, data are means±s.d.). FIG. 13F shows CD19 expression on primary CLL cells.

FIGS. 14A-14C illustrate comparison of CD8α TRAC-1XX-iT cells and peripheral blood lymphocytes. FIG. 14A shows representative examples of lymphoid phenotype marker expression in CD8α TRAC-1XX-iT (red), CD8ab αβTCR-T (blue), CD4 αβTCR-T (orange), γδTCR-T (green) and NK cells (purple). CD8αβ TRAC-1XX-iT cells are the same as represented in FIG. 5A. FIG. 14B shows variability of lymphoid phenotype marker expression in CD8αβ TRAC-1XX-iT cells (n=34 biological replicates, data are means±s.d). Biological replicates shown are samples utilized in RNA analysis. FIG. 14C shows principal Component Analysis comparing TRAC-1XX CD8αβ αβTCR-T cells (CD8, n=4), TRAC-1XX CD4 αβTCR-T cells (CD4, n=3), γRV-1XX γδTCR-T cells (γδ, n=4), γRV-1XX NK cells (NK, n=4) and CD8αβ TRAC-1XX-iT cells (iT CD8αβ, n=4).

FIGS. 15A-15K illustrate TRAC-1XX-iT have improved persistence and function over CAR-iT cells. Functional comparison of healthy-donor peripheral blood TRAC-1XX CD8αβ αβTCR-T (CD8 TRAC-1XX), CAR-iT and TRAC-1XX-iT cells. CD8 TRAC-1XX cell doses represent number of CAR+ cells utilized in the assay. FIG. 15A shows CAR and CD3 expression in CD8 TRAC-1XX, CAR-iT and TRAC-1XX-iT cells (black line) compared to unstained control (grey filled histogram). FIG. 15B shows 18 h Incucyte cytotoxicity assay with NLR⁺CD19^{-/-}-NALM6 target cells (n=3 technical replicates). FIG. 15C shows 4 h intracellular cytokine detection in unstimulated control (left panel), NALM6 CD19^{-/-} target cells (at a 1:1 E:T, middle panel) or PMA/Ionomycin activated (right panel) (n=3 technical replicates). FIG. 15D shows 24 h cytokine secretion using NALM6-CD19−/− as target cells at a 1:1 E:T (n=11-18 biological replicates, upper panel) or unstimulated control (n=11-18 biological replicates, lower panel). FIG. 15E shows schematic representation of the NALM6 in vivo tumor model. FIG. 15F shows tumor burden (total flux in photons per second) of NALM6-bearing, untreated mice or mice treated with 4×10⁶ TRAC-1XX-iT (middle) or CAR-iT (right) cells (n=7, line=one mouse). FIG. 15G shows Kaplan-Meier analysis of overall survival. FIGS. 15H and 15I show enumeration of T cells (FIG. 15H) and tumor cells (FIG. 15I) in the bone marrow, spleen and blood 12 days post T cell infusion (n=4 mice). FIG. 15J shows schematic representation of the NALM6 in vivo tumor model. FIG. 15K shows Kaplan-Meier analysis of overall survival. * P<0.05, ** P<0.001, *** P<0.001, Welch's 2-sample t test (FIGS. 15H and 15I) log-rank Mantel-Cox test (FIGS. 15G and 15K). All data are means ±s.d.

FIGS. 16A-16D illustrate TRAC-1XX-iT function compared to healthy donor peripheral blood-derived CD8 TRAC-1XX T cells. In vivo functional comparison of healthy-donor peripheral blood TRAC-1XX CD8αβ αβTCR-T (CD8 TRAC-1XX), TRAC-1XX-iT cells. CD8 TRAC-1XX cell doses represent number of CART cells utilized in the assay. FIG. 16A shows CAR and CD3 expression in CD8 TRAC-1XX and TRAC-1XX-iT cells (black line) compared to unstained control (grey filled histogram). FIG. 16B shows enumeration of tumor cells in the bone marrow and T cells in bone marrow, spleen and blood 6- or 12-days post T cell infusion (n=2-3 mice). FIG. 16C shows phenotype of CD8 cells prior to infusion (day 0, n=1) and of cells derived from the bone marrow on day 6 (n=3 mice) and 12 (n=3 mice). FIG. 16D shows Kaplan-Meier analysis of overall survival. * P<0.05, ** P<0.001, *** P<0.001 Welch's 2-sample t test (FIG. 16B) log-rank Mantel-Cox test (FIG. 16D). All data are means±s.d.

FIGS. 17A and 17B illustrate flow cytometry gating strategy for iT cells. FIG. 17A shows gating strategy applied to identify live, CD45⁺CD7+iT cells. FIG. 17B shows gating strategy applied to identify CD4, CD8α and CD8β expression levels. Gating is set based on stained PBMC controls (left panels) and stained in CD45⁺ lymphoid precursor cells (D20, WT-TiPS and CAR-TiPS respectively) to adjust for autofluorescence.

FIGS. 18A-18C illustrate the development and specificity of 19E3 and 12D11 antibodies. FIG. 18A shows the flow cytometric assessment of antibody specificity against CAR+PG13 fibroblast. Expressions of the CD19-targeting second-generation 1928z CAR, first-generation 19z1 CAR, and first-generation PSMA-targeting Pz1 CAR were confirmed with the polyclonal goat-anti-mouse F(ab)′ fragment. FIG. 18B shows staining with 19E3 antibody specific to SJ25C1 recognition in the 1928z and 19z1 CARs. FIG. 18C shows staining with 12D11 antibody, having scFv-independent CAR recognition, for detecting both CD19 and PSMA-targeting CARs.

FIGS. 19A-19C illustrate the development and differentiation of iPSC-derived CAR+ T cells contacted with the 19E3 and 12D11 antibodies. FIG. 19A shows flow cytometric analysis of end-stage differentiated T cells contacted with 19E3 or 12D11 anti-idiotype antibodies and resulting in the maturation and development of effector phenotype (e.g., CD2+, CD56lo, CD45RA+, CD62L+, CCR7lo, CXCR4+, and CD25+) compared to stronger stimulation with CD19-protein expressing feeder cells K562-CD19. FIG. 19B shows 19E3-maturated T cells having superior cytolytic capacity over T cells developed in the presence of K562-CD19 cells. FIG. 19C shows 19E3-maturated T cells produced cytokines (e.g., IFNγ, Granzyme B, CD107a, and TNFα) in a stimulation-dependent manner.

FIGS. 20A-20C illustrate the effects of the 19E3 and 12D11 antibodies on the development of iPSC-derived CAR⁺ T cells. FIG. 20A shows a schematic outline of the experimental approach for in vitro evaluation of iPSC-derived CAR⁺ T cell maturation. Premature T cells were subjected to various dosages of 19E3 antibody (from 0 μg/ml to 9 μg/ml) over 42 days. FIG. 20B shows a flow cytometry analysis of T cells subjected to various dosages of 19E3 antibodies. Analyzed cell surface expression markers included CD4, CD8α, and CD8ab. FIG. 20C shows that 19E3 increased cell expansion and cell viability of T cells.

FIGS. 21A-21C illustrate that T cell proliferation is enhanced by co-stimulation with a 4-1BB agonist. FIG. 21A shows a schematic outlining the in vitro experimental approach to evaluate the effects of the 19E3 and 12D11 antibodies and urelumab. FIG. 21B shows that 19E3 and urelumab increased iT cell expansion and viability compared to 19E3 or urelumab alone and that these increases were comparable to iT cells obtained using 3T3-CD19-41BBL feeder-cells. FIG. 21C shows a heatmap of cell expansion (expressed as fold change) in response to various dosages of 19E3 and urelumab.

FIGS. 22A-22D illustrate that 19E3 and urelumab induced CAR T maturation in a dose-dependent manner. FIG. 22A shows a schematic of the experimental approach to assess the effects of 19E3 and Urelumab. FIG. 22B shows heatmaps of cell expansion and viability presence of various dosages of 19E3 and urelumab. FIG. 22C shows a flow cytometric analysis of the T cells obtained in presence of 19E3 and urelumab. FIG. 22D shows the cell growth and effector phenotype in response to 19E3 and different doses of urelumab.

FIGS. 23A and 23B illustrate the polyfunctionality of T cells obtained using the 19E3 and 12D11 antibodies. FIG. 23A shows cytotoxic activity of iT cells obtained using the 19E3 and 12D11 antibodies and different doses of urelumab against firefly luciferase (FFLuc)-expressing NALM6 CD19⁺ at the indicated effector-to-target (E:T) ratios. FIG. 23B shows cytokine secretion profiles of iT cells obtained using the 19E3 and 12D11 antibodies and different doses of urelumab.

FIGS. 24A-24E illustrate in vivo effects of iT cells obtained using the 19E3 and 12D11 antibodies and different doses of urelumab. FIG. 24A shows a schematic representation of the in vivo tumor model and experimental conditions. FIGS. 24B-24D show the results of tumor burden analysis using bioluminescence imaging (total flux) in response to iT cells obtained using the 19E3 and 12D11 antibodies and different doses of urelumab. FIG. 24B shows the tumor burden effect using 8×10⁶ cells. FIG. 24C shows the tumor burden effect using 4×10⁶ cells. FIG. 24D shows the tumor burden effect using 2×10⁶ cells. FIG. 24E shows Kaplan-Meier analysis of overall survival.

### 5. DETAILED DESCRIPTION OF THE INVENTION

The presently disclosed subject matter provides improved methods and composition useful to generate cells with enhanced activity and efficacy for immunotherapy (e.g., T cell immunotherapy). The presently disclosed subject matter is based, in part, on the unexpected discovery that chimeric antigen receptors (CAR) can improve the differentiation of pluripotent stem cells into T cells (e.g., induced T cells). Surprisingly, despite lacking expression of their endogenous TCR, T cells produced by the presently disclosed methods acquire conventional CD4 or CD8 T cell phenotype and do not show exhaustion markers. In certain embodiments, the presently disclosed methods can be used to manufacture CAR-T cells for use in the treatment of cancer.

The presently disclosed subject matter provides a multistage method of differentiating a pluripotent stem cell from a differentiated cell (e.g., induced T cell). The presently disclosed subject matter also provides compositions used in the methods disclosed herein as well as cells generated using the methods disclosed herein.

Traditionally, T cells differentiated from iPS have the downside of acquiring an innate-like CD8αβ-double-negative (DN) or CD8αα single-positive (SP) phenotype. In contrast, the presently disclosed subject matter addresses this problem by introducing into the pluripotent stem cell a nucleic acid composition (e.g., polynucleotide) encoding for an antigen-recognizing receptor (e.g., CAR) inserted into the genome of the cell (e.g., TRAC locus). The presently disclosed subject matter, thus, provides novel methods of enabling differentiating pluripotent stem cells to functional T cells (e.g., induced T cells) with high efficiency. Furthermore, the presently disclosed subject matter also provides a method using feeder-free conditions that removes hurdles for regulatory approvals.

Non-limiting embodiments of the presently disclosed subject matter are described by the present specification and Examples.

For purposes of clarity of disclosure and not by way of limitation, the detailed description is divided into the following subsections:
5.1. Definitions;
5.2. First Antigen-Recognizing Receptor;
5.3. Manufacturing of Induced T Cells (iTs);
5.4. Induced T Cells;
5.5. Formulation and Administration;
5.6. Methods of Treatment;
5.7. Kits;
5.8. Exemplary Embodiments.

### 5.1 Definitions

Unless defined otherwise, all technical and scientific terms used herein have the meaning commonly understood by a person skilled in the art. The following references provide one skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refers to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage. In certain embodiments, the immunoresponsive cell is a monocyte.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane-bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigens. While the effects of these co-stimulatory signals may vary, they generally result in increased gene expression in order to generate long lived, proliferative, and anti-apoptotic T cells that robustly respond to antigens for complete and sustained eradication.

By “engages a CAR” is meant induction of phosphorylation of CD3 Chains cluster in response to ligand binding and immunoreceptor tyrosine-based inhibition motifs (ITAMs) upon which a signal transduction cascade is produced. In certain embodiments, the term “engages a CAR” refers to induction of phosphorylation of the ITAM1 domain of a CD3ζ polypeptide (e.g., a CD3ζ polypeptide comprised in a CAR). In certain embodiments, the term “engages a HIT” refers to induction of phosphorylation of the ITAM1 domain of an endogenous CD3ζ polypeptide (e.g., a CD3ζ polypeptide associated with the HIT receptor).

The term “antigen-recognizing receptor” as used herein refers to a receptor that is capable of activating an immune or immunoresponsive cell (e.g., a T-cell) in response to its binding to an antigen.

As used herein, the term “feeder cells” refers to a first type of cells that can be co-cultured with cells of a second type of cells in order to provide an environment that improves differentiation and growth of the second type of cells. In certain embodiments, the feeder cells can generate a conditioned medium that is enriched in growth factors, nutrients, and signals responsible for the growth and differentiation of the second type of cells. In certain embodiments, the feeder cells can be from the same species or from a different species as the cells they are supporting (e.g., second type of cells). For example, but without any limitation, certain types of human cells, including pluripotent stem cells, can be supported by primary cultures of mouse embryonic fibroblasts, or immortalized mouse embryonic fibroblasts. In certain embodiments, the feeder cells can be inactivated (e.g., by irradiation). Non-limiting examples of feeder cells include endothelial cells, stromal cells (e.g., fibroblasts), and leukemic cells.

As used herein, the term “feeder-free” refers to a cell culture system or environment (e.g., cell culture medium) that lacks feeder cells. In certain embodiments, a feeder free system has not been pre-conditioned by the cultivation of feeder cells. As used herein, the term “pre-conditioned medium” refers to a medium or cell culture system which has been harvested after feeder cells have been cultivated for a period of time (e.g., 2 days). This pre-conditioned medium contains growth factors and cytokines secreted by the feeder cells. In certain embodiments, however, even such pre-conditioning is unnecessary in view of the strategies described herein.

The terms “induced pluripotent stem cells” or “iPS,” as used herein, refer to stem cells that are produced from fully differentiated cells which have been reprogrammed into cells capable of differentiation into tissues of the three germs layers (e.g., ectoderm, mesoderm, endoderm). The resulting iPS are not naturally occurring as they include genomic engineering and reprogramming. Non-limiting examples of methods to obtain induced pluripotent stem cells can be found in Valamehr et al., Stem Cell Reports 2, 366-381 (2014); Themeli et al., Nat Biotechnol 31, 928-933 (2013), Mansilla-Soto et al., Nat Med 28, 345-352 (2022), and Eyquem et al., Nature 543, 113-117 (2017), the content of each of which is incorporated by reference in their entirety. In certain embodiments, the pluripotent stem cells are induced pluripotent stem cells (iPS). In certain embodiments, the pluripotent stem cells are T cell-derived induced pluripotent stem cells (TiPS).

As used herein, the terms “induced T cell” or “iT” refers to differentiated T cells produced from induced pluripotent stem cells (e.g., T cell-derived pluripotent stem cells). These induced T cells (iTs) are not pluripotent stem cells as they have acquired specialized features and functions. For example, but without any limitation, induced T cells are capable of eliciting an immune response upon binding with a non-self antigen.

As used herein, the term “differentiation” refers to a process whereby an unspecialized cell (e.g., a pluripotent stem cell) acquires the features of a specialized cell such as a lymphocyte, a neuron cell, a hepatocyte, or a muscle cell. Differentiation is controlled by the interaction of a cell's genes with the physical and chemical conditions outside the cell, usually through signaling pathways involving proteins embedded in the cell surface.

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” a cell or cells with a compound (e.g., at least one inhibitor, activator, and/or inducer) refers to providing the compound in a location that permits the cell or cells access to the compound. The contacting may be accomplished using any suitable method.

For example, contacting can be accomplished by adding the compound, in concentrated form, to a cell or population of cells, for example in the context of a cell culture, to achieve the desired concentration. Contacting may also be accomplished by including the compound as a component of a formulated culture medium.

As used herein, the term “in vitro” refers to an artificial environment and to processes or reactions that occur within an artificial environment. In vitro environments exemplified are, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

The term “hematopoietic precursor,” as used herein, refers to CD34+ cells capable of giving rise to both mature myeloid and lymphoid cell types (e.g., T cells, NK cells, and B cells).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is Composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4^{th} U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs regions are delineated using the PyIgClassify system (Adolf-Bryfogle et al., Nucleic acids research 43.D1 (2015): D432-D438).

As used herein, the term “Fe fusion protein” refers to homodimers in which an Fc domain of an antibody is covalently linked to another protein. Fc is the crystallizable fragment derived from Ig which has five classes including IgG, IgA, IgD, IgM, and IgE in human (Schroeder and Cavacini L, J Allergy Clin Immunol (2010) 125(2 Suppl 2):S41-52). Fc plays multiple roles in activation and recruiting of immune leukocytes, triggering of antibody-dependent cell-mediated cytotoxicity (ADCC) and antibody-dependent cell-mediated phagocytosis (Ravetch and Bolland, Annu Rev Immunol (2001) 19:275-90; Woof and Burton, Nat Rev Immunol (2004) 4(2):89-99). In addition, Fc can bind to the serum complement molecule (C1q) to initiate the assembly of membrane attack complex formed by complement cascade proteins to destroy target cells, which is termed complement-dependent cytotoxicity (CDC) (Walport, N Engl J Med (2001) 344(14):1058-66; Walport, N Engl J Med (2001) 344(15):1140-4). Overall, Fc plays important roles in biological and pharmacological properties including, among other functions, increased stability and aggregation resistance, acquired multivalent binding to the target, enhanced Fc-mediated effector functions, extended serum half-life, and modulated immunogenicity. Additional information regarding Fc proteins can be found in Beck and Reichert, MAbs. Vol. 3. No. 5. Taylor & Francis (2011)., the content of which is incorporated by reference in its entirety.

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker is a G4S linker. In certain embodiments, the linker comprises or consists of the amino acid sequence set forth in SEQ ID NO: 1, which is provided below:

[SEQ ID NO: 1]
GGGGSGGGGS

In certain embodiments, the linker comprises or consists of the amino acid sequence set forth in SEQ ID NO: 2, which is provided below:

[SEQ ID NO: 2]
GGGGSGGGGSGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGGGGGS

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “Fe fusion protein” refers to homodimers in which an Fc domain of an antibody is covalently linked to another protein. Fc is the crystallizable fragment derived from Ig which has five classes including IgG, IgA, IgD, IgM, and IgE in human (Schroeder and Cavacini L, J Allergy Clin Immunol (2010) 125(2 Suppl 2):S41-52). Fc plays multiple roles in activation and recruiting of immune leukocytes, triggering of antibody-dependent cell-mediated cytotoxicity (ADCC) and antibody-dependent cell-mediated phagocytosis (Ravetch and Bolland, Annu Rev Immunol (2001) 19:275-90; Woof and Burton, Nat Rev Immunol (2004) 4(2):89-99). In addition, Fc can bind to the serum complement molecule (C1q) to initiate the assembly of membrane attack complex formed by complement cascade proteins to destroy target cells, which is termed complement-dependent cytotoxicity (CDC) (Walport, N Engl J Med (2001) 344(14):1058-66; Walport, N Engl J Med (2001) 344(15):1140-4). Overall, Fc plays important roles in biological and pharmacological properties including, among other functions, increased stability and aggregation resistance, acquired multivalent binding to the target, enhanced Fc-mediated effector functions, extended serum half-life, and modulated immunogenicity. Additional information regarding Fc proteins can be found in Beck and Reichert, MAbs. Vol. 3. No. 5. Taylor & Francis (2011)., the content of which is incorporated by reference in its entirety.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis), and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to a chimeric receptor” or “specifically binds to a CAR” is intended to refer to an antibody that binds to an antigen-binding domain of a CAR (e.g., the extracellular antigen-binding domain of a CAR) with a dissociation constant (K_{D}) of about 1×10⁻⁸ M or less, about 5×10⁻⁹ M or less, about 1×10⁻⁹ M or less, about 5×10⁻¹⁰ M or less, about 1×10⁻¹⁰ M or less, about 5×10⁻¹¹ M or less, about 1×10⁻¹¹ M or less, about 5×10⁻¹² M or less, or about 1×10⁻¹² M or less.

An “antibody that competes for binding” or “antibody that cross-competes for binding” with a reference antibody for binding to an antigen, e.g., an antigen-binding domain (e.g., the extracellular antigen-binding domain of a CAR), refers to an antibody that blocks binding of the reference antibody to the antigen (e.g., an antigen-binding domain, e.g., the extracellular antigen-binding domain of a CAR) in a competition assay by 50% or more, and conversely, the reference antibody blocks binding of the antibody to the antigen (e.g., an antigen-binding domain, e.g., the extracellular antigen-binding domain of a CAR) in a competition assay by 50% or more. An exemplary competition assay is described in “Antibodies”, Harlow and Lane (Cold Spring Harbor Press, Cold Spring Harbor, NY).

As used herein, “isotype” refers to the antibody class (e.g., IgM or IgG1) that is encoded by the heavy chain constant region genes.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e−3 and e−100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv703) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of a presently disclosed CAR (e.g., the presently disclosed exemplary CD19-targeted CAR, e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

“Inhibitor” as used herein, refers to a compound or molecule (e.g., small molecule, peptide, peptidomimetic, natural compound, siRNA, anti-sense nucleic acid, aptamer, or antibody) that interferes with (e.g., reduces, decreases, suppresses, eliminates, or blocks) the signaling function of the molecule or pathway (e.g., BMP signaling pathway).

“Activators,” as used herein, refer to compounds that increase, induce, stimulate, activate, facilitate, or enhance activation the signaling function of the molecule or pathway, e.g., Notch signaling, etc.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 5.2 First Antigen-Recognizing Receptor

The presently disclosed subject matter provides induced T cells (e.g., prepared through the methods disclosed herein) including an antigen-recognizing receptor. In certain embodiments, the antigen-recognizing receptor targets an antigen. In certain embodiments, the antigen can be a tumor antigen or a pathogen antigen. In certain embodiments, the antigen-recognizing receptor is a chimeric receptor. In certain embodiments, the chimeric receptor is a chimeric antigen receptor (CAR).

### 5.2.1 Antigen

In certain embodiments, the antigen is a tumor antigen. Any tumor antigen (antigenic peptide) can be used in the tumor-related embodiments described herein. Sources of antigen include, but are not limited to, cancer proteins. The antigen can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the antigen is a pathogen antigen. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, Corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 5.2.2 Chimeric Receptors

In certain embodiments, the first antigen-recognizing receptor is a chimeric receptor. In certain embodiments, the first chimeric receptor is a chimeric antigen receptor (CAR). In certain embodiments, the first chimeric receptor is a TCR like fusion molecule.

### 5.2.2.1 Chimeric Antigen Receptors (CARs)

In certain embodiments, the first chimeric receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4+ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40, NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the first antigen-recognizing receptor is a CAR comprising an extracellular antigen-binding domain that binds to the antigen and an intracellular signaling domain. In certain embodiments, the CAR further comprises a transmembrane domain. In certain embodiments, the CAR further comprises a hinger/spacer region.

In certain embodiments, the extracellular antigen-binding domain of the CAR (for example, an scFv) binds to the first antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹ M or less, or about 1×10⁻¹⁰ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, andCyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In certain embodiments, the first antigen-recognizing receptor is a CAR that comprises a transmembrane domain. Different transmembrane domains result in different receptor stability. After antigen recognition, receptors cluster and a signal are transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having an NCBI Reference No: NP_006130 (SEQ ID NO: 7), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 7. In certain embodiments, the transmembrane domain of the CAR comprises a CD28 polypeptide that comprises or consists of amino acids 153 to 179 of SEQ ID NO: 7. SEQ ID NO: 7 is provided below.

[SEQ ID NO: 7]
MLRLLLALNLEPSIQVTGNKILVKQSPMLVAYDNAVNLSCKYSYNLFSR
EFRASLHKGLDSAVEVCVVYGNYSQQLQVYSKTGFNCDGKLGNESVTFY
LQNLYVNQTDIYFCKIEVMYPPPYLDNEKSNGTIIHVKGKHLCPSPLFP
GPSKPFWVLVVVGGVLACYSLLVTVAFIIFWVRSKRSRLLHSDYMNMTP
RRPGPTRKHYQPYAPPRDFAAYRS

In certain embodiments, the first antigen-recognizing receptor is a CAR that further comprises a hinge/spacer region that links the extracellular antigen-binding domain to the transmembrane GP-27, DNA domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof. The hinge/spacer region can be the hinge region from IgG1, the CH₂CH₃ region of immunoglobulin and portions of CD3, a portion of a CD28 polypeptide (e.g., a portion of SEQ ID NO: 7), a portion of a CD8 polypeptide, or a synthetic spacer sequence.

In certain embodiments, the first antigen-recognizing receptor is a CAR that further comprises a hinge/spacer region comprising a native or modified hinge region of a CD28 polypeptide. In certain embodiments, the hinge/spacer region of the first antigen-recognizing receptor (e.g., a CAR) comprises a CD28 polypeptide comprising or consisting of amino acids 114 to 152 of SEQ ID NO: 7.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8α polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8α polypeptide, a CD8b polypeptide, an ICOS polypeptide, an ICAM-1 polypeptide, a CTLA-4 polypeptide, a CD27 polypeptide, a CD40 polypeptide, an NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8α polypeptide and the transmembrane domain comprises a CD8α polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

In certain embodiments, the first antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99%, or about 100% identical or homologous to the amino acid sequence having an NCBI Reference No: NP_932170 (SEQ ID NO: 8) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 8, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 8. SEQ ID NO: 8 is provided below:

[SEQ ID NO: 8]
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the intracellular signaling domain of the CAR comprises a modified CD3ζ polypeptide. In certain embodiments, the modified CD3ζ polypeptide comprises one, two, or three ITAMs. In certain embodiments, the modified CD3ζ polypeptide comprises a native ITAM1. In certain embodiments, the native ITAM1 comprises or consists of the amino acid sequence set forth in SEQ ID NO: 9.

[SEQ ID NO: 9]
QNQLYNELNLGRREEYDVLDKR

An exemplary nucleic acid sequence encoding the amino acid sequence of SEQ ID NO: 9 is set forth in SEQ ID NO: 10, which is provided below.

[SEQ ID NO: 10]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 11, which is provided below.

[SEQ ID NO: 11]
QNQLFNELNLGRREEFDVLDKR

An exemplary nucleic acid sequence encoding the amino acid sequence of SEQ ID NO: 11 is set forth in SEQ ID NO: 12, which is provided below.

[SEQ ID NO: 12]
CAGAACCAGCTCTTTAACGAGCTCAATCTAGGACGAAGAGAGGAGTTCG
ATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 13, which is provided below.

[SEQ ID NO: 13]
QEGLYNELQKDKMAEAYSEIGMK

An exemplary nucleic acid sequence encoding the amino acid sequence of SEQ ID NO: 13 is set forth in SEQ ID NO: 14, which is provided below.

[SEQ ID NO: 14]
CAGGAAGGCCTGTACAATGAACTGCAGAAAGATAAGATGGCGGAGGCCT
ACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 15, which is provided below.

[SEQ ID NO: 15]
QEGLFNELQKDKMAEAFSEIGMK

An exemplary nucleic acid sequence encoding the amino acid sequence of SEQ ID NO: 15 is set forth in SEQ ID NO: 16, which is provided below.

[SEQ ID NO: 16]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 17, which is provided below.

HDGLYQGLSTATKDTYDALHMQ [ SEQ ID NO: 17]

An exemplary nucleic acid sequence encoding the amino acid sequence of SEQ ID NO: 17 is set forth in SEQ ID NO: 18, which is provided below.

[SEQ ID NO: 18]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 19, which is provided below.

[SEQ ID NO: 19]
HDGLFQGLSTATKDTEDALHMQ

An exemplary nucleic acid sequence encoding the amino acid sequence of SEQ ID NO: 19 is set forth in SEQ ID NO: 20, which is provided below.

[SEQ ID NO: 20]
CACGATGGCCTTTTCCAGGGGCTCAGTACAGCCACCAAGGACACCTTCG
ACGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 9, an ITAM2 variant consisting of the amino acid sequence set forth in SEQ ID NO: 15, and an ITAM3 variant consisting of the amino acid sequence set forth in SEQ ID NO: 19. In certain embodiments, the CAR is designated as “1XX”. In certain embodiments, the modified CD3ζ polypeptide comprises or consists of the amino acid sequence set forth in SEQ ID NO: 21. SEQ ID NO: 21 is provided below:

[SEQ ID NO: 21]
RVKFSRSADAPAYQQGQNQLYNELNLGRREEYDVLDKRRGRDPEMGGKP
RRKNPQEGLFNELQKDKMAEAFSEIGMKGERRRGKGHDGLFQGLSTATK
DTEDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 21 or a functional fragment thereof, and/or may optionally comprise up to one or up to two or up to three conservative amino acid substitutions.

An exemplary nucleic acid sequence encoding the amino acid sequence of SEQ ID NO: 21 is set forth in SEQ ID NO: 22, which is provided below.

[SEQ ID NO: 22]
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTTCAATGAACTGCAGAAAGATA
AGATGGCGGAGGCCTTCAGTGAGATTGGGATGAAAGGCGAGCGCCGGAG
GGGCAAGGGGCACGATGGCCTTTTCCAGGGGCTCAGTACAGCCACCAAG
GACACCTTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the first antigen-recognizing receptor comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 7 or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 7, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 7. In certain embodiments, the intracellular signaling domain of the CAR comprises a co-stimulatory signaling region that comprises a CD28 polypeptide comprising or consisting of amino acids 180 to 220 of SEQ ID NO: 7.

An exemplary nucleic acid sequence encoding the amino acid sequence of amino acids 180 to 220 of SEQ ID NO: 7 is set forth in SEQ ID NO: 23, which is provided below.

[SEQ ID NO: 23]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the intracellular signaling domain of the first antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having an NCBI Reference No: NP_031668.3 (or SEQ ID NO: 24) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 24, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 24. In certain embodiments, the co-stimulatory signaling region of the CAR comprises a CD28 polypeptide that comprises or consists of amino acids 178 to 218 of SEQ ID NO: 24. SEQ ID NO: 24 is provided below.

[SEQ ID NO: 24]
MTLRLLFLALNFFSVQVTENKILVKQSPLLVVDSNEVSLSCRYSYNLLA
KEFRASLYKGVNSDVEVCVGNGNFTYQPQFRSNAEFNCDGDEDNETVTF
RLWNLHVNHTDIYFCKIEFMYPPPYLDNERSNGTIIHIKEKHLCHTQSS
PKLFWALVVVAGVLFCYGLLVTVALCVIWTNSRRNRLLQSDYMNMTPRR
PGLTRKPYQPYAPARDFAAYRP

In certain embodiments, the intracellular signaling domain of the CAR comprises a cO stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having an NCBI Ref. No.: NP_001552 (SEQ ID NO: 25) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 25 which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 25. In certain embodiments, the co-stimulatory signaling region of the CAR comprises a 4-1BB polypeptide comprising or consisting of the amino acid sequence of amino acids 214 to 255 of SEQ ID NO: 25. SEQ ID NO: 25 is provided below.

[SEQ ID NO: 25]
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTENDQKRGICRPWTNCSLDGKSVLVNG
TKERDVVCGPSPADLSPGASSVTPPAPAREPGHSPQIISFFLALTSTALL
FLLFELTLRFSVVKRGRKKLLYIFKQPFMRPVQTTQEEDGCSCRFPEEEE
GGCEL

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human IL-2 signal sequence comprising or consisting of the amino acid sequence set forth in SEQ ID NO: 26), a mouse IL-2 signal sequence (e.g., a mouse IL-2 signal sequence comprising or consisting of the amino acid sequence set forth in SEQ ID NO: 27); a human kappa leader sequence (e.g., a human kappa leader sequence comprising or consisting of the amino acid sequence set forth in SEQ ID NO: 28), a mouse kappa leader sequence (e.g., a mouse kappa leader sequence comprising or consisting of the amino acid sequence set forth in SEQ ID NO: 29); a human CD8 leader sequence (e.g., a human CD8 leader sequence comprising or consisting of the amino acid sequence set forth in SEQ ID NO: 30); a truncated human CD8 signal peptide (e.g., a truncated human CD8 signal peptide comprising or consisting of the amino acid sequence set forth in SEQ ID NO: 31); a human albumin signal sequence (e.g., a human albumin signal sequence comprising or consisting of the amino acid sequence set forth in SEQ ID NO: 32); and a human prolactin signal sequence (e.g., a human prolactin signal sequence comprising or consisting of the amino acid sequence set forth in SEQ ID NO: 33). SEQ ID Nos: 26-33 are provided below.

| [SEQ ID NO: 26] | |
|---|---|
| | MYRMQLLSCIALSLALVINS |
| [SEQ ID NO: 27] | |
| | MYSMQLASCVTLTLVLLVNS |
| [SEQ ID NO: 28] | |
| | METPAQLLELLLLWLPDTTG |
| [SEQ ID NO: 29] | |
| | METDTLLLWVLLLWVPGSTG |
| [SEQ ID NO: 30] | |
| | MALPVTALLLPLALLLHAARP |
| [SEQ ID NO: 31] | |
| | MALPVTALLLPLALLLHA |
| [SEQ ID NO: 32] | |
| | MKWVTFISLLESSAYS |
| [SEQ ID NO: 33] | |
| | MDSKGSSQKGSRLLLLLVVSNLLLCQGVVS |

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 30 or SEQ ID NO: 31.

### 5.2.2.2 TCR Like Fusion Molecule

In certain embodiments, the first antigen-recognizing receptor is a TCR like fusion molecule. Non-limiting examples of TCR fusion molecules include HLA-Independent TCR-based Chimeric Antigen Receptor (also known as “HIT”, e.g., those disclosed in International Patent Application No. PCT/US2019/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprise a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT”).

In certain embodiments, the first antigen-binding chain comprises an antigen-binding fragment of a heavy chain variable region (VH) of an antibody. In certain embodiments, the second antigen-binding chain comprises an antigen-binding fragment of a light chain variable region (VL) of an antibody. In certain embodiments, the first antigen-binding chain comprises an antigen-binding fragment of a VH of an antibody, and the second antigen-binding chain comprises an antigen-binding fragment of a VL of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide, and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a VH of an antibody and a TRAC polypeptide, and the second antigen-binding chain comprises a VL of an antibody and a TRBC polypeptide.

In certain embodiments, the first antigen-binding chain comprises a VH of an antibody and a TRBC polypeptide, and the second antigen-binding chain comprises a VL of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell (e.g., an induced T cell). In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 80 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 80. SEQ ID NO: 80 is provided below.

[SEQ ID NO: 80]
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGENLLMTLRLWSS

An exemplary nucleotide sequence encoding the amino acid sequence of SEQ ID NO: 80 is set forth in SEQ ID NO: 81, which is provided below.

[SEQ ID NO: 81]
ATCCAGAACCCTGACCCTGCCGTGTACCAGCTGAGAGACTCTAAATCCAG
TGACAAGTCTGTCTGCCTATTCACCGATTTTGATTCTCAAACAAATGTGT
CACAAAGTAAGGATTCTGATGTGTATATCACAGACAAAACTGTGCTAGAC
ATGAGGTCTATGGACTTCAAGAGCAACAGTGCTGTGGCCTGGAGCAACAA
ATCTGACTTTGCATGTGCAAACGCCTTCAACAACAGCATTATTCCAGAAG
ACACCTTCTTCCCCAGCCCAGAAAGTTCCTGTGATGTCAAGCTGGTCGAG
AAAAGCTTTGAAACAGATACGAACCTAAACTTTCAAAACCTGTCAGTGAT
TGGGTTCCGAATCCTCCTCCTGAAAGTGGCCGGGTTTAATCTGCTCATGA
CGCTGCGGCTGTGGTCCAGC

In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 82 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 82. SEQ ID NO: 82 is provided below.

[SEQ ID NO: 82]
IPNIQNPDPAVYQLRDSKSSDKSVCLFTDFDSQTNVSQSKDSDVYITDKT
VLDMRSMDFKSNSAVAWSNKSDEACANAENNSIIPEDTFFPSPESSCDVK
LVEKSFETDTNLNFQNLSVIGFRILLLKVAGENLLMTLRLWSS

An exemplary nucleotide sequence encoding the amino acid sequence of SEQ ID NO: 82 is set forth in SEQ ID NO: 83, which is provided below.

[SEQ ID NO: 83]
ATTCCCAATATCCAGAACCCTGACCCTGCCGTGTACCAGCTGAGAGACTC
TAAATCCAGTGACAAGTCTGTCTGCCTATTCACCGATTTTGATTCTCAAA
CAAATGTGTCACAAAGTAAGGATTCTGATGTGTATATCACAGACAAAACT
GTGCTAGACATGAGGTCTATGGACTTCAAGAGCAACAGTGCTGTGGCCTG
GAGCAACAAATCTGACTTTGCATGTGCAAACGCCTTCAACAACAGCATTA
TTCCAGAAGACACCTTCTTCCCCAGCCCAGAAAGTTCCTGTGATGTCAAG
CTGGTCGAGAAAAGCTTTGAAACAGATACGAACCTAAACTTTCAAAACCT
GTCAGTGATTGGGTTCCGAATCCTCCTCCTGAAAGTGGCCGGGTTTAATC
TGCTCATGACGCTGCGGCTGTGGTCCAGC

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 34) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 34. SEQ ID NO: 34 is provided below.

[SEQ ID NO: 34]
ATATCCAGAACCCTGACCCTGCCGTGTACCAGCTGAGAGACTCTAAATCCAGTGACAAGTCTGTCTGCCTATT
CACCGATTTTGATTCTCAAACAAATGTGTCACAAAGTAAGGATTCTGATGTGTATATCACAGACAAAACTGTG
CTAGACATGAGGTCTATGGACTTCAAGAGCAACAGTGCTGTGGCCTGGAGCAACAAATCTGACTTTGCATGTG
CAAACGCCTTCAACAACAGCATTATTCCAGAAGACACCTTCTTCCCCAGCCCAGGTAAGGGCAGCTTTGGTGC
CTTCGCAGGCTGTTTCCTTGCTTCAGGAATGGCCAGGTTCTGCCCAGAGCTCTGGTCAATGATGTCTAAAACT
CCTCTGATTGGTGGTCTCGGCCTTATCCATTGCCACCAAAACCCTCTTTTTACTAAGAAACAGTGAGCCTTGT
TCTGGCAGTCCAGAGAATGACACGGGAAAAAAGCAGATGAAGAGAAGGTGGCAGGAGAGGGCACGTGGCCCAG
CCTCAGTCTCTCCAACTGAGTTCCTGCCTGCCTGCCTTTGCTCAGACTGTTTGCCCCTTACTGCTCTTCTAGG
CCTCATTCTAAGCCCCTTCTCCAAGTTGCCTCTCCTTATTTCTCCCTGTCTGCCAAAAAATCTTTCCCAGCTC
ACTAAGTCAGTCTCACGCAGTCACTCATTAACCCACCAATCACTGATTGTGCCGGCACATGAATGCACCAGGT
GTTGAAGTGGAGGAATTAAAAAGTCAGATGAGGGGTGTGCCCAGAGGAAGCACCATTCTAGTTGGGGGAGCCC
ATCTGTCAGCTGGGAAAAGTCCAAATAACTTCAGATTGGAATGTGTTTTAACTCAGGGTTGAGAAAACAGCTA
CCTTCAGGACAAAAGTCAGGGAAGGGCTCTCTGAAGAAATGCTACTTGAAGATACCAGCCCTACCAAGGGCAG
GGAGAGGACCCTATAGAGGCCTGGGACAGGAGCTCAATGAGAAAGGAGAAGAGCAGCAGGCATGAGTTGAATG
AAGGAGGCAGGGCCGGGTCACAGGGCCTTCTAGGCCATGAGAGGGTAGACAGTATTCTAAGGACGCCAGAAAG
CTGTTGATCGGCTTCAAGCAGGGGAGGGACACCTAATTTGCTTTTCTTTTTTTTTTTTTTTTTTTTTTTTTTT
TTTGAGATGGAGTTTTGCTCTTGTTGCCCAGGCTGGAGTGCAATGGTGCATCTTGGCTCACTGCAACCTCCGC
CTCCCAGGTTCAAGTGATTCTCCTGCCTCAGCCTCCCGAGTAGCTGAGATTACAGGCACCCGCCACCATGCCT
GGCTAATTTTTTGTATTTTTAGTAGAGACAGGGTTTCACTATGTTGGCCAGGCTGGTCTCGAACTCCTGACCT
CAGGTGATCCACCCGCTTCAGCCTCCCAAAGTGCTGGGATTACAGGCGTGAGCCACCACACCCGGCCTGCTTT
TCTTAAAGATCAATCTGAGTGCTGTACGGAGAGTGGGTTGTAAGCCAAGAGTAGAAGCAGAAAGGGAGCAGTT
GCAGCAGAGAGATGATGGAGGCCTGGGCAGGGTGGTGGCAGGGAGGTAACCAACACCATTCAGGTTTCAAAGG
TAGAACCATGCAGGGATGAGAAAGCAAAGAGGGGATCAAGGAAGGCAGCTGGATTTTGGCCTGAGCAGCTGAG
TCAATGATAGTGCCGTTTACTAAGAAGAAACCAAGGAAAAAATTTGGGGTGCAGGGATCAAAACTTTTTGGAA
CATATGAAAGTACGTGTTTATACTCTTTATGGCCCTTGTCACTATGTATGCCTCGCTGCCTCCATTGGACTCT
AGAATGAAGCCAGGCAAGAGCAGGGTCTATGTGTGATGGCACATGTGGCCAGGGTCATGCAACATGTACTTTG
TACAAACAGTGTATATTGAGTAAATAGAAATGGTGTCCAGGAGCCGAGGTATCGGTCCTGCCAGGGCCAGGGG
CTCTCCCTAGCAGGTGCTCATATGCTGTAAGTTCCCTCCAGATCTCTCCACAAGGAGGCATGGAAAGGCTGTA
GTTGTTCACCTGCCCAAGAACTAGGAGGTCTGGGGTGGGAGAGTCAGCCTGCTCTGGATGCTGAAAGAATGTC
TGTTTTTCCTTTTAGAAAGTTCCTGTGATGTCAAGCTGGTCGAGAAAAGCTTTGAAACAGGTAAGACAGGGGT
CTAGCCTGGGTTTGCACAGGATTGCGGAAGTGATGAACCCGCAATAACCCTGCCTGGATGAGGGAGTGGGAAG
AAATTAGTAGATGTGGGAATGAATGATGAGGAATGGAAACAGCGGTTCAAGACCTGCCCAGAGCTGGGTGGGG
TCTCTCCTGAATCCCTCTCACCATCTCTGACTTTCCATTCTAAGCACTTTGAGGATGAGTTTCTAGCTTCAAT
AGACCAAGGACTCTCTCCTAGGCCTCTGTATTCCTTTCAACAGCTCCACTGTCAAGAGAGCCAGAGAGAGCTT
CTGGGTGGCCCAGCTGTGAAATTTCTGAGTCCCTTAGGGATAGCCCTAAACGAACCAGATCATCCTGAGGACA
GCCAAGAGGTTTTGCCTTCTTTCAAGACAAGCAACAGTACTCACATAGGCTGTGGGCAATGGTCCTGTCTCTC
AAGAATCCCCTGCCACTCCTCACACCCACCCTGGGCCCATATTCATTTCCATTTGAGTTGTTCTTATTGAGTC
ATCCTTCCTGTGGTAGCGGAACTCACTAAGGGGCCCATCTGGACCCGAGGTATTGTGATGATAAATTCTGAGC
ACCTACCCCATCCCCAGAAGGGCTCAGAAATAAAATAAGAGCCAAGTCTAGTCGGTGTTTCCTGTCTTGAAAC
ACAATACTGTTGGCCCTGGAAGAATGCACAGAATCTGTTTGTAAGGGGATATGCACAGAAGCTGCAAGGGACA
GGAGGTGCAGGAGCTGCAGGCCTCCCCCACCCAGCCTGCTCTGCCTTGGGGAAAACCGTGGGTGTGTCCTGCA
GGCCATGCAGGCCTGGGACATGCAAGCCCATAACCGCTGTGGCCTCTTGGTTTTACAGATACGAACCTAAACT
TTCAAAACCTGTCAGTGATTGGGTTCCGAATCCTCCTCCTGAAAGTGGCCGGGTTTAATCTGCTCATGACGCT
GCGGCTGTGGTCCAGCTGAGGTGAGGGGCCTTGAAGCTGGGAGTGGGGTTTAGGGACGCGGGTCTCTGGGTGC
ATCCTAAGCTCTGAGAGCAAACCTCCCTGCAGGGTCTTGCTTTTAAGTCCAAAGCCTGAGCCCACCAAACTCT
CCTACTTCTTCCTGTTACAAATTCCTCTTGTGCAATAATAATGGCCTGAAACGCTGTAAAATATCCTCATTTC
AGCCGCCTCAGTTGCACTTCTCCCCTATGAGGTAGGAAGAACAGTTGTTTAGAAACGAAGAAACTGAGGCCCC
ACAGCTAATGAGTGGAGGAAGAGAGACACTTGTGTACACCACATGCCTTGTGTTGTACTTCTCTCACCGTGTA
ACCTCCTCATGTCCTCTCTCCCCAGTACGGCTCTCTTAGCTCAGTAGAAAGAAGACATTACACTCATATTACA
CCCCAATCCTGGCTAGAGTCTCCGCACCCTCCTCCCCCAGGGTCCCCAGTCGTCTTGCTGACAACTGCATCCT
GTTCCATCACCATCAAAAAAAAACTCCAGGCTGGGTGCGGGGGCTCACACCTGTAATCCCAGCACTTTGGGAG
GCAGAGGCAGGAGGAGCACAGGAGCTGGAGACCAGCCTGGGCAACACAGGGAGACCCCGCCTCTACAAAAAGT
GAAAAAATTAACCAGGTGTGGTGCTGCACACCTGTAGTCCCAGCTACTTAAGAGGCTGAGATGGGAGGATCGC
TTGAGCCCTGGAATGTTGAGGCTACAATGAGCTGTGATTGCGTCACTGCACTCCAGCCTGGAAGACAAAGCAA
GATCCTGTCTCAAATAATAAAAAAAATAAGAACTCCAGGGTACATTTGCTCCTAGAACTCTACCACATAGCCC
CAAACAGAGCCATCACCATCACATCCCTAACAGTCCTGGGTCTTCCTCAGTGTCCAGCCTGACTTCTGTTCTT
CCTCATTCCAGATCTGCAAGATTGTAAGACAGCCTGTGCTCCCTCGCTCCTTCCTCTGCATTGCCCCTCTTCT
CCCTCTCCAAACAGAGGGAACTCTCCTACCCCCAAGGAGGTGAAAGCTGCTACCACCTCTGTGCCCCCCCGGC
AATGCCACCAACTGGATCCTACCCGAATTTATGATTAAGATTGCTGAAGAGCTGCCAAACACTGCTGCCACCC
CCTCTGTTCCCTTATTGCTGCTTGTCACTGCCTGACATTCACGGCAGAGGCAAGGCTGCTGCAGCCTCCCCTG
GCTGTGCACATTCCCTCCTGCTCCCCAGAGACTGCCTCCGCCATCCCACAGATGATGGATCTTCAGTGGGTTC
TCTTGGGCTCTAGGTCCTGCAGAATGTTGTGAGGGGTTTATTTTTTTTTAATAGTGTTCATAAAGAAATACAT
AGTATTCTTCTTCTCAAGACGTGGGGGGAAATTATCTCATTATCGAGGCCCTGCTATGCTGTGTATCTGGGCG
TGTTGTATGTCCTGCTGCCGATGCCTTC

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 35 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 35. SEQ ID NO: 35 is provided below.

[SEQ ID NO: 35]
DLKNVEPPEVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG

An exemplary nucleotide sequence encoding the amino acid sequence of SEQ ID NO: 35 is set forth in SEQ ID NO: 36, which is provided below.

[SEQ ID NO: 36]
GATCTGAAAAACGTGTTCCCTCCTGAAGTGGCTGTCTTTGAACCATCCGA
GGCCGAGATTTCCCATACCCAGAAAGCAACTCTGGTCTGTCTGGCCACTG
GATTCTACCCCGATCACGTGGAACTGTCTTGGTGGGTGAACGGCAAGGAA
GTCCATTCCGGAGTCTCTACCGACCCTCAGCCCCTCAAGGAGCAGCCTGC
TCTCAACGATTCTCGGTACTGCCTGTCATCTCGACTGAGAGTGTCTGCCA
CCTTCTGGCAGAACCCTAGAAACCACTTTCGGTGTCAGGTCCAGTTTTAC
GGCCTGAGCGAGAACGATGAGTGGACACAGGATAGAGCCAAACCTGTGAC
ACAGATTGTGAGCGCCGAGGCTTGGGGACGAGCCGATTGTGGCTTCACAT
CCGAGTCTTACCAGCAGGGAGTGCTGTCTGCTACAATCCTCTACGAAATT
CTCCTGGGGAAGGCCACCCTGTACGCTGTCCTCGTGTCTGCTCTGGTGCT
CATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 37 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 37. SEQ ID NO: 37 is provided below.

[SEQ ID NO: 37]
LNKVFPPEVAVFEPSEAEISHTQKATLVCLATGFFPDHVELSWWVNGKEV
HSGVSTDPQPLKEQPALNDSRYCLSSRLRVSATFWQNPRNHFRCQVQFYG
LSENDEWTQDRAKPVTQIVSAEAWGRADCGFTSVSYQQGVLSATILYEIL
LGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 38 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 38. SEQ ID NO: 38 is provided below.

[SEQ ID NO: 38]
DLNKVFPPEVAVFEPSEAEISHTQKATLVCLATGFFPDHVELSWWVNGKE
VHSGVSTDPQPLKEQPALNDSRYCLSSRLRVSATFWQNPRNHERCQVQFY
GLSENDEWTQDRAKPVTQIVSAEAWGRADCGFTSVSYQQGVLSATILYEI
LLGKATLYAVLVSALVLMAMVKRKDF

An exemplary nucleotide sequence encoding the amino acid sequence of SEQ ID NO: 38 is set forth in SEQ ID NO: 39, which is provided below.

[SEQ ID NO: 39]
GACCTGAACAAGGTGTTCCCACCCGAGGTCGCTGTGTTTGAGCCATCAGA
AGCAGAGATCTCCCACACCCAAAAGGCCACACTGGTGTGCCTGGCCACAG
GCTTCTTCCCCGACCACGTGGAGCTGAGCTGGTGGGTGAATGGGAAGGAG
GTGCACAGTGGGGTCAGCACAGACCCGCAGCCCCTCAAGGAGCAGCCCGC
CCTCAATGACTCCAGATACTGCCTGAGCAGCCGCCTGAGGGTCTCGGCCA
CCTTCTGGCAGAACCCCCGCAACCACTTCCGCTGTCAAGTCCAGTTCTAC
GGGCTCTCGGAGAATGACGAGTGGACCCAGGATAGGGCCAAACCCGTCAC
CCAGATCGTCAGCGCCGAGGCCTGGGGTAGAGCAGACTGTGGCTTTACCT
CGGTGTCCTACCAGCAAGGGGTCCTGTCTGCCACCATCCTCTATGAGATC
CTGCTAGGGAAGGCCACCCTGTATGCTGTGCTGGTCAGCGCCCTTGTGTT
GATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI GenBank ID: 28639, NG_001333.2, range 645749 to 647196 (TRBC1, SEQ ID NO: 40), NCBI GenBank ID: 28638, NG_001333.2 range 655095 to 656583 (TRBC2, SEQ ID NO:41) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 40. In certain embodiments, the TRBC polypeptide comprises or consists of the amino acid sequence encoded by the nucleotide sequence of SEQ ID NO: 41. SEQ ID NOs: 40 and 41 are provided below.

[SEQ ID NO: 40]
AGGACCTGAACAAGGTGTTCCCACCCGAGGTCGCTGTGTTTGAGCCATCA
GAAGCAGAGATCTCCCACACCCAAAAGGCCACACTGGTGTGCCTGGCCAC
AGGCTTCTTCCCCGACCACGTGGAGCTGAGCTGGTGGGTGAATGGGAAGG
AGGTGCACAGTGGGGTCAGCACAGACCCGCAGCCCCTCAAGGAGCAGCCC
GCCCTCAATGACTCCAGATACTGCCTGAGCAGCCGCCTGAGGGTCTCGGC
CACCTTCTGGCAGAACCCCCGCAACCACTTCCGCTGTCAAGTCCAGTTCT
ACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCT
GGGGAGATGCCTGGAGGAGATTAGGTGAGACCAGCTACCAGGGAAAATGG
AAAGATCCAGGTAGCAGACAAGACTAGATCCAAAAAGAAAGGAACCAGCG
CACACCATGAAGGAGAATTGGGCACCTGTGGTTCATTCTTCTCCCAGATT
CTCAGCCCAACAGAGCCAAGCAGCTGGGTCCCCTTTCTATGTGGCCTGTG
TAACTCTCATCTGGGTGGTGCCCCCCATCCCCCTCAGTGCTGCCACATGC
CATGGATTGCAAGGACAATGTGGCTGACATCTGCATGGCAGAAGAAAGGA
GGTGCTGGGCTGTCAGAGGAAGCTGGTCTGGGCCTGGGAGTCTGTGCCAA
CTGCAAATCTGACTTTACTTTTAATTGCCTATGAAAATAAGGTCTCTCAT
TTATTTTCCTCTCCCTGCTTTCTTTCAGACTGTGGCTTTACCTCGGGTAA
GTAAGCCCTTCCTTTTCCTCTCCCTCTCTCATGGTTCTTGACCTAGAACC
AAGGCATGAAGAACTCACAGACACTGGAGGGTGGAGGGTGGGAGAGACCA
GAGCTACCTGTGCACAGGTACCCACCTGTCCTTCCTCCGTGCCAACAGTG
TCCTACCAGCAAGGGGTCCTGTCTGCCACCATCCTCTATGAGATCCTGCT
AGGGAAGGCCACCCTGTATGCTGTGCTGGTCAGCGCCCTTGTGTTGATGG
CCATGGTAAGCAGGAGGGCAGGATGGGGCCAGCAGGCTGGAGGTGACACA
CTGACACCAAGCACCCAGAAGTATAGAGTCCCTGCCAGGATTGGAGCTGG
GCAGTAGGGAGGGAAGAGATTTCATTCAGGTGCCTCAGAAGATAACTTGC
ACCTCTGTAGGATCACAGTGGAAGGGTCATGCTGGGAAGGAGAAGCTGGA
GTCACCAGAAAACCCAATGGATGTTGTGATGAGCCTTACTATTTGTGTGG
TCAATGGGCCCTACTACTTTCTCTCAATCCTCACAACTCCTGGCTCTTAA
TAACCCCCAAAACTTTCTCTTCTGCAGGTCAAGAGAAAGGATTTCTGA
[SEQ ID NO: 41]
AGGACCTGAAAAACGTGTTCCCACCCGAGGTCGCTGTGTTTGAGCCATCA
GAAGCAGAGATCTCCCACACCCAAAAGGCCACACTGGTATGCCTGGCCAC
AGGCTTCTACCCCGACCACGTGGAGCTGAGCTGGTGGGTGAATGGGAAGG
AGGTGCACAGTGGGGTCAGCACAGACCCGCAGCCCCTCAAGGAGCAGCCC
GCCCTCAATGACTCCAGATACTGCCTGAGCAGCCGCCTGAGGGTCTCGGC
CACCTTCTGGCAGAACCCCCGCAACCACTTCCGCTGTCAAGTCCAGTTCT
ACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCT
GGGGAGATGCCTGGAGGAGATTAGGTGAGACCAGCTACCAGGGAAAATGG
AAAGATCCAGGTAGCGGACAAGACTAGATCCAGAAGAAAGCCAGAGTGGA
CAAGGTGGGATGATCAAGGTTCACAGGGTCAGCAAAGCACGGTGTGCACT
TCCCCCACCAAGAAGCATAGAGGCTGAATGGAGCACCTCAAGCTCATTCT
TCCTTCAGATCCTGACACCTTAGAGCTAAGCTTTCAAGTCTCCCTGAGGA
CCAGCCATACAGCTCAGCATCTGAGTGGTGTGCATCCCATTCTCTTCTGG
GGTCCTGGTTTCCTAAGATCATAGTGACCACTTCGCTGGCACTGGAGCAG
CATGAGGGAGACAGAACCAGGGCTATCAAAGGAGGCTGACTTTGTACTAT
CTGATATGCATGTGTTTGTGGCCTGTGAGTCTGTGATGTAAGGCTCAATG
TCCTTACAAAGCAGCATTCTCTCATCCATTTTTCTTCCCCTGTTTTCTTT
CAGACTGTGGCTTCACCTCCGGTAAGTGAGTCTCTCCTTTTTCTCTCTAT
CTTTCGCCGTCTCTGCTCTCGAACCAGGGCATGGAGAATCCACGGACACA
GGGGCGTGAGGGAGGCCAGAGCCACCTGTGCACAGGTGCCTACATGCTCT
GTTCTTGTCAACAGAGTCTTACCAGCAAGGGGTCCTGTCTGCCACCATCC
TCTATGAGATCTTGCTAGGGAAGGCCACCTTGTATGCCGTGCTGGTCAGT
GCCCTCGTGCTGATGGCCATGGTAAGGAGGAGGGTGGGATAGGGCAGATG
ATGGGGGCAGGGGATGGAACATCACACATGGGCATAAAGGAATCTCAGAG
CCAGAGCACAGCCTAATATATCCTATCACCTCAATGAAACCATAATGAAG
CCAGACTGGGGAGAAAATGCAGGGAATATCACAGAATGCATCATGGGAGG
ATGGAGACAACCAGCGAGCCCTACTCAAATTAGGCCTCAGAGCCCGCCTC
CCCTGCCCTACTCCTGCTGTGCCATAGCCCCTGAAACCCTGAAAATGTTC
TCTCTTCCACAGGTCAAGAGAAAGGATTCCAGAGGCTAG

In certain embodiments, the TCR like fusion molecule comprises a hinge/spacer region that links the first antigen binding chain to the constant domain. In certain embodiments, the TCR like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH2CH3 region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 42. In certain embodiments, the hinge/spacer region comprises or consists of amino acids 1 to 3 of the sequence set forth in SEQ ID NO: 42. An exemplary nucleic acid sequence encoding the amino acid sequence of SEQ ID NO: 42 is set forth in SEQ ID NO: 43. SEQ ID Nos: 42 and 43 are provided below.

| [SEQ ID NO: 42] | |
|---|---|
| | IPNIQNPDPA |
| [SEQ ID NO: 43] | |
| | ATTCCCAATATCCAGAACCCTGACCCTGCC |

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 44. In certain embodiments, the hinge/spacer region comprises or consists of amino acid 1 to 2 of the sequence set forth in SEQ ID NO: 44. An exemplary nucleic acid sequence encoding the amino acid sequence of SEQ ID NO: 44 is set forth in SEQ ID NO: 45. SEQ ID Nos: 44 and 45 are provided below.

| [SEQ ID NO: 44] | |
|---|---|
| | LEDLKNVEPPE |
| [SEQ ID NO: 45] | |
| | CTGGAGGATCTGAAAAACGTGTTCCCTCCTGAA |

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 8 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 8, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 8. In certain embodiments, the CD3ζ polypeptide comprises or consists of amino acids 52 to 164 of SEQ ID NO: 8.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 9 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 9.

In certain embodiments, the TCR like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR like fusion molecule is capable of associating with a CD3 complex (also known as “T-cell co-receptor”). In certain embodiments, the TCR like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR like fusion molecule replaces a native and/or an endogenous TCR in the CD3/TCR complex. In certain embodiments, the CD3 complex comprises a CD37 chain, a CD36 chain, and two CD3F chains.

In certain embodiments, the CD37 chain comprises or consists of an amino acid sequence that is at least about 85%, about 90%, about 95%, about 96%, about 97%, about 98%, about 99%, or about 100% homologous or identical to the amino acid sequence having an NCBI reference number: NP_000064.1 (SEQ ID NO: 46) or a fragment thereof, and/or may optionally comprise up to one or up to two or up to three conservative amino acid substitutions. SEQ ID NO: 46 is provided below.

[SEQ ID NO: 46]
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGFLFAEIVSIFVLAVGVYFIAGQDGVRQSRASDK
QTLLPNDQLYQPLKDREDDQYSHLQGNQLRRN

In certain embodiments, the CD36 chain comprises or consists of an amino acid sequence that is at least about 85%, about 90%, about 95%, about 96%, about 97%, about 98%, about 99% or about 100% homologous or identical to the amino acid sequence having an NCBI reference number: NP_000723.1 (SEQ ID NO: 47) or a fragment thereof, or the amino acid sequence having an NCBI reference number: NP_001035741.1 (SEQ ID NO: 48) or a fragment thereof, and/or may optionally comprise up to one or up to two or up to three conservative amino acid substitutions. SEQ ID NOS: 47 and 48 are provided below.

[SEQ ID NO: 47]
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
[SEQ ID NO: 48]
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQVYQPLRDRDDAQYSHLGGNWARNK

In certain embodiments, the CD3F chain comprises or consists of an amino acid sequence that is at least about 85%, about 90%, about 95%, about 96%, about 97%, about 98%, about 99%, or about 100% homologous or identical to the amino acid sequence having an NCBI reference number: NP_000724.1 (SEQ ID NO: 49) or a fragment thereof, and/or may optionally comprise up to one or up to two or up to three conservative amino acid substitutions. SEQ ID NO: 49 is provided below.

[SEQ ID NO: 49]
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

In certain embodiments, the TCR like fusion molecule exhibits a greater antigen sensitivity than a CAR targeting the same antigen. In certain embodiments, the TCR like fusion molecule is capable of inducing an immune response when binding to an antigen that has a low density on the surface of a tumor cell. In certain embodiments, cells comprising the TCR like fusion molecule can be used to treat a subject having tumor cells with a low expression level of a surface antigen, e.g., from a relapse of a disease, wherein the subject received treatment that leads to residual tumor cells. In certain embodiments, the tumor cells have a low density of a target molecule on the surface of the tumor cells. In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

Various TCR like fusion molecules are disclosed in International Patent Application Publication No. WO2019/133969, which is incorporated by reference hereby in its entirety.

### 5.3 Manufacturing of Induced T Cells (iT)

The presently disclosed subject matter provides methods and compositions for producing modified induced T cells (iT). The presently disclosed methods are based, in part, on the unexpected discovery that chimeric antigen receptors (CAR) or TCR like fusion proteins can improve the differentiation of pluripotent stem cells into induced T cells. Surprisingly, despite lacking expression of their endogenous TCR, induced T cells produced by the presently disclosed methods acquire conventional CD4 or CD8 T cell phenotype and do not show exhaustion markers. The presently disclosed subject matter provides methods for inducing differentiation of pluripotent stem cells, comprising introducing into the pluripotent stem cells a polynucleotide encoding a chimeric receptor (e.g., CAR).

### 5.3.1 Pluripotent Stem Cells

The presently disclosed subject matter provides methods for inducing differentiation of pluripotent stem cells, comprising obtaining and/or culturing of pluripotent stem cells.

As used herein, the term “pluripotent stem cells” refers to cells having the ability to form all lineage of the soma. For example, but without any limitation, embryonic stem cells are pluripotent stem cells capable to differentiate into cells from each of the three germ layers (e.g., ectoderm, mesoderm, endoderm). In certain embodiments, the pluripotent stem cells are induced pluripotent stem cells.

In certain embodiments, the pluripotent stem cell contains an introduced heterologous nucleic acid, where said nucleic acid may encode a desired nucleic acid or protein product or have informational value (see, for example, U.S. Pat. No. 6,312,911, which is incorporated by reference in its entirety). Non-limiting examples of protein products include markers detectable via in vivo imaging studies. Non-limiting examples of markers include fluorescent proteins (such as green fluorescent protein (GFP), blue fluorescent protein (EBFP, EBFP2, Azurite, mKalama1), cyan fluorescent protein (ECFP, Cerulean, CyPet, mTurquoise2), and yellow fluorescent protein derivatives (YFP, Citrine, Venus, YPet, EYFP)), β-galactosidase (LacZ), chloramphenicol acetyltransferase (cat), neomycin phosphotransferase (neo), enzymes (such as oxidases and peroxidases), and antigenic molecules. As used herein, the terms “reporter gene” or “reporter construct” refer to genetic constructs comprising a nucleic acid encoding a protein that is easily detectable or easily assayable, such as a colored protein, fluorescent protein such as GFP, or an enzyme such as beta-galactosidase (lacZ gene).

### 5.3.2 Delivery of the First Antigen-Recognizing Receptor into Pluripotent Stem Cells

The presently disclosed subject matter provides methods for inducing differentiation of pluripotent stem cells, comprising introducing nucleic acid compositions encoding a first antigen-recognizing receptor (e.g., a CAR disclosed in Section 5.2) into pluripotent stem cells.

Also provided are pluripotent stem cells comprising such nucleic acid compositions. In certain embodiments, the nucleic acid composition further comprises a promoter that is operably linked to the first antigen-recognizing receptor. In certain embodiments, the promoter is endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, the promoter is an inducible promoter. In certain embodiments, the inducible promoter is selected from a NFAT transcriptional response element (TRE) promoter, a CD69 promoter, a CD25 promoter, and an IL-2 promoter.

In certain embodiments, the nucleic acid composition is a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acid compositions can be administered to introduced and/or delivered into cells by art-known methods or as described herein. Genetic modification of a cell (e.g., a T cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter-specific for a target cell type of interest. Non-viral vectors may be used as well.

The polynucleotide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114, or GALV envelope and any other known in the art.

Possible methods of transduction also include direct co-culture of the pluripotent stem cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a pluripotent stem cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for the genetic modification of a pluripotent stem cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a pluripotent stem cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but are not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic nanoparticles, and cell-penetrating peptides).

In certain embodiments, the first antigen-recognizing receptor is delivered to the pluripotent stem cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the first antigen-recognizing receptor is delivered to the pluripotent stem cell by a non-viral method. Any targeted genome editing methods can also be used to deliver the first antigen-recognizing receptor to the pluripotent stem cell. In certain embodiments, the first antigen-recognizing receptor is delivered to the pluripotent stem cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the antigen-recognizing receptor to the pluripotent stem cell.

In certain embodiments, a CRISPR system is used to deliver the antigen-recognizing receptor to the pluripotent stem cell. Clustered regularly-interspaced short palindromic repeats (CRISPR) system is a genome-editing tool discovered in prokaryotic cells. When utilized for genome editing, the system includes Cas9 (a protein able to modify DNA utilizing crRNA as its guide), CRISPR RNA (crRNA, which contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a pluripotent stem cell. The repair template carrying the CAR expression cassette needs also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into pluripotent stem cells. In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises a CRISPRoff system. Additional details on the CRISPR systems of the presently disclosed subject matter can be found in Anzalone et al., Nature biotechnology 38.7 (2020): 824-844 and in Nunez et al., Cell 184.9 (2021): 2503-2519, the contents of each of which are incorporated by reference in their entireties.

In certain embodiments, zinc-finger nucleases are used to deliver the antigen-recognizing receptor to the pluripotent stem cell. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying the CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to deliver the antigen-recognizing receptor to the pluripotent stem cell. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In certain embodiments, upon delivery of the first antigen-recognizing receptor to the pluripotent stem cell, the first antigen-recognizing receptor is integrated at a locus within the genome of the pluripotent stem cell, e.g., a TRAC locus, a TRBC locus, a TRDC locus, or a TRGC locus. In certain embodiments, the locus is a TRAC locus. In certain embodiments, the expression of the first antigen-recognizing receptor is under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

### 5.3.3 Gene Disruption of TCR Locus

In certain embodiments, the presently disclosed methods further comprise introducing a gene disruption of a T cell receptor (TCR) locus. In certain embodiments, the gene disruption of the TCR locus results in a non-functional TCR. In certain embodiments, the gene disruption of the TCR locus results in knockout of the TCR gene expression.

Any methods to generate the gene disruption as disclosed in Section 5.3.2 can be used to generate the gene disruption of the TCR locus. In certain embodiments, the gene disruption of the TCR locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the TCR locus is a TRAC locus, a TRBC locus, a TRDC locus, or a TRGC locus. In certain embodiments, the TCR locus is a TRAC locus. In certain embodiments, the gene disruption of the TRAC locus can be a disruption of the coding region of the TRAC locus and/or a disruption of the non-coding region of the TRAC locus. In certain embodiments, the gene disruption of the TRAC locus comprises a disruption of the coding region of the TRAC locus. In certain embodiments, the gene disruption of the TRAC locus comprises an insertion at the coding region of the TRAC locus. In certain embodiments, the gene disruption of the TRAC locus comprises an insertion of an exogenous polynucleotide (e.g., encoding a CAR). In certain embodiments, the gene disruption of the TRAC locus comprises an in-frame insertion of an exogenous polynucleotide (e.g., encoding a CAR).

Upon delivery and insertion of an exogenous polynucleotide (e.g., encoding a CAR) into the TCR locus (e.g., TRAC locus) of the pluripotent stem cell, the expression of the exogenous polynucleotide (e.g., encoding a CAR) is under the control of an endogenous TCR promoter (e.g., TRAC promoter).

### 5.3.4 Cell Culture Media

The present disclosure provides methods for inducing differentiation of pluripotent stem cells including the first antigen-recognizing receptor, comprising contacting the pluripotent stem cells with a first cell culture medium to obtain a hematopoietic precursor. Furthermore, in certain embodiments, the present disclosure provides methods for inducing differentiation of hematopoietic precursors, comprising contacting the hematopoietic precursors with a second cell culture medium to obtain an induced T cell.

### 5.3.4.1 First Cell Culture Medium

The present disclosure provides methods for inducing differentiation of pluripotent stem cells, comprising contacting the pluripotent stem cells with a first cell culture medium to obtain a hematopoietic precursor.

In certain embodiments, the first cell culture medium comprises an activator of the bone morphogenic protein (BMP) pathway. Non-limiting examples of activators of the BMP pathway include BMP-2, BMP-4, isoliquiritigenin, 4′-hydroxychalcone, diosmetin, apigenin, biochanin A, biochanin A diacetate, luteolin, and BMP signaling agonist sb4. In certain embodiments, the activator of the BMP pathway is BMP-4.

In certain embodiments, the pluripotent stem cells are contacted with the activator of the BMP pathway for at least about 4 days, or at least about 10 days. In certain embodiments, the activator of the BMP pathway is added every day or every other day to the first cell culture medium comprising the pluripotent stem cells from day 0 through day 4. In certain embodiments, the activator of the BMP pathway is added every day or every other day to the first cell culture medium comprising the pluripotent stem cells from day 0 through day 10.

In certain embodiments, the concentration of the activator of the BMP pathway contacted with the pluripotent stem cells is between about 1 ng/ml and about 100 ng/ml, between about 10 ng/ml and about 100 ng/ml, between about 20 ng/ml and about 100 ng/ml, between about 30 ng/ml and about 100 ng/ml, between about 40 ng/ml and about 100 ng/ml, between about 50 ng/ml and about 100 ng/ml, between about 60 ng/ml and about 100 ng/ml, between about 70 ng/ml and about 100 ng/ml, between about 80 ng/ml and about 100 ng/ml, between about 90 ng/ml and about 100 ng/ml, between about 10 ng/ml and about 50 ng/ml, between 10 ng/ml and about 40 ng/ml, between 10 ng/ml and about 30 ng/ml, between 10 ng/ml and about 20 ng/ml, between about 20 ng/ml and about 50 ng/ml, between about 30 ng/ml and about 50 ng/ml, between about 20 ng/ml and about 40 ng/ml, or between about 25 ng/ml and about 35 ng/ml. In certain embodiments, the concentration of the activator of the BMP pathway contacted with the pluripotent stem cells is about 30 ng/ml. In certain embodiments, the concentration of the activator of the BMP pathway contacted with the pluripotent stem cells is about 5 ng/ml.

In certain embodiments, the first cell culture medium further comprises a fibroblast growth factor (FGF). In certain embodiments, the FGF is basic fibroblast growth factor (bFGF).

In certain embodiments, the pluripotent stem cells are contacted with the FGF for at least about 4 days, or at least about 10 days. In certain embodiments, the FGF is added every day or every other day to the first cell culture medium comprising the pluripotent stem cells from day 0 through day 4. In certain embodiments, the FGF is added every day or every other day to the first cell culture medium comprising the pluripotent stem cells from day 0 through day 10.

In certain embodiments, the concentration of the FGF contacted with the pluripotent stem cells is between about 1 ng/ml and about 100 ng/ml, between about 1 ng/ml and about 10 ng/ml, between about 2 ng/ml and about 10 ng/ml, between about 3 ng/ml and about 10 ng/ml, between about 4 ng/ml and about 10 ng/ml, between about 5 ng/ml and about 10 ng/ml, between about 6 ng/ml and about 10 ng/ml, between about 7 ng/ml and about 10 ng/ml, between about 8 ng/ml and about 10 ng/ml, between about 9 ng/ml and about 10 ng/ml, between about 1 ng/ml and about 5 ng/ml, between about 2 ng/ml and about 5 ng/ml, between about 3 ng/ml and about 5 ng/ml, between about 4 ng/ml and about 5 ng/ml, or between about 4 ng/ml and about 6 ng/ml. In certain embodiments, the concentration of the FGF contacted with the pluripotent stem cells is about 5 ng/ml. In certain embodiments, the concentration of the FGF contacted with the pluripotent stem cells is about 10 ng/ml.

In certain embodiments, the first cell culture medium further comprises hematopoietic cytokines. Non-limiting example of hematopoietic cytokines include SCF, FLT3L, IL-3, VEGF, IL-6, IL-11, TPO, IGF-1, EPO, SHH, angiotensin II, and AGTR1.

In certain embodiments, the pluripotent stem cells are contacted with the hematopoietic cytokines for at least about 4 days, or at least about 6 days. In certain embodiments, the hematopoietic cytokines are added every day or every other day to the first cell culture medium comprising the pluripotent stem cells from day 4 through day 10. In certain embodiments, the hematopoietic cytokines are added every day or every other day to the first cell culture medium comprising the pluripotent stem cells from day 6 through day 10.

In certain embodiments, the hematopoietic cytokine is SCF. In certain embodiments, the concentration of SCF contacted with the pluripotent stem cells is between about 10 ng/ml and about 250 ng/ml, between about 50 ng/ml and about 250 ng/ml, between about 100 ng/ml and about 250 ng/ml, between about 150 ng/ml and about 250 ng/ml, between about 200 ng/ml and about 250 ng/ml, between about 50 ng/ml and about 200 ng/ml, between about 50 ng/ml and about 150 ng/ml, between about 50 ng/ml and about 100 ng/ml, or between about 75 ng/ml and about 125 ng/ml. In certain embodiments, the concentration of SCF contacted with the pluripotent stem cells is about 100 ng/ml. In certain embodiments, the concentration of SCF contacted with the pluripotent stem cells is about 50 ng/ml.

In certain embodiments, the hematopoietic cytokine is FLT3L. In certain embodiments, the concentration of FLT3L contacted with the pluripotent stem cells is between about 1 ng/ml and about 100 ng/ml, between about 10 ng/ml and about 100 ng/ml, between about 20 ng/ml and about 100 ng/ml, between about 10 ng/ml and about 50 ng/ml, between about 10 ng/ml and about 40 ng/ml, between about 10 ng/ml and about 30 ng/ml, between about 15 ng/ml and about 30 ng/ml, or between about 15 ng/ml and about 25 ng/ml. In certain embodiments, the concentration of FLT3L contacted with the pluripotent stem cells is about 20 ng/ml.

In certain embodiments, the hematopoietic cytokine is IL-3. In certain embodiments, the concentration of IL-3 contacted with the pluripotent stem cells is between about 1 ng/ml and about 100 ng/ml, between about 10 ng/ml and about 100 ng/ml, between about 20 ng/ml and about 100 ng/ml, between about 10 ng/ml and about 50 ng/ml, between about 10 ng/ml and about 40 ng/ml, between about 10 ng/ml and about 30 ng/ml, between about 15 ng/ml and about 30 ng/ml, or between about 15 ng/ml and about 25 ng/ml. In certain embodiments, the concentration of IL-3 contacted with the pluripotent stem cells is about 20 ng/ml.

In certain embodiments, the hematopoietic cytokine is VEGF. In certain embodiments, the concentration of VEGF contacted with the pluripotent stem cells is between about 1 ng/ml and about 100 ng/ml, between about 10 ng/ml and about 100 ng/ml, between about 20 ng/ml and about 100 ng/ml, between about 10 ng/ml and about 50 ng/ml, between about 10 ng/ml and about 40 ng/ml, between about 10 ng/ml and about 30 ng/ml, between about 15 ng/ml and about 30 ng/ml, or between about 15 ng/ml and about 25 ng/ml. In certain embodiments, the concentration of VEGF contacted with the pluripotent stem cells is about 20 ng/ml. In certain embodiments, the concentration of VEGF contacted with the pluripotent stem cells is about 10 ng/ml.

In certain embodiments, the hematopoietic cytokine is IL-6. In certain embodiments, the concentration of IL-6 contacted with the pluripotent stem cells is between about 1 ng/ml and about 100 ng/ml, between about 10 ng/ml and about 100 ng/ml, between about 20 ng/ml and about 100 ng/ml, between about 10 ng/ml and about 50 ng/ml, between about 10 ng/ml and about 40 ng/ml, between about 10 ng/ml and about 30 ng/ml, between about 15 ng/ml and about 30 ng/ml, or between about 15 ng/ml and about 25 ng/ml. In certain embodiments, the concentration of IL-6 contacted with the pluripotent stem cells is about 10 ng/ml.

In certain embodiments, the hematopoietic cytokine is IL-11. In certain embodiments, the concentration of IL-11 contacted with the pluripotent stem cells is between about 1 ng/ml and about 100 ng/ml, between about 10 ng/ml and about 100 ng/ml, between about 20 ng/ml and about 100 ng/ml, between about 10 ng/ml and about 50 ng/ml, between about 10 ng/ml and about 40 ng/ml, between about 10 ng/ml and about 30 ng/ml, between about 15 ng/ml and about 30 ng/ml, or between about 15 ng/ml and about 25 ng/ml. In certain embodiments, the concentration of IL-11 contacted with the pluripotent stem cells is about 10 ng/ml.

### 5.3.4.2 Second Cell Culture Medium

In certain embodiments, the methods disclosed herein also comprise contacting the hematopoietic precursor (e.g., obtained using the first cell culture medium described in Section 5.3.4.1) with a second cell culture medium to obtain an induced T cell.

In certain embodiments, the second cell culture medium comprises Notch ligand. In certain embodiments, the Notch ligand is a polypeptide or a functional fragment thereof capable of activating the Notch intracellular signaling. In certain embodiments, the Notch ligand is selected from the group consisting of a DLL-4 polypeptide, a JAG-1 polypeptide, a DLL-1 polypeptide, a JAG-2 polypeptide, and a combination thereof. In certain embodiments, the Notch ligand is a DLL-4 polypeptide. In certain embodiments, the DLL-4 polypeptide comprises or consists of an amino acid sequence that is at least about 80% identical, at least about 85% identical, at least about 90% identical, at least about 95% identical, at least about 96% identical, at least about 97% identical, at least about 98% identical, at least about 99% identical to the amino acid sequence set forth in SEQ ID NO: 50. In certain embodiments, the DLL-4 polypeptide comprises or consists of the amino acid sequence set forth in SEQ ID NO: 50, which is outlined below:

[SEQ ID NO: 50]
MAAASRSASGWALLLLVALWQQRAAGSGVFQLQLQEFINERGVLASGRPC
EPGCRTFFRVCLKHFQAVVSPGPCTFGTVSTPVLGTNSFAVRDDSSGGGR
NPLQLPFNFTWPGTFSLIIEAWHAPGDDLRPEALPPDALISKIAIQGSLA
VGQNWLLDEQTSTLTRLRYSYRVICSDNYYGDNCSRLCKKRNDHFGHYVC
QPDGNLSCLPGWTGEYCQQPICLSGCHEQNGYCSKPAECLCRPGWQGRLC
NECIPHNGCRHGTCSTPWQCTCDEGWGGLFCDQDLNYCTHHSPCKNGATC
SNSGQRSYTCTCRPGYTGVDCELELSECDSNPCRNGGSCKDQEDGYHCLC
PPGYYGLHCEHSTLSCADSPCFNGGSCRERNQGANYACECPPNFTGSNCE
KKVDRCTSNPCANGGQCLNRGPSRMCRCRPGFTGTYCELHVSDCARNPCA
HGGTCHDLENGLMCTCPAGFSGRRCEVRTSIDACASSPCFNRATCYTDLS
TDTFVCNCPYGFVGSRCEFPVGLPPSFPWVAVSLGVGLAVLLVLLGMVAV
AVRQLRLRRPDDGSREAMNNLSDFQKDNLIPAAQLKNTNQKKELEVDCGL
DKSNCGKQQNHTLDYNLAPGPLGRGTMPGKFPHSDKSLGEKAPLRLHSEK
PECRISAICSPRDSMYQSVCLISEERNECVIATEV

In certain embodiments, the Notch ligand is a JAG-1 polypeptide. In certain embodiments the JAG-1 polypeptide comprises or consists of an amino acid sequence that is at least about 80% identical, at least about 85% identical, at least about 90% identical, at least about 95% identical, at least about 96% identical, at least about 97% identical, at least about 98% identical, at least about 99% identical to the amino acid sequence set forth in SEQ ID NO: 51. In certain embodiments, the JAG-1 polypeptide comprises or consists of the amino acid sequence set forth in SEQ ID NO: 51, which is outlined below:

[SEQ ID NO: 51]
MRSPRTRGRSGRPLSLLLALLCALRAKVCGASGQFELEILSMQNVNGELQ
NGNCCGGARNPGDRKCTRDECDTYFKVCLKEYQSRVTAGGPCSFGSGSTP
VIGGNTFNLKASRGNDRNRIVLPFSFAWPRSYTLLVEAWDSSNDTVQPDS
IIEKASHSGMINPSRQWQTLKQNTGVAHFEYQIRVTCDDYYYGFGCNKFC
RPRDDFFGHYACDQNGNKTCMEGWMGPECNRAICRQGCSPKHGSCKLPGD
CRCQYGWQGLYCDKCIPHPGCVHGICNEPWQCLCETNWGGQLCDKDLNYC
GTHQPCLNGGTCSNTGPDKYQCSCPEGYSGPNCEIAEHACLSDPCHNRGS
CKETSLGFECECSPGWTGPTCSTNIDDCSPNNCSHGGTCQDLVNGFKCVC
PPQWTGKTCQLDANECEAKPCVNAKSCKNLIASYYCDCLPGWMGQNCDIN
INDCLGQCQNDASCRDLVNGYRCICPPGYAGDHCERDIDECASNPCLNGG
HCQNEINRFQCLCPTGFSGNLCQLDIDYCEPNPCQNGAQCYNRASDYFCK
CPEDYEGKNCSHLKDHCRTTPCEVIDSCTVAMASNDTPEGVRYISSNVCG
PHGKCKSQSGGKFTCDCNKGFTGTYCHENINDCESNPCRNGGTCIDGVNS
YKCICSDGWEGAYCETNINDCSQNPCHNGGTCRDLVNDFYCDCKNGWKGK
TCHSRDSQCDEATCNNGGTCYDEGDAFKCMCPGGWEGTTCNIARNSSCLP
NPCHNGGTCVVNGESFTCVCKEGWEGPICAQNTNDCSPHPCYNSGTCVDG
DNWYRCECAPGFAGPDCRININECQSSPCAFGATCVDEINGYRCVCPPGH
SGAKCQEVSGRPCITMGSVIPDGAKWDDDCNTCQCLNGRIACSKVWCGPR
PCLLHKGHSECPSGQSCIPILDDQCFVHPCTGVGECRSSSLQPVKTKCTS
DSYYQDNCANITFTFNKEMMSPGLTTEHICSELRNLNILKNVSAEYSIYI
ACEPSPSANNEIHVAISAEDIRDDGNPIKEITDKIIDLVSKRDGNSSLIA
AVAEVRVQRRPLKNRTDFLVPLLSSVLTVAWICCLVTAFYWCLRKRRKPG
SHTHSASEDNTTNNVREQLNQIKNPIEKHGANTVPIKDYENKNSKMSKIR
THNSEVEEDDMDKHQQKARFAKQPAYTLVDREEKPPNGTPTKHPNWTNKQ
DNRDLESAQSLNRMEYIV

In certain embodiments, the Notch ligand is expressed by a feeder cell. In certain embodiments, the feeder cell is an OP-9 cell. In certain embodiments, the feeder cell is transduced with a polynucleotide encoding a DLL-4 polypeptide, a JAG-1 polypeptide, a DLL-1 polypeptide, a JAG-2 polypeptide, or a combination thereof. In certain embodiments, the polynucleotide encodes a DLL-4 polypeptide. In certain embodiments, the DLL-4 polypeptide comprises or consists of an amino acid sequence that is at least about 80% identical, at least about 85% identical, at least about 90% identical, at least about 95% identical, at least about 96% identical, at least about 97% identical, at least about 98% identical, at least about 99% identical to the amino acid sequence set forth in SEQ ID NO: 50. In certain embodiments, the DLL-4 polypeptide comprises or consists of the amino acid sequence set forth in SEQ ID NO: 50.

In certain embodiments, the polynucleotide encodes a JAG-1 polypeptide. In certain embodiments, the JAG-1 polypeptide comprises or consists of an amino acid sequence that is at least about 80% identical, at least about 85% identical, at least about 90% identical, at least about 95% identical, at least about 96% identical, at least about 97% identical, at least about 98% identical, at least about 99% identical to the amino acid sequence set forth in SEQ ID NO: 51. In certain embodiments, the JAG-1 polypeptide comprises or consists of the amino acid sequence set forth in SEQ ID NO: 51.

In certain embodiments, the hematopoietic precursors are contacted with the Notch ligand for up to about 10 days, or up to about 25 days. For example, but without any limitation, the hematopoietic precursors are co-cultured with feeder cells expressing the Notch ligand for up to about 10 days or up to about 25 days.

In certain embodiments, the second cell culture medium further comprises growth factors and cytokines. Non-limiting examples of growth factors and cytokines of the second cell culture medium are SCF, FLT3L, IL-3, IL-7, IL-6, IL-11, TPO, IGF-1, EPO, SHH, angiotensin II, and AGTR1.

In certain embodiments, the second cell culture medium further comprises SCF. In certain embodiments, the concentration of SCF contacted with the hematopoietic precursors is between about 10 ng/ml and about 100 ng/ml, between about 10 ng/ml and about 90 ng/ml, between about 10 ng/ml and about 80 ng/ml, between about 10 ng/ml and about 70 ng/ml, between about 10 ng/ml and about 60 ng/ml, between about 10 ng/ml and about 50 ng/ml, between about 10 ng/ml and about 40 ng/ml, between about 20 ng/ml and about 50 ng/ml, or between about 20 ng/ml and about 40 ng/ml. In certain embodiments, the concentration of SCF contacted with the hematopoietic precursors is about 30 ng/ml. In certain embodiments, the hematopoietic precursors are contacted with SCF for at least about 10 days, or at least about 25 days. In certain embodiments, SCF is added every day or every other day to the second cell culture medium comprising the hematopoietic precursors from day 1 through day 10. In certain embodiments, SCF is added every day or every other day to the second cell culture medium comprising the hematopoietic precursors from day 11 through day 25.

In certain embodiments, the second cell culture medium further comprises TPO. In certain embodiments, the concentration of TPO contacted with the hematopoietic precursors is between about 1 ng/ml and about 100 ng/ml, between about 1 ng/ml and about 50 ng/ml, between about 1 ng/ml and about 25 ng/ml, between about 1 ng/ml and about 20 ng/ml, between about 1 ng/ml and about 15 ng/ml, between about 2.5 ng/ml and about 15 ng/ml, between about 5 ng/ml and about 15 ng/ml, between about 7.5 ng/ml and about 15 ng/ml, or between about 7.5 ng/ml and about 12.5 ng/ml. In certain embodiments, the concentration of TPO contacted with the hematopoietic precursors is about 10 ng/ml. In certain embodiments, the hematopoietic precursors are contacted with TPO for at least about 10 days. In certain embodiments, TPO is added every day or every other day to the second cell culture medium comprising the hematopoietic precursors from day 1 through day 10.

In certain embodiments, the second cell culture medium further comprises IL-3. In certain embodiments, the concentration of IL-3 contacted with the hematopoietic precursors is between about 1 ng/ml and about 100 ng/ml, between about 1 ng/ml and about 50 ng/ml, between about 1 ng/ml and about 25 ng/ml, between about 1 ng/ml and about 20 ng/ml, between about 1 ng/ml and about 15 ng/ml, between about 1 ng/ml and about 10 ng/ml, between about 1 ng/ml and about 7.5 ng/ml, between about 1 ng/ml and about 5 ng/ml, or between about 2.5 ng/ml and about 7.5 ng/ml. In certain embodiments, the concentration of IL-3 contacted with the hematopoietic precursors is about 5 ng/ml. In certain embodiments, the hematopoietic precursors are contacted with IL-3 for at least about 10 days. In certain embodiments, IL-3 is added every day or every other day to the second cell culture medium comprising the hematopoietic precursors from day 1 through day 10.

In certain embodiments, the second cell culture medium further comprises IL-7. In certain embodiments, the concentration of IL-7 contacted with the hematopoietic precursors is between about 1 ng/ml and about 100 ng/ml, between about 1 ng/ml and about 75 ng/ml, between about 1 ng/ml and about 50 ng/ml, between about 1 ng/ml and about 25 ng/ml, between about 1 ng/ml and about 20 ng/ml, between about 1 ng/ml and about 15 ng/ml, between about 1 ng/ml and about 10 ng/ml, between about 5 ng/ml and about 15 ng/ml, or between about 2.5 ng/ml and about 7.5 ng/ml. In certain embodiments, the concentration of IL-7 contacted with the hematopoietic precursors is about 10 ng/ml. In certain embodiments, the hematopoietic precursors are contacted with IL-7 for at least about 10 days, or at least about 25 days. In certain embodiments, IL-7 is added every day or every other day to the second cell culture medium comprising the hematopoietic precursors from day 1 through day 10. In certain embodiments, IL-7 is added every day or every other day to the second cell culture medium comprising the hematopoietic precursors from day 11 through day 25.

In certain embodiments, the second cell culture medium further comprises FLT3L. In certain embodiments, the concentration of FLT3L contacted with the hematopoietic precursors is between about 1 ng/ml and about 100 ng/ml, between about 1 ng/ml and about 75 ng/ml, between about 1 ng/ml and about 50 ng/ml, between about 1 ng/ml and about 25 ng/ml, between about 1 ng/ml and about 20 ng/ml, between about 1 ng/ml and about 15 ng/ml, between about 1 ng/ml and about 10 ng/ml, between about 5 ng/ml and about 15 ng/ml, or between about 2.5 ng/ml and about 7.5 ng/ml. In certain embodiments, the concentration of FLT3L contacted with the hematopoietic precursors is about 10 ng/ml. In certain embodiments, the hematopoietic precursors are contacted with FLT3L for at least about 10 days, or at least about 25 days. In certain embodiments, FLT3L is added every day or every other day to the second cell culture medium comprising the pluripotent stem cells from day 1 through day 10. In certain embodiments, FLT3L is added every day or every other day to the second cell culture medium comprising the hematopoietic precursors from day 11 through day 25.

### 5.3.5 Stimulation and Expansion

As outlined above, pluripotent stem cells are contacted with cell culture media (e.g., the ones described herein) to differentiate and grow into induced T cells. To achieve sufficient therapeutic doses for adoptive cell therapies, the presently disclosed induced T cells are subjected to one or more rounds of stimulation and expansion. In certain embodiments, the presently disclosed subject matter provides methods including stimulating and expanding induced T cells. In certain embodiments, the presently disclosed subject matter provides a method of expanding a population of induced T cells.

In certain embodiments, stimulation and expansion can be achieved by contacting the induced T cells including the CAR with a polypeptide that engages the CAR. In certain embodiments, the polypeptide that engages the CAR is an antibody or an antigen-binding fragment thereof.

In certain embodiments, stimulation and expansion can be achieved by contacting the induced T cells including the CAR with a polypeptide that stimulates the CAR. In certain embodiments, the polypeptide that stimulates the CAR is an antibody or an antigen-binding fragment thereof.

In certain embodiments, stimulation and expansion can be achieved by contacting the induced T cells including the CAR with a polypeptide that activates the CAR. In certain embodiments, the polypeptide that activates the CAR is an antibody or an antigen-binding fragment thereof.

In certain embodiments, the antibody or antigen-binding fragment thereof binds to a extracellular antigen-binding domain of the CAR. In certain embodiments, the antibody or antigen-binding fragment thereof binds to a scFv of the CAR.

In certain non-limiting embodiments, the polypeptide that activates a CAR can be a 19E3 antibody or an antigen-binding fragment thereof. In certain non-limiting embodiments, the polypeptide that activates a CAR can be a 12D11 antibody or an antigen-binding fragment thereof.

In certain non-limiting embodiments, the polypeptide that activates a CAR can be a 22G3 antibody or an antigen-binding fragment thereof.

In certain embodiments, the antibody or antigen-binding fragment thereof binds to an idiotypic variable domain of the scFv of the CAR. Additional information on antibody or antigen-binding fragment thereof binding to an idiotypic variable domain can be found in Naveed et al., Translational Medicine Communications 3, no. 1 (2018): 1-7, which is incorporated herein by reference in its entirety.

In certain embodiments, the polypeptide that engages the CAR is an antigen-containing polypeptide. In certain embodiments, the antigen-containing polypeptide can be a recombinant antigen polypeptide or a fragment thereof (e.g., one disclosed in Section 5.2). For example, but without any limitation, the recombinant antigen polypeptide can be a CD19 polypeptide or a fragment thereof that can engage the CAR.

In certain embodiments, the polypeptide that stimulates the CAR is an antigen-containing polypeptide. In certain embodiments, the antigen-containing polypeptide can be a recombinant antigen polypeptide or a fragment thereof (e.g., one disclosed in Section 5.2). For example, but without any limitation, the recombinant antigen polypeptide can be a CD19 polypeptide or a fragment thereof that can engage the CAR.

In certain embodiments, the polypeptide that activates the CAR is an antigen-containing polypeptide. In certain embodiments, the antigen-containing polypeptide can be a recombinant antigen polypeptide or a fragment thereof (e.g., one disclosed in Section 5.2). For example, but without any limitation, the recombinant antigen polypeptide can be a CD19 polypeptide or a fragment thereof that can engage the CAR.

In certain embodiments, the antigen-containing polypeptide can be an Fc-fusion protein. In certain embodiments, the Fc-fusion protein includes a Fc fragment linked with a recombinant antigen polypeptide or a fragment thereof (e.g., one disclosed in Section 5.2). For example, but without any limitation, the Fc-fusion protein can be a recombinant CD19-Fc chimera protein (R&D System®, no. 9269-CD).

In certain embodiments, the presently disclosed methods further comprise contacting the induced T cells including the CAR with an antibody that binds to 4-1BB. In certain embodiments, the antibody that binds to 4-1BB is a humanized antibody. In certain embodiments, the antibody that binds to 4-1BB is urelumab. In certain embodiments, the urelumab comprises a heavy chain and a light chain. In certain embodiments, the heavy chain of urelumab comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence of SEQ ID NO: 54. In certain embodiments, the heavy chain of urelumab comprises to the amino acid sequence of SEQ ID NO: 54. SEQ ID NO: 54 is outlined below:

[SEQ ID NO: 54]
QVQLQQWGAGLLKPSETLSLTCAVYGGSFSGYYWSWIRQSPEKGLEWIGE
INHGGYVTYNPSLESRVTISVDTSKNQFSLKLSSVTAADTAVYYCARDYG
PGNYDWYFDLWGRGTLVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK

In certain embodiments, the light chain of urelumab comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence of SEQ ID NO: 55. In certain embodiments, the light chain of urelumab comprises to the amino acid sequence of SEQ ID NO: 55. SEQ ID NO: 55 is outlined below:

[SEQ ID NO: 55]
EIVLTQSPATLSLSPGERATLSCRASQSVSSYLAWYQQKPGQAPRLLIYD
ASNRATGIPARFSGSGSGTDFTLTISSLEPEDFAVYYCQQRSNWPPALTF
CGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTH
QGLSSPVTKSFNRGEC

In certain embodiments, the concentration of urelumab is between about 1 ng/ml and about 20 ng/ml, between about 1 μg/ml and about 15 μg/ml, between about 1 μg/ml and about 10 μg/ml, between about 1 μg/ml and about 7.5 μg/ml, between about 1 μg/ml and about 5 μg/ml, between about 2 μg/ml and about 10 μg/ml, between about 1 μg/ml and about 7.5 μg/ml, between about 2 μg/ml and about 5 μg/ml, or between about 2 μg/ml and about 4 μg/ml. In certain embodiments, the concentration of urelumab contacted with the induced T cells is about 3 μg/ml.

In certain embodiments, the presently disclosed methods further comprise contacting the induced T cells including the CAR with IL-7. In certain embodiments, the concentration of IL-7 is between about 1 ng/ml and about 100 ng/ml, between about 1 ng/ml and about 75 ng/ml, between about 1 ng/ml and about 50 ng/ml, between about 1 ng/ml and about 25 ng/ml, between about 1 ng/ml and about 20 ng/ml, between about 1 ng/ml and about 15 ng/ml, between about 1 ng/ml and about 10 ng/ml, between about 5 ng/ml and about 15 ng/ml, or between about 2.5 ng/ml and about 7.5 ng/ml. In certain embodiments, the concentration of IL-7 contacted with the induced T cells is about 5 ng/ml.

In certain embodiments, the presently disclosed methods further comprise contacting the induced T cells including the CAR with IL-21. In certain embodiments, the concentration of IL-21 is between about 10 ng/ml and about 100 ng/ml, between about 10 ng/ml and about 90 ng/ml, between about 10 ng/ml and about 80 ng/ml, between about 10 ng/ml and about 70 ng/ml, between about 10 ng/ml and about 60 ng/ml, between about 10 ng/ml and about 50 ng/ml, between about 10 ng/ml and about 40 ng/ml, between about 20 ng/ml and about 50 ng/ml, or between about 20 ng/ml and about 40 ng/ml. In certain embodiments, the concentration of IL-21 contacted with the induced T cells is about 25 ng/ml.

In certain embodiments, stimulation and expansion can be achieved by contacting the induced T cells including the HIT with a polypeptide that engages the HIT. In certain embodiments, the polypeptide that engages the HIT is an antibody or an antigen-binding fragment thereof.

In certain embodiments, stimulation and expansion can be achieved by contacting the induced T cells including the HIT with a polypeptide that stimulates the HIT. In certain embodiments, the polypeptide that stimulates the HIT is an antibody or an antigen-binding fragment thereof.

In certain embodiments, stimulation and expansion can be achieved by contacting the induced T cells including the HIT with a polypeptide that activates the HIT. In certain embodiments, the polypeptide that activates the HIT is an antibody or an antigen-binding fragment thereof.

In certain embodiments, the antibody or antigen-binding fragment thereof binds to an antigen-binding domain of the HIT. In certain embodiments, the antibody or antigen-binding fragment thereof binds to an scFv of the HIT. In certain non-limiting embodiments, the polypeptide that activates a HIT can be a 19E3 antibody or an antigen-binding fragment thereof. In certain non-limiting embodiments, the polypeptide that activates a HIT can be a 12D11 antibody or an antigen-binding fragment thereof. In certain non-limiting embodiments, the polypeptide that activates a HIT can be a 22G3 antibody or an antigen-binding fragment thereof.

In certain embodiments, the antibody or antigen-binding fragment thereof binds to an idiotypic variable domain of the first antigen-binding chain of the HIT or the second antigen-binding chain of the HIT. In certain embodiments, the antibody or antigen-binding fragment thereof binds to an idiotypic variable domain of the first antigen-binding chain of the HIT and the second antigen-binding chain of the HIT. Additional information on antibody or antigen-binding fragment thereof binding to an idiotypic variable domain can be found in Naveed et al., Translational Medicine Communications 3, no. 1 (2018): 1-7, which is incorporated herein by reference in its entirety.

In certain embodiments, the polypeptide that engages the HIT is an antigen-containing polypeptide. In certain embodiments, the antigen-containing polypeptide can be a recombinant antigen polypeptide or a fragment thereof (e.g., one disclosed in Section 5.2). For example, but without any limitation, the recombinant antigen polypeptide can be a CD19 polypeptide or a fragment thereof that can engage the HIT.

In certain embodiments, the polypeptide that stimulates the HIT is an antigen-containing polypeptide. In certain embodiments, the antigen-containing polypeptide can be a recombinant antigen polypeptide or a fragment thereof (e.g., one disclosed in Section 5.2). For example, but without any limitation, the recombinant antigen polypeptide can be a CD19 polypeptide or a fragment thereof that can engage the HIT.

In certain embodiments, the polypeptide that activates the HIT is an antigen-containing polypeptide. In certain embodiments, the antigen-containing polypeptide can be a recombinant antigen polypeptide or a fragment thereof (e.g., one disclosed in Section 5.2). For example, but without any limitation, the recombinant antigen polypeptide can be a CD19 polypeptide or a fragment thereof that can engage the HIT.

In certain embodiments, the antigen-containing polypeptide can be an Fc-fusion protein. In certain embodiments, the Fc-fusion protein includes an Fc fragment linked with a recombinant antigen polypeptide or a fragment thereof (e.g., one disclosed in Section 5.2). For example, but without any limitation, the Fc-fusion protein can be a recombinant CD19-Fc chimera protein (R&D System®, no. 9269-CD).

In certain embodiments, the presently disclosed methods further comprise contacting the induced T cells including the HIT with an antibody that binds to 4-1BB. In certain embodiments, the antibody that binds to 4-1BB is a humanized antibody. In certain embodiments, the antibody that binds to 4-1BB is urelumab. In certain embodiments, the urelumab comprises a heavy chain and a light chain. In certain embodiments, the heavy chain of urelumab comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence of SEQ ID NO: 54. In certain embodiments, the heavy chain of urelumab comprises to the amino acid sequence of SEQ ID NO: 54. In certain embodiments, the light chain of urelumab comprises an amino acid sequence that is at least about 80%, at least about 85%, at least about 90%, at least about 95%, at least about 96%, at least about 97%, at least about 98%, or at least about 99%, at least about 100% homologous or identical to the amino acid sequence of SEQ ID NO: 55. In certain embodiments, the light chain of urelumab comprises to the amino acid sequence of SEQ ID NO: 55. In certain embodiments, the concentration of urelumab is between about 1 ng/ml and about 20 ng/ml. In certain embodiments, the concentration of urelumab contacted with the induced T cells is about 3 μg/ml.

In certain embodiments, the presently disclosed methods further comprise contacting the induced T cells including the HIT with IL-7. In certain embodiments, the concentration of IL-7 is between about 1 ng/ml and about 100 ng/ml. In certain embodiments, the concentration of IL-7 contacted with the induced T cells is about 5 ng/ml. In certain embodiments, the presently disclosed methods further comprise contacting the induced T cells including the HIT with IL-21. In certain embodiments, the concentration of IL-21 is between about 10 ng/ml and about 100 ng/ml. In certain embodiments, the concentration of IL-21 contacted with the induced T cells is about 25 ng/ml.

### 5.3.5.1. Antibodies and Antigen-Binding Thereof

In certain embodiments, stimulation and expansion can be achieved by contacting the induced T cells including the CAR with a polypeptide that engages the CAR. In certain embodiments, the polypeptide that engages the CAR is an antibody or an antigen-binding fragment thereof.

In certain embodiments, stimulation and expansion can be achieved by contacting the induced T cells including the CAR with a polypeptide that stimulates the CAR. In certain embodiments, the polypeptide that stimulates the CAR is an antibody or an antigen-binding fragment thereof.

In certain embodiments, stimulation and expansion can be achieved by contacting the induced T cells including the CAR with a polypeptide that activates the CAR. In certain embodiments, the polypeptide that activates the CAR is an antibody or an antigen-binding fragment thereof.

In certain embodiments, stimulation and expansion can be achieved by contacting the induced T cells including the HIT with a polypeptide that engages the HIT. In certain embodiments, the polypeptide that engages the HIT is an antibody or an antigen-binding fragment thereof.

In certain embodiments, stimulation and expansion can be achieved by contacting the induced T cells including the HIT with a polypeptide that stimulates the HIT. In certain embodiments, the polypeptide that stimulates the HIT is an antibody or an antigen-binding fragment thereof.

In certain embodiments, stimulation and expansion can be achieved by contacting the induced T cells including the HIT with a polypeptide that activates the HIT. In certain embodiments, the polypeptide that activates the HIT is an antibody or an antigen-binding fragment thereof.

In certain embodiments, the antibody or an antigen-binding fragment thereof that engages, stimulates, or activates the chimeric receptor (e.g., a CAR) is an scFv, an scFv-Fc fusion protein, or a full-length human IgG with V_{H} and V_{L} regions or CDRs selected from Table 1. In certain embodiments, the antibody or an antigen-binding fragment thereof is designated as “CAR1” or “19E3”.

In certain embodiments, the antibody or an antigen-binding fragment thereof comprises a V_{H} comprising a CDR1 comprising the amino acid sequence set forth in SEQ ID NO: 60 or a conservative modification thereof, a CDR2 comprising the amino acid sequence set forth in SEQ ID NO: 61 or a conservative modification thereof, and a CDR3 comprising the amino acid sequence set forth in SEQ ID NO: 62 or a conservative modification thereof. SEQ ID NOs: 60-62 are provided in Table 1.

In certain embodiments, the antibody or an antigen-binding fragment thereof comprises a V_{L} comprising a CDR1 comprising the amino acid sequence set forth in SEQ ID NO: 63 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification thereof. SEQ ID NOs: 63-65 are provided in Table 1.

In certain embodiments, the antibody or an antigen-binding fragment thereof comprises a V_{H} comprising a CDR1 comprising the amino acid sequence set forth in SEQ ID NO: 60 or a conservative modification thereof, a CDR2 comprising the amino acid sequence set forth in SEQ ID NO: 61 or a conservative modification thereof, and a CDR3 comprising the amino acid sequence set forth in SEQ ID NO: 62 or a conservative modification thereof; and a V_{L} Comprising a CDR1 comprising the amino acid sequence set forth in SEQ ID NO: 63 or a conservative modification thereof, a CDR2 comprising the amino acid sequence set forth in SEQ ID NO: 64 or a conservative modification, and a CDR3 comprising the amino acid sequence set forth in SEQ ID NO: 65 or a conservative modification thereof.

In certain embodiments, the antibody or an antigen-binding fragment thereof comprises a V_{H} comprising a CDR1 comprising the amino acid sequence set forth in SEQ ID NO: 60, a CDR2 comprising the amino acid sequence set forth in SEQ ID NO: 61, a V_{H} CDR3 comprising the amino acid sequence set forth in SEQ ID NO: 62; and a V_{L} comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65.

In certain embodiments, the antibody or an antigen-binding fragment thereof comprises a V_{H} comprising the amino acid sequence set forth in SEQ ID NO: 66. An exemplary nucleotide sequence encoding the amino acid sequence of SEQ ID NO: 66 is set forth in SEQ ID NO: 68. In certain embodiments, the antibody or an antigen-binding fragment thereof comprises a V_{L} comprising the amino acid sequence set forth in SEQ ID NO: 67. An exemplary nucleotide sequence encoding the amino acid sequence of SEQ ID NO: 67 is set forth in SEQ ID NO: 69. SEQ ID NO: 66-69 are provided in Table 1.

In certain embodiments, the antibody or an antigen-binding fragment thereof comprises a V_{H} comprising the amino acid sequence set forth in SEQ ID NO: 66 and a V_{L} comprising the amino acid sequence set forth in SEQ ID NO: 67.

In certain embodiments, the antibody or an antigen-binding fragment thereof comprises a V_{H} comprising the amino acid sequence set forth in SEQ ID NO: 66, and a V_{L} comprising the amino acid sequence set forth in SEQ ID NO: 67. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the variable regions are linked one after another such that a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1

| CDRS | 1 | 2 | 3 |
|---|---|---|---|
| V_{H} | GFTFSDFD | INGGSGVI | AREELGRRYYFDY |
| | [SEQ ID NO: 60] | [SEQ ID NO: 61] | [SEQ ID NO: 62] |
| V_{L} | SEHSSYN | LKSDGSH | GAGYTISGQYGYV |
| | [SEQ ID NO: 63] | [SEQ ID NO: 64] | [SEQ ID NO: 65] |

| | |
|---|---|
| Full V_{H} | MNSGLKLVFFVLILKGVQCEVQLVESGGGLVQTGKSLKLSCEASGFTFSDFDMNWVR |
| | QAPGKGLEWVAYINGGSGVIFYADAVKGRFTISRDNAKNLLFLQMNNLKSEDSAMYY |
| | CAREELGRRYYFDYWGQGTMVTVSSATTTAPSVYPLAPACDSTTSTTNTVTLGCLVK |
| | GYFPEPVTVSWNSGALTSGVHTFPSVLHSGLYSLSSSVTVPSSTW [SEQ ID NO: 66] |
| Full V_{L} | MAWIPLLFFLLHCTGSFSQPVLTQSPSASASLSGSVKLTCTLSSEHSSYNIAWYQQH |
| | PDKAPKYVMYLKSDGSHFKGDGIPDRFSGSSSGAHRYLSISNVQSEDDATYFCGAGY |
| | TISGQYGYVFDSGTQLTVLGGPKSSPKVTVFPPSPEELRTNKATLVCLVNDFYPGSA |
| | TVTWKANGATINDGVKTTKPSKQGQNYMTSSYLSLTADQWRSH [SEQ ID NO: 67] |
| DNA for | ATGAACTCAGGACTCAAATTGGTTTTCTTTGTCCTTATTCTGAAAGGTGTCCAGTGT |
| Full V_{H} | GAGGTGCAGTTGGTGGAGTCTGGGGGAGGCTTAGTACAGACTGGAAAGTCCCTGAAA |
| | CTCTCATGTGAGGCCTCTGGATTCACCTTCAGTGACTTTGACATGAACTGGGTCCGC |
| | CAGGCTCCAGGGAAGGGGCTGGAGTGGGTCGCATACATTAATGGTGGTAGTGGTGTT |
| | ATCTTTTATGCTGACGCTGTGAAGGGCCGATTCACCATCTCCAGAGACAACGCCAAG |
| | AACTTACTGTTCCTACAGATGAACAATCTCAAGTCTGAGGACTCAGCCATGTATTAC |
| | TGTGCAAGAGAGGAACTGGGACGGAGGTACTACTTTGATTACTGGGGCCAAGGAACC |
| | ATGGTCACCGTCTCCTCAGCCACAACAACAGCCCCATCTGTCTATCCCTTGGCCCCT |
| | GCCTGTGACAGCACAACCAGCACCACGAACACGGTGACCCTGGGATGCCTGGTCAAG |
| | GGCTATTTCCCTGAGCCGGTGACCGTAAGCTGGAACTCTGGAGCCCTGACCAGCGGC |
| | GTGCACACCTTCCCATCTGTCCTGCATTCTGGGCTCTACTCCCTCAGCAGCTCAGTG |
| | ACTGTACCTTCCAGCACCTGG [SEQ ID NO: 68] |
| DNA for | ATGGCCTGGATTCCTCTCCTCTTCTTCCTCCTTCATTGCACAGGGTCTTTCTCTCAA |
| Full V_{L} | CCTGTGTTGACTCAGTCACCCTCTGCCTCTGCCTCCCTGAGTGGCTCAGTCAAACTC |
| | ACCTGCACCCTGAGTAGTGAGCACAGCTCCTACAACATAGCATGGTACCAGCAACAT |
| | CCAGACAAGGCTCCCAAGTATGTGATGTACCTTAAGAGTGATGGAAGCCACTTCAAG |
| | GGAGATGGGATCCCTGATCGCTTCTCTGGCTCCAGCTCTGGGGCTCATCGCTACTTA |
| | AGCATCTCCAATGTCCAGTCTGAAGATGATGCTACCTATTTCTGTGGTGCAGGTTAT |
| | ACCATTTCTGGACAATATGGGTATGTTTTTGACAGCGGAACCCAGCTCACCGTCCTA |
| | GGTGGACCCAAGTCTTCTCCCAAAGTCACAGTGTTTCCACCTTCACCTGAGGAGCTC |
| | CGGACAAACAAAGCCACACTGGTGTGTCTGGTTAATGACTTCTACCCGGGTTCTGCA |
| | ACAGTGACCTGGAAGGCAAATGGAGCAACTATCAATGATGGGGTGAAGACTACAAAG |
| | CCTTCCAAACAGGGCCAAAACTACATGACCAGCAGCTACCTAAGTTTGACAGCAGAC |
| | CAGTGGAGATCCCAC [SEQ ID NO: 69] |

In certain embodiments, the antibody or an antigen-binding fragment thereof that engages the chimeric receptor (e.g., a CAR) is an scFv, an scFv-Fc fusion protein, or a full-length human IgG with V_{H} and V_{L} regions or CDRs selected from Table 2. In certain embodiments, the antibody or an antigen-binding fragment thereof is designated as “CAR2” or “12D11”.

In certain embodiments, the antibody or an antigen-binding fragment thereof comprises a V_{H} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof. SEQ ID NOs: 70-72 are provided in Table 2.

In certain embodiments, the antibody or an antigen-binding fragment thereof comprises a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof. SEQ ID NOs: 73-75 are provided in Table 2.

In certain embodiments, the antibody or an antigen-binding fragment thereof comprises a V_{H} comprising a CDR1 comprising the amino acid sequence set forth in SEQ ID NO: 70 or a conservative modification thereof, a CDR2 comprising the amino acid sequence set forth in SEQ ID NO: 71 or a conservative modification thereof, and a CDR3 comprising the amino acid sequence set forth in SEQ ID NO: 72 or a conservative modification thereof; and a V_{L} comprising a CDR1 comprising the amino acid sequence set forth in SEQ ID NO: 73 or a conservative modification thereof, a CDR2 comprising the amino acid sequence set forth in SEQ ID NO: 74 or a conservative modification thereof, and a CDR3 comprising the amino acid sequence set forth in SEQ ID NO: 75 or a conservative modification thereof.

In certain embodiments, the antibody or an antigen-binding fragment thereof comprises a V_{H} comprising a CDR1 comprising the amino acid sequence set forth in SEQ ID NO: 70, a CDR2 comprising the amino acid sequence set forth in SEQ ID NO: 71, a V_{H} CDR3 comprising the amino acid sequence set forth in SEQ ID NO: 72; and a V_{L} comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

In certain embodiments, the antibody or an antigen-binding fragment thereof comprises a V_{H} comprising the amino acid sequence set forth in SEQ ID NO: 76, as shown in Table 2. An exemplary nucleotide sequence encoding the amino acid sequence of SEQ ID NO: 76 is set forth in SEQ ID NO: 78. In certain embodiments, the antibody or an antigen-binding fragment thereof comprises a V_{L} comprising the amino acid sequence set forth in SEQ ID NO: 77. An exemplary nucleotide sequence encoding the amino acid sequence of SEQ ID NO: 77 is set forth in SEQ ID NO: 79. In certain embodiments, the antibody or an antigen-binding fragment thereof comprises a V_{H} Comprising the amino acid sequence set forth in SEQ ID NO: 76 and a V_{L} comprising the amino acid sequence set forth in SEQ ID NO: 77. SEQ ID NO: 76-79 are provided in Table 2.

In certain embodiments, the antibody or an antigen-binding fragment thereof comprises a V_{H} comprising the amino acid sequence set forth in SEQ ID NO: 76, and a V_{L} comprising the amino acid sequence set forth in SEQ ID NO: 77. In certain embodiments, the V_{H} and V_{L} are linked via a linker. In certain embodiments, the linker comprises the amino acid sequence set forth in SEQ ID NO: 1, SEQ ID NO: 2, SEQ ID NO: 3, SEQ ID NO: 4, SEQ ID NO: 5, or SEQ ID NO: 6.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2

| CDRs | 1 | 2 | 3 |
|---|---|---|---|
| V_{H} | GFDLSDYE | IYPQSYDYAT | TKDGDVGF |
| | [SEQ ID NO: 70] | [SEQ ID NO: 71] | [SEQ ID NO: 72] |
| V_{L} | QDISNE | NGK | LQHYSFPLT |
| | [SEQ ID NO: 73] | [SEQ ID NO: 74] | [SEQ ID NO: 75] |

| | |
|---|---|
| Full V_{H} | MGLGLQWVFFVALLKGVHCAVRLLESGGGLVKPEGALKLSCVASGFDLSDYFMGWVRQAPGK |
| | GLEWVAHIYPQSYDYATYYSGSVQGRFTISRDDSRSMVYLQMNNLRTEDTATYYCTKDGDVG |
| | FWGQGTMVTVSSATTTAPSVYPLAPACDSTTSTTNTVTLGCLVKGYFPEPVTVSWNSGALTS |
| | GVHTFPSVLHSGLYSLSSSVTVPSSTW [SEQ ID NO: 76] |
| Full V_{L} | MANKSPAQALAILLLWLSGVRCDIQVTQSPTLLSASLGDKVTINCLASQDISNELNWYQQKS |
| | GQSPTLLIYNGKNLQSGVPSRFSGQYSGRSFTISINNVEPEDVATYFCLQHYSFPLTFGDGS |
| | KLEMKRADAKPTVSIFPPSSEQLGTGSATLVCFVNNFYPKDINVKWKVDGSEKRDGVLQSVT |
| | DQDSKDSTYS [SEQ ID NO: 77] |
| DNA for | ATGGGATTGGGACTGCAGTGGGTTTTCTTTGTTGCTCTTTTAAAAGGTGTCCACTGTGCGGT |
| Full V_{H} | GCGGCTTCTGGAGTCGGGTGGAGGATTAGTGAAGCCTGAGGGGGCACTGAAACTCTCCTGTG |
| | TGGCCTCTGGATTCGACTTAAGTGACTATTTCATGGGCTGGGTCCGCCAGGCTCCAGGGAAG |
| | GGGCTGGAGTGGGTTGCTCACATATACCCTCAAAGTTATGATTATGCAACCTATTACTCGGG |
| | TTCGGTGCAAGGCAGATTCACCATCTCCAGAGATGATTCCCGAAGCATGGTCTACCTGCAAA |
| | TGAACAACCTGAGAACTGAGGACACGGCCACTTATTACTGTACAAAAGACGGGGACGTGGGT |
| | TTCTGGGGCCAAGGAACCATGGTCACCGTCTCCTCAGCCACAACAACAGCCCCATCTGTCTA |
| | TCCCTTGGCCCCTGCCTGTGACAGCACAACCAGCACCACGAACACGGTGACCCTGGGATGCC |
| | TGGTCAAGGGCTATTTCCCTGAGCCGGTGACCGTAAGCTGGAACTCTGGAGCCCTGACCAGC |
| | GGCGTGCACACCTTCCCATCTGTCCTGCATTCTGGGCTCTACTCCCTCAGCAGCTCAGTGAC |
| | TGTACCTTCCAGCACCTGG [SEQ ID NO: 78] |
| DNA for | ATGGCCAACAAGTCTCCTGCTCAGGCACTGGCAATTTTGTTACTGTGGCTGTCAGGTGTCAG |
| Full V_{L} | ATGTGACATTCAAGTGACACAATCTCCCACCCTCCTGTCAGCATCTCTAGGAGACAAAGTGA |
| | CCATCAATTGCCTGGCAAGTCAGGACATTAGCAATGAGTTAAACTGGTACCAGCAGAAGTCA |
| | GGACAATCTCCTACACTGTTGATTTATAATGGAAAAAATTTGCAGTCTGGTGTCCCGTCAAG |
| | GTTCAGTGGCCAGTATTCAGGGAGAAGTTTCACTATCAGCATCAACAATGTGGAACCTGAAG |
| | ATGTTGCAACTTATTTTTGTCTTCAGCATTACAGTTTTCCGCTCACGTTCGGTGATGGCTCC |
| | AAGCTGGAGATGAAACGGGCTGATGCTAAGCCAACCGTCTCCATCTTCCCACCATCCAGTGA |
| | GCAGTTGGGCACTGGAAGCGCCACACTTGTGTGCTTCGTGAACAACTTCTACCCCAAAGACA |
| | TCAATGTCAAGTGGAAAGTAGATGGCAGTGAAAAACGAGATGGCGTCCTGCAGAGTGTCACT |
| | GATCAGGACAGCAAAGACAGCACCTACAGC [SEQ ID NO: 79] |

### 5.3.5.2 Monoclonal Antibodies

The antibodies disclosed herein (e.g., 19E3 and 12D11) specifically bind to an antigen-binding domain (e.g., an extracellular antigen-binding domain of a CAR). The V_{H} amino acid sequences of presently disclosed antibodies are set forth in SEQ ID NOs: 66 and 76. The V_{L} amino acid sequences of the presently disclosed antibodies are set forth in SEQ ID NOs: 67 and 77.

Given that each of the presently disclosed antibodies can bind to a chimeric receptor (e.g., a CAR), the V_{H} and V_{L} sequences can be “mixed and matched” to create other binding molecules targeting chimeric receptors. Binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) comprising an amino acid sequence selected from SEQ ID NOs: 66 and 76; and (b) a light chain variable region (V_{L}) comprising an amino acid sequence selected from SEQ ID NOs: 67 and 77; wherein the antibody or antigen-binding fragment specifically binds to a chimeric receptor, e.g., the extracellular antigen-binding domain of a CAR. In certain embodiments, the V_{H} and V_{L} are selected from the group consisting of:
(a) a heavy chain variable region comprising the amino acid sequence set forth in SEQ ID NO: 66, and a light chain variable region comprising the amino acid sequence set forth in SEQ ID NO: 67; or
(b) a heavy chain variable region comprising the amino acid sequence set forth in SEQ ID NO: 76, and a light chain variable region comprising the amino acid sequence set forth in SEQ ID NO: 77.

In certain embodiments, the presently disclosed subject matter provides antibodies or antigen-binding fragments thereof that comprise the heavy chain and light chain CDR1s, CDR2s and CDR3s of the presently disclosed antibodies.

The amino acid sequences of the V_{H} CDR1s of the presently disclosed antibodies are shown in SEQ ID NOs: 60 and 70. The amino acid sequences of the V_{H} CDR2s of the presently disclosed antibodies are set forth in SEQ ID NOs: 61 and 71. The amino acid sequences of the V_{H} CDR3s of the presently disclosed antibodies are set forth in SEQ ID NOs: 62 and 72.

The amino acid sequences of the V_{L} CDR1s of the presently disclosed antibodies are set forth in SEQ ID NOs: 63 and 73. The amino acid sequences of the V_{L} CDR2s of the presently disclosed antibodies are set forth in SEQ ID NOs: 64 and 74. The amino acid sequences of the V_{L} CDR3s of the presently disclosed antibodies are set forth in SEQ ID NOs: 65 and 75. The CDR regions are delineated using the IMGT system. In certain embodiments, the CDR regions are delineated using the IMGT numbering system accessible at http://www.imgt.org/IMGT_vquest/input.

Given that each of these antibodies or antigen-binding fragments thereof can bind to an antigen-binding domain (e.g., an extracellular antigen-binding domain of a CAR) and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V_{L} CDR1, CDR2, and CDR3) to create other binding molecules. Binding of such “mixed and matched” antibodies can be tested using the binding assays described above. When V_{H} CDR sequences are mixed and matched, the CDR1, CDR2, and/or CDR3 sequence from a particular V_{H} sequence is replaced with a structurally similar CDR sequence(s). Likewise, when V_{L} CDR sequences are mixed and matched, the CDR1, CDR2, and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein.

In certain embodiments, the presently disclosed subject matter provides an antibody or an antigen-binding fragment thereof comprising:
(a) a heavy chain variable region CDR1 comprising an amino acid sequence selected from SEQ ID NOs: 60 and 70;
(b) a heavy chain variable region CDR2 comprising an amino acid sequence selected from SEQ ID NOs: 61 and 71;
(c) a heavy chain variable region CDR3 comprising an amino acid sequence selected from SEQ ID NOs: 62 and 72;
(d) a light chain variable region CDR1 comprising an amino acid sequence selected from SEQ ID NOs: 63 and 73;
(e) a light chain variable region CDR2 comprising an amino acid sequence selected from SEQ ID Nos: 64 and 74; and
(f) a light chain variable region CDR3 comprising an amino acid sequence selected from SEQ ID NOs: 65 and 75.

In certain embodiments, the antibody or antigen-binding fragment thereof comprises:
(a) a heavy chain variable region CDR1 comprising the amino acid sequence set forth in SEQ ID NO: 60;
(b) a heavy chain variable region CDR2 comprising the amino acid sequence set forth in SEQ ID NO: 61;
(c) a heavy chain variable region CDR3 comprising the amino acid sequence set forth in SEQ ID NO: 62;
(d) a light chain variable region CDR1 comprising the amino acid sequence set forth in SEQ ID NO: 63;
(e) a light chain variable region CDR2 comprising the amino acid sequence set forth in SEQ ID NO: 64; and
(f) a light chain variable region CDR3 comprising the amino acid sequence set forth in SEQ ID NO: 65.

In certain embodiments, the antibody or antigen-binding fragment thereof comprises:
(a) a heavy chain variable region CDR1 comprising the amino acid sequence set forth in SEQ ID NO: 70;
(b) a heavy chain variable region CDR2 comprising the amino acid sequence set forth in SEQ ID NO: 71;
(c) a heavy chain variable region CDR3 comprising the amino acid sequence set forth in SEQ ID NO: 72;
(d) a light chain variable region CDR1 comprising the amino acid sequence set forth in SEQ ID NO: 73;
(e) a light chain variable region CDR2 comprising the amino acid sequence set forth in SEQ ID NO: 74; and
(f) a light chain variable region CDR3 comprising the amino acid sequence set forth in SEQ ID NO: 75.

The constant region/framework region of the antibodies or antigen-fragments thereof disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation, etc., the number of cysteine residues, effector cell function, effector cell function, complement function or introduction of a conjugation site).

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs.

### 5.3.5.3 Homologous Antibodies

In certain embodiments, a presently disclosed antibody or antigen-binding fragment thereof comprises heavy and light chain variable regions comprising amino acid sequences that are homologous or identical to the amino acid sequences of the antibodies described herein (e.g., 19E3 and 12D11 antibodies), and wherein the antibodies or antigen-binding fragments thereof retain the desired functional properties of the antibodies or antigen-binding fragments thereof of the presently disclosed subject matter.

For example, the presently disclosed subject matter provides an antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 66 and SEQ ID NO: 76;
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 67 and SEQ ID NO: 77.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

5.3.5.4 Antibodies with Conservative Modifications

In certain embodiments, a presently disclosed antibody or an antigen-binding fragment thereof comprises a heavy chain variable region comprising CDR1, CDR2, and CDR3 sequences and a light chain variable region comprising CDR1, CDR2, and CDR3 sequences, wherein one or more of these CDR sequences comprise specified amino acid sequences based on the preferred antibodies described herein (e.g., 19E3 and 12D11 antibodies), or a conservative modification thereof, and wherein the antibodies retain the desired functional properties of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises an amino acid sequence selected from the amino acid sequences of SEQ ID NOs: 62 and 72, and conservative modifications thereof;
(b) the light chain variable region CDR3 sequence comprises an amino acid sequence selected from the amino acid sequence of SEQ ID NOs: 65 and 75, and conservative modifications thereof.

In certain embodiments, the heavy chain variable region CDR3 sequence comprises an amino acid sequence selected from the amino acid sequences of SEQ ID NOs: 62 and 72, and conservative modifications thereof; and the light chain variable region CDR3 sequence comprises an amino acid sequence selected from the amino acid sequences of SEQ ID NOs: 65 and 75, and conservative modifications thereof.

In certain embodiments, the heavy chain variable region CDR2 sequence comprises an amino acid sequence selected from the amino acid sequences of SEQ ID NOs: 61 and 71, and conservative modifications thereof; and the light chain variable region CDR2 sequence comprises an amino acid sequence selected from the amino acid sequences of SEQ ID NOs: 64 and 74, and conservative modifications thereof.

In certain embodiments, the heavy chain variable region CDR1 sequence comprises an amino acid sequence selected from the amino acid sequences of SEQ ID NOs: 60 and 70, and conservative modifications thereof; and the light chain variable region CDR1 sequence comprises an amino acid sequence selected from the amino acid sequences of SEQ ID NOs: 63 and 73, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions, and deletions. Modifications can be introduced into an antibody of the presently disclosed subject matter by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 3. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence, or a V_{L} sequence, can have up to about one, up to about two, up to about three, up to about four, up to about five, up to about six, up to about seven, up to about eight, up to about nine or up to about ten amino acid residues that are modified and/or substituted.

TABLE 3

| | Exemplary conservative amino acid |
|---|---|
| Original Residue | Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Len, Ile;
neutral hydrophilic: Cys, Ser, Thr, Asn, Gln;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

5.3.5.5 Antibodies that Cross-Compete for Binding to Chimer Receptor with the Presently Disclosed Antibodies

The presently disclosed subject matter provides antibodies or antigen-binding fragments thereof that cross-compete with any of the disclosed antibodies for binding to an antigen-binding domain (e.g., an extracellular antigen-binding domain of a CAR). For example, and not by way of limitation, the cross-competing antibodies can bind to the same epitope region, e.g., same epitope, adjacent epitope, or overlapping as any of the antibodies or antigen-binding fragments thereof of the presently disclosed subject matter. In certain embodiments, the reference antibody or reference antigen-binding fragments thereof for cross-competition studies can be any one of the antibodies or antigen-binding fragments thereof disclosed herein, e.g., 19E3 or 12D11.

Such cross-competing antibodies can be identified based on their ability to cross-compete with any one of the presently disclosed antibodies or antigen-binding fragments thereof in standard binding assays. For example, Biacore analysis, ELISA assays, or flow cytometry can be used to demonstrate cross-competition with the antibodies of the presently disclosed subject matter. The ability of a test antibody to inhibit the binding of, for example, any one of the presently disclosed antibodies (e.g., 19E3 or 12D11) to an antigen-binding domain (e.g., an extracellular antigen-binding domain of a CAR) demonstrates that the test antibody can compete with any one of the presently disclosed antibodies or antigen-binding fragments thereof for binding to an antigen-binding domain (e.g., an extracellular antigen-binding domain of a CAR) and thus binds to the same epitope region on an antigen-binding domain (e.g., an extracellular antigen-binding domain of a CAR) as any one of the presently disclosed antibodies or antigen-binding fragments thereof. In certain embodiments, the cross-competing antibody or antigen-binding fragment thereof binds to the same epitope on an antigen-binding domain (e.g., an extracellular antigen-binding domain of a CAR) as any one of the presently disclosed antibodies or antigen-binding fragments thereof (e.g., 19E3 or 12D11).

### 5.3.5.6. Characterization of Antibody Binding to Antigen

Antibodies or antigen-binding fragments thereof of the presently disclosed subject can be tested for binding to a chimeric receptor by, for example, standard ELISA. To determine if the selected antibodies bind to unique epitopes, each antibody can be biotinylated using commercially available reagents (Pierce, Rockford, IL). Competition studies using unlabeled monoclonal antibodies and biotinylated monoclonal antibodies can be performed using chimeric receptor coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. IgGs can be further tested for reactivity with a chimeric receptor (e.g., antigen) by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ)

### 5.3.5.7. Multi-Specific Molecules

The presently disclosed subject matter provides multi-specific molecules comprising a presently disclosed antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed antibody or antigen-binding fragment thereof can be derivatized or linked to more than one other functional molecules to generate multi-specific molecules that bind to more than two different binding sites and/or target molecules. To create a multi-specific molecule, a presently disclosed antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprise at least a first binding specificity for a chimeric receptor and a second binding specificity for a second target epitope region. The second target epitope region can be an epitope of a chimeric receptor, or a different epitope, e.g., a different antigen. In certain embodiments, the multi-specific molecule comprises a first binding specificity for an antigen-binding domain (e.g., an extracellular antigen-binding domain of a CAR), a second binding specificity for a second target, and a third binding specificity for a third target. In certain embodiments, the second target is an antigen expressed on the surface of an immune cell (e.g., a T cell, or a human immune effector cell).

In certain embodiments, the multi-specific molecule comprises a first binding specificity for a chimeric receptor and a second binding specificity for a 4-1BB polypeptide. In certain embodiments, the first binding specificity is a binding specificity of the 19E3 antibody and the second binding specificity is a binding specificity of urelumab. In certain embodiments, the first binding specificity is a binding specificity of the 12D11 antibody and the second binding specificity is a binding specificity of urelumab.

The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb×mAb, mAb×Fab, Fab×F(ab′)₂, or ligand x Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific to the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 5.3.5.8. Antibody Fragments

The presently disclosed subject matter provides antibody fragments of an antibody disclosed herein. In certain embodiments, the antibody fragment comprises an scFv as disclosed herein in Section 5.3.5.1. In certain embodiments, the antibody fragment is an scFv as disclosed herein in Section 5.3.5.1. In certain embodiments, the antibody fragment is a Fab fragment, a Fab′ fragment, a Fab′-SH fragment, or a F(ab′)2 fragment.

In certain embodiments, the antibody fragment is a “Fab” fragments. “Fab” fragments can be produced by papain digestion of full length antibodies. Traditionally, Fab fragments contain the heavy-chain variable domain (VH) and light-chain variable domain (VH) and also the constant domain of the light chain (CL) and the first constant domain of the heavy chain (CH1).

In certain embodiments, the antibody fragment is a “Fab′” fragments. Fab′ fragments can be distinguished from Fab fragments because including additional residues at the carboxy terminus of the CH1 domain. In certain embodiments, the Fab′ fragments include one or more cysteines from the antibody hinge region.

In certain embodiments, the antibody fragment is a “Fab′-SH” fragments. Fab′-SH are Fab′ fragments where at least one cysteine residue of the constant domains includes a free thiol group.

In certain embodiments, the antibody fragment is a “F(ab′)2” fragment. F(ab′)2 fragments can be obtained by pepsin digestion of full length antibodies. F(ab′)2 fragments have two antigen-binding sites (e.g., two Fab fragments) and a portion of the Fc region.

In certain embodiments, the antibody fragment is a single-domain antibody. Single-domain antibodies are antibody fragments including the heavy chain variable domain or a portion thereof of an antibody or the light chain variable domain or a portion thereof of an antibody.

### 5.3.5.9. Chimeric and Humanized Antibodies

The presently disclosed subject matter further provides chimeric and/or humanized versions of an antibody, or a fragment thereof, disclosed herein.

In certain embodiments, the antibody is a chimeric antibody. In certain embodiments, the chimeric antibody comprises a variable region derived from a non-human species (e.g., a variable region derived from a mouse, a rat, a hamster, a rabbit, or a non-human primate) and a human constant region. Alternatively or additionally, a chimeric antibody can be a “class-switched” antibody. In certain embodiments, the class-switched antibody is an antibody wherein the class or subclass has been modified from that of the parent antibody.

In certain embodiments, the antibody provided herein is a humanized antibody. In certain embodiments, the humanized antibody comprises at least one variable domain. In certain embodiments, the variable domain comprises CDRs derived from a non-human antibody, e.g., a mouse antibody.

In certain embodiments, the variable domain comprises framework regions (FR) derived from human antibody sequences. In certain embodiments, the FR includes substitutions and/or modifications. In certain embodiments, residues in a humanized antibody can be substituted with corresponding residues from a non-human antibody (e.g., the antibody from which the CDR residues are derived), to restore or improve antibody specificity or affinity.

In certain embodiments, the humanized antibody can also include a human constant region. In certain embodiments, humanization of a non-human antibody, e.g. a mouse antibody, reduces immunogenicity of the antibody in humans. In certain embodiments, humanization of a non-human antibody, e.g. a mouse antibody, does not impair the specificity and affinity of the parental non-human antibody.

### 5.3.6 Storage of Cells

The presently disclosed methods also include cryopreservation of the induced T cells obtained by the methods disclosed herein. In certain embodiments, the induced T cells are viable upon thawing. As used herein, the term “cryopreservation” refers to a process that preserves organelles, cells, tissues, or any other biological constructs by cooling the samples to very low temperatures. In certain embodiments, the cryopreservation includes cooling to sub-zero temperatures (e.g., −196° C.). In certain embodiments, the cryopreservation includes the use of cryoprotective agents. Non-limiting examples of cryoprotective agents include dimethyl sulfoxide (DMSO) glycerol, polyvinylpyrrolidone, and polyethylene glycol. In certain embodiments, the cryopreservation comprises controlled rate cooling. In certain embodiments, the controlled rate of cooling is from about 1° C. to about 3° C./minute. In certain embodiments, the controlled rate cooling stops once a temperature of −80° C. has been reached.

### 5.3.7 Exemplary Methods

In certain embodiments, the presently disclosed methods comprise contacting the induced T cells with a polypeptide that engages the CAR and an antibody or antigen-binding fragment thereof that binds to 4-1BB, to thereby obtaining an expanded population of induced T cells. In certain embodiments, the polypeptide that engages the CAR is an antigen-containing polypeptide. In certain embodiments, the antigen-containing polypeptide is an Fc-fusion protein. In certain embodiments, the Fc-fusion protein comprises a recombinant antigen polypeptide or a fragment thereof. In certain embodiments, the antigen-binding fragment thereof that binds to 4-1BB is urelumab.

In certain embodiments, the presently disclosed methods comprise contacting the induced T cells with a polypeptide that engages the CAR and an antibody or antigen-binding fragment thereof that binds to 4-1BB, to thereby obtaining an expanded population of induced T cells. In certain embodiments, the polypeptide that engages the CAR is an antigen-containing polypeptide. In certain embodiments, the antigen-containing polypeptide is a recombinant antigen polypeptide or a fragment thereof. In certain embodiments, the antigen-binding fragment thereof that binds to 4-1BB is urelumab.

In certain embodiments, the presently disclosed methods comprise contacting the induced T cells with a polypeptide that engages the CAR and an antibody or antigen-binding fragment thereof that binds to 4-1BB, to thereby obtaining an expanded population of induced T cells. In certain embodiments, the polypeptide that engages the CAR is an antibody or antigen-binding fragment thereof that binds to a scFv of the CAR. In certain embodiments, the antigen-binding fragment thereof that binds to 4-1BB is urelumab.

In certain embodiments, the presently disclosed methods comprise contacting the induced T cells with a polypeptide that engages the CAR and an antibody or antigen-binding fragment thereof that binds to 4-1BB, to thereby obtaining an expanded population of induced T cells. In certain embodiments, the polypeptide that engages the CAR is an antibody or antigen-binding fragment thereof that binds to binds to an idiotypic variable domain of a scFv of the CAR. In certain embodiments, the antigen-binding fragment thereof that binds to 4-1BB is urelumab.

In certain embodiments, the presently disclosed methods comprise contacting the induced T cells with a polypeptide that engages the CAR and an antibody or antigen-binding fragment thereof that binds to 4-1BB, to thereby obtaining an expanded population of induced T cells. In certain embodiments, the polypeptide that engages the CAR is antibody or an antigen-binding fragment thereof that engages the chimeric receptor (e.g., a CAR). In certain embodiments, the antibody or an antigen-binding fragment thereof that engages the chimeric receptor (e.g., a CAR) is 19E3. In certain embodiments, the antigen-binding fragment thereof that binds to 4-1BB is urelumab.

In certain embodiments, the presently disclosed methods comprise contacting the induced T cells with a polypeptide that engages the CAR and an antibody or antigen-binding fragment thereof that binds to 4-1BB, to thereby obtaining an expanded population of induced T cells. In certain embodiments, the polypeptide that engages the CAR is antibody or an antigen-binding fragment thereof that engages the chimeric receptor (e.g., a CAR). In certain embodiments, the antibody or an antigen-binding fragment thereof that engages the chimeric receptor (e.g., a CAR) is 12D11. In certain embodiments, the antigen-binding fragment thereof that binds to 4-1BB is urelumab.

### 5.4. Induced T Cells

The presently disclosed subject matter provides induced T cells prepared through the methods disclosed in Section 5.3.

The induced T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells can be used for obtaining induced pluripotent stem cells and be genetically modified for the improved manufacturing methods disclosed herein. The induced T cell can be a CD4⁺ T cell, a CD8⁺ T cell, a double-positive (DP) T cell, or a double-negative (DN) T cell. In certain embodiments, the induced T cell is a CD4⁺ induced T cell. In certain embodiments, the induced T cell is a CD8⁺ induced T cell. In certain embodiments, the CD8⁺ induced T cell is CD4 independent. In certain embodiments, the induced T cell is a CD3⁺, TCR⁻ T cell. In certain embodiments, the induced T cell is a CD4⁺, CD3⁺, and TCR⁻ T cell. In certain embodiments, the induced T cell is a CD8⁺, CD3⁺, and TCR⁻ T cell. In certain embodiments, the induced T cell is: a CD3⁺, TCR⁻, CD25⁺, CD28⁺, CD69⁺, CD56⁺, CD45RA⁺ T cell. In certain embodiments, the induced T cell is a CD3⁺, TCR⁻, CD4⁻, CD8αα⁻ T cell. In certain embodiments, the induced T cell is a CD3⁺, TCR⁻, CD4⁻, CD8αβ⁻ T cell. In certain embodiments, the induced T cells is a CD3⁺, TCR⁻, CD4⁻, CD8αα⁺ T cell. In certain embodiments, the induced T cell is a CD3⁺, TCR⁻, CD4⁻, CD8αβ⁺ T cell. In certain embodiments, the induced T cell is a CD3⁺, TCR⁻, CD4⁺, CD8αα⁻ T cell. In certain embodiments, the induced T cell is CD3⁺, TCR⁻, CD4⁺, CD8αβ⁻ T cell. In certain embodiments, the induced T cell is a CD3⁺, TCR⁻, CD4⁺, CD8αα⁺ T cell. In certain embodiments, the induced T cell is a CD3⁺, TCR⁻, CD4⁺, CD8αβ⁺ T cell.

Types of human lymphocytes that can be modified using any of the presently disclosed methods include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the induced T cell is autologous. In certain embodiments, the induced T cell is non-autologous. In certain embodiments, the induced T cell is allogeneic.

### 5.4.1. Second Antigen-Recognizing Receptors

The presently disclosed subject matter provides induced T cells (e.g., prepared through the methods disclosed in Section 5.3). In certain embodiments, the induced T cells further include a second antigen-recognizing receptor. In certain embodiments, the second antigen-recognizing receptor targets an antigen. In certain embodiments, the antigen can be a tumor antigen or a pathogen antigen. In certain embodiments, the second antigen-recognizing receptor is a chimeric receptor. In certain embodiments, the second chimeric receptor is a chimeric antigen receptor (CAR). In certain embodiments, the second antigen-recognizing receptor is a TCR like fusion molecule. In certain embodiments, the second antigen-recognizing receptor is a T Cell Receptor (TCR). In certain embodiments, the second antigen-recognizing receptor is a chimeric costimulatory receptor (CCR).

### 5.4.1.1. Second Antigens

In certain embodiments, the second antigen is a tumor antigen, e.g., one disclosed in Section 5.2.1. In certain embodiments, the second antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11

In certain embodiments, the second antigen is a pathogen antigen, e.g., one disclosed in Section 5.2.1.

### 5.4.1.2 CARs

In certain embodiments, the second antigen-recognizing receptor is a CAR, e.g., one described in Section 5.2.2.1, provided that the extracellular antigen-binding domain of the CAR binds to a second antigen. In certain embodiments, second antigen-recognizing receptor is a CAR that comprises an extracellular antigen-binding domain that binds to a second antigen, and an intracellular signaling domain. In certain embodiments, the CAR further comprises a transmembrane domain.

### 5.4.1.3 Chimeric Co-Stimulating Receptors (CCRs)

In certain embodiments, a presently disclosed induced T cell comprising a presently disclosed antigen-recognizing receptor further comprises chimeric co-stimulating receptor (CCR). The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a costimulatory signal to the cell but does not alone deliver an activation signal to the induced T cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both a first antigen (e.g., antigen targeted by a CAR) and the second antigen is restricted to the targeted cells (e.g., cancerous tissue or cancerous cells, or LSCs, or AML HSPCs). Similar to a CAR, the extracellular antigen-binding domain can be an scFv, a Fab, an F(ab)₂, or a fusion protein with a heterologous sequence to form the extracellular antigen-binding domain.

In certain embodiments, the induced T cell comprising a first antigen-recognizing receptor (e.g., a CAR) and a second antigen-recognizing receptor (e.g., a CAR, a TCR, or a TCR like fusion molecule) exhibits a greater degree of cytolytic activity against cells that are positive for both the first antigen and the second antigen as compared to against cells that are singly positive for the first antigen. In certain embodiments, the cell comprising the first antigen-recognizing receptor (e.g., a CAR) and the second antigen-recognizing receptor (e.g., a CAR, a TCR, or a TCR like fusion molecule) exhibits substantially no or negligible cytolytic activity against cells that are singly positive for the first antigen.

In certain embodiments, the first antigen recognizing receptor (e.g., a CAR, a TCR, or a TCR like fusion molecule) binds to the first antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the first antigen recognizing receptor (e.g., a CAR, a TCR, or a TCR like fusion molecule) binds to the first antigen with a low binding avidity. In certain embodiments, the first antigen recognizing receptor (e.g., a CAR, a TCR, or a TCR like fusion molecule) binds to the first antigen at an epitope of low accessibility. In certain embodiments, the first antigen recognizing receptor (e.g., a CAR, a TCR, or a TCR like fusion molecule) binds to the first antigen with a binding affinity that is lower compared to the binding affinity with which the second antigen-recognizing receptor (e.g., a CCR) binds to the second antigen. In certain embodiments, the CCR binds to the second antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 5.4.1.4 T Cell Receptors (TCRs)

In certain embodiments, the second antigen-recognizing receptor is a TCR. A TCR is a disulfide-linked heterodimeric protein consisting of two variable chains expressed as part of a complex with the invariant CD3 chain molecules. A TCR is found on the surface of T cells and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the second antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 5.4.1.5 TCR Like Fusion Molecules

In certain embodiments, the second antigen-recognizing receptor is a TCR-like fusion, e.g., one described in Section, 5.2.2.2, provided that the extracellular antigen-binding domain of the TCR like fusion molecule binds to a second antigen.

In certain embodiments, the second antigen-recognizing receptor is a TCR-like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”.

In certain embodiments, the second antigen-recognizing receptor is a TCR-like fusion molecule that comprises a first antigen binding chain comprising a V_{H} of an antibody and a constant domain comprising a TRAC polypeptide; and a second antigen binding chain comprising a V_{L} of an antibody and a constant domain comprising a TRBC polypeptide. In certain embodiments, the first antigen binding chain is designated as “V_{H}-TRAC chain”. In certain embodiments, the second antigen binding chain is designated as “V_{L}-TRBC chain”.

### 5.4.2 Co-Stimulatory Ligands

In certain embodiments, a presently disclosed induced T cell further comprises at least one recombinant or exogenous co-stimulatory ligand. For example, a presently disclosed induced T cell can be further transduced with at least one co-stimulatory ligand, such that the cell expresses or is induced to express the first antigen-recognizing receptor and the at least one co-stimulatory ligand. The at least one co-stimulatory ligand provides a co-stimulation signal to the cell.

Non-limiting examples of co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share several common features. The majority of TNF superfamily members are synthesized as type II transmembrane proteins (extracellular C-terminus) containing a short cytoplasmic segment and a relatively long extracellular region. Non-limiting examples of TNF superfamily members include nerve growth factor (NGF), CD40L (also known as “CD154”), 4-1BBL, TNF-α, OX40L, CD70, Fas ligand (FasL), CD30L, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Non-limiting examples of immunoglobulin superfamily ligands include CD80, CD86, and ICOSLG. In certain embodiments, the at least one co-stimulatory ligand is selected from the group consisting of 4-1BBL, CD80, CD86, CD70, GITRL, CD40L, OX40L, CD30L, TNFRSF14, ICOSLG, TRAIL, and combinations thereof.

In certain embodiments, the induced T cell further comprises one exogenous co-stimulatory ligand that is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a Uniprot Reference No: P41273-1 (SEQ ID NO: 52) or a fragment thereof, and/or may optionally comprise up to one or up to two or up to three conservative amino acid substitutions. In certain embodiments, the 4-1BBL comprises or consists of an amino acid sequence that is a consecutive portion of the amino acid sequence of SEQ ID NO: 52. SEQ ID NO: 52 is provided below.

[SEQ ID NO: 52]
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

An exemplary nucleotide sequence encoding the amino acid sequence of SEQ ID NO: 52 is set forth in SEQ ID NO: 53.

[SEQ ID NO: 53]
ATGGAATACGCCTCTGACGCTTCACTGGACCCCGAAGCCCCGTGGCCTCC
CGCGCCCCGCGCTCGCGCCTGCCGCGTACTGCCTTGGGCCCTGGTCGCGG
GGCTGCTGCTGCTGCTGCTGCTCGCTGCCGCCTGCGCCGTCTTCCTCGCC
TGCCCCTGGGCCGTGTCCGGGGCTCGCGCCTCGCCCGGCTCCGCGGCCAG
CCCGAGACTCCGCGAGGGTCCCGAGCTTTCGCCCGACGATCCCGCCGGCC
TCTTGGACCTGCGGCAGGGCATGTTTGCGCAGCTGGTGGCCCAAAATGTT
CTGCTGATCGATGGGCCCCTGAGCTGGTACAGTGACCCAGGCCTGGCAGG
CGTGTCCCTGACGGGGGGCCTGAGCTACAAAGAGGACACGAAGGAGCTGG
TGGTGGCCAAGGCTGGAGTCTACTATGTCTTCTTTCAACTAGAGCTGCGG
CGCGTGGTGGCCGGCGAGGGCTCAGGCTCCGTTTCACTTGCGCTGCACCT
GCAGCCACTGCGCTCTGCTGCTGGGGCCGCCGCCCTGGCTTTGACCGTGG
ACCTGCCACCCGCCTCCTCCGAGGCTCGGAACTCGGCCTTCGGTTTCCAG
GGCCGCTTGCTGCACCTGAGTGCCGGCCAGCGCCTGGGCGTCCATCTTCA
CACTGAGGCCAGGGCACGCCATGCCTGGCAGCTTACCCAGGGCGCCACAG
TCTTGGGACTCTTCCGGGTGACCCCCGAAATCCCAGCCGGACTCCCTTCA
CCGAGGTCGGAA

In certain embodiments, the induced T cell further comprises one exogenous co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 56) or a fragment thereof, and/or may optionally comprise up to one or up to two or up to three conservative amino acid substitutions.

In certain embodiments, the CD80 comprises or consists of an amino acid sequence that is a consecutive portion of the amino acid sequence of SEQ ID NO: 56. SEQ ID NO: 56 is provided below.

[SEQ ID NO: 56]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFAPRCRERRRNERLRRESVRPV

An exemplary nucleotide sequence encoding the amino acid sequence of SEQ ID NO: 56 is set forth in SEQ ID NO: 57. SEQ ID NO: 57 is provided below.

[SEQ ID NO: 57]
ATGGGCCACACACGGAGGCAGGGAACATCACCATCCAAGTGTCCATACCT
CAATTTCTTTCAGCTCTTGGTGCTGGCTGGTCTTTCTCACTTCTGTTCAG
GTGTTATCCACGTGACCAAGGAAGTGAAAGAAGTGGCAACGCTGTCCTGT
GGTCACAATGTTTCTGTTGAAGAGCTGGCACAAACTCGCATCTACTGGCA
AAAGGAGAAGAAAATGGTGCTGACTATGATGTCTGGGGACATGAATATAT
GGCCCGAGTACAAGAACCGGACCATCTTTGATATCACTAATAACCTCTCC
ATTGTGATCCTGGCTCTGCGCCCATCTGACGAGGGCACATACGAGTGTGT
TGTTCTGAAGTATGAAAAAGACGCTTTCAAGCGGGAACACCTGGCTGAAG
TGACGTTATCAGTCAAAGCTGACTTCCCTACACCTAGTATATCTGACTTT
GAAATTCCAACTTCTAATATTAGAAGGATAATTTGCTCAACCTCTGGAGG
TTTTCCAGAGCCTCACCTCTCCTGGTTGGAAAATGGAGAAGAATTAAATG
CCATCAACACAACAGTTTCCCAAGATCCTGAAACTGAGCTCTATGCTGTT
AGCAGCAAACTGGATTTCAATATGACAACCAACCACAGCTTCATGTGTCT
CATCAAGTATGGACATTTAAGAGTGAATCAGACCTTCAACTGGAATACAA
CCAAGCAAGAGCATTTTCCTGATAACCTGCTCCCATCCTGGGCCATTACC
TTAATCTCAGTAAATGGAATTTTTGTGATATGCTGCCTGACCTACTGCTT
TGCCCCAAGATGCAGAGAGAGAAGGAGGAATGAGAGATTGAGAAGGGAAA
GTGTACGCCCTGTA

In certain embodiments, the induced T cell further comprises two exogenous co-stimulatory ligands that are 4-1BBL and CD80. In certain embodiments, the induced T cell further comprises two exogenous co-stimulatory ligands that are 4-1BBL and CD80, wherein the 4-1BBL comprises or consists of the amino acid sequence set forth in SEQ ID NO: 52, and the CD80 comprises or consists of the amino acid sequence set forth in SEQ ID NO: 56.

Receptor-comprising cells comprising at least one exogenous co-stimulatory ligand are described in U.S. Pat. No. 8,389,282, which is incorporated by reference in its entirety.

### 5.4.3 Fusion Polypeptides

In certain embodiments, a presently disclosed induced T cell further comprises a fusion polypeptide. For example, a presently disclosed induced T cell can be further transduced with the fusion polypeptide, such that the cell expresses or is induced to express the first antigen-recognizing receptor and the fusion polypeptide. The fusion polypeptide provides a co-stimulation signal to the cell. The fusion polypeptides are capable of enhancing the activity and/or efficacy of a cell comprising the first antigen-recognizing receptor (e.g., a CAR or a TCR like fusion molecule). In certain embodiments, the fusion polypeptide comprises a) an extracellular domain and a transmembrane domain of a co-stimulatory ligand, and b) an intracellular domain of a first co-stimulatory molecule.

Non-limiting examples of the co-stimulatory ligand include tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof. In certain embodiments, the co-stimulatory ligand is selected from the group consisting of 4-1BBL, OX40L, CD70, GITRL, CD40L, CD80, CD86, ICOSLG, and combinations thereof.

In certain embodiments, the fusion polypeptide comprises an extracellular domain and a transmembrane domain of a co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 56 or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 56.

In certain embodiments, the extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 1-242 of SEQ ID NO: 56. In certain embodiments, the extracellular domain of CD80 comprises or consists of amino acids 1-242 of SEQ ID NO: 56 or a functional fragment thereof. A functional fragment can be a consecutive portion of amino acids 1-242 of SEQ ID NO: 56, which is at least about 50, at least about 75, at least about 100, at least about 125, at least about 150, at least about 175, or at least about 200, or at least about 220 amino acids in length. In certain embodiments, the functional fragment retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of amino acids 1-242 of SEQ ID NO: 56.

In certain embodiments, the transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 243-263 of SEQ ID NO: 56. In certain embodiments, the transmembrane domain of CD80 comprises or consists of amino acids 243-263 of SEQ ID NO: 56 or a fragment thereof. Such fragments can be at least about 5, at least about 10, at least about 15, or at least about 20 amino acids in length. In certain embodiments, the transmembrane domain of CD80 comprises or consists of amino acids 243-263 of SEQ ID NO: 56.

Non-limiting examples of co-stimulatory molecules include CD28, 4-1BB, OX40, ICOS, DAP-10, CD27, CD40, NKG2D, CD2, and combinations thereof.

In certain embodiments, the fusion polypeptide comprises an extracellular domain and a transmembrane domain of a co-stimulatory molecule that is 4-1BB. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 25 or a fragment thereof, and/or may optionally comprise up to one or up to two or up to three conservative amino acid substitutions. In certain embodiments, the 4-1BB comprises or consists of an amino acid sequence that is a consecutive portion of the amino acid sequence of SEQ ID NO: 25. In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 214-255 of SEQ ID NO: 25 or a fragment thereof, and/or may optionally comprise up to one or up to two or up to three conservative amino acid substitutions. In certain embodiments, the intracellular domain of 4-1BB comprises or consists of amino acids 214-255 of SEQ ID NO: 25 or a functional fragment thereof. Such functional fragment can be a consecutive portion of amino acids 214-255 of SEQ ID NO: 25, which is at least about 20, at least about 25, at least about 30, at least about 35, or at least about 40 amino acids in length. In certain embodiments, the functional fragment of amino acids 214-255 of SEQ ID NO: 25 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of amino acids 214-255 of SEQ ID NO: 25.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 7 or a fragment thereof, and/or may optionally comprise up to one or up to two or up to three conservative amino acid substitutions. In certain embodiments, the CD28 comprises or consists of an amino acid sequence that is a consecutive portion of the amino acid sequence of SEQ ID NO: 7. In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 180 to 219 of SEQ ID NO: 7 or a fragment thereof, and/or may optionally comprise up to one or up to two or up to three conservative amino acid substitutions. In certain embodiments, the intracellular domain of CD28 comprises or consists of amino acids 180 to 219 of SEQ ID NO: 7 or a functional fragment thereof. A functional fragment of amino acids 180 to 219 of SEQ ID NO: 7 can be a consecutive portion of amino acids 180 to 219 of SEQ ID NO: 7, which is at least about 20, at least about 25, at least about 30, or at least about 35 amino acids in length. In certain embodiments, such functional fragment retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of amino acids 180 to 219 of SEQ ID NO: 7.

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecules can be the same or different from each other.

In certain embodiments, the fusion polypeptide comprises an extracellular domain and a transmembrane domain of a co-stimulatory ligand that is CD80, and an intracellular domain of a co-stimulatory molecule that is 4-1BB. In certain embodiments, the fusion polypeptide comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 58. In certain embodiments, the fusion polypeptide comprises or consists of the amino acid sequence set forth in SEQ ID NO: 58. SEQ ID NO: 58 is provided below.

[SEQ ID NO: 58]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFKRGRKKLLYIFKQPFMRPVQTTQEEDGCSCRFP
EEEEGGCEL

In certain embodiments, the fusion polypeptide comprises an extracellular domain and a transmembrane domain of a co-stimulatory ligand that is CD80, an intracellular domain of a first co-stimulatory molecule that is 4-1BB, and an intracellular domain of a second co-stimulatory molecule that is CD28.

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 59. In certain embodiments, the fusion polypeptide comprises or consists of the amino acid sequence set forth in SEQ ID NO: 59. SEQ ID NO: 59 is provided below.

[SEQ ID NO: 59]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTINHSFMCLIKYGHLRVNQTFNWNTTKQEHFPDNLLPSWAIT
LISVNGIFVICCLTYCFRSKRSRLLHSDYMNMTPRRPGPTRKHYQPYAPP
RDFAAYRKRGRKKLLYIFKQPFMRPVQTTQEEDGCCRFPEEEEGGCEL

Various modified fusion polypeptides are disclosed in International Patent Application No. PCT/US20/42753, which is incorporated by reference hereby in its entirety.

### 5.4.4. Gene Disruption of CD70 Locus

The presently disclosed subject matter provides induced T cells comprising a gene disruption of a CD70 locus. A gene disruption of a CD70 locus in induced T cells disclosed herein can improve at least one activity of the induced T cells, e.g., cytotoxicity, cell proliferation, and/or cell persistence.

The gene disruption of the CD70 locus can result in a non-functional CD70 protein or a knockout of the CD70 gene expression. In certain embodiments, the gene disruption of the CD70 locus results in knockout of the CD70 gene expression.

Any methods to generate the gene disruption as disclosed above (e.g., any methods to generate the gene disruption disclosed in Section 5.3.2) can be used to generate the gene disruption of the CD70 locus. In certain embodiments, the gene disruption of the CD70 locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CD70 locus can be a disruption of the coding region of the CD70 locus and/or a disruption of the non-coding region of the CD70 locus. In certain embodiments, the gene disruption of the CD70 locus comprises a disruption of the coding region of the CD70 locus. In certain embodiments, the gene disruption of the CD70 locus comprises an insertion at the coding region of the CD70 locus. Human CD70 protein comprises three exons: exon 1, exon 2, and exon 3. In certain embodiments, the gene disruption of the CD70 locus comprises a disruption at one or more of exon 1, exon 2, and exon 3 of the CD70 locus. In certain embodiments, the gene disruption of the CD70 locus comprises a disruption at exon 1 of the CD70 locus. In certain embodiments, the gene disruption of the CD70 locus comprises an insertion at exon 1 of the CD70 locus.

### 5.4.5. Gene Disruption of CD52 Locus

The presently disclosed subject matter provides induced T cells comprising a gene disruption of a CD52 locus. A gene disruption of a CD52 locus in induced T cells disclosed herein can improve at least one activity of the induced T cells, e.g., cytotoxicity, cell proliferation, and/or cell persistence.

The gene disruption of the CD52 locus can result in a non-functional CD52 protein or a knockout of the CD52 gene expression. In certain embodiments, the gene disruption of the CD52 locus results in knockout of the CD52 gene expression.

Any methods to generate the gene disruption as disclosed above (e.g., any methods to generate the gene disruption disclosed in Section 5.3.2) can be used to generate the gene disruption of the CD52 locus. In certain embodiments, the gene disruption of the CD52 locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CD52 locus can be a disruption of the coding region of the CD52 locus and/or a disruption of the non-coding region of the CD52 locus. In certain embodiments, the gene disruption of the CD52 locus comprises a disruption of the coding region of the CD52 locus. In certain embodiments, the gene disruption of the CD52 locus comprises an insertion at the coding region of the CD52 locus. Human CD52 protein comprises two exons: exon 1 and exon 2. In certain embodiments, the gene disruption of the CD52 locus comprises a disruption at one or both of exon 1 and exon 2 of the CD52 locus. In certain embodiments, the gene disruption of the CD52 locus comprises a disruption at exon 1 of the CD52 locus. In certain embodiments, the gene disruption of the CD52 locus comprises an insertion at exon 1 of the CD52 locus.

### 5.4.6. Gene Disruption of PD1 Locus

The presently disclosed subject matter provides induced T cells comprising a gene disruption of a PD1 locus. A gene disruption of a PD1 locus in induced T cells disclosed herein can improve at least one activity of the induced T cells, e.g., cytotoxicity, cell proliferation, and/or cell persistence.

The gene disruption of the PD1 locus can result in a non-functional PD1 protein or a knockout of the PD1 gene expression. In certain embodiments, the gene disruption of the PD1 locus results in knockout of the PD1gene expression.

Any methods to generate the gene disruption as disclosed above (e.g., any methods to generate the gene disruption disclosed in Section 5.3.2) can be used to generate the gene disruption of the PD1 locus. In certain embodiments, the gene disruption of the PD1 locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the PD1 locus can be a disruption of the coding region of the PD1 locus and/or a disruption of the non-coding region of the PD1 locus. In certain embodiments, the gene disruption of the PD1 locus comprises a disruption of the coding region of the PD1 locus. In certain embodiments, the gene disruption of the PD1 locus comprises an insertion at the coding region of the PD1 locus. Human PD1 protein comprises six exons: exon 1, exon 2, exon 3, exon 4, exon 5, and exon 6. In certain embodiments, the gene disruption of the PD1 locus comprises a disruption at one or more of exon 1, exon 2, exon 3, exon 4, exon 5, and exon 6 of the PD1 locus. In certain embodiments, the gene disruption of the PD1 locus comprises a disruption at exon 1 of the PD1 locus. In certain embodiments, the gene disruption of the PD1 locus comprises an insertion at exon 1 of the PD1 locus.

### 5.4.7. Gene Disruption of CD38 Locus

The presently disclosed subject matter provides induced T cells comprising a gene disruption of a CD38 locus. A gene disruption of a CD38 locus in induced T cells disclosed herein can improve at least one activity of the induced T cells, e.g., cytotoxicity, cell proliferation, and/or cell persistence.

The gene disruption of the CD38 locus can result in a non-functional CD38 protein or a knockout of the CD38 gene expression. In certain embodiments, the gene disruption of the CD38 locus results in knockout of the CD38 gene expression.

Any methods to generate the gene disruption as disclosed above (e.g., any methods to generate the gene disruption disclosed in Section 5.3.2) can be used to generate the gene disruption of the CD38 locus. In certain embodiments, the gene disruption of the CD38 locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CD38 locus can be a disruption of the coding region of the CD38 locus and/or a disruption of the non-coding region of the CD38 locus. In certain embodiments, the gene disruption of the CD38 locus comprises a disruption of the coding region of the CD38 locus. In certain embodiments, the gene disruption of the CD38 locus comprises an insertion at the coding region of the CD38 locus. Human CD38 protein comprises eight exons: exon 1, exon 2, exon 3, exon 4, exon 5, exon 6, exon 7, and exon 8. In certain embodiments, the gene disruption of the CD38 locus comprises a disruption at one or more of exon 1, exon 2, exon 3, exon 4, exon 5, exon 6, exon 7, and exon 8 of the CD38 locus. In certain embodiments, the gene disruption of the CD38 locus comprises a disruption at exon 1 of the CD38 locus. In certain embodiments, the gene disruption of the CD38 locus comprises an insertion at exon 1 of the CD38 locus.

### 5.4.8. Gene Disruption of PLZF Locus

The presently disclosed subject matter provides induced T cells comprising a gene disruption of a PLZF locus. A gene disruption of a PLZF locus in induced T cells disclosed herein can improve at least one activity of the induced T cells, e.g., cytotoxicity, cell proliferation, and/or cell persistence.

The gene disruption of the PLZF locus can result in a non-functional PLZF protein or a knockout of the PLZF gene expression. In certain embodiments, the gene disruption of the PLZF locus results in knockout of the PLZF gene expression.

Any methods to generate the gene disruption as disclosed above (e.g., any methods to generate the gene disruption disclosed in Section 5.3.2) can be used to generate the gene disruption of the PLZF locus. In certain embodiments, the gene disruption of the PLZF locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the PLZF locus can be a disruption of the coding region of the PLZF locus and/or a disruption of the non-coding region of the PLZF locus. In certain embodiments, the gene disruption of the PLZF locus comprises a disruption of the coding region of the PLZF locus. In certain embodiments, the gene disruption of the PLZF locus comprises an insertion at the coding region of the PLZF locus. Human PLZF protein comprises six exons: exon 1, exon 2, exon 3, exon 4, exon 5, and exon 6. In certain embodiments, the gene disruption of the PLZF locus comprises a disruption at one or more of exon 1, exon 2, exon 3, exon 4, exon 5, and exon 6 of the PLZF locus. In certain embodiments, the gene disruption of the PLZF locus comprises a disruption at exon 1 of the PLZF locus. In certain embodiments, the gene disruption of the PLZF locus comprises an insertion at exon 1 of the PLZF locus.

### 5.4.9. Gene Disruption of SOX13 Locus

The presently disclosed subject matter provides induced T cells comprising a gene disruption of a SOX13 locus. A gene disruption of a SOX13 locus in induced T cells disclosed herein can improve at least one activity of the induced T cells, e.g., cytotoxicity, cell proliferation, and/or cell persistence. The gene disruption of the SOX13 locus can result in a non-functional SOX13 protein or a knockout of the SOX13 gene expression. In certain embodiments, the gene disruption of the SOX13 locus results in knockout of the SOX13 gene expression.

Any methods to generate the gene disruption as disclosed above (e.g., any methods to generate the gene disruption disclosed in Section 5.3.2) can be used to generate the gene disruption of the PD1 locus. In certain embodiments, the gene disruption of the SOX13 locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the SOX13 locus can be a disruption of the coding region of the SOX13 locus and/or a disruption of the non-coding region of the SOX13 locus. In certain embodiments, the gene disruption of the SOX13 locus comprises a disruption of the coding region of the SOX13 locus. In certain embodiments, the gene disruption of the SOX13 locus comprises an insertion at the coding region of the SOX13 locus. Human SOX13 protein comprises fourteen (14) exons: exon 1, exon 2, exon 3, exon 4, exon 5, exon 6, exon 7, exon 8, exon 9, exon 10, exon 11, exon 12, exon 13, and exon 14. In certain embodiments, the gene disruption of the SOX13 locus comprises a disruption at one or more of exon 1, exon 2, exon 3, exon 4, exon 5, exon 6, exon 7, exon 8, exon 9, exon 10, exon 11, exon 12, exon 13, and exon 14 of the SOX13 locus. In certain embodiments, the gene disruption of the SOX13 locus comprises a disruption at exon 1 of the SOX13 locus. In certain embodiments, the gene disruption of the SOX13 locus comprises an insertion at exon 1 of the SOX13 locus.

### 5.5 Formulations and Administration

The presently disclosed subject matter provides compositions comprising presently disclosed cells (e.g., disclosed in Section 5.4). In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10¹ and about 10⁷, between about 10¹ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10′ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells)) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 5.6 Methods of Treatment

The presently disclosed subject matter provides various methods of using the presently disclosed cells (e.g., produced by the methods disclosed in Section 5.3) or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenstrom macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the neoplasm or tumor is a lymphocytic disorder. In certain embodiments, the lymphocytic disorder is selected from the group consisting acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia.

In certain embodiments, the neoplasm or tumor is a lymphoma. In certain embodiments, the lymphoma is Hodgkin's lymphoma or non-Hodgkin's lymphoma.

In certain embodiments, the neoplasm or tumor is a B cell malignancy. In certain embodiments, the B cell malignancy is selected from the group consisting of B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma. In certain embodiments, the neoplasm or tumor is B cell lymphoma. In certain embodiments, the B cell lymphoma is relapsed or refractory (R/R) B cell lymphoma.

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

In certain embodiments, the myeloid disorder is acute myeloid leukemia (AML). In certain embodiments, the first and/or second antigens are independently selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an infectious disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into the presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion or eradicated at the earliest signs of toxicity.

### 5.7 Kits

The presently disclosed subject matter provides kits for inducing differentiation of pluripotent stem cells to induced T cells. In certain embodiments, the kit comprises (a) a nucleic acid composition encoding a chimeric antigen receptor (CAR), (b) a polypeptide that engages the CAR, and (c) an antibody or antigen-binding fragment thereof that binds 4-1BB. In certain embodiments, the kit comprises (a) a nucleic acid composition encoding a chimeric antigen receptor (CAR), (b) a polypeptide that stimulates the CAR, and (c) an antibody or antigen-binding fragment thereof that binds 4-1BB. In certain embodiments, the kit comprises (a) a nucleic acid composition encoding a chimeric antigen receptor (CAR), (b) a polypeptide that activates the CAR, and (c) an antibody or antigen-binding fragment thereof that binds 4-1BB.

In certain embodiments, the polypeptide that engages the CAR is an antibody or antigen-binding fragment thereof. In certain embodiments, the antibody or antigen-binding fragment thereof binds to a scFv of the CAR. In certain embodiments, the antibody or antigen-binding fragment thereof binds to an idiotypic variable domain of a scFv of the CAR.

In certain embodiments, the polypeptide that engages the CAR is an antigen-containing polypeptide. In certain embodiments, the antigen-containing polypeptide is an antigen or a fragment thereof. In certain embodiments, the antigen-containing polypeptide is an Fc-fusion protein.

In certain embodiments, the polypeptide that stimulates the CAR is an antibody or antigen-binding fragment thereof. In certain embodiments, the antibody or antigen-binding fragment thereof binds to a scFv of the CAR. In certain embodiments, the antibody or antigen-binding fragment thereof binds to an idiotypic variable domain of a scFv of the CAR.

In certain embodiments, the polypeptide that stimulates the CAR is an antigen-containing polypeptide. In certain embodiments, the antigen-containing polypeptide is an antigen or a fragment thereof. In certain embodiments, the antigen-containing polypeptide is an Fc-fusion protein.

In certain embodiments, the polypeptide that activates the CAR is an antibody or antigen-binding fragment thereof. In certain embodiments, the antibody or antigen-binding fragment thereof binds to a scFv of the CAR. In certain embodiments, the antibody or antigen-binding fragment thereof binds to an idiotypic variable domain of a scFv of the CAR.

In certain embodiments, the polypeptide that activates the CAR is an antigen-containing polypeptide. In certain embodiments, the antigen-containing polypeptide is an antigen or a fragment thereof. In certain embodiments, the antigen-containing polypeptide is an Fc-fusion protein.

In certain embodiments, the kit further comprises instructions for inducing differentiation of the pluripotent stem cells into induced T cells.

In certain embodiments, the instructions comprise contacting the pluripotent stem cells with the cell culture media in a specific sequence. In certain embodiments, the instructions comprise contacting the pluripotent stem cells according to the methods disclosed herein (see Section 5.3).

The presently disclosed subject matter also provides kits for inducing and/or enhancing an immune response and/or treating and/or preventing a neoplasm or a pathogen infection (e.g., an autoimmune disease or an infectious disease) in a subject. In certain embodiments, the kit comprises an effective amount of presently disclosed cells (e.g., induced T cell including a first antigen-recognizing receptor), a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding antigen-recognizing receptors (e.g., a CAR, a TCR, or a TCR like fusion molecule) directed toward an antigen of interest in expressible form, which may optionally be comprised in the same or different vectors.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.8 EXEMPLARY EMBODIMENTS

A1. In certain non-limiting embodiments, the presently disclosed subject matter provides a method of expanding a population of induced T cells, the method comprising:
(a) contacting an induced T cell comprising an antigen-recognizing receptor with a polypeptide that engages the antigen-recognizing receptor and an agonist of 4-1BB, and
(b) culturing the induced T cell to thereby produce an expanded population of induced T cells; wherein the antigen-recognizing receptor is a chimeric antigen receptor (CAR) or a TCR like fusion protein (HIT).

A2. The foregoing method of A1, wherein the polypeptide that engages the antigen-recognizing receptor is an antibody or antigen-binding fragment thereof.

A3. The foregoing method of A2, wherein the antibody or antigen-binding fragment thereof binds to a scFv of the CAR.

A4. The foregoing method of A2 or A3, wherein the antibody or antigen-binding fragment thereof binds to an idiotypic variable domain of a scFv of the CAR.

A5. The foregoing method of A2, wherein the antibody or antigen-binding fragment thereof binds to an antigen-binding chain of the HIT.

A6. The foregoing method of A2 or A3, wherein the antibody or antigen-binding fragment thereof binds to an idiotypic variable domain of an antigen-binding chain of the HIT.

A7. The foregoing method of any one of A2-A6, wherein the antibody or antigen-binding fragment thereof comprises:
(a) a heavy variable region comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62; and a light variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; or
(b) a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

A8. The foregoing method of any one of A2-A7, wherein the antigen-recognizing receptor binds to CD19 and the antibody or antigen-binding fragment thereof comprises a heavy variable region comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62; and a light variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65.

A9. The foregoing method of A2-A7, wherein the antigen-recognizing receptor binds to CD19 and the antibody or antigen-binding fragment thereof comprises a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

A10. The foregoing method of A2-A7, wherein the antigen-recognizing receptor binds to PSMA and the antibody or antigen-binding fragment thereof comprises a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

A11. The foregoing method of A1, wherein the polypeptide that engages the antigen-recognizing receptor is an antigen-containing polypeptide.

A12. The foregoing method of A11, wherein the antigen-containing polypeptide is an antigen or a fragment thereof.

A13. The method of A11 or A12, wherein the antigen-containing polypeptide is an Fc-fusion protein.

A14. The method of any one of A1-A13, wherein the agonist of 4-1BB is an antibody or antigen-binding fragment thereof that binds 4-1BB.

A15. The foregoing method of A14, wherein the antibody or antigen-binding fragment thereof that binds 4-1BB is urelumab.

A16. The foregoing method of A14 or A15, wherein the antibody or antigen-binding fragment thereof that binds 4-1BB comprises a heavy chain comprising an amino acid sequence that at least about 80%, at least about 85%, at least about 90%, at least about 95%, at least about 96%, at least about 97%, at least about 98%, or at least about 99%, at least about 100% identical to the amino sequence set forth in SEQ ID NO: 54, and a light chain comprising an amino acid sequence that at least about 80%, at least about 85%, at least about 90%, at least about 95%, at least about 96%, at least about 97%, at least about 98%, or at least about 99%, at least about 100% identical to the amino sequence set forth in SEQ ID NO: 55.

A17. The foregoing method of A16, wherein the antibody or antigen-binding fragment thereof binds 4-1BB comprises a heavy chain comprising the amino sequence set forth in SEQ ID NO: 54, and a light chain comprising the amino sequence set forth in SEQ ID NO: 55.

A18. The foregoing method of any one of A1-A17, wherein the antigen-recognizing receptor binds to a first antigen that is a tumor antigen or a pathogen antigen.

A19. The foregoing method of A18, wherein the first antigen is a tumor antigen or a pathogen antigen A20. The foregoing method of A18 or A19, wherein the first antigen is selected from the group consisting of CD19, CD70, IL1RAP, ABCG2, AChR, ACKR6, ADAMTS13, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

A21. The foregoing method of any one of A1-A20, wherein the HIT comprises an extracellular antigen-binding domain that binds to the first antigen and is capable of delivering an activation signal to the cell.

A22. The foregoing method of any one of A1-A20, wherein the CAR comprises an extracellular antigen-binding domain that binds to the first antigen and an intracellular signaling domain that is capable of delivering an activation signal to the cell.

A23. The foregoing method of A22, wherein the intracellular signaling domain comprises a native CD3ζ polypeptide or a modified CD3ζ polypeptide.

A24. The foregoing method of A23, wherein the modified CD3ζ polypeptide comprises a native ITAM1, an ITAM2 variant consisting of two loss-of-function mutations, and an ITAM3 variant consisting of two loss-of-function mutations.

A25. The foregoing method of A23 or A24, wherein the modified CD3ζ polypeptide comprises or consists of the amino acid sequence set forth in SEQ ID NO: 21.

A26. The foregoing method of any one of A1-A25, wherein the antigen-recognizing receptor is encoded by a polynucleotide integrated at a locus within the genome of the induced T cell.

A27. The foregoing method of A26, wherein the locus is selected from the group consisting of a TRAC locus, a TRBC locus, a TRDC locus, and a TRGC locus.

A28. The foregoing method of A26 or A27, wherein the locus is a TRAC locus or a TRBC locus.

A29. The foregoing method of claim 26, wherein the locus is a TRAC locus.

A30. The foregoing method of any one of A1-A29, wherein culturing comprises contacting the induced T cell comprising a chimeric receptor with IL-7, IL-21, or a combination thereof.

A31. The foregoing method of any one of A1-A30, wherein the induced T cell is a cytotoxic T lymphocyte (CTL), a regulatory T cell, or a Natural Killer T (NKT) cell.

A32. The foregoing method of any one of A1-A31, wherein the induced T cell is
(a) CD3+, TCR−;
(b) CD4+, CD3+, and TCR−; or
(c) CD8+, CD3+, and TCR−.

A33. The foregoing method of A32, wherein the induced T cell is:
(a) CD3+, TCR−, CD25+, CD28+, CD69+, CD56+, CD45RA+;
(b) CD3+, TCR−, CD4−, CD8αα−;
(c) CD3+, TCR−, CD4−, CD8αβ−;
(d) CD3+, TCR−, CD4−, CD8αα+;
(e) CD3+, TCR−, CD4−, CD8αβ+;
(f) CD3+, TCR−, CD4+, CD8αα−;
(h) CD3+, TCR−, CD4+, CD8αβ−;
(i) CD3+, TCR−, CD4+, CD8αα+; or
(j) CD3+, TCR−, CD4+, CD8αβ+.

A34. The foregoing method of any one of A1-A33, wherein the induced T cell further comprises a gene disruption at a second locus selected from the group consisting of a CD52 locus, a CD70 locus, a PD1 locus, a CD38 locus, a PLZF locus, a SOX13 locus, and a combination thereof.

A35. The foregoing method of any one of A1-A34, wherein the induced T cell further comprises a second antigen-recognizing receptor that targets a second antigen.

A36. The foregoing method of A35, wherein the second antigen-recognizing receptor is a chimeric antigen receptor (CAR), a chimeric costimulatory receptor (CCR), a T cell receptor (TCR), or a TCR like fusion molecule.

A37. The foregoing method of A35 or A36, wherein the second antigen is a tumor antigen or a pathogen antigen.

A38. The foregoing method of any one of A35-A37, wherein the second antigen is independently selected from the group consisting of CD19, CD70, IL1RAP, ABCG2, AChR, ACKR6, ADAMTS13, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

A39. The foregoing method of any one of A23-A38, wherein the intracellular signaling domain of the CAR further comprises at least one costimulatory signaling region.

A40. The foregoing method of A39, wherein the at least one costimulatory signaling region comprises at least an intracellular domain of a co-stimulatory molecule or a portion thereof.

A41. The foregoing method of A40, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

B1. In certain non-limiting embodiments, the presently disclosed subject matter provides a method of obtaining and expanding a population of induced T cells, the method comprising:
(a) introducing into a pluripotent stem cell a polynucleotide encoding an antigen-recognizing receptor, wherein the antigen-recognizing receptor is a chimeric antigen receptor (CAR) or a TCR like fusion protein (HIT);
(b) contacting the pluripotent stem cell with a first cell culture medium comprising an activator of the bone morphogenic protein pathway to differentiate the pluripotent stem cell into a hematopoietic precursor;
(c) contacting the pluripotent stem cell with a second cell culture medium comprising a Notch ligand to differentiate the hematopoietic precursor into induced T cell; and
(d) expanding the induced T cell with the method of any one of A1-A41.

B2. The foregoing method of B1, wherein the pluripotent stem cell is an induced pluripotent stem cell.

B3. The foregoing method of B1 or B2, wherein the pluripotent stem cell is a T cell-derived induced pluripotent stem cell.

B4. The foregoing method of any one of B1-B3, wherein the activator of the bone morphogenic protein pathway is a BMP-4 polypeptide (BMP-4).

B5. The foregoing method of any one of B1-B4, wherein the first cell culture medium further comprises a fibroblast growth factor.

B6. The foregoing method of B5, wherein the fibroblast growth factor is a basic fibroblast growth factor (bFGF).

B7. The foregoing method of any one of B1-B6, wherein the pluripotent stem cell is in contact with the first cell culture medium for up to about 4 days.

B8. The foregoing method of any one of B1-B7, wherein the first cell culture medium further comprises VEGF, SCF, FLT3L, IL3, IL-6, IL-11, TPO, IGF-1, EPO, SHH, angiotensin II, AGTR1, or a combination thereof.

B9. The foregoing method of any one of B1-B8, the pluripotent stem cell is in contact with the first cell culture medium for up to about 10 days.

B10. The foregoing method of any one of B1-B9, wherein the Notch ligand is a DLL-1 polypeptide, a DLL-4 polypeptide, a JAG-1 polypeptide, a JAG-2 polypeptide, or a combination thereof.

B11. The foregoing method of B10, wherein the Notch ligand is expressed by a feeder cell.

B12. The foregoing method of any one of B1-B11, wherein the second cell culture medium further comprises SCF, FLT3L, IL-3, IL-7, IL-6, IL-11, TPO, IGF-1, EPO, SHH, angiotensin II, AGTR1, or a combination thereof.

B13. The foregoing method of any one of B1-B12, wherein the hematopoietic precursor is in contact with the second cell culture medium for up to about 25 days.

C1. In certain non-limiting embodiments, the presently disclosed subject matter provides an induced T cell obtained by the method of any one of A1-A41 or B1-B13.

D1. In certain non-limiting embodiments, the presently disclosed subject matter provides a composition comprising the induced T cell obtained by the method of any one of A1-A41 or B1-B13.

D2. The foregoing composition of D1, which is a pharmaceutical composition further comprising a pharmaceutically acceptable excipient.

E1. In certain non-limiting embodiments, the presently disclosed subject matter provides a method of reducing tumor burden in a subject, the method comprising administering to the subject an effective amount of the induced T cell produced by the method of any one of A1-A41 or B1-B13 or the composition of D1 or D2.

E2. The foregoing method of claim E1, wherein the method reduces the number of tumor cells, reduces tumor size, and/or eradicates the tumor in the subject.

E3. In certain non-limiting embodiments, the presently disclosed subject matter provides a method of preventing and/or treating a neoplasm or a tumor in the subject, administering to the subject an effective amount of the induced T cell produced by the method of any one of A1-A41 or B1-B13 or the composition of D1 or D2.

E4. The foregoing method of any one of E1-E3, wherein the neoplasm or tumor is cancer.

E5. The foregoing method of any one of E1-E4, wherein the neoplasm or tumor is a solid tumor.

E6. The foregoing method of any one of E1-E4, wherein the neoplasm or tumor is a blood cancer.

E7. The foregoing method of E6, wherein the blood cancer is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

E8. In certain non-limiting embodiments, the presently disclosed subject matter provides a method of preventing and/or treating a pathogen infection in a subject, the method comprising administering to the subject an effective amount of the induced T cell produced by the method of any one of A1-A41 or B1-B13 or the composition of D1 or D2.

E9. In certain non-limiting embodiments, the presently disclosed subject matter provides a method of preventing and/or treating an autoimmune disease in a subject, the method comprising administering to the subject an effective amount of the induced T cell produced by the method of any one of A1-A41 or B1-B13 or the composition of D1 or D2.

E10. In certain non-limiting embodiments, the presently disclosed subject matter provides a method of preventing and/or treating an infectious disease in a subject, the method comprising administering to the subject an effective amount of the induced T cell produced by the method of any one of A1-A41 or B1-B13 or the composition of D1 or D2.

F1. In certain non-limiting embodiments, the presently disclosed subject matter provides a kit comprising the induced T cell produced by the method of any one of A1-A41 or B1-B13 or the composition of D1 or D2.

F2. The foregoing kit of F1, wherein the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

G1. The composition of D1 or D2 or the kit of F1 or F2 for use in reducing tumor burden, treating and/or preventing a neoplasm or a tumor, preventing and/or treating a pathogen infection, preventing and/or treating an autoimmune disease, and/or preventing and/or treating an infectious disease, in a subject.

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

T cells that are engineered to express a chimeric antigen receptor (CAR) can direct potent therapeutic responses in patients with chemorefractory hematologic malignancies (June & Sadelain, N Engl J Med 379, 64-73 (2018)). CARs are synthetic receptors that redirect T cell specificity and augment T cell functions to overcome tumor resistance. CAR T cells are now under investigation in a range of diseases, including solid tumors, infectious disease, autoimmunity and senescence-associated pathologies. CAR T cells are generally produced in autologous fashion, which is effective but presents some challenges. Manufacturing time is critical for patients with rapidly progressing disease; cell product variability is high because patient T cells may be reduced in number or functionality due to progressive disease or prior therapies; manufacturing processes and release testing are required on an individual basis and costly. Alternate T cell sources are under investigation to enable ‘off-the-shelf’ cellular therapy, including donor-derived lymphoid progenitors, T cells lacking allo-reactive potential (e.g., virus-specific T cells, γδTCR-T cells, invariant NKT cells and T cell receptor (TCR)-edited T cells) and pluripotent stem cell-derived T cells (Themeli et al., Cell Stem Cell 16, 357-366 (2015); Depil et al., Nat Rev Drug Discov 19, 185-199 (2020)). The use of allogeneic T cells harvested from healthy donors is the most explored alternative source, with some promising clinical results already obtained (Benjamin, et al., Lancet 396, 1885-1894 (2020)). However, maintaining product consistency after genetic engineering and manufacturing large cell batches of sufficient cell purity to avert graft versus host disease (GvHD) remain a challenge (Qasim et al., Sci Transl Med 9 (2017)). Pluripotent stem cells provide an attractive solution to overcome challenges associated with the use of autologous or allogeneic blood cells. Their self-renewing capacity should facilitate the selection of clones of a desired genotype, eventually combining multiple edits to enhance anti-tumor functions and pre-empt allo-reactivity and allo-rejection, and support large-scale production (Themeli et al., Nat Biotechnol 31, 928-933 443 (2013); Wang et al., Nat Biomed Eng 5, 429-440 (2021)).

It was previously demonstrated that CAR T cells can be generated from reprogrammed TiPS (Themeli et al., Nat Biotechnol 31, 928-933 (2013)). These original TiPS were retrovirally transduced to express a CD19-specific CAR (CAR-TiPS), and re-differentiated into T cells. The CAR T cells induced therefrom (CAR-TiPS-iT) homogeneously expressed both the transduced CAR (1928z) and the endogenous apTCR. The CAR-TiPS-iT cells were specific for CD19, highly lytic and controlled tumor growth in an intra-peritoneal lymphoma model in NSG mice (Themeli et al., Nat Biotechnol 31, 928-933 (2013)). Despite expressing their endogenous apTCR, these CAR-TiPS-iT cells did not acquire a conventional CD4 or CD8αβ T cell phenotype, but rather an innate-like CD8αβ-double-negative (DN) or CD8αα single-positive (SP) phenotype.

Transcriptional analyses confirmed that these CAR-TiPS-iT cells more closely resembled T6-T cells than αβ-T cells (Themeli et al., Nat Biotechnol 31, 928-933 (2013)). These findings were recently corroborated by Harada et al., who found that constitutive expression of a LMP2-specific CAR in TiPS likewise yielded DN and CD8α T cell phenotypes (Harada et al., Mol Ther (2021)). In another report, the lymphoid differentiation of induced pluripotent stem cells expressing a GPC3-specific CAR failed to induce characteristic T cell markers such as CD5 and CD3 and rather produced natural killer (NK) or innate lymphoid cells (ILC) (Ueda et al., Cancer Sci 111, 1478-1490 (2020)). These reports raise the questions of why T lineage commitment is deferred towards innate phenotypes and what is required to induce CD8αβ CAR T cells. Here, the impact of TCR and CAR expression on the commitment of TiPS-derived lymphoid progenitors to adaptive T cell lineages is analyzed.

Physiological αβ TCR-T cell development is regulated by TCR gene recombination, Notch and pre-TCR/TCR signaling (Yui & Rothenberg, Nat Rev Immunol 14, 529-545 (2014)). The TCR and Notch are central to the adoption of either γδTCR or αβTCR-T cell fates. The earlier rearrangement of the γ- and δ-chains results in maturation of γδTCR-T cells with a DN or CD8αα SP phenotype (Pardoll et al., J Immunol 140, 4091-4096 (1988)), whereas successful pre-TCR and αβTCR assembly drive progression from the DN to the CD4⁺CD8⁺ double positive (DP) stage before yielding SP T cells (Fehling et al., Nature 375, 795-798 (1995); Hogquist et al., J Exp Med 177, 1469-1473 (1993)). The presently disclosed subject matter shows that premature αβTCR or constitutive CAR expression interfere with DP formation, depending on the strength of Notch stimulation. Delaying CAR expression through TRAC promoter-controlled expression and calibration of CAR signaling through CD3ζ immunoreceptor tyrosine activation motif (ITAM) mutations enable DP CART iT cell development. In absence of an αβTCR, the CAR drives T cell maturation and yields CD8αβ⁺ CAR⁺ iT cells that mediate durable remissions in a systemic leukemia model.

DLL4 Stimulation Facilitates CD4⁺CD8αβ+DP T Cell Development from WT-TiPS but not CAR-TiPS

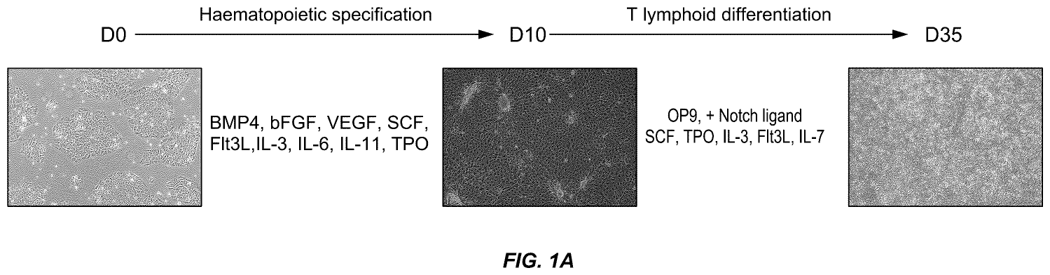

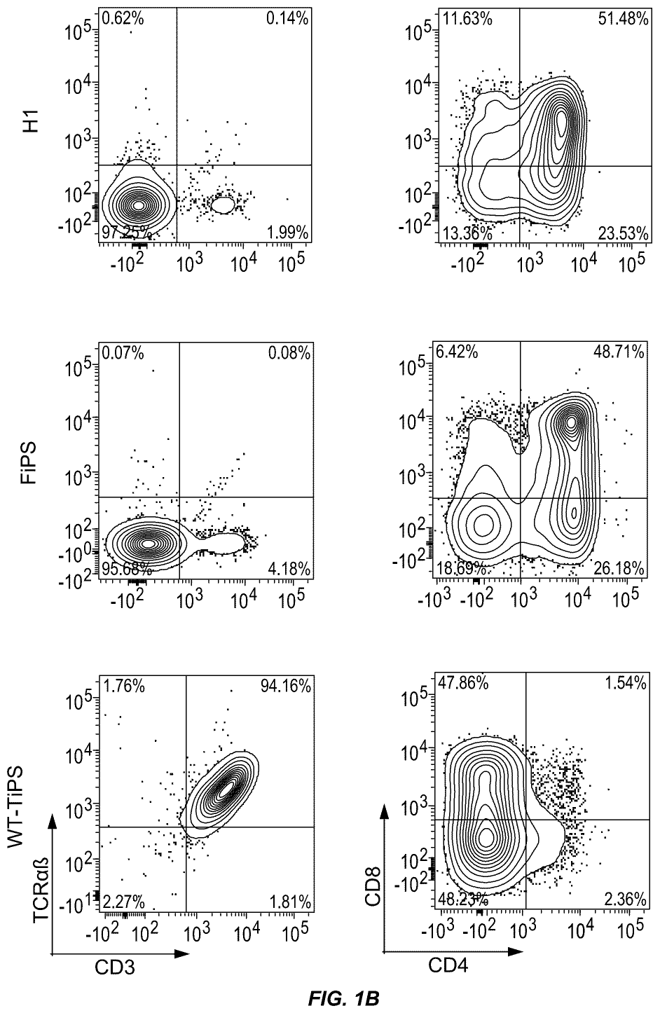

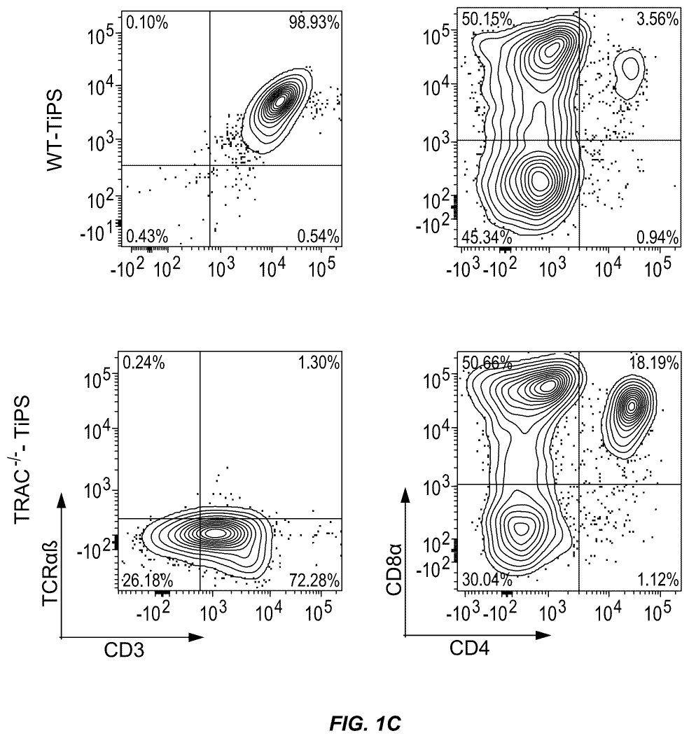

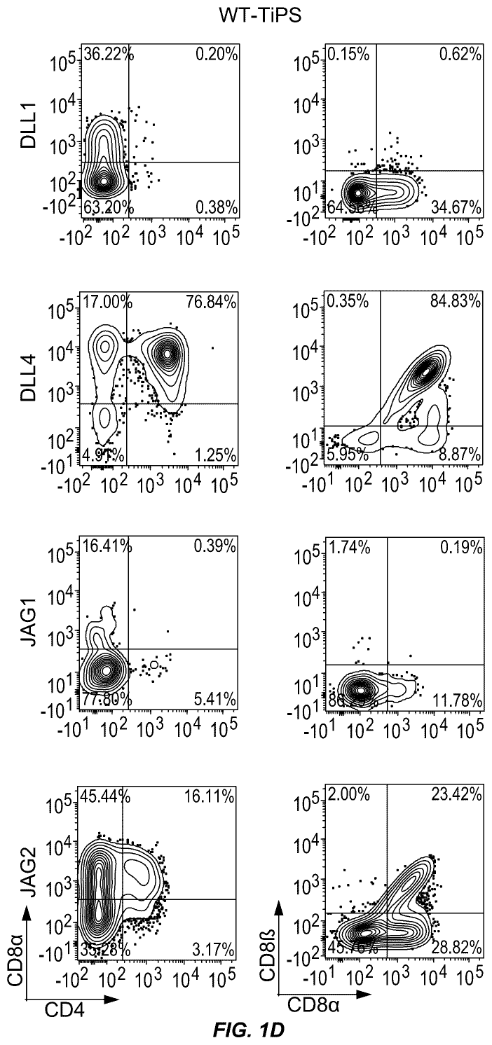

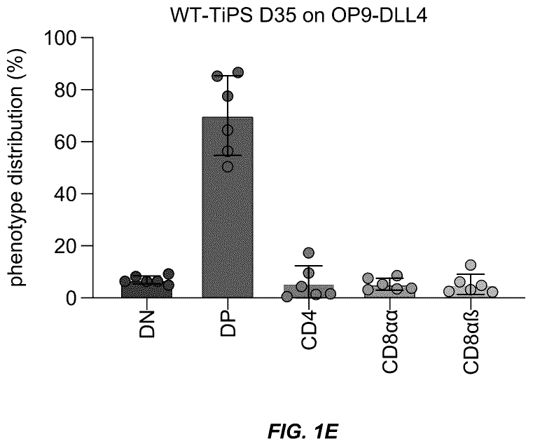

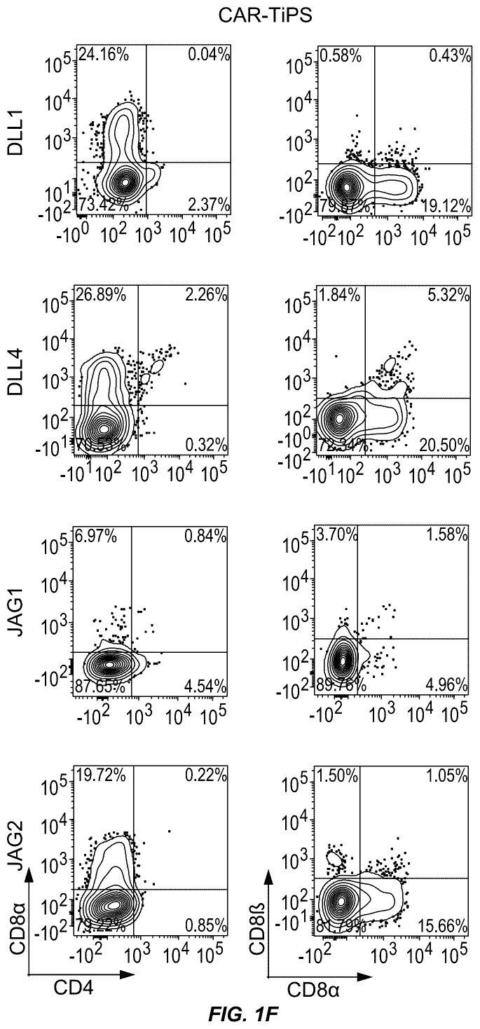

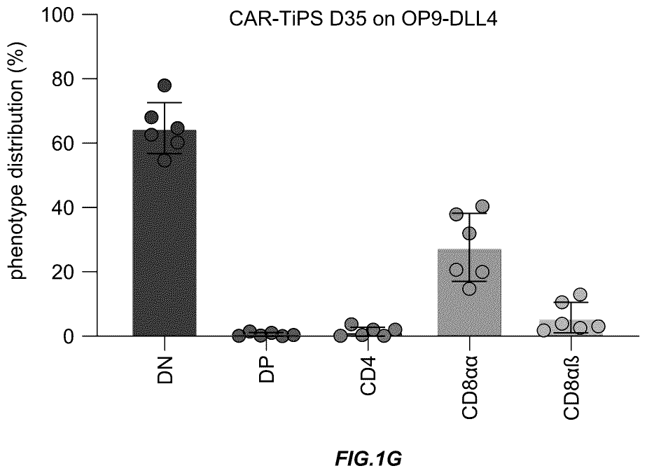

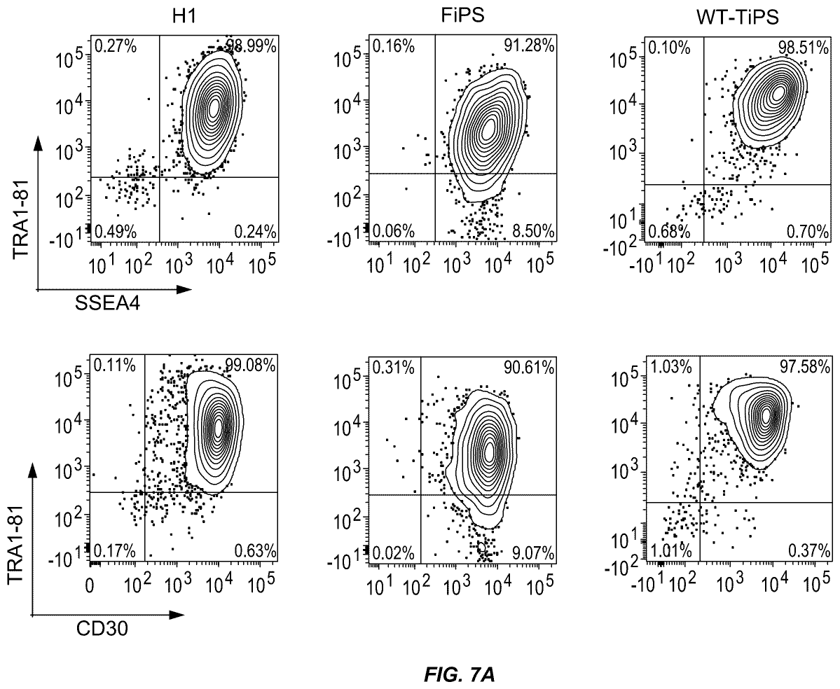

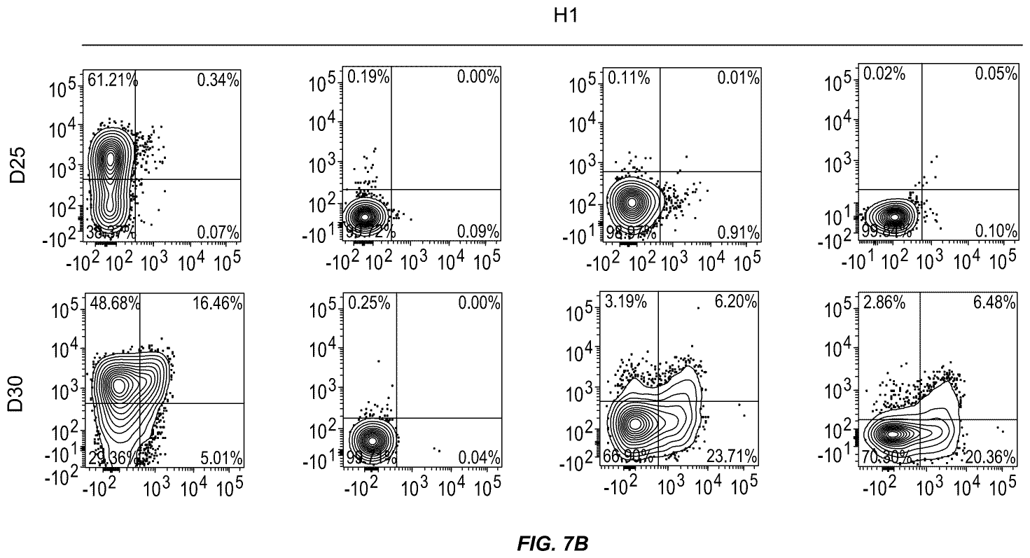

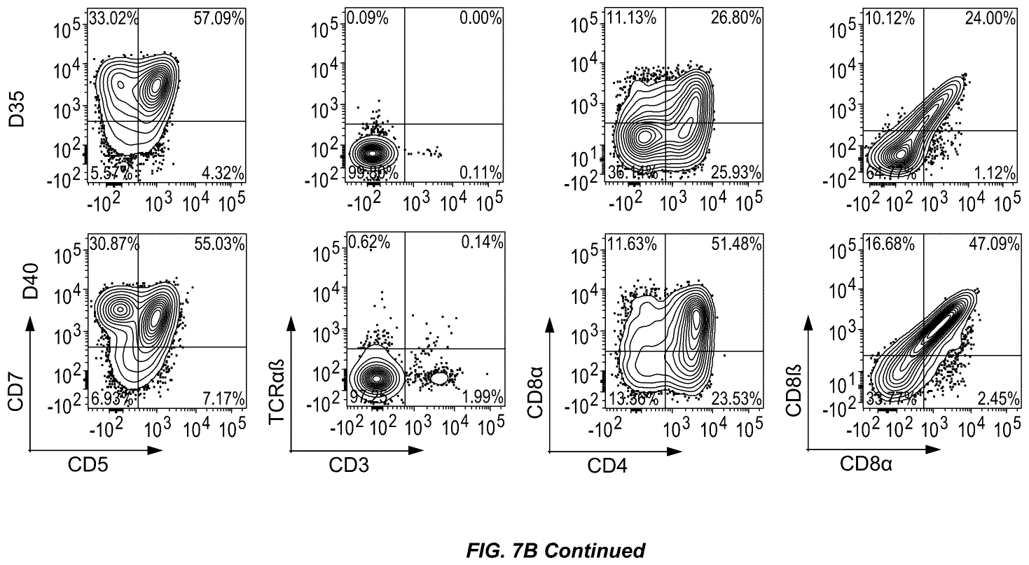

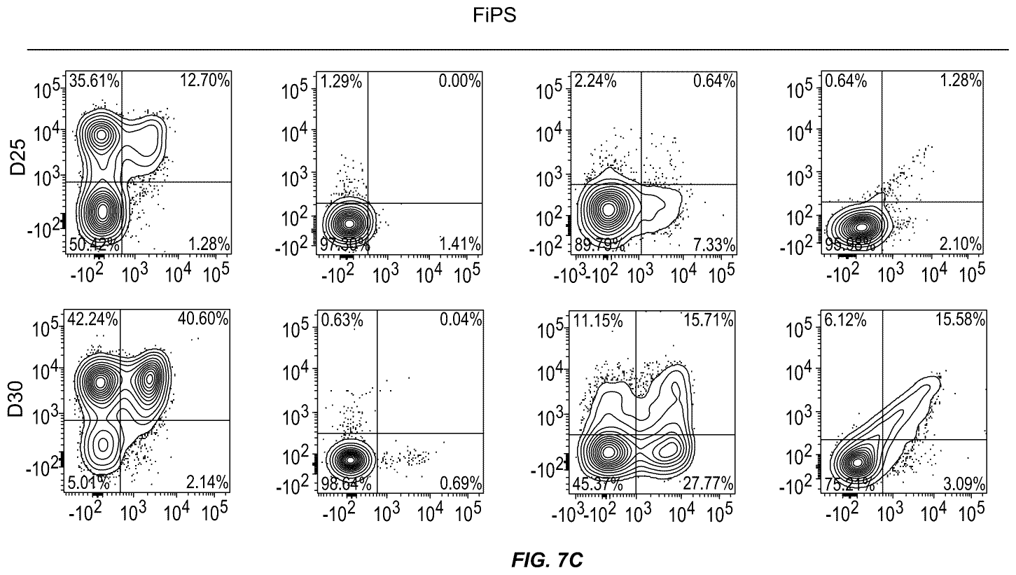

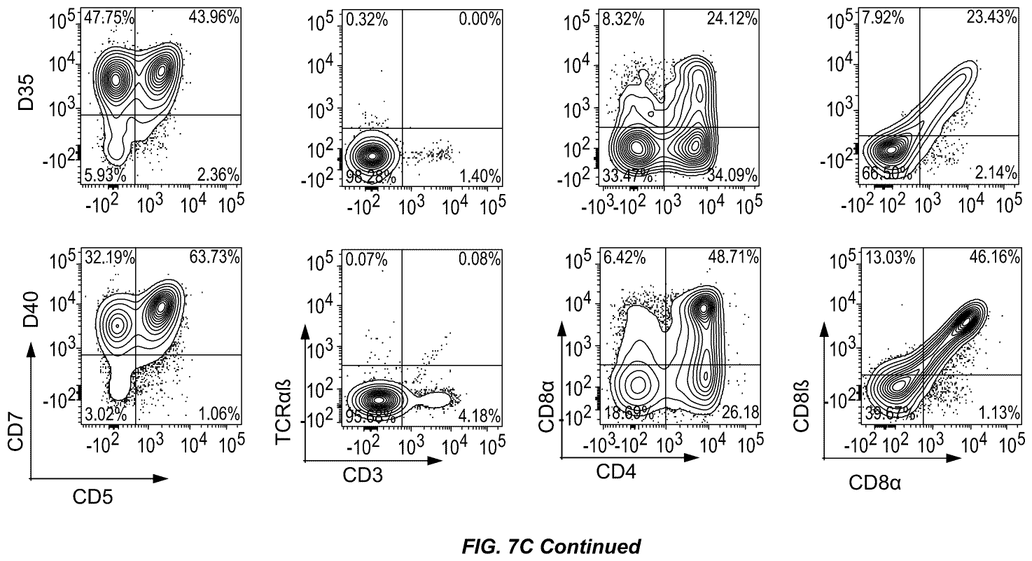

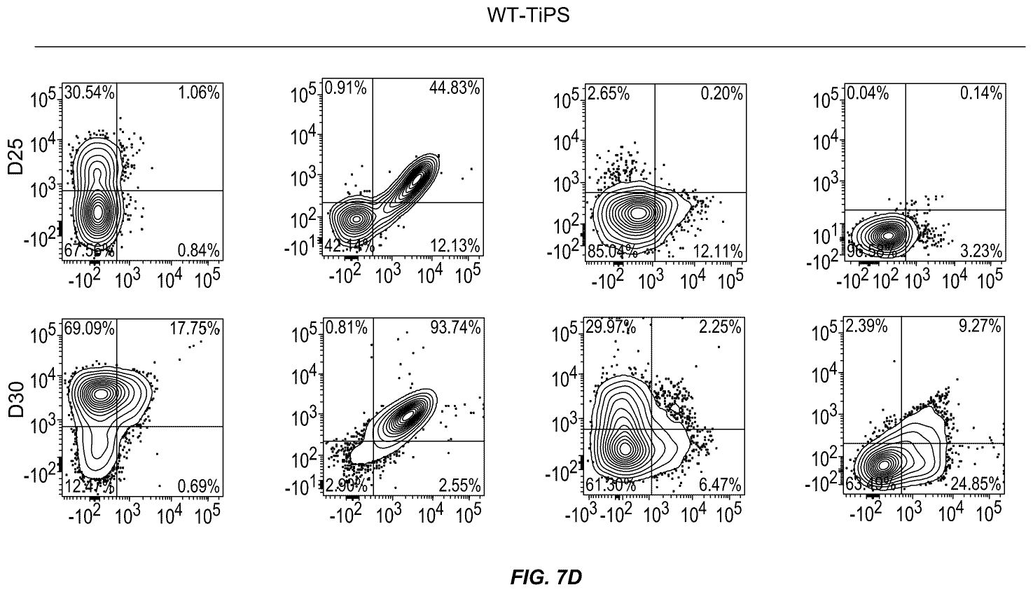

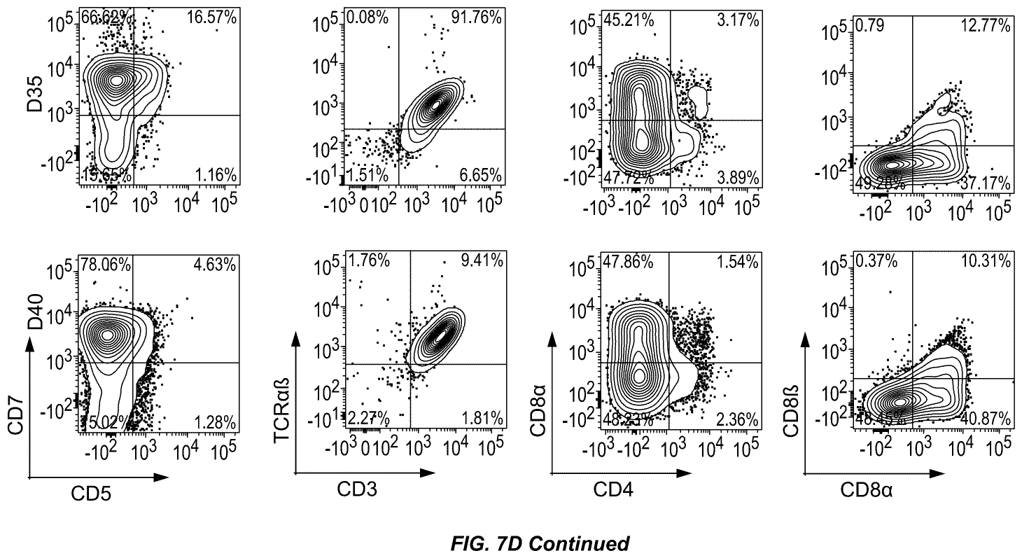

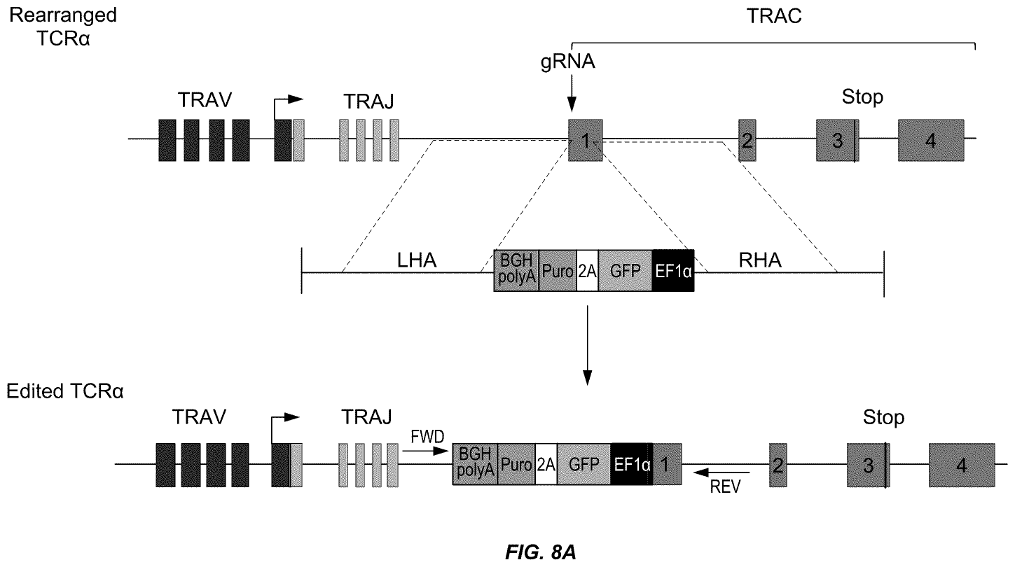

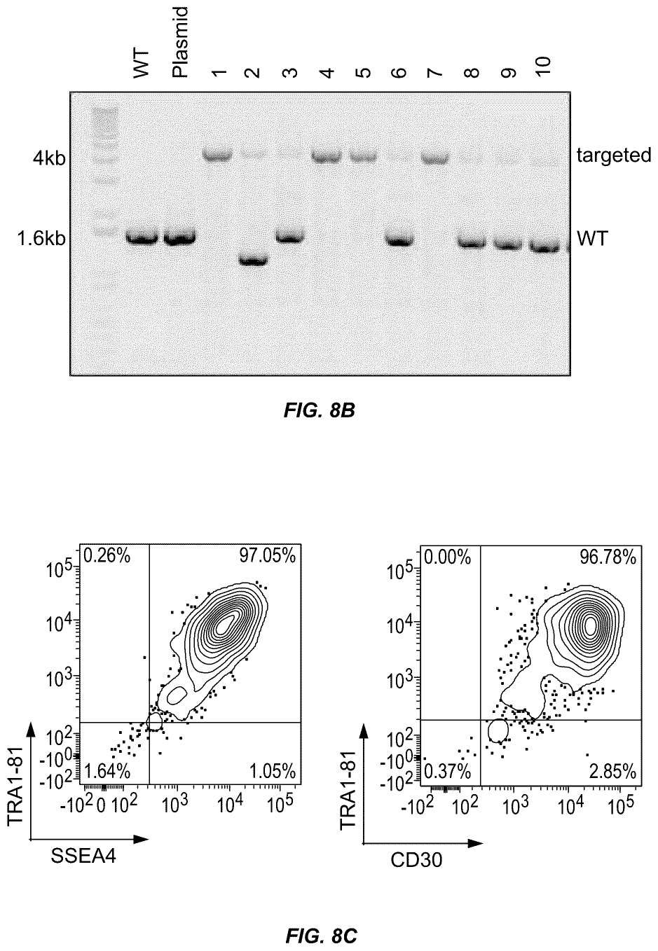

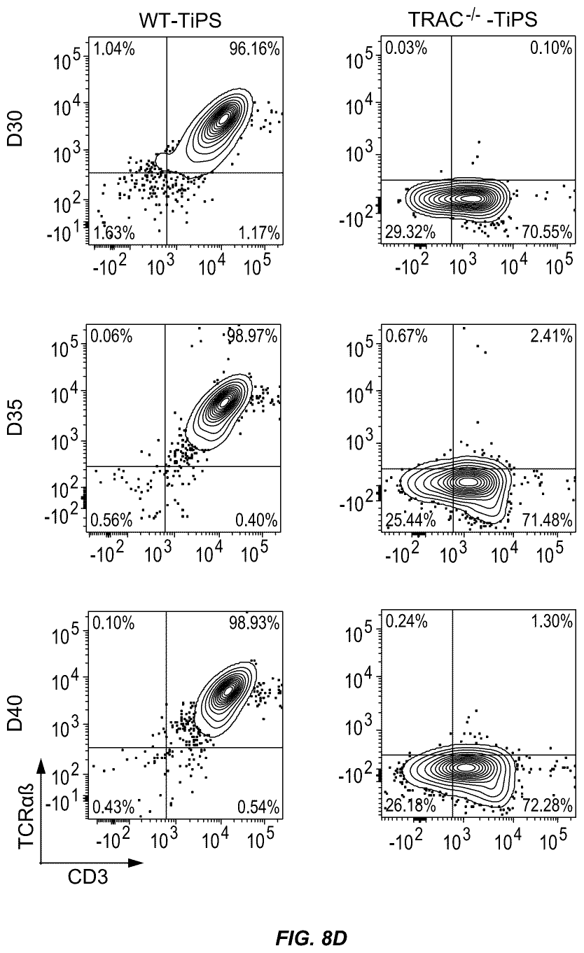

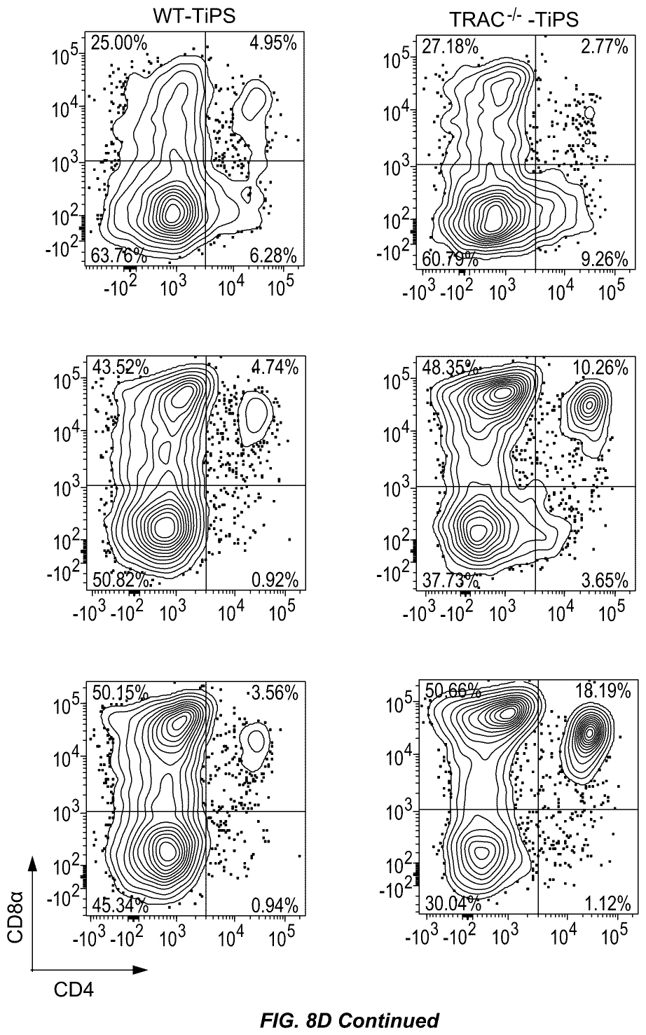

The presently disclosed subject matter determined the yield of CD4⁺CD8αβ+DP αβTCR-T cell precursors from pluripotent stem cells using the OP9-mDLL1 stromal cell line in the differentiation protocol shown in FIG. 1A. The human embryonic stem (ES) cell line H1 and fibroblast-derived iPS (FiPS) cells consistently yielded a DP population, typically arising by day 35 and followed by the appearance of CD3⁺ cells by day 40 (FIGS. 1B and 7A-7C). When differentiating unmodified, wild-type TiPS (WT-TiPS, FIG. 7A) under the same conditions, very few DP cells were induced (FIG. 1B). Nonetheless, CD3⁺ αβTCR+ cells were generated, appearing much earlier, typically by day 25 (FIGS. 1B and 7D) and eschewing the DP intermediate state. As the variable (V), diversity (D), and junctional (J) αβTCR genes are pre-rearranged in WT-TiPS, in contrast to their germline configuration in ES and FiPS cells, it was reasoned that V gene transcription, which normally precedes VDJ recombination, would result in the premature expression of rearranged αβTCR genes in TiPS. It was further hypothesized that the early formation of an αβTCR would mimic the earlier timing of a productive 76TCR rearrangement, and thus bypass DP cell formation and impart an innate phenotype (Pardoll et al., J Immunol 140, 4091-4096 (1988)). To preclude early TCR assembly, TCRα chain expression was abolished by disrupting the TRAC locus (FIGS. 8A-8C). Disrupting the TRAC locus (TRAC^{-/-}-TiPS) indeed allowed for increased DP cell formation from TRAC^{-/-}-TiPS compared to WT-TiPS (FIGS. 1C, 2D, and 8D), supporting the notion that early TCR signaling prevents αβTCR-T cell lineage commitment (Baldwin et al., J Exp Med 202, 111-121 (2005). The altered lineage commitment also corroborated that the presently disclosed differentiation protocol (FIG. 1A) supports DP cell development and pointed to the critical importance of the timing of TCR assembly in determining the fate of TiPS-derived T cells.

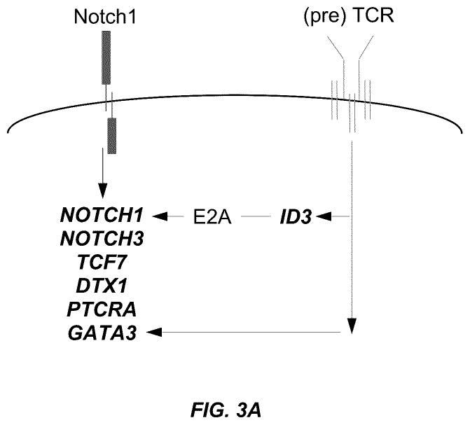

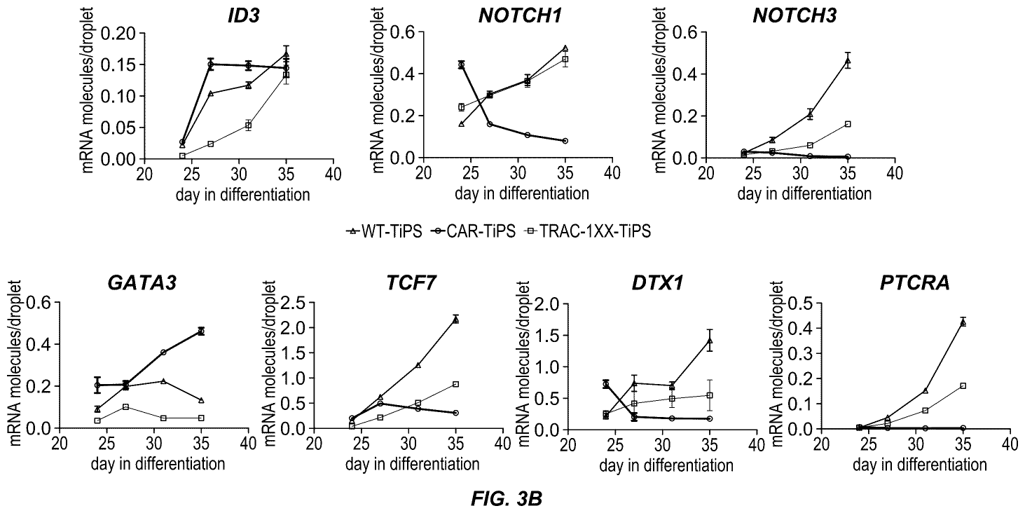

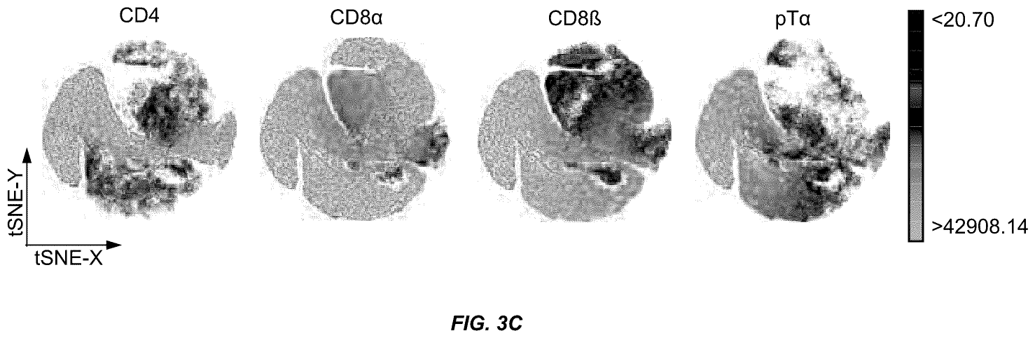

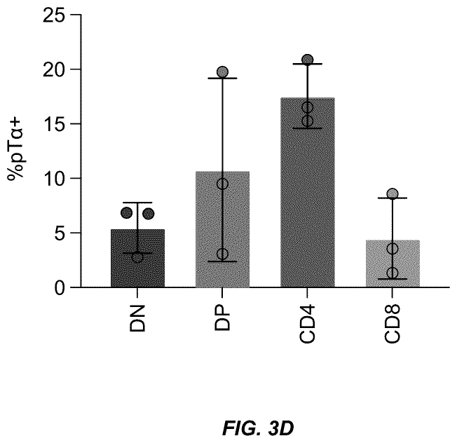

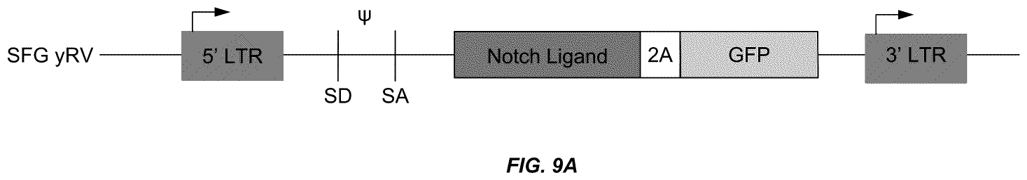

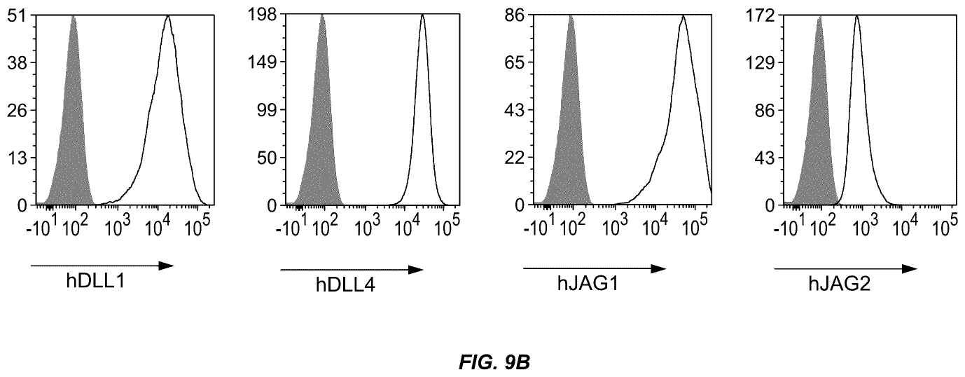

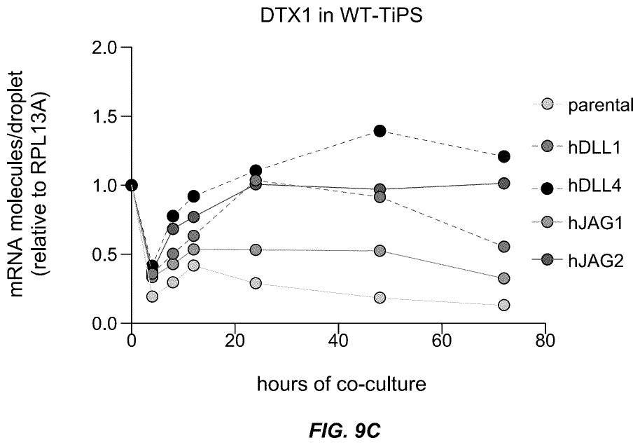

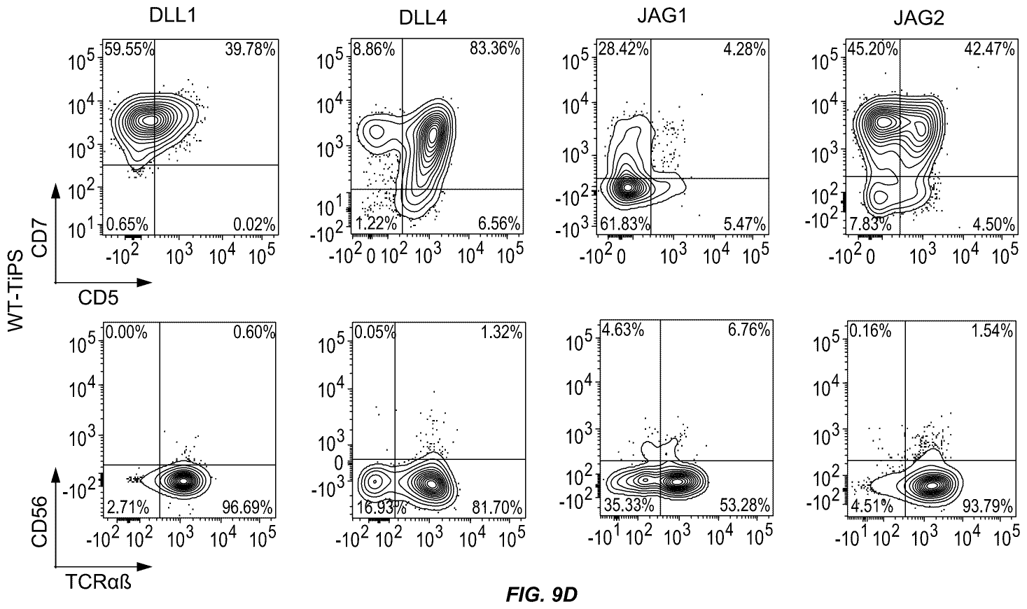

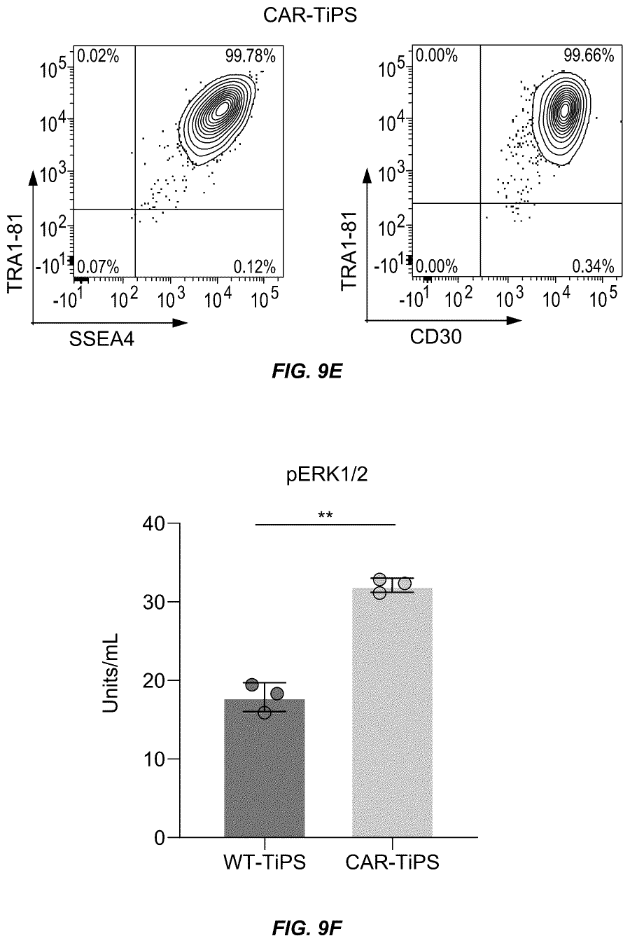

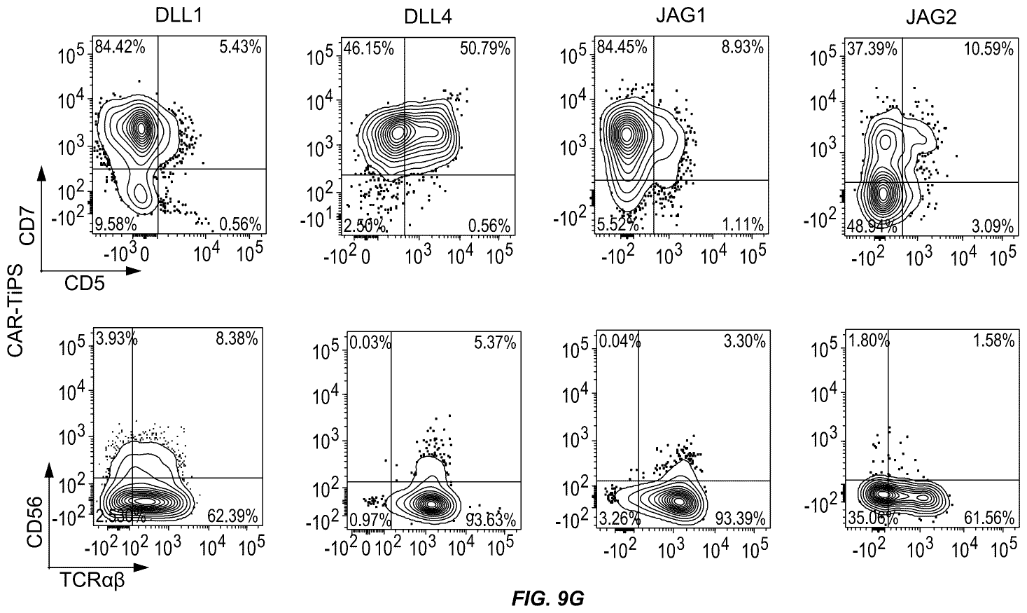

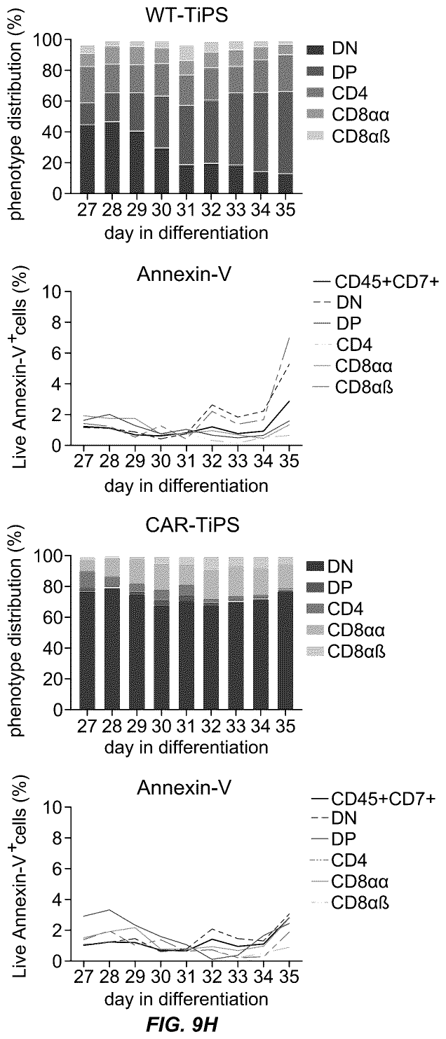

As Notch signaling also plays a critical role at the c43- versus γδ-lineage commitment junction (Washburn et al., Cell 88, 833-843 (1997)), its role was investigated by first assessing different Notch ligands for their ability to support T cell differentiation from TiPS. OP9 stromal cells were engineered to express either one of the four human Notch ligands, Delta-like ligand 1 (DLL1), Delta-like ligand 4 (DLL4), Jagged-1 (JAG1) or Jagged-2 (JAG2) (FIGS. 9A and 9B). These ligands displayed a gradation in their level of Notch signaling induction (Extended Data FIG. 3c) and, correspondingly, their ability to support T lineage commitment and DP formation from WT-TiPS (FIGS. 1D and 9D). DLL1 and JAG1 were unable to support DP T cell development, in contrast to JAG2 and DLL4, the latter showing the greatest efficiency in both T lineage commitment (CD7⁺CD5⁺ positive cells, FIG. 9D) and DP formation (FIGS. 1D and 1E). Interestingly, DLL4 has been previously shown to efficiently support in vitro T cell differentiation after TCR gene rearrangement (Mohtashami et al., J Immunol 185, 867-876 (2010)) and induces the strongest signaling from Notchi (Van de Walle et al., J Exp Med 210, 683-697 (2013)) (FIG. 9C). These findings support a model wherein DP cell formation depends on an intricate interaction between TCR and Notch stimulation, in which a potent DLL4-mediated signal is required in WT-TiPS because of the earlier expression of a functional TCR, whereas DLL1 suffices for H1 and TRAC^{-/-}-TiPS. The requirement for stronger Notch engagement in the context of earlier TCR assembly suggests that the TCR interferes with Notch signaling, which can be over-ridden by more potent Notch ligands, and that a strong activation signal from the CAR (Ramello et al., Sci Signal 12 (2019); Maluski et al., J Clin Invest 129, 5108-5122 (2019)) and the TCR would offset stronger Notch signaling.

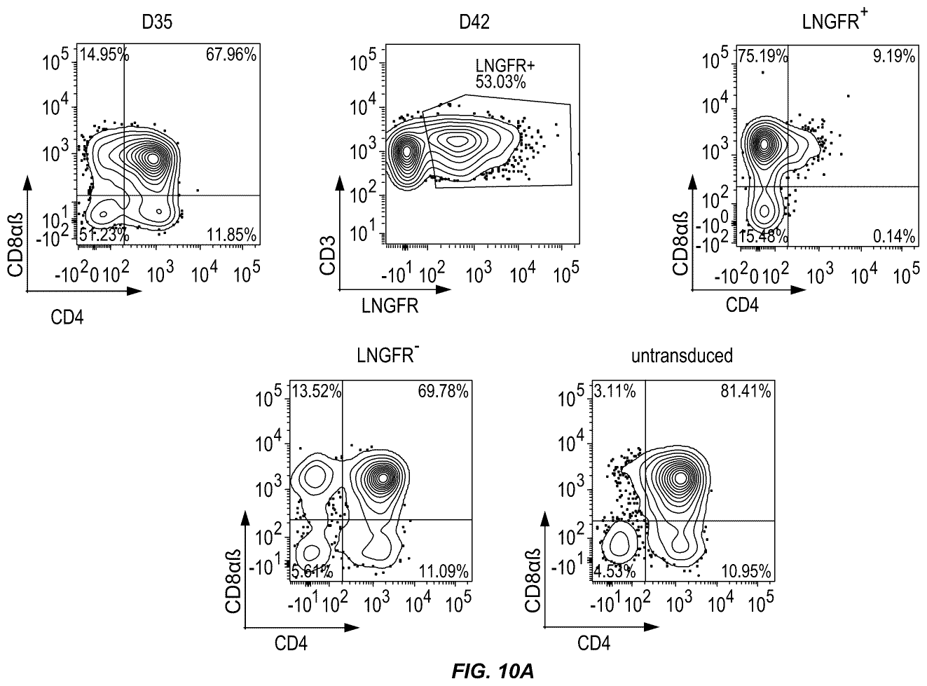

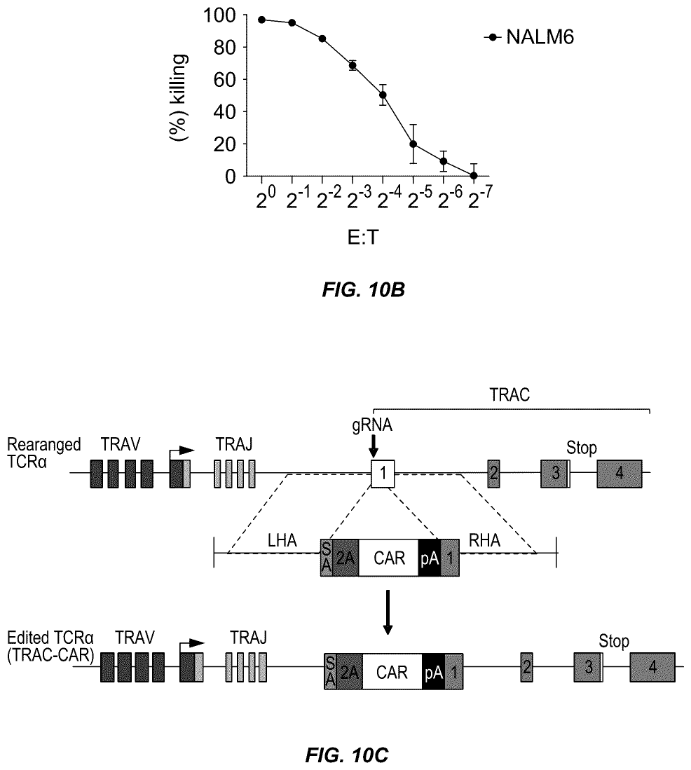

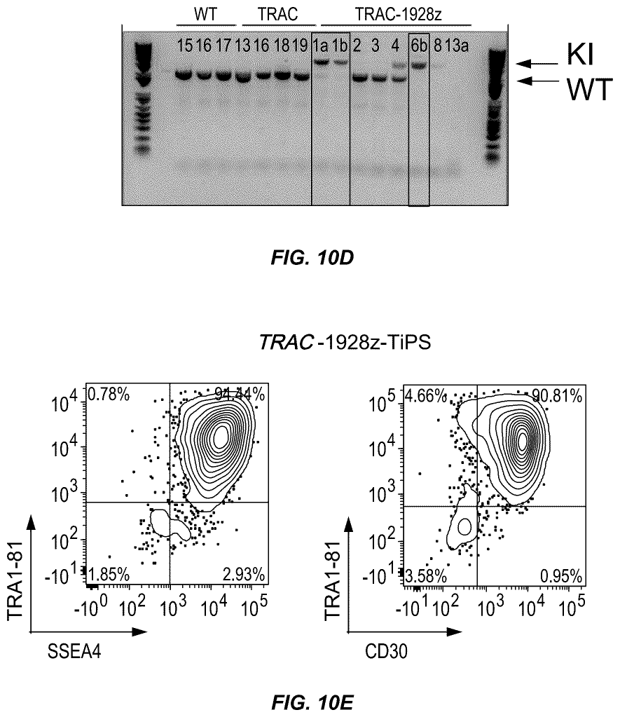

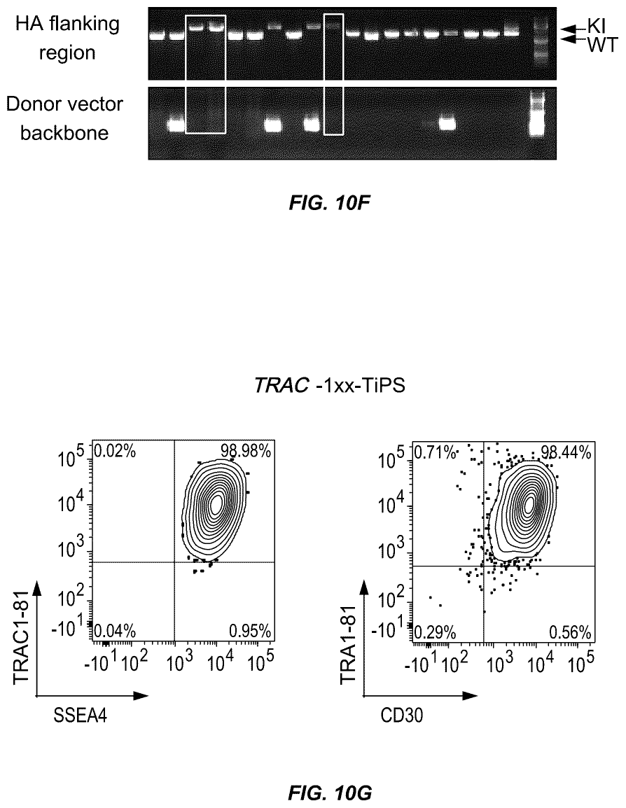

Consistent with this model, it was found that TiPS that constitutively expressed the 1928z CAR (CAR-TiPS, FIG. 9E), had increased levels of ERK1/2 phosphorylation (FIG. 9F), induced fewer CD7+CD5+ cells, and did not produce DP cells, even in the presence of DLL4, instead generating DN and CD8αα T cells (FIGS. 1F, 1G, and 9G). To assess whether premature CAR signaling induces apoptosis in emerging DP cells, apoptotic cells were measured at the different developmental stages (DN, CD4 induced single positive (ISP), DP, CD8αβ SP) in WT-TiPS and CAR-TiPS from D27-D35, when the induction of the DP population occurs in WT-TiPS (FIG. 9H). Levels of apoptosis were uniformly low (<5%) in both WT-TiPS and CAR-TiPS, and similar in all different developmental stages, indicating that the lack of DP establishment from CAR-TiPS is not due to global apoptosis of the DP population. To verify whether the development of CD8αβ T cells is feasible in the presence of a CAR, we transduced the CAR into DP cells arising from WT-TiPS on D35. Delaying the onset of CAR expression in this manner resulted in the development of functional SP cells, including CD8α3 CAR T cells (FIGS. 10A and 10B). This finding not only established that CD8αβ CAR T cells can be generated from TiPS, but also confirmed that the lack of DP formation from CAR-TiPS was likely due to interference with DP commitment arising from the early CAR expression afforded by the constitutive Ubiquitin C promoter in CAR-TiPS.

### Regulated CAR Expression Facilitates CD4⁺CD8α #+DP T Cell Development

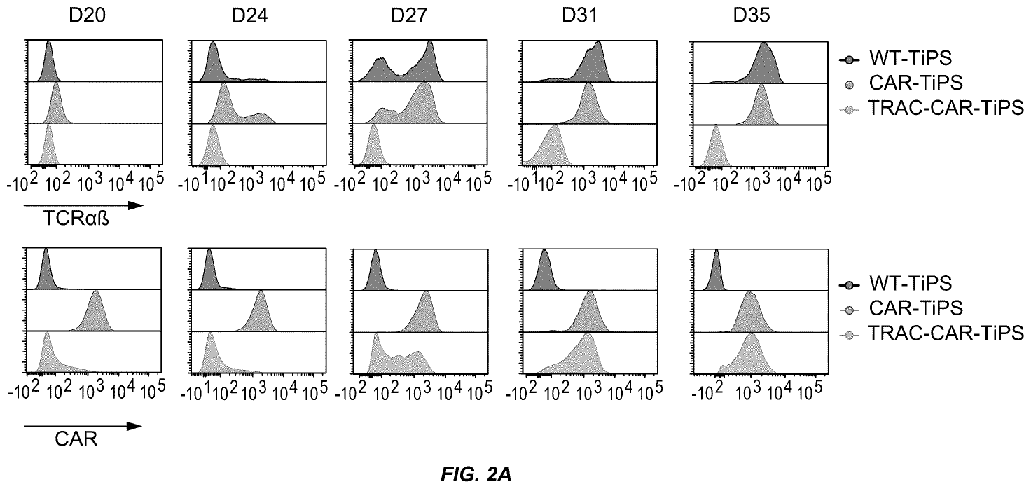

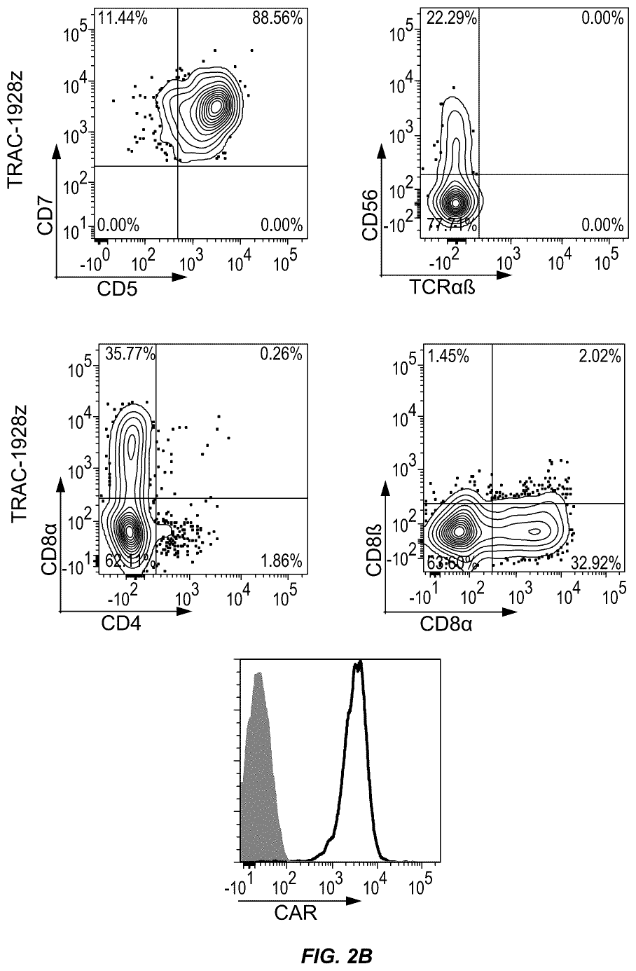

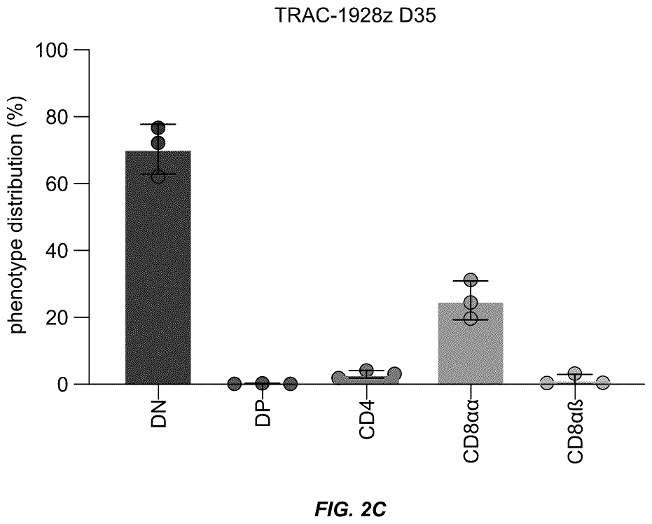

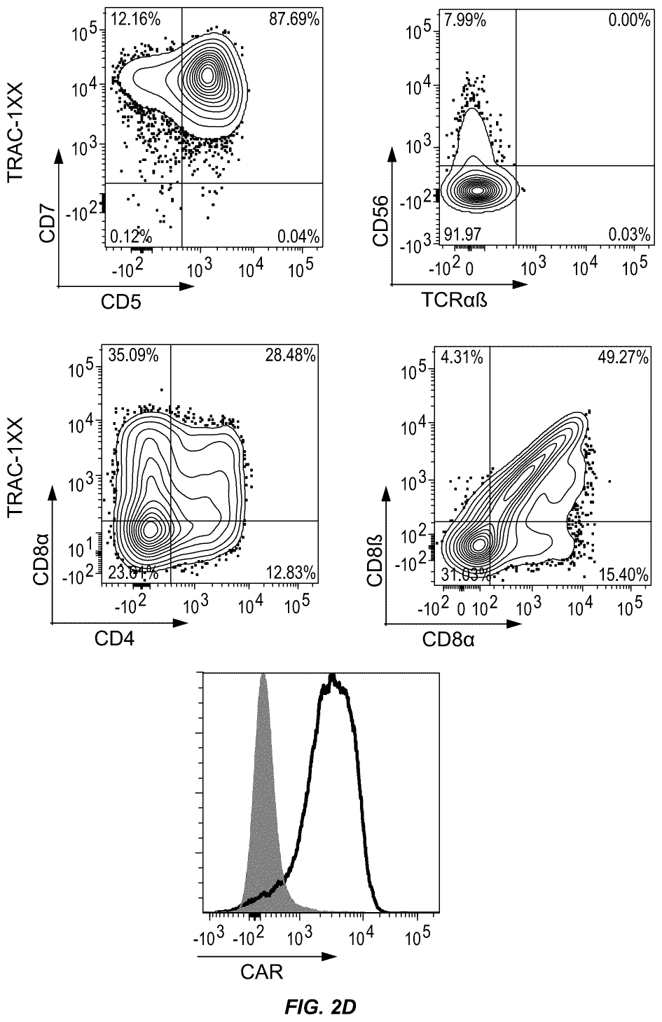

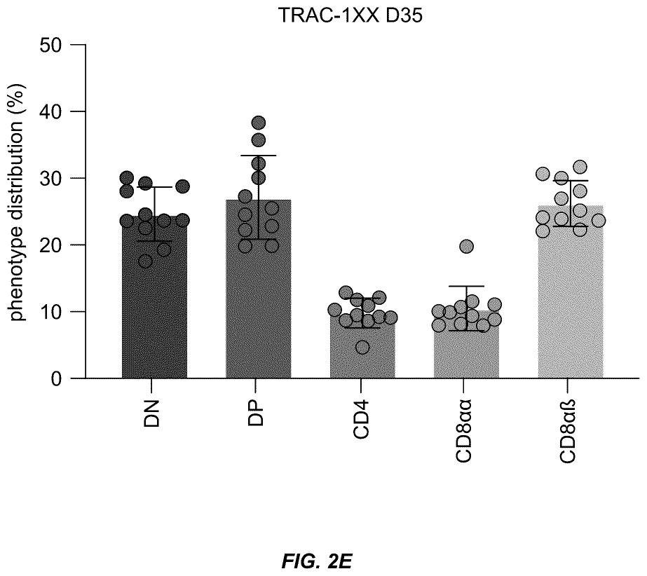

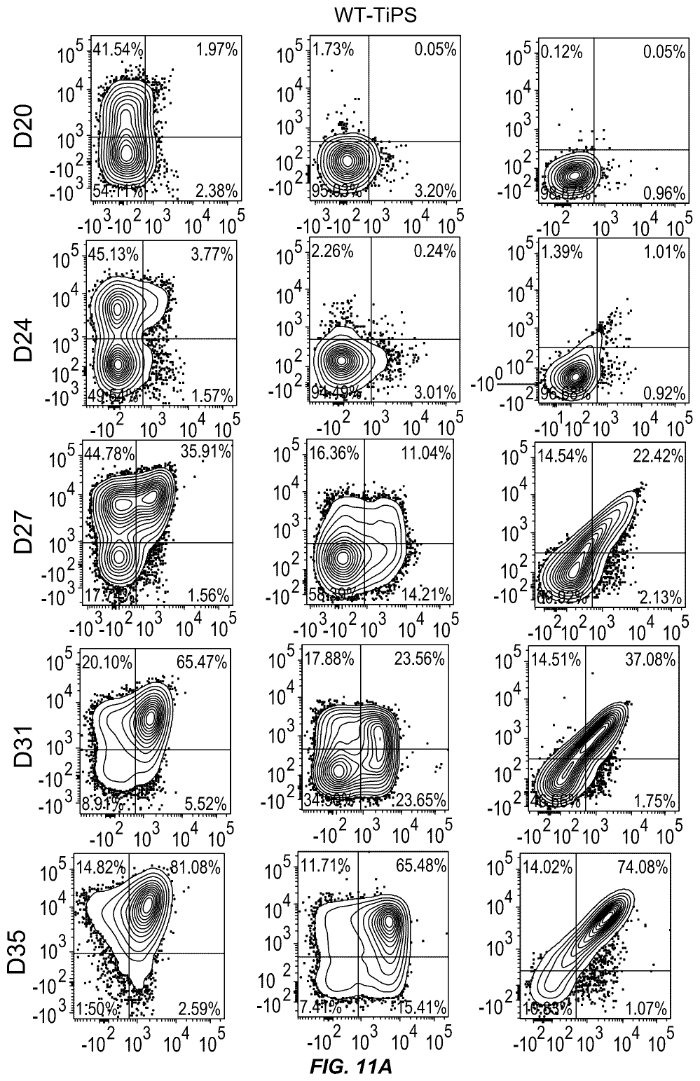

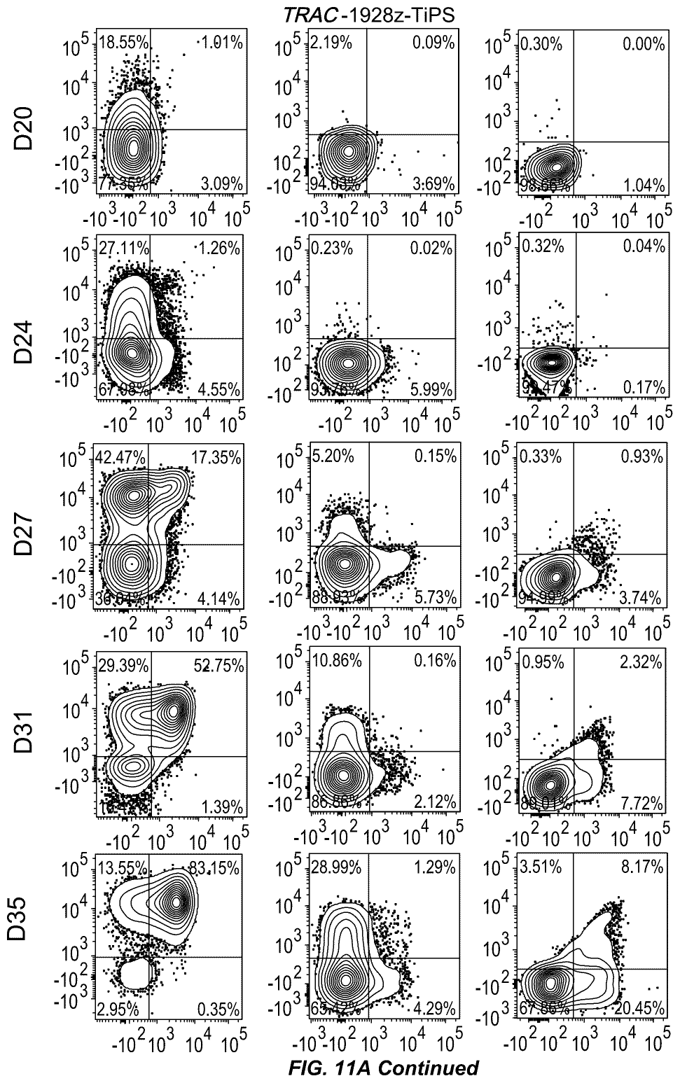

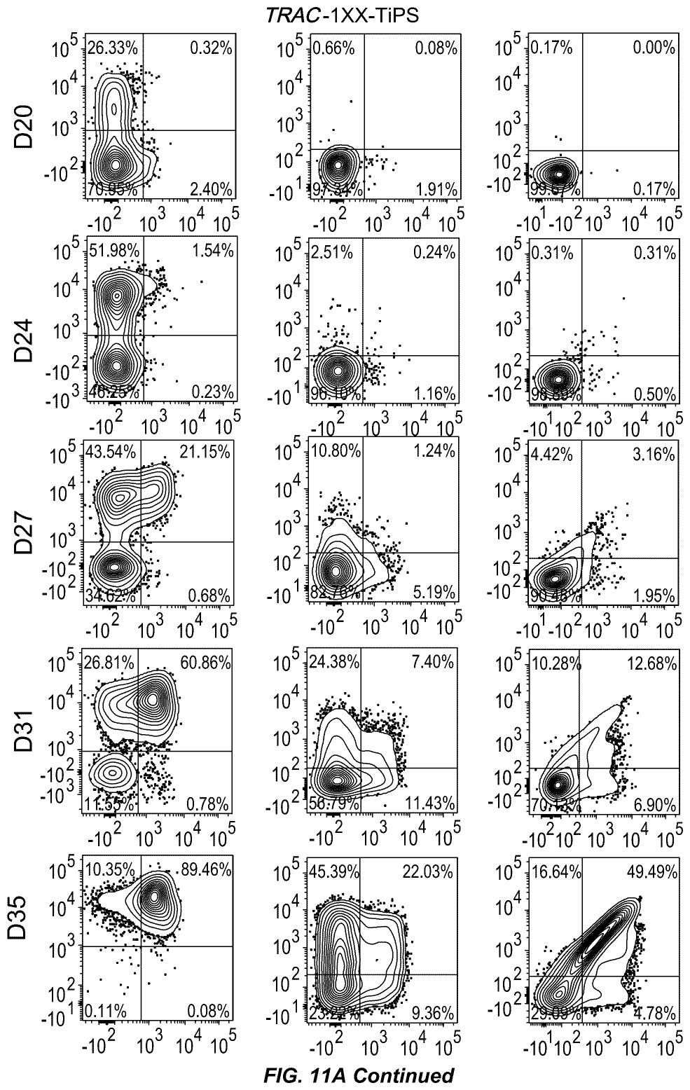

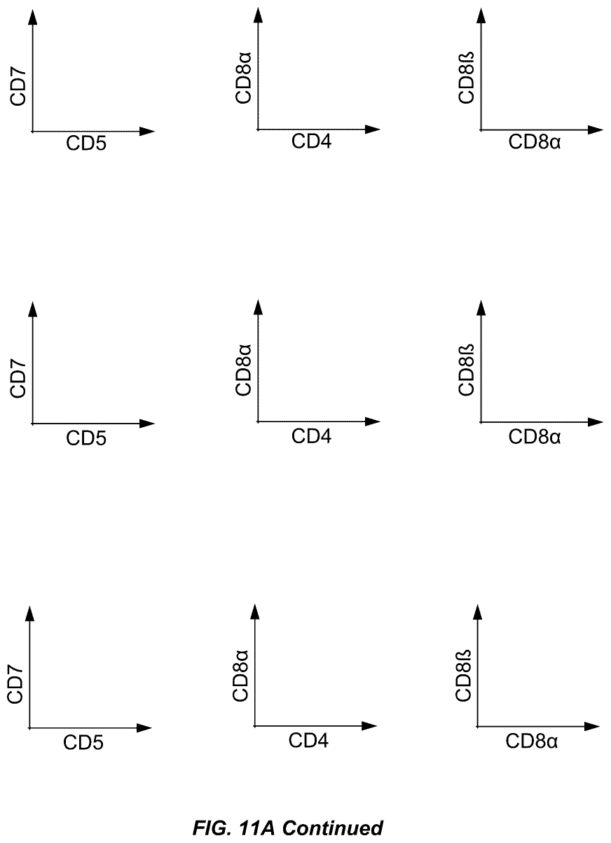

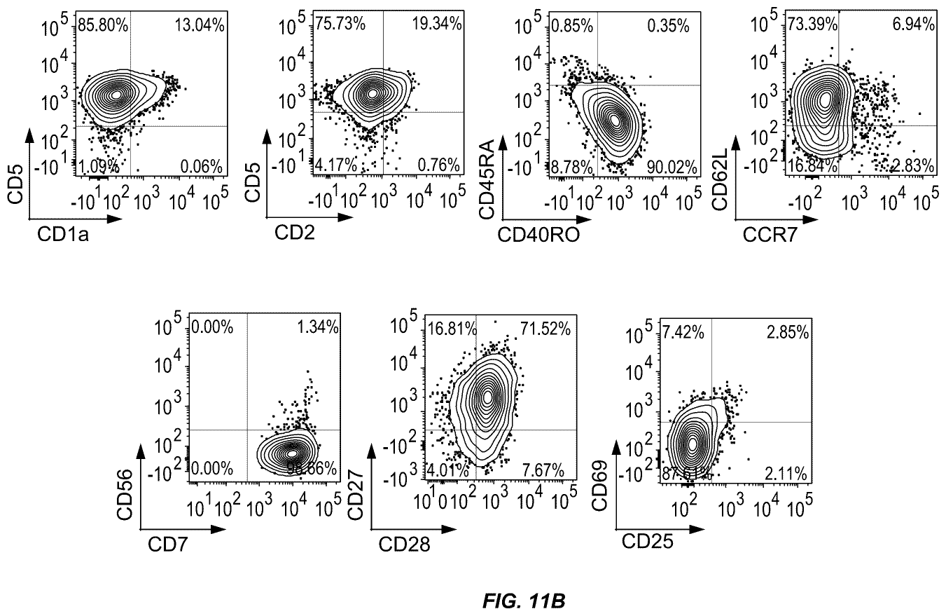

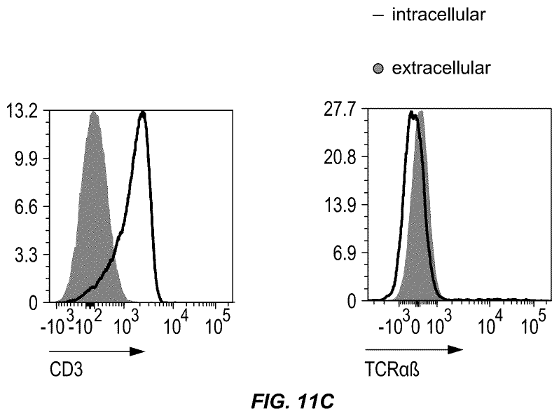

To restrict CAR expression, the sequence encoding CAR was placed under the transcriptional control of the TRAC promoter (TRAC-1928z-TiPS, FIGS. 10C-10E). CAR expression from the TRAC locus not only resulted in the expected absence of TCR expression throughout differentiation (FIG. 2A, top panel), but also showed the remarkable similarity in temporal cell surface expression between TRAC-CAR and the αβTCR in both WT-TiPS and CAR-TiPS (FIG. 2A). The differentiation of TRAC-1928z-TiPS towards early T cell lineage commitment improved, as reflected in a greater CD7+CD5+ population (FIG. 2B) compared to CAR-TiPS (FIG. 9G). DP induction, however, was still not enhanced (FIGS. 2B, 2C, and 11A).

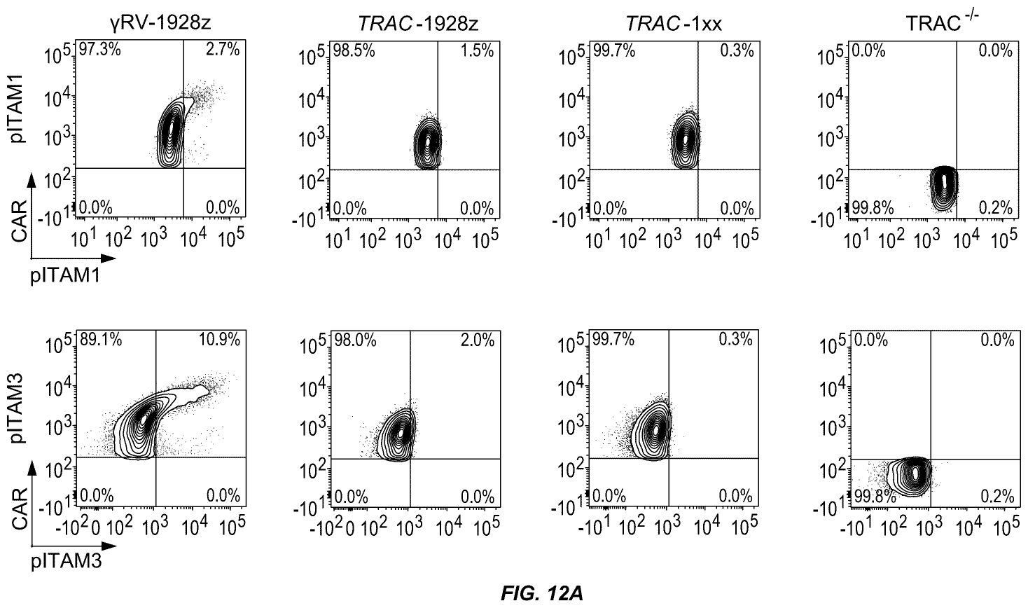

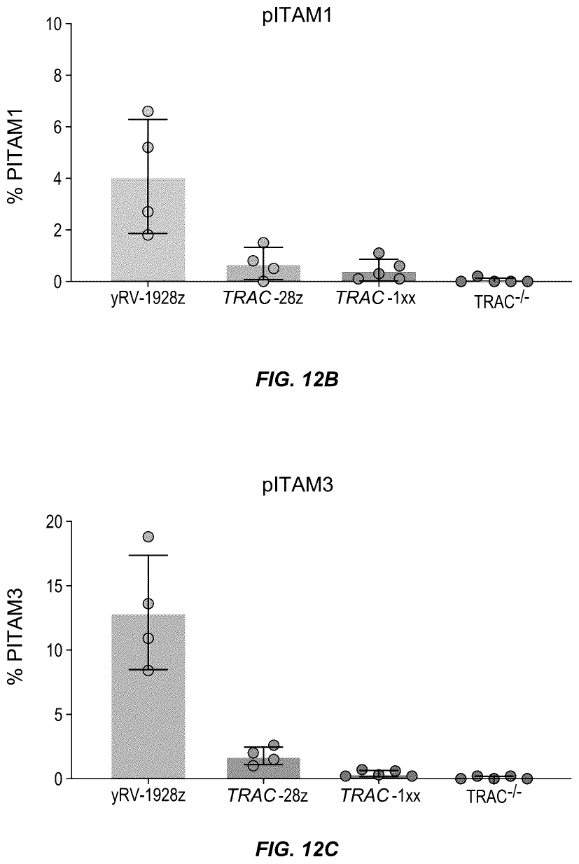

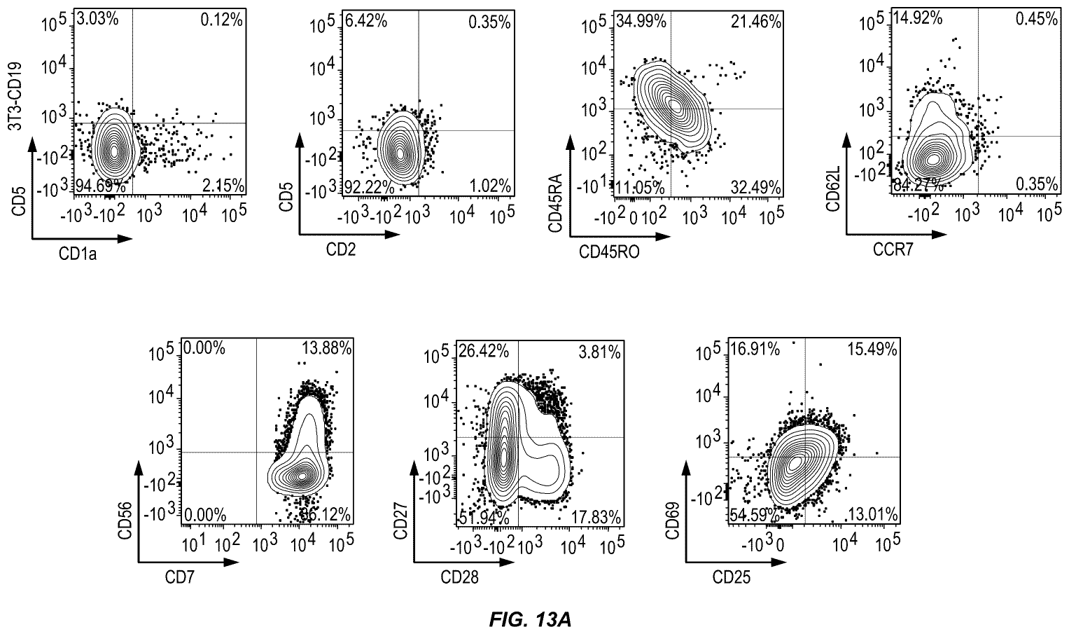

It was hypothesized that despite its delayed onset of expression, the TRAC-encoded 1928z still interfered enough to prevent DP commitment. It was therefore sought to attenuate CAR signaling strength by substituting the 1928z with 1928z-1XX, a CAR in which the second and third ITAM have been inactivated. In PBMC-derived T cells, CAR expression through the endogenous TRAC promoter reduces signaling in absence of antigen exposure and the mutation of the second and third ITAMs pre-empts their phosphorylation. Phosphorylation of ITAM1 and ITAM3 is readily detected in retrovirally expressed CARs (γRV-1928z) in the absence of antigen, increasingly in T cells with the highest CAR expression (FIGS. 12A-12C). TRAC-encoded 1928z showed reduced phosphorylation of ITAM1 and ITAM3, while the latter was abolished in TRAC-1XX T cells (FIGS. 12A-12C).

Upon their differentiation, TRAC-1XX-TiPS (FIGS. 10F and 10G) not only maintained the same heightened propensity to induce CD7 and CD5 expression as TRAC-1928z-TiPS, but additionally increased their progression to the DP stage (FIGS. 2D, 2E, and 11A). By day 35, these DP cells express CD1a, CD2 and CD45RO, consistent with the phenotype of human DP thymocytes (Res et al., J Exp Med 185, 141-151 (1997); Haynes et al., J Immunol 141, 3776-3784 (1988); Fujii et al., Eur J Immunol 22, 1843-1850 (1992)) (FIG. 11B). Intracellular CD3 confirmed their T lineage commitment despite the absence of CD3/αβTCR expression at their cell surface (FIG. 11C).

### CAR Expression Affects Notch and TCR Downstream Target Gene Induction

To further support the hypothesis that the induction to the DP stage is controlled by CAR and Notch interactions, the impact of CAR regulation on Notch target transcript levels was assessed during the T lineage commitment phase of the presently disclosed in vitro differentiation protocol. Expression level of several genes which have been reported to be associated with T lymphoid development and αβ/γδ lineage commitment was assessed, including NOTCH1, NOTCH3, ID3, TCF7, DTX1, GATA3 and βTCRA (FIG. 3A). Constitutive CAR expression did indeed grossly perturb the expression pattern observed in WT-TiPS. CAR-TiPS show an early increase in the expression of the (pre)TCR target ID3 between D24 and D27 (FIG. 3B), with correspondingly reduced levels of NOTCH1, NOTCH3 and Notch targets TCF7, DTX1 and βTCRA. In the TRAC-1XX-TiPS, ID3 expression was decreased while NOTCH1, NOTCH3 and their downstream targets increased to levels nearing those found in WT-TiPS (FIG. 3B). GATA3 expression, which is a direct target gene of not just Notch but also the pTα, was noticeably upregulated in CAR-TiPS relative to WT-TiPS and reduced in TRAC-1XX-TiPS (FIG. 3B), suggesting that constitutive CAR expression results in stronger GATA3 induction compared to the TCR or pTα.

Notably, one of the key Notch targets, βTCRA, which encodes the pTa, is repressed in CAR-TiPS, but induced in differentiating WT-TiPS as well as in TRAC-1XX-TiPS (FIG. 3B). In thymic development, pTα pairs with the rearranged β-chain to allow for a chain rearrangement and progression to the DP stage (Fehling et al., Nature 375, 795-798 (1995)). To assess whether PTCRA induction in TRAC-1XX-TiPS was associated with pTα protein expression, cell surface-protein levels were measured by flow cytometry and found cell surface pTα expression in the induced-CD4 SP and DP populations (FIGS. 3C and 3D), consistent with its physiological pattern (Dolens et al., EMBO Rep 21, e49006 (2020)).

### CAR Engagement Facilitates Maturation to CD8α03 Single Positive T Cells

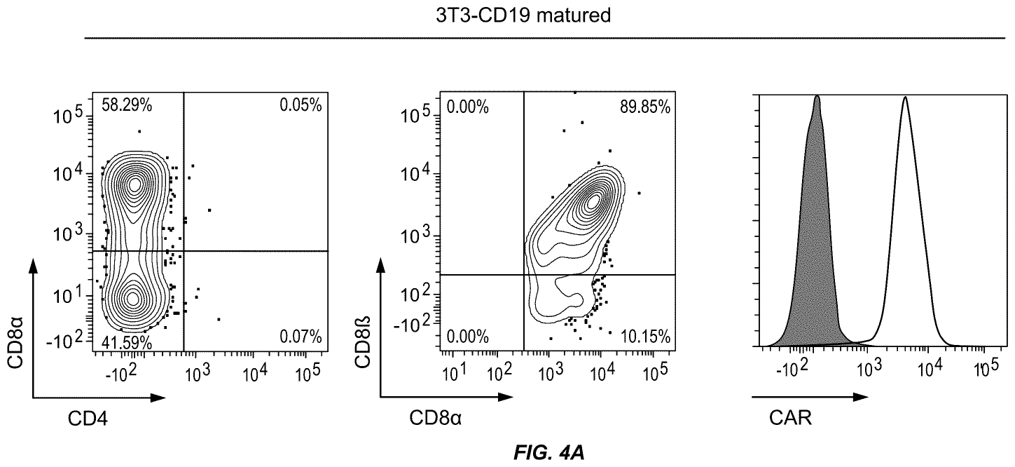

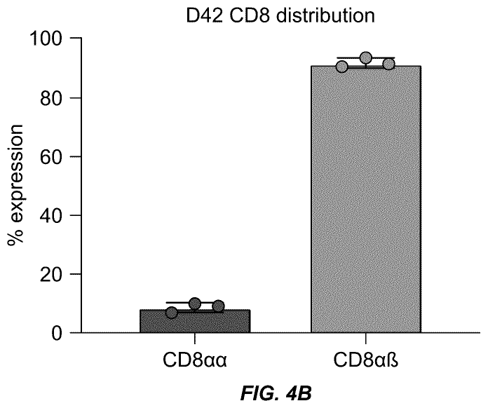

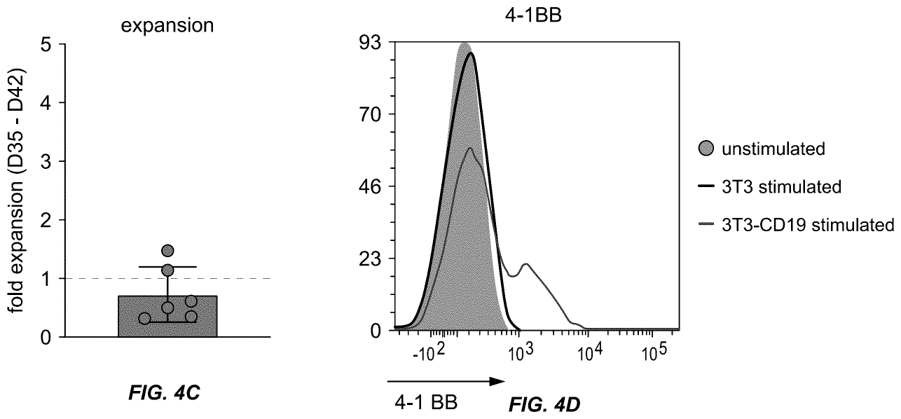

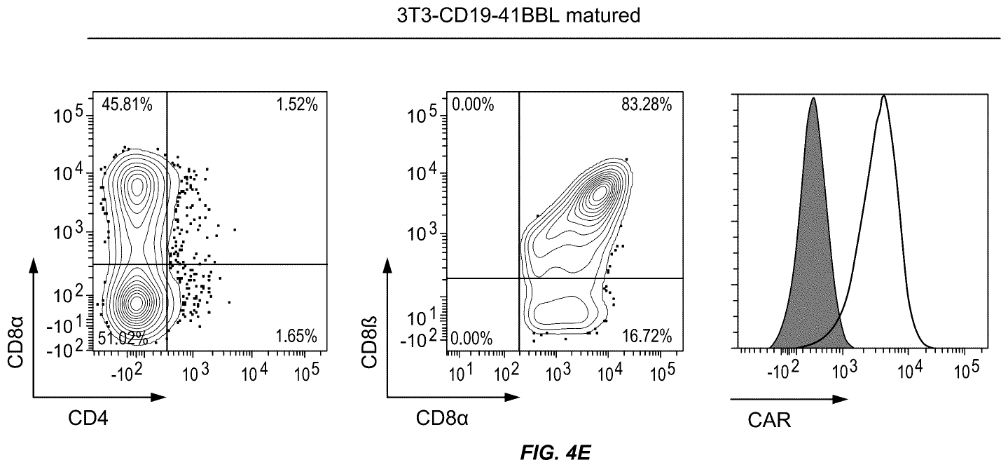

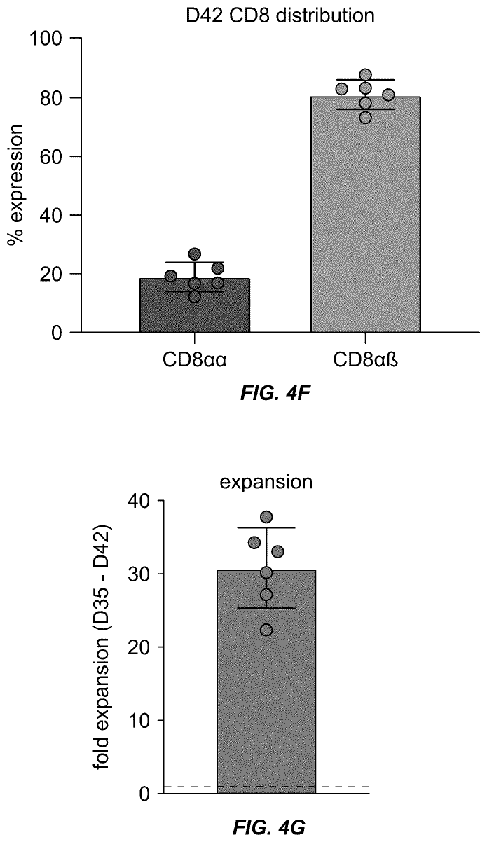

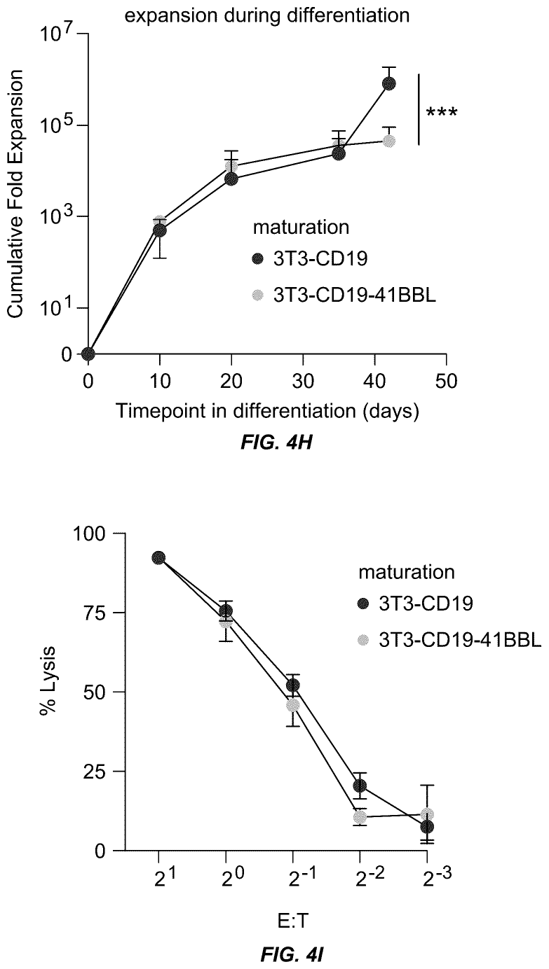

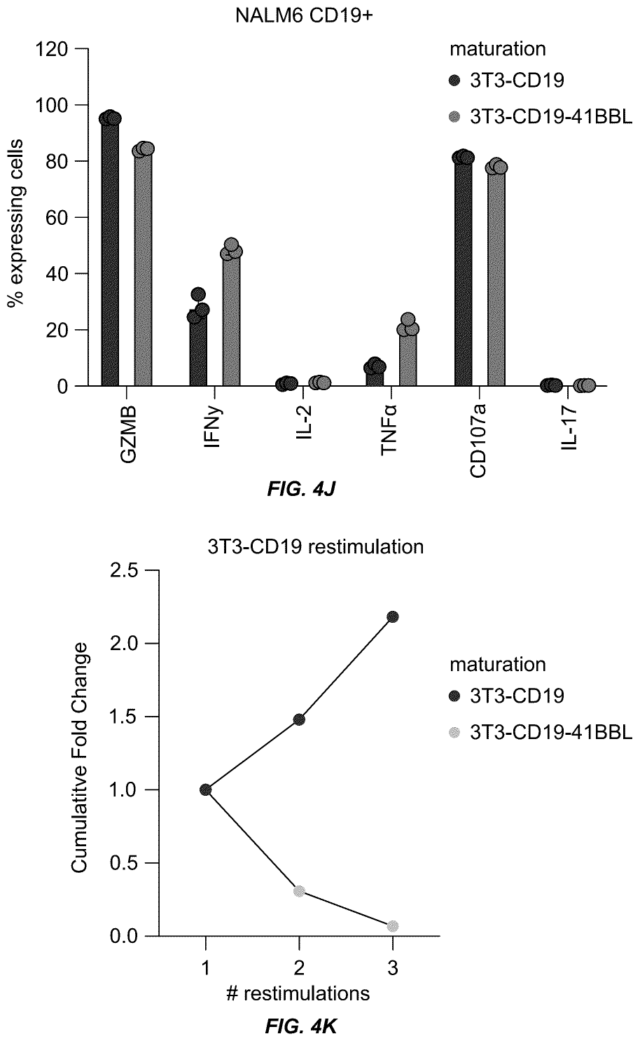

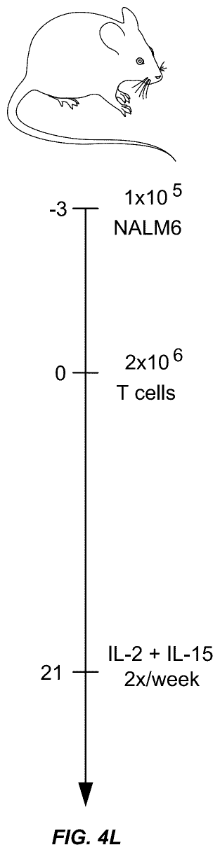

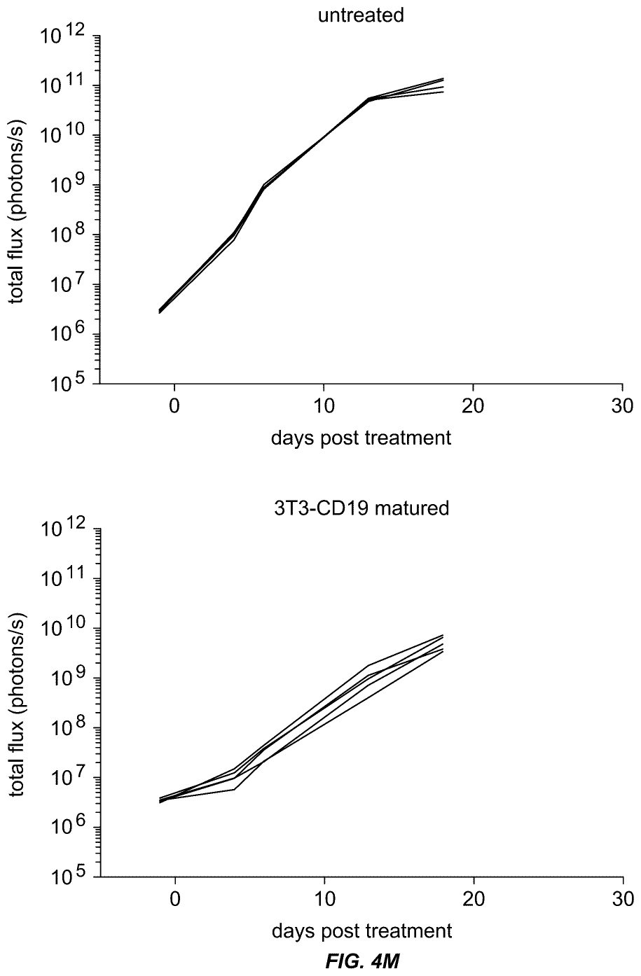

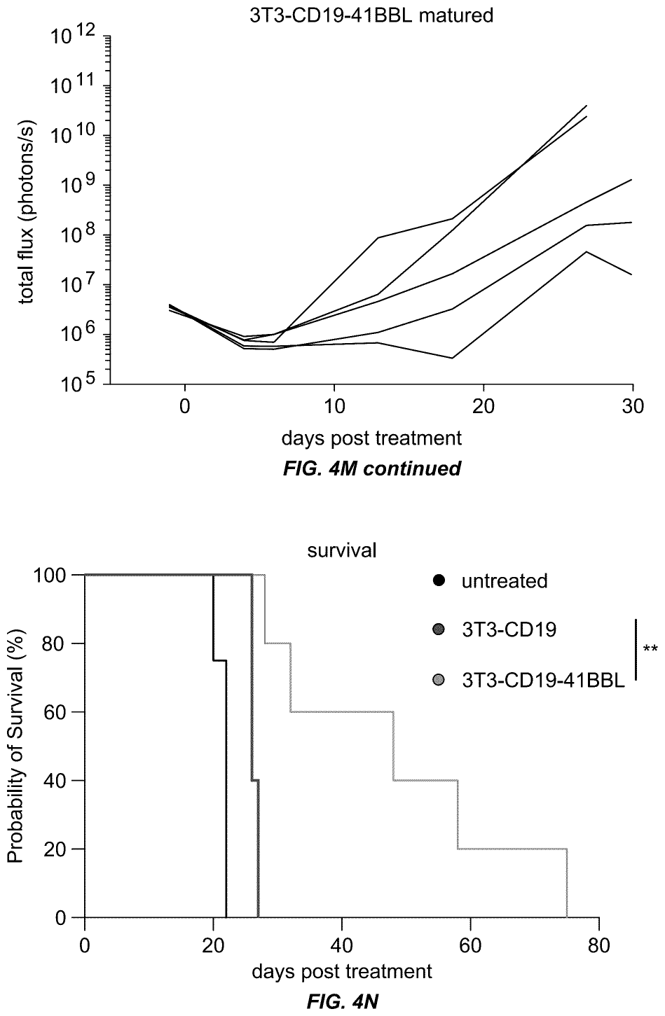

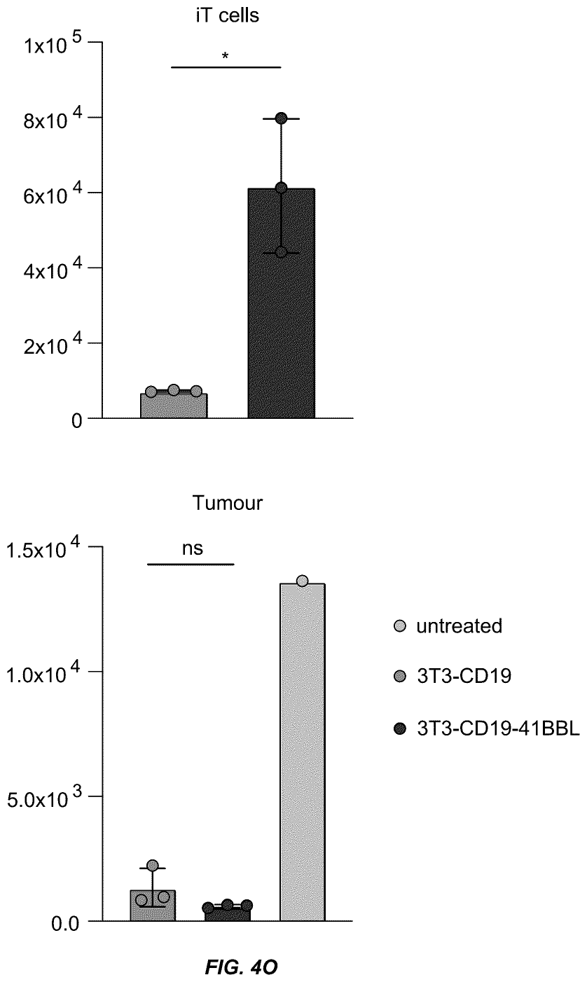

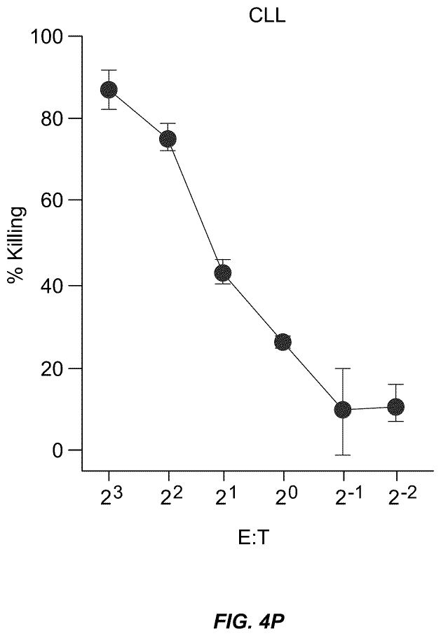

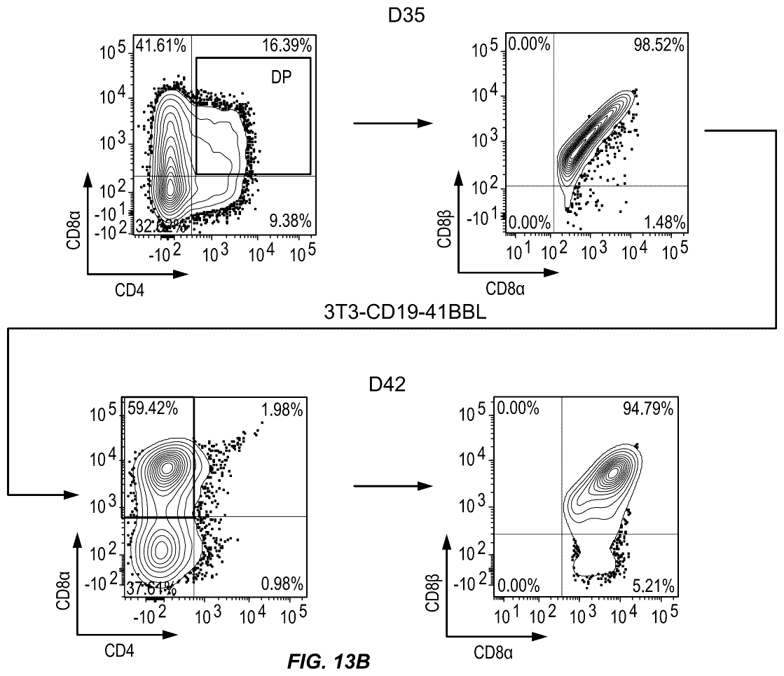

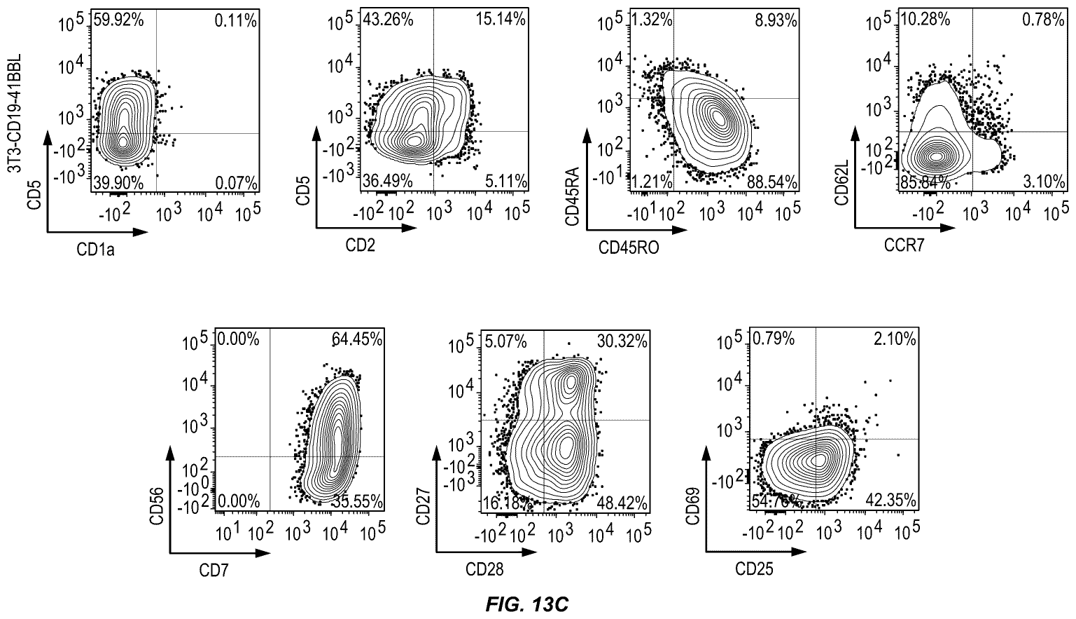

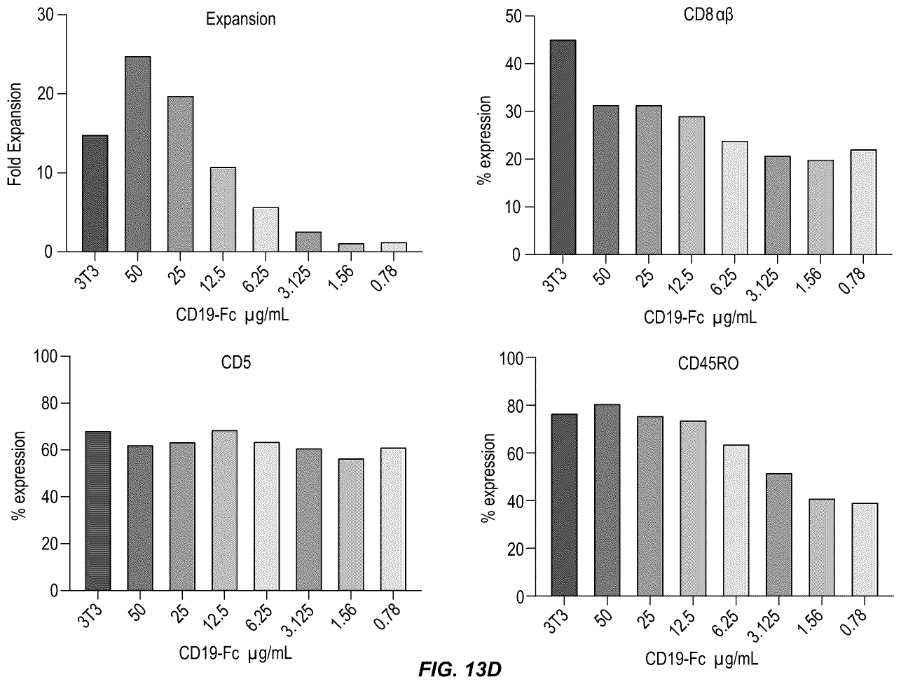

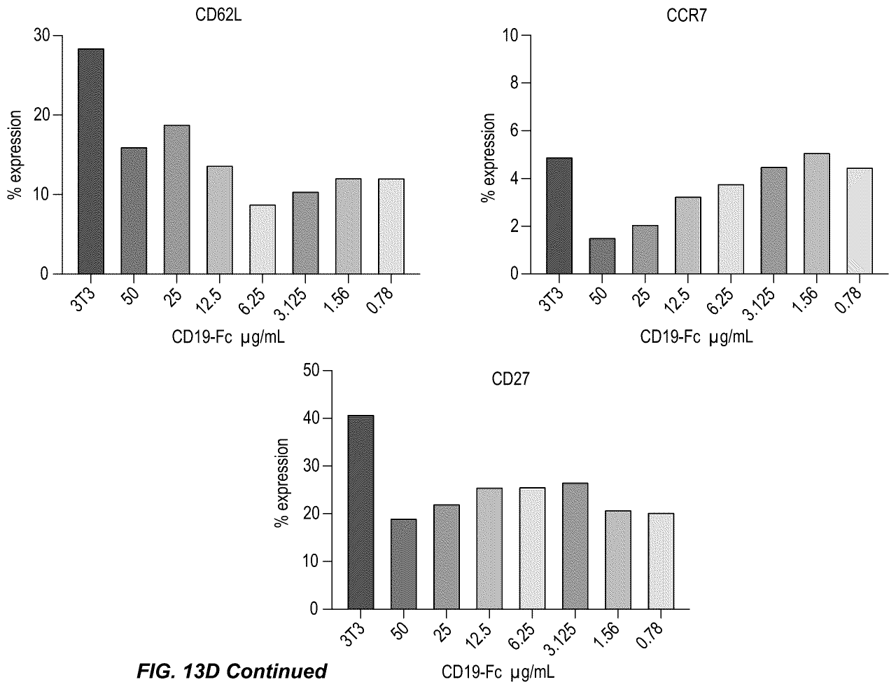

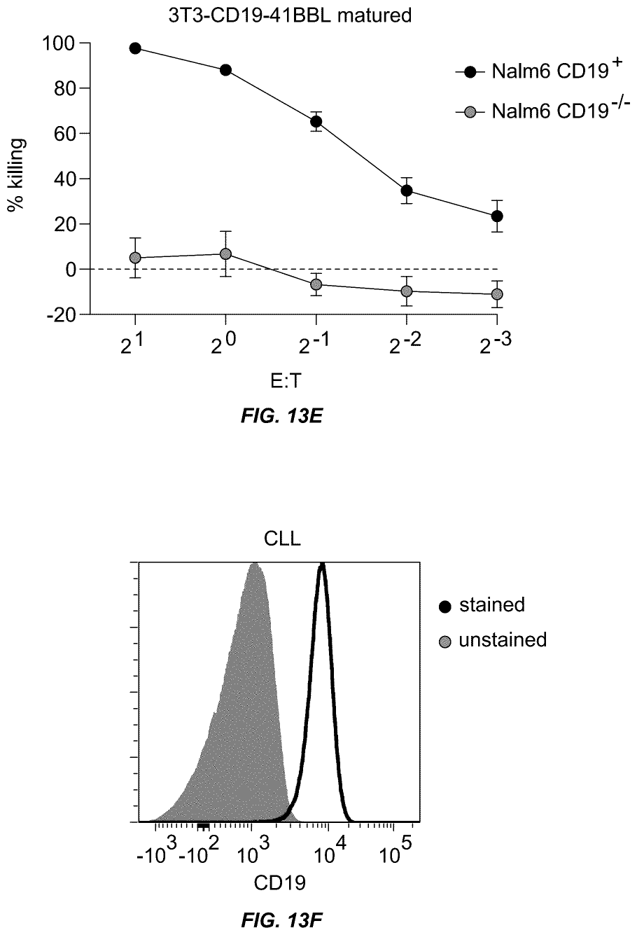

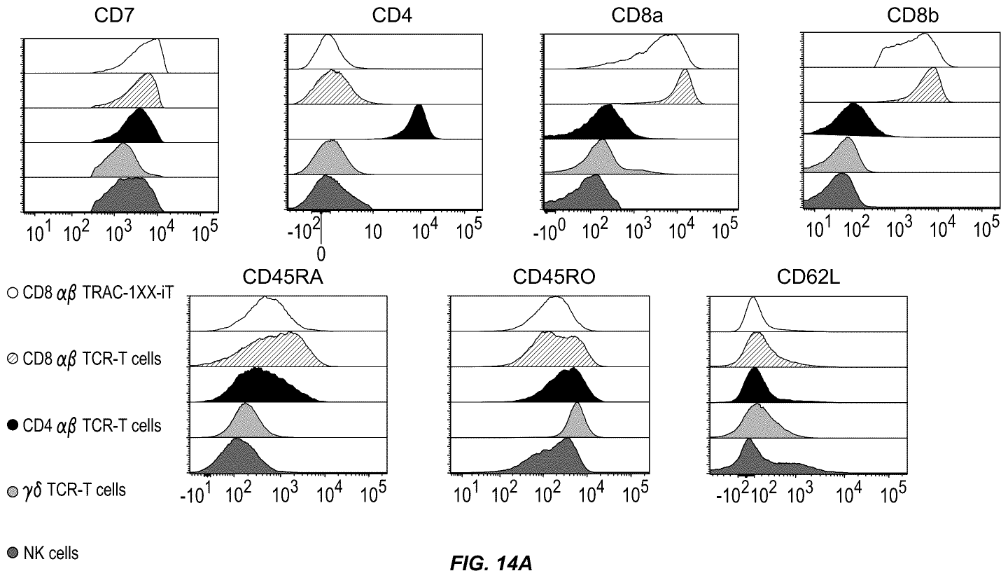

Having established that CAR regulation allows for DP cell development in the absence of a TCR, it was set out to determine whether the CAR could substitute for the TCR in further driving maturation of DP cells into CD8αβ SP T cells. In the absence of a TCR-dependent positive selection process, D35 DP T cells were stimulated on cells expressing the CAR target antigen (NIH/3T3 fibroblasts expressing CD19) (Brentjens et al., Nat Med 9, 279-286 (2003)). CAR engagement was required for T cell survival as co-culture with parental fibroblasts lacking CD19 did not yield viable iT cells. Phenotypic analysis on CD19 stimulated TRAC-1XX-iT cells after a week (day 42) showed that CD4 and CD1a expression had waned and that a population of CD8αβ SP T cells was induced, consistent with maturation from the DP to the SP stage (FIGS. 4A and 13A). Some DN cells and a small population of CD8αα T cells still coexisted (FIG. 4B). The matured SP D42 TRAC-1XX-iT cells displayed a phenotype resembling activated T cells, including the upregulation of CD25, CD69, CD56 and transition from CD45RO to CD45RA (FIG. 13A). However, the cells also downregulated CD5 and expanded poorly (FIGS. 4C and 13A).

Analyzing the phenotype and induction of activation and costimulatory markers upon antigen exposure, it was found that 4-1BB was induced transiently 8 h after antigen exposure (FIG. 4D) and hypothesized that engaging the 4-1BB pathway at this stage may promote T cell expansion. Thus, the 3T3-CD19 cells were engineered to co-express 4-1BB ligand (3T3-CD19-41BBL). When D35 TRAC-1XX-iT cells were stimulated on 3T3-CD19-41BBL, they maintained the ability to form SP cells (FIGS. 4E and 4F) and expanded 30-fold (FIG. 4G). To confirm that the resulting SP cells at D42 were indeed derived from D35 DP precursors, the DP cells were sorted at D35 and then exposed to 3T3-CD19-41BBL. After 7 days, the DP cells had lost CD4 expression and matured to CD8αβ SP cells (FIG. 13B). Phenotypically, 3T3-CD19-41BBL matured cells retained CD5 and CD2 expression, more so than 3T3-CD19 matured cells, and showed higher expression of CD45RO, CD28 and CD56 (FIG. 13C).

To determine whether CD19 levels may influence acquisition of an effector-like phenotype of the TRAC-1XX-iT cells, iT cells were matured on titrated levels of recombinant CD19 (FIG. 13D). Increasing CD19 positively affected the expansion and CD8ab SP iT cell content, but did not reduce the effector-like phenotype. To determine whether exposure to 4-1BB costimulation qualitatively affected TRAC-1XX-iT maturation, the functions of the 3T3-CD19 and 3T3-CD19-41BBL matured TRAC-1XX-iT cells were compared. In vitro cytolytic function (FIG. 4I) and cytokine production (FIG. 4J) were similar between the two groups. However, whereas 3T3-CD19 matured cells failed to expand upon repeated exposure to antigen, 3T3-CD19-41BBL maturation improved their expansion and survival (FIG. 4K). The presently disclosed subject matter proceeded to compare these two populations in the NALM6 leukemia model (FIG. 4L), wherein iT cells matured on 3T3-CD19-41BBL showed improved tumor control and survival (FIGS. 4M-40), which was associated with an increased persistence of TRAC-1XX-iT cells (FIG. 4O). The cytolytic function of 3T3-CD19-41BBL matured TRAC-1XX-iT cells was also demonstrated in vitro to be antigen specific (FIG. 13E), and responsive not only to NALM6, but also to primary patient-derived CD19⁺ CLL cells (FIGS. 4P and 13F).

### TRAC-1XX-iT Cells Overall Resemble Peripheral-Blood Derived CD8α T Cells

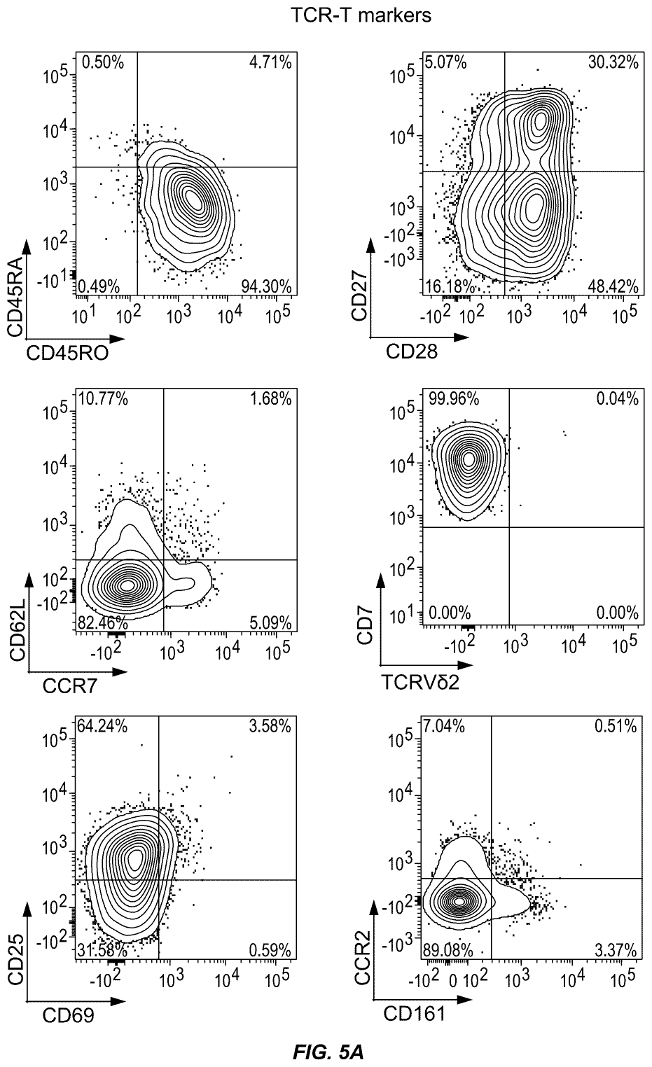

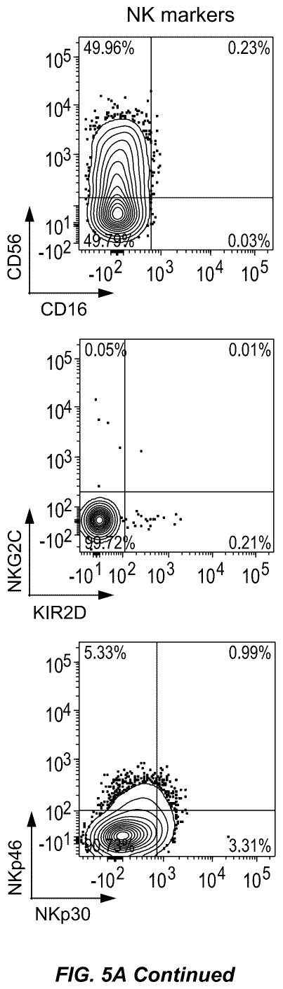

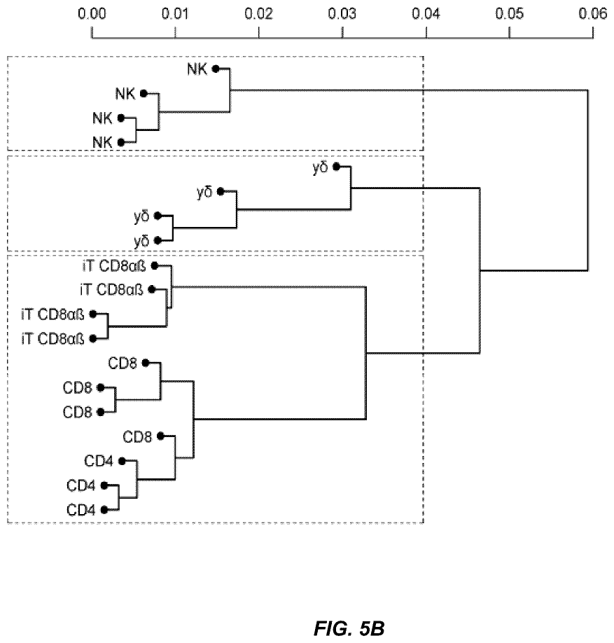

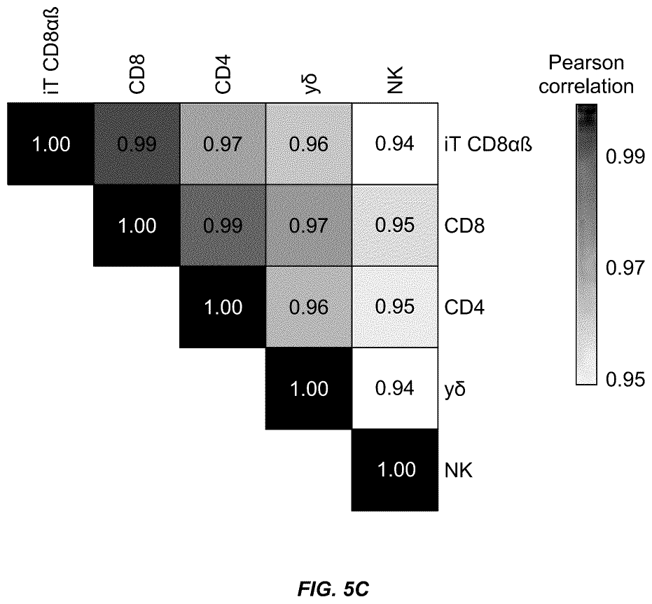

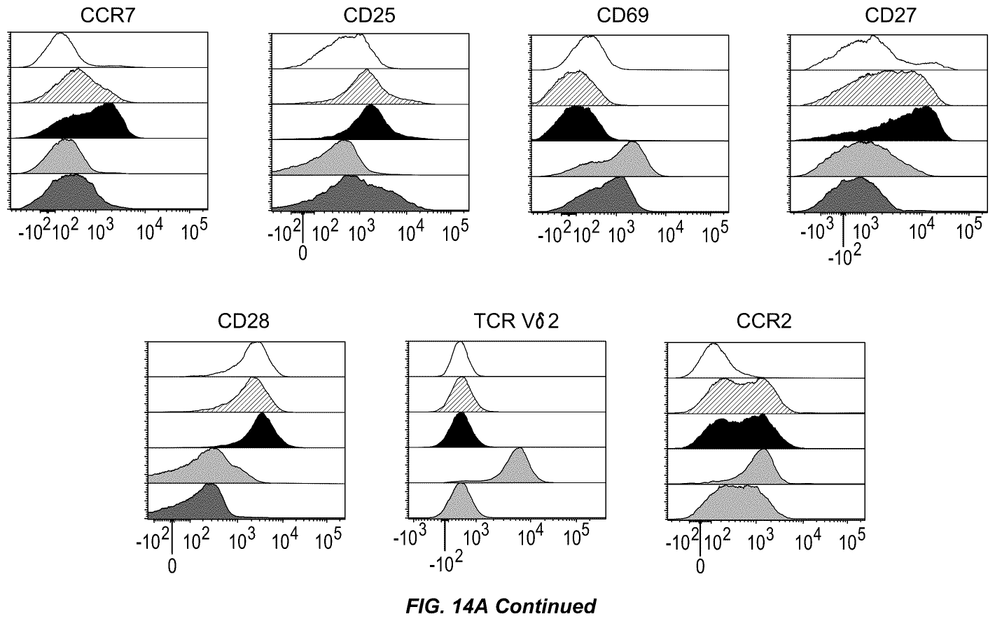

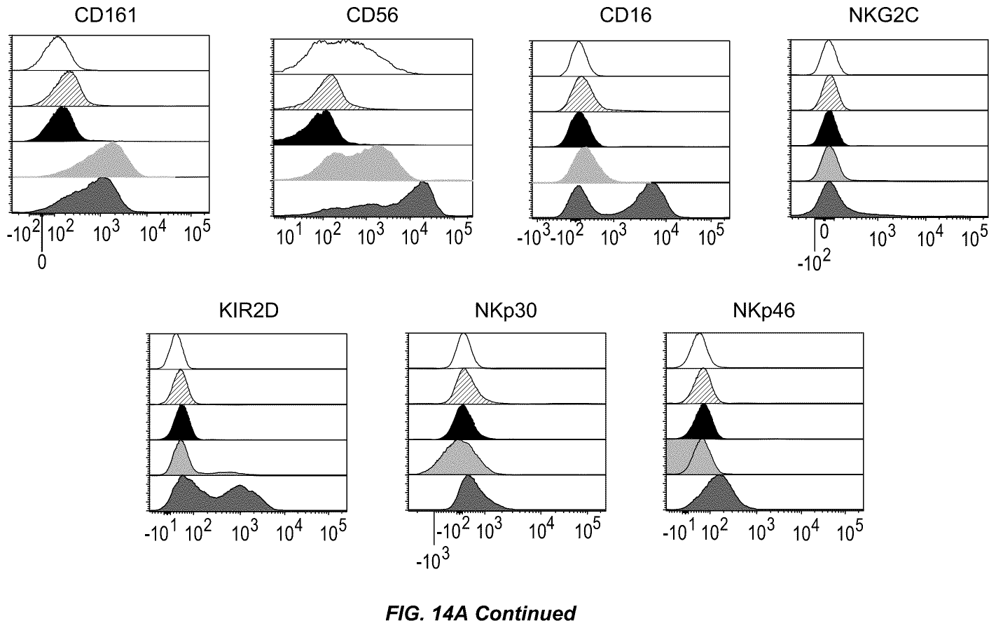

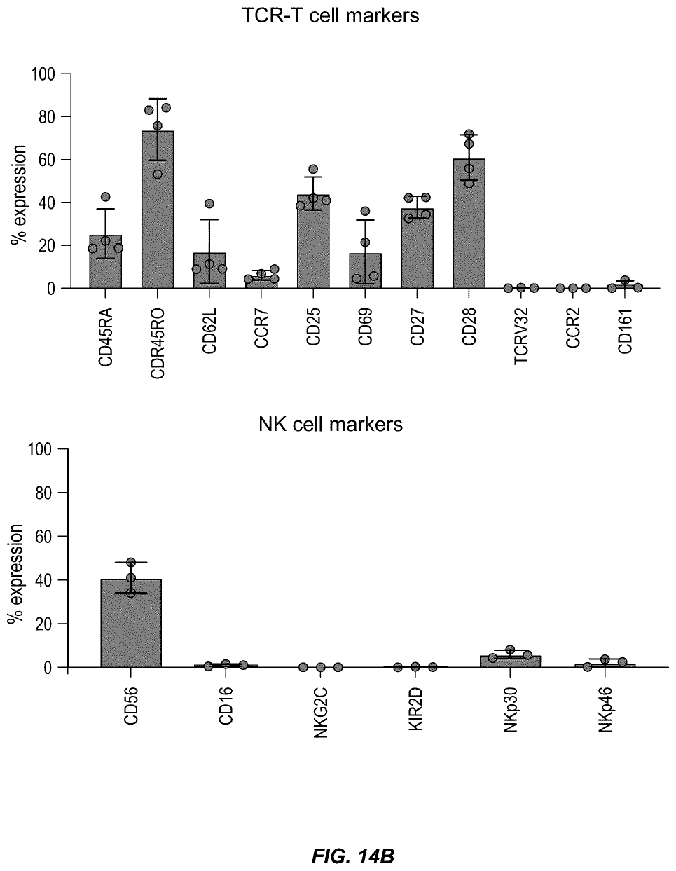

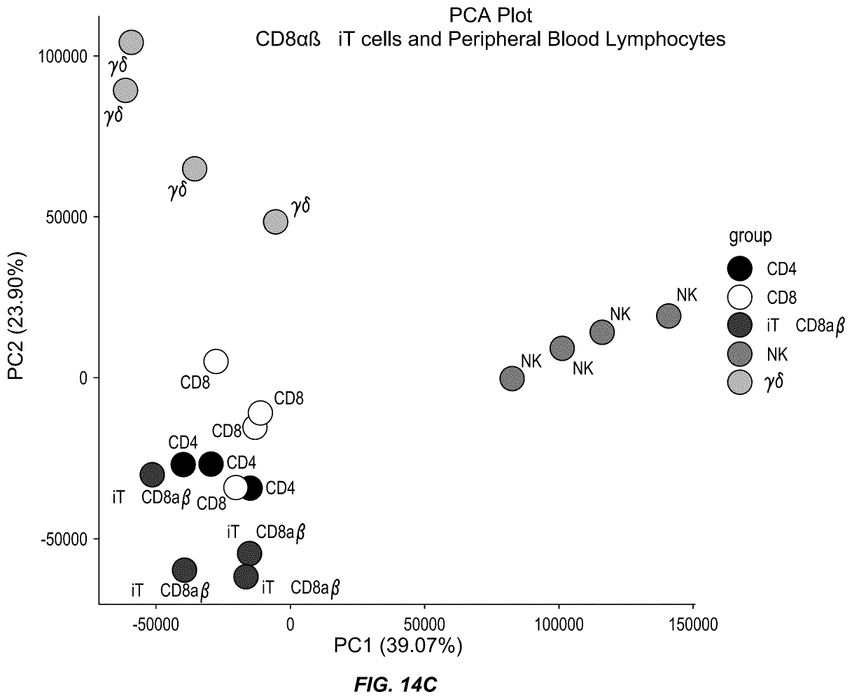

Having established that T cell commitment, differentiation, maturation, and expansion of CD8α CAR T cells can be driven by CAR expression in the absence of a TCR, it was sought out to compare the resulting CD8ab TRAC-1XX-iT cells (CD8ab iT) to naturally occurring peripheral blood lymphocytes. Analysis of cell-surface markers associated with αβTCR-T cells, NK cells or T6TCR-T cells, showed that CD8α iT cells express classical T cell markers including CD45RO, CD25, CD27, CD28 and low levels of CD62L and CCR7 (FIGS. 5A, 14A, and 14B). The stimulated cells expressed the T cell activation/NK cell marker CD56 but lack canonical NK markers such as CD16 and KIR2D (FIGS. 5A, 14A, and 14B). They also did not express 76TCR-T cell associated markers including the γδTCR and CD161 (FIGS. 5A, 14A, and 14B). To assess the nature of the cells more closely, the transcriptomic profile of the CD8αβ iT cells was compared to that of healthy donor PBMC-derived lymphocytes. To enhance comparability, CD4 αβTCR-T (CD4), CD8 αβTCR-T (CD8), γδTCR-T (γδT) and NK cells were engineered to express the 1928z-1XX CAR (either through TRAC targeted integration in CD4 and CD8 or retroviral expression in 76T and NK cells) and purified for the CAR⁺ populations. Unsupervised hierarchical clustering analysis based on a dissimilarity matrix showed that, based on overall gene expression, the CD8αβ iT cells were most related to the CD4 and CD8 T cells (FIG. 5B). Principal component (PC) analysis confirmed that within the first two PCs, CD8αβ iT cell cluster more closely with peripheral blood 76TCR-T cells than γδTCR-T cells (FIG. 14C). Pearson's correlation distinguished that CD8αβ iT cells are more closely related to the peripheral blood CD8 T cells (r=0.99, FIG. 5C).

### TRAC-1XX-iT Cells Achieve Tumor Control in a Systemic In Vivo Leukemia Model

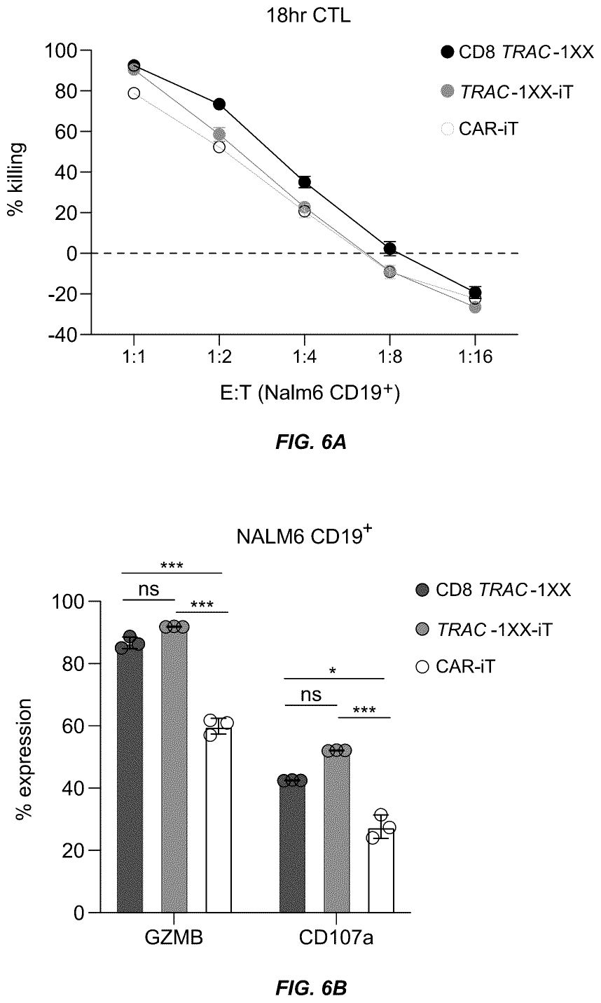

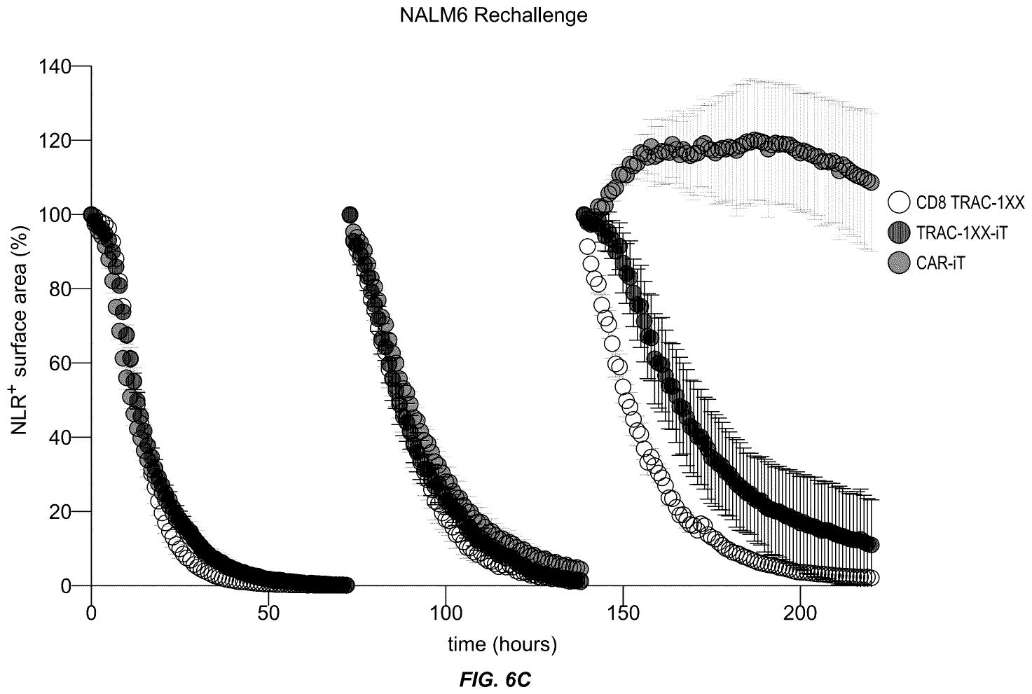

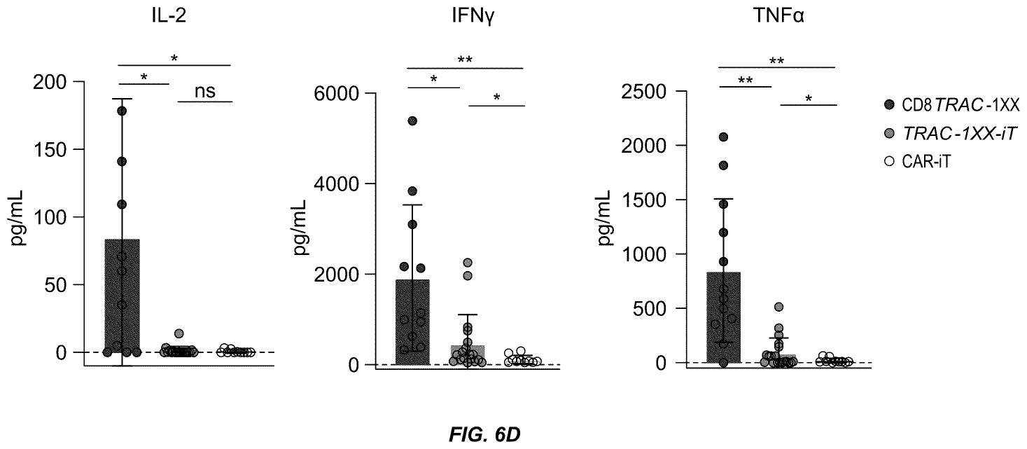

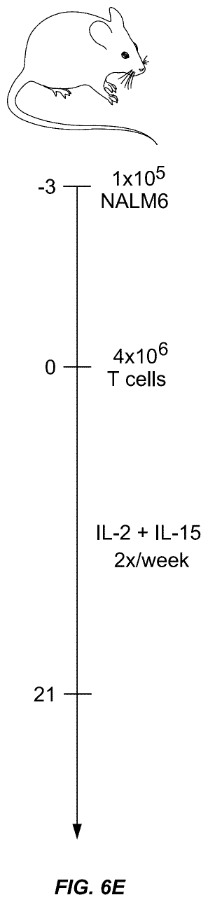

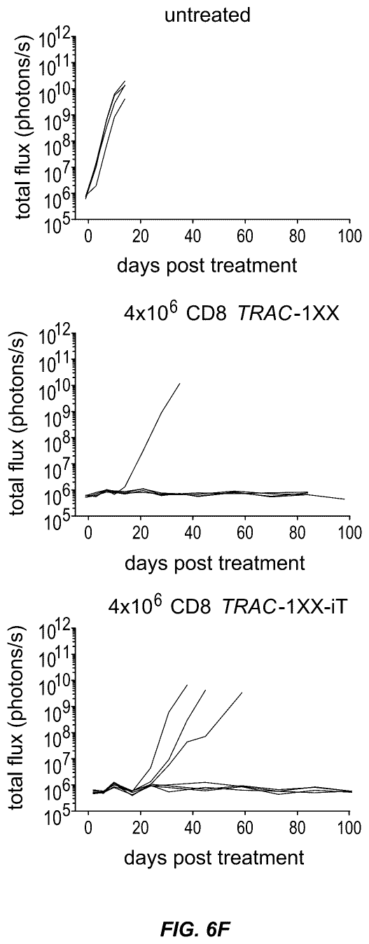

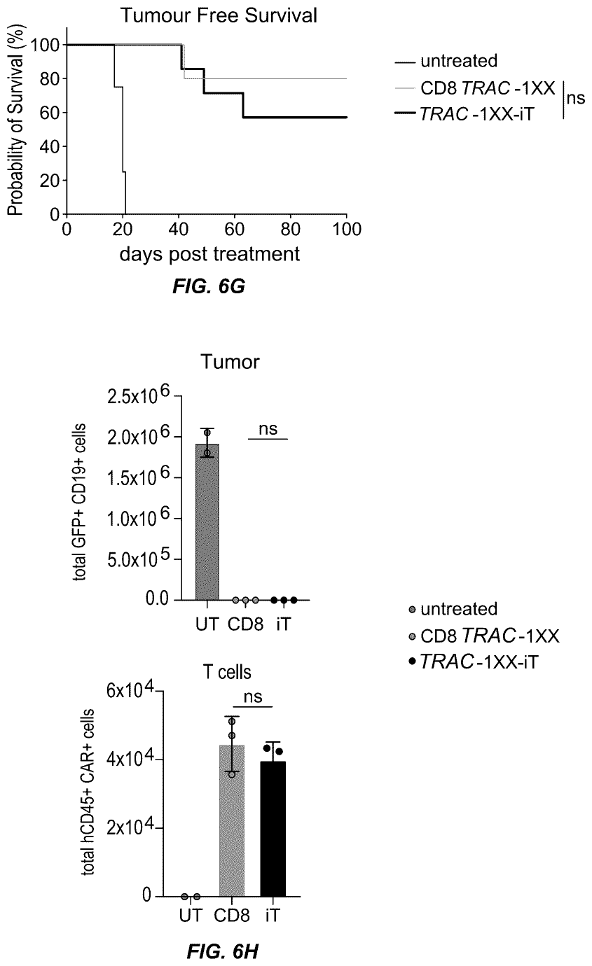

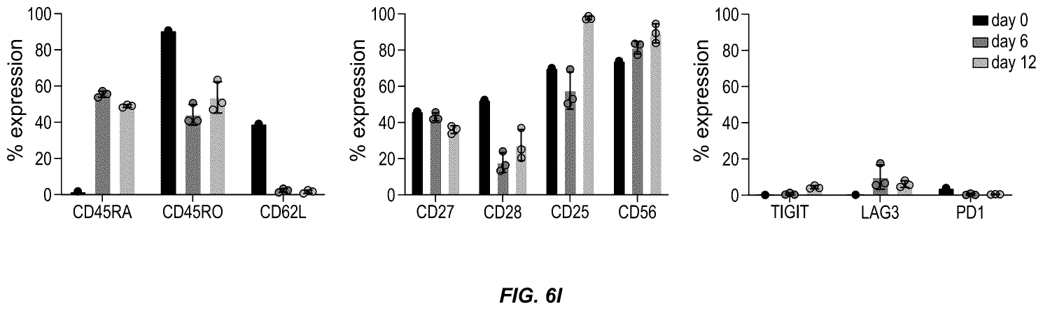

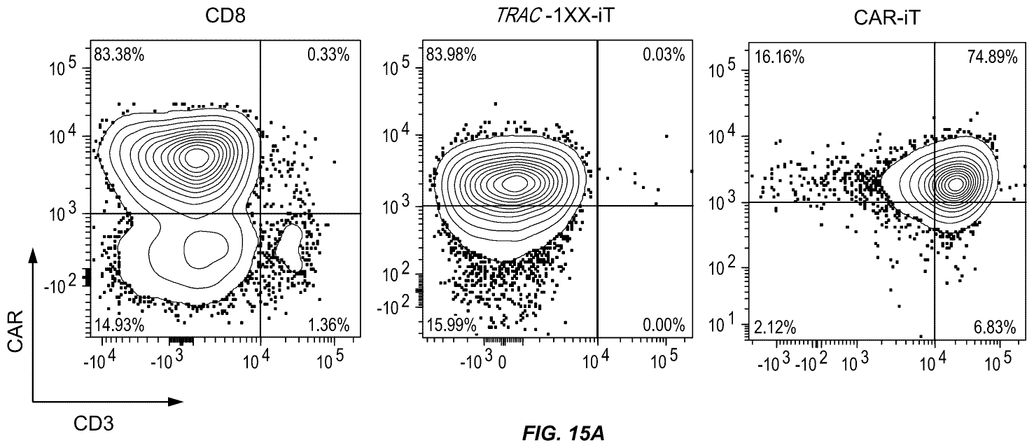

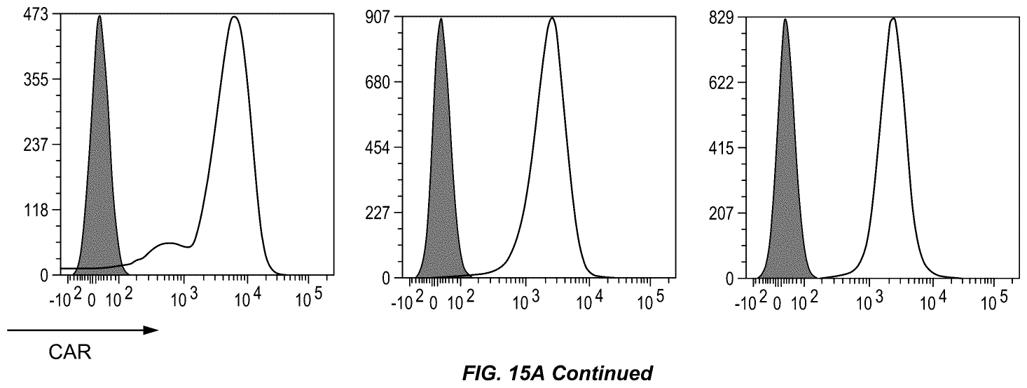

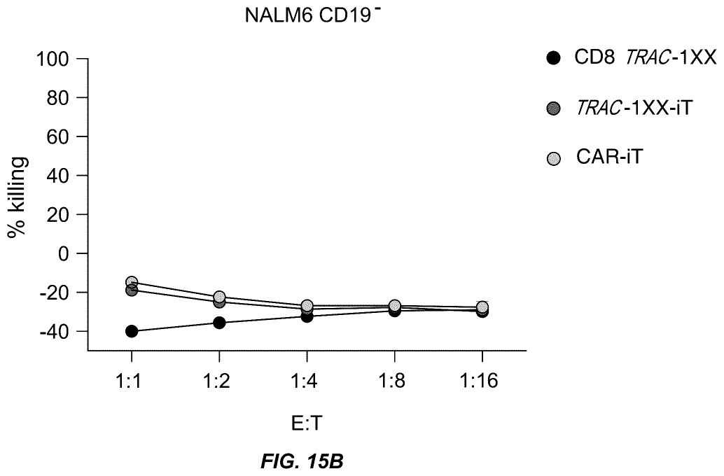

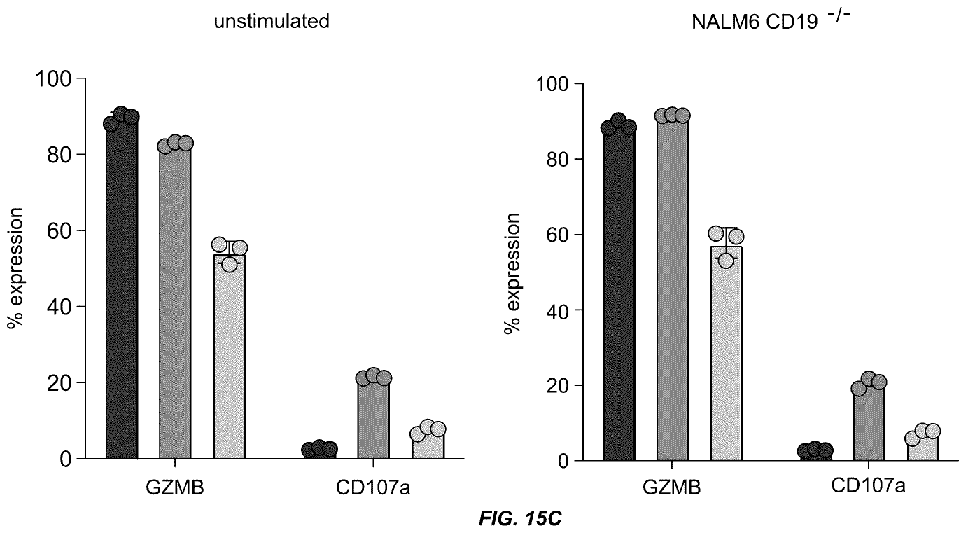

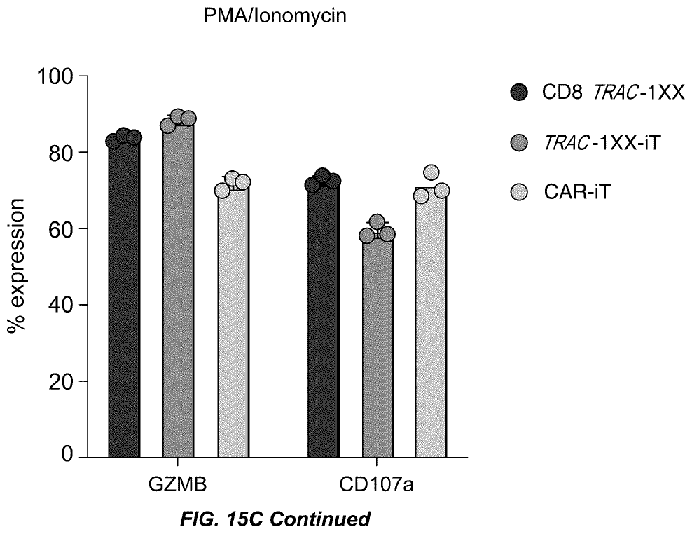

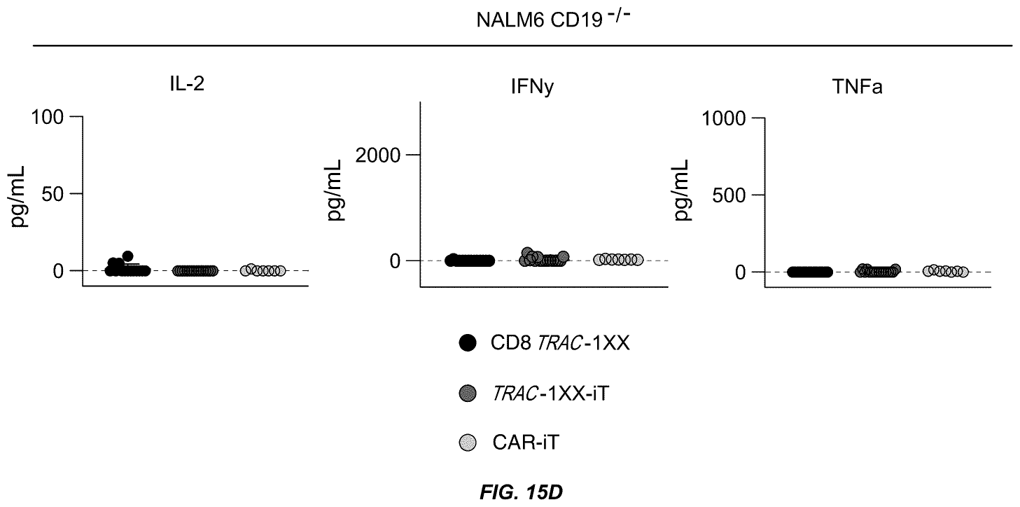

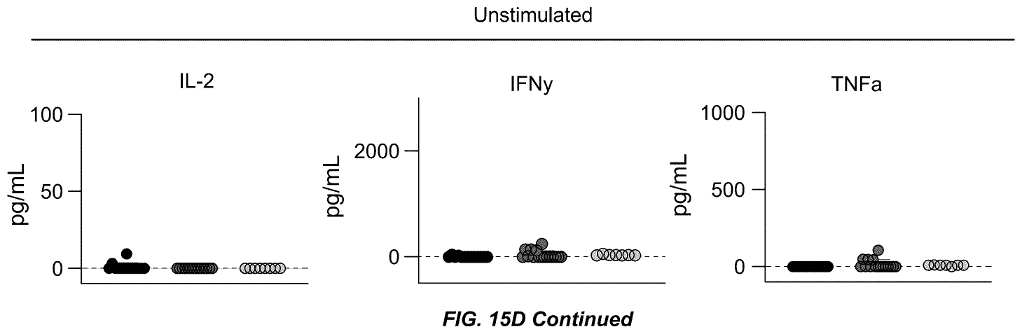

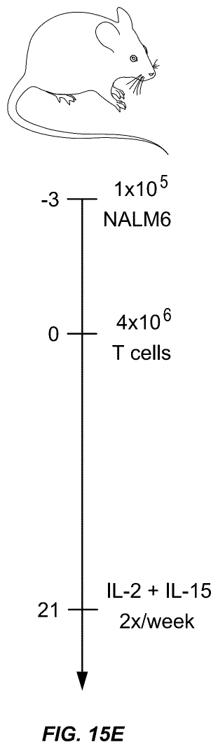

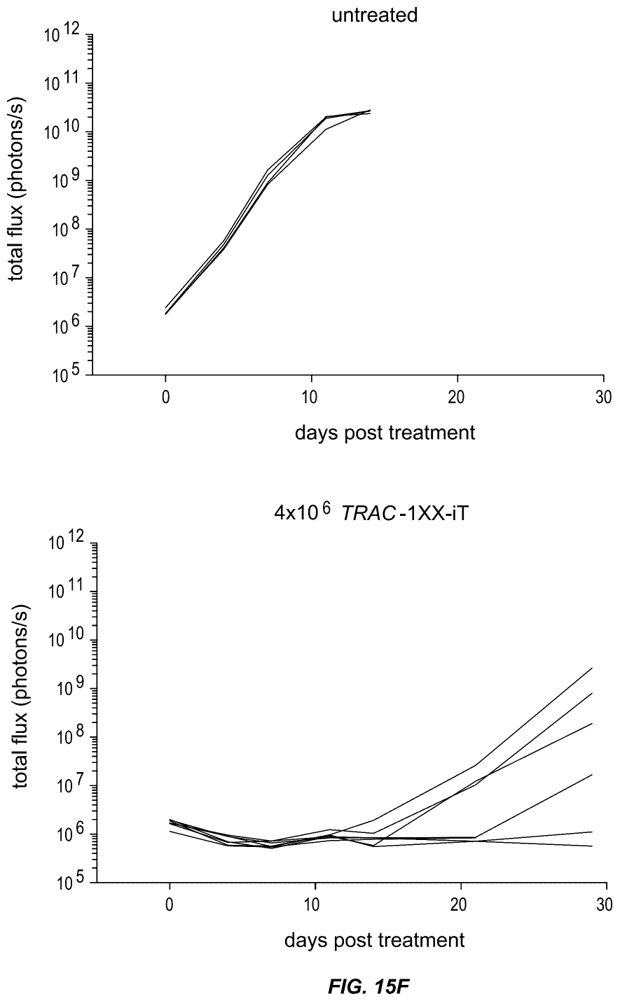

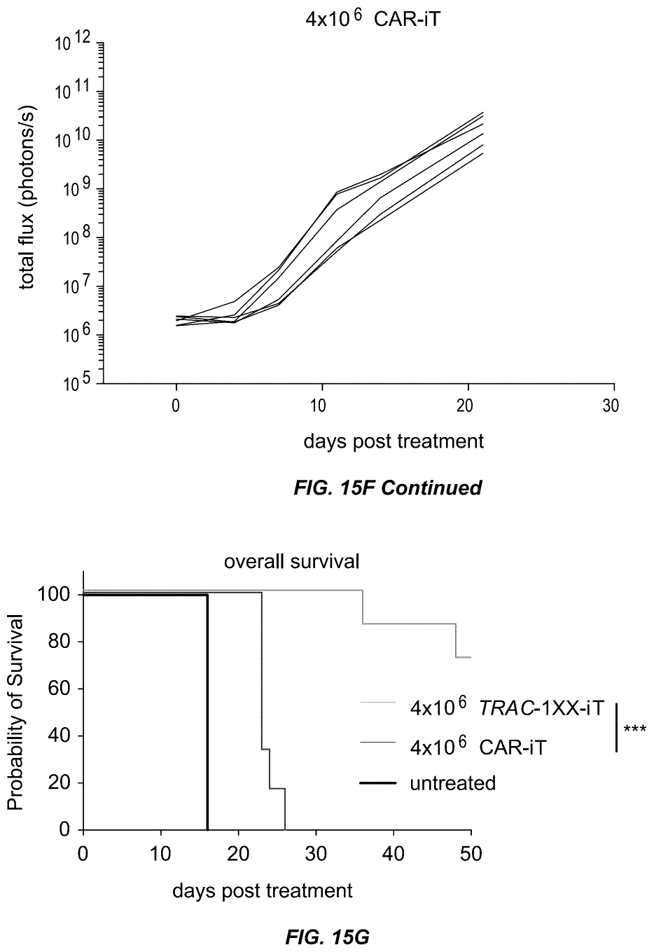

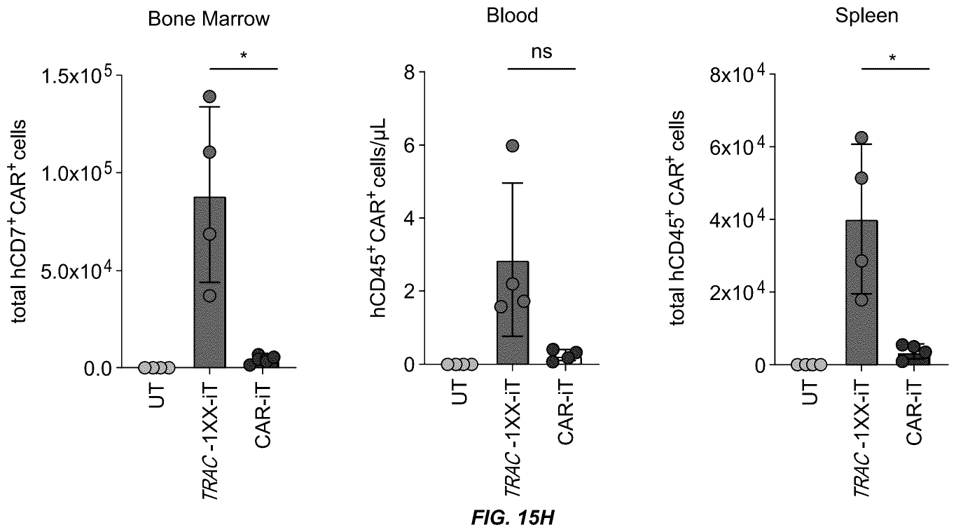

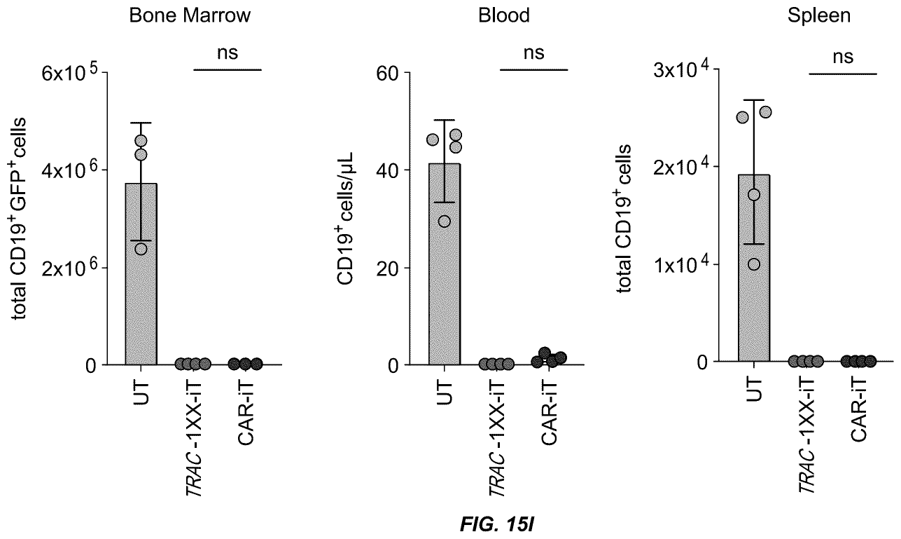

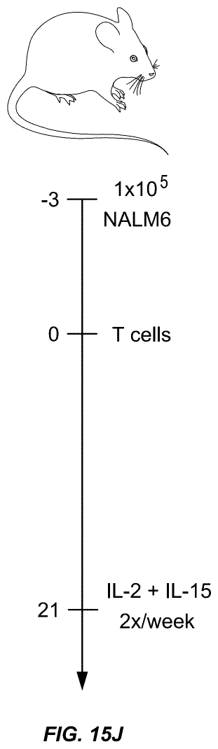

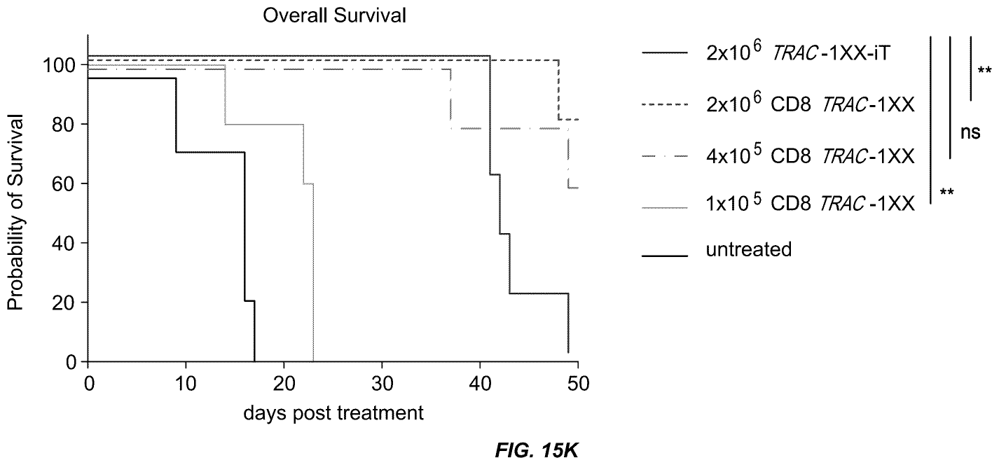

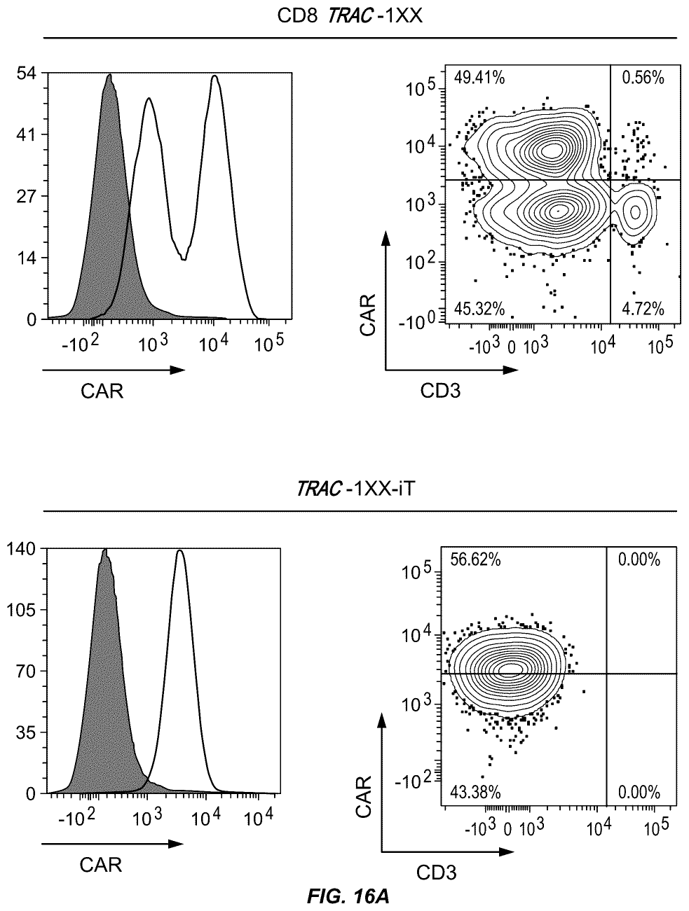

To determine whether the αβTCR-T lineage commitment of TRAC-1XX-iT cells enhanced their functional capabilities, their in vitro and in vivo functions were compared to CAR-iT cells and healthy-donor PBMC-derived CD8⁺ TRAC-1XX αβTCR-T cells (CD8 TRAC-1XX) (FIG. 15A). In vitro cytolytic activity was antigen-specific and similar between the three cell populations in a 18 h cytotoxicity assay (FIGS. 15A and 15B). However, Granzyme B and CD107a production was reduced in CAR-iT cells (FIGS. 6B and 15C). Importantly, TRAC-1XX-iT and CD8 TRAC-1XX cells were able to control repeated in vitro exposure to tumor cells, whereas CAR-iT cells failed after a third challenge (FIG. 6C). In vitro cytokine secretion was reduced in both iT populations compared to CD8 TRAC-1XX T cells (FIGS. 6D and 15D). TRAC-1XX-iT cells were able to produce IFNγ and TNFα, whereas minimal secretion was detected in CAR-iT cells. Notably, both TRAC-1XX-iT and CAR-iT cells lacked the ability to produce IL-2 in response to antigen (FIGS. 6D and 15D). To determine whether the differences in in vitro function between the TRAC-1XX-iT and CAR-iT cells translated into differences in in vivo tumor control, these were compared in the systemic NALM6 leukemia model (FIG. 15E), wherein TRAC-1XX-iT cells showed improved tumor control (FIGS. 15F and 15G), associated with increased iT cell persistence in the bone marrow, spleen and blood (FIGS. 15H and 15I).

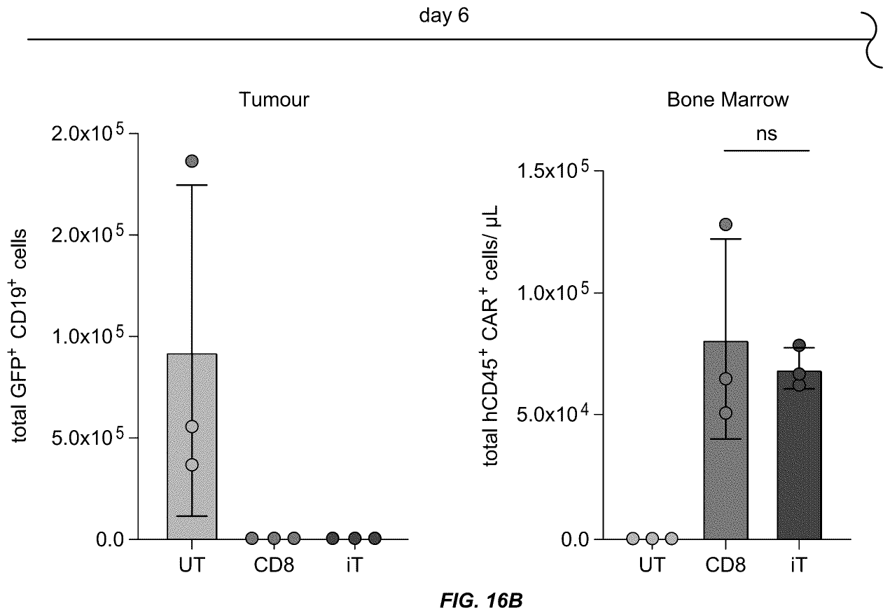

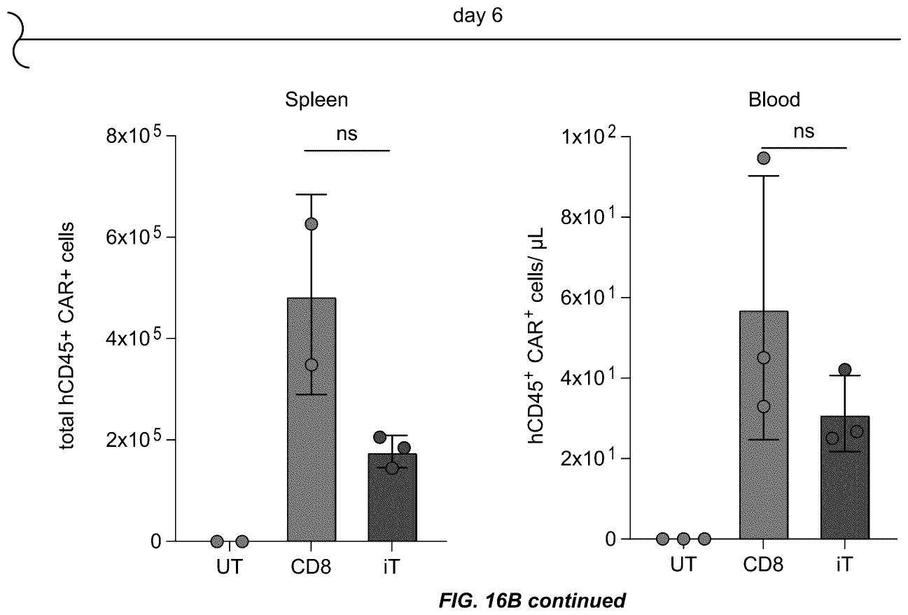

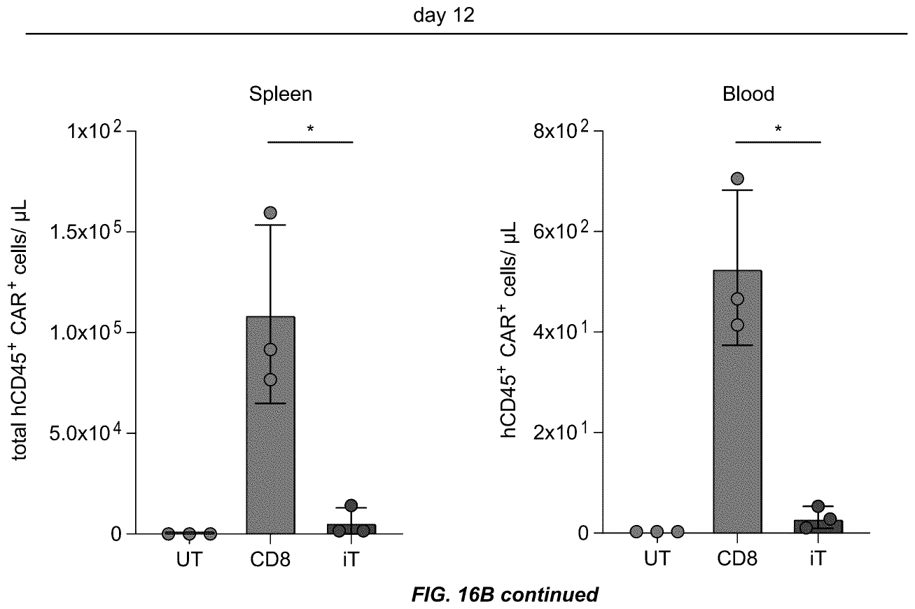

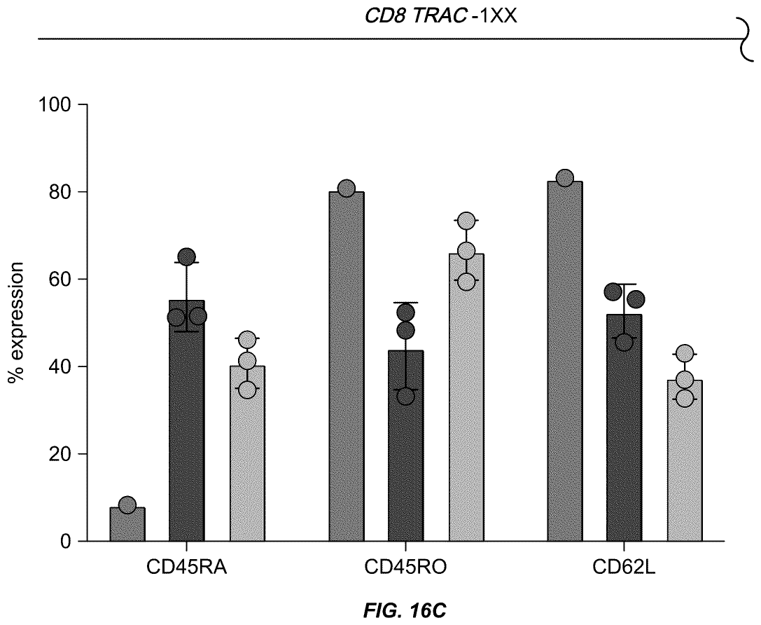

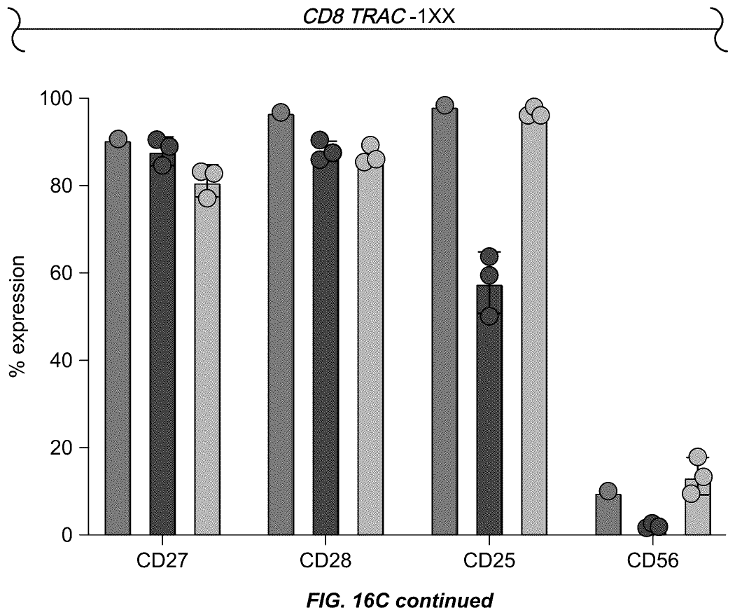

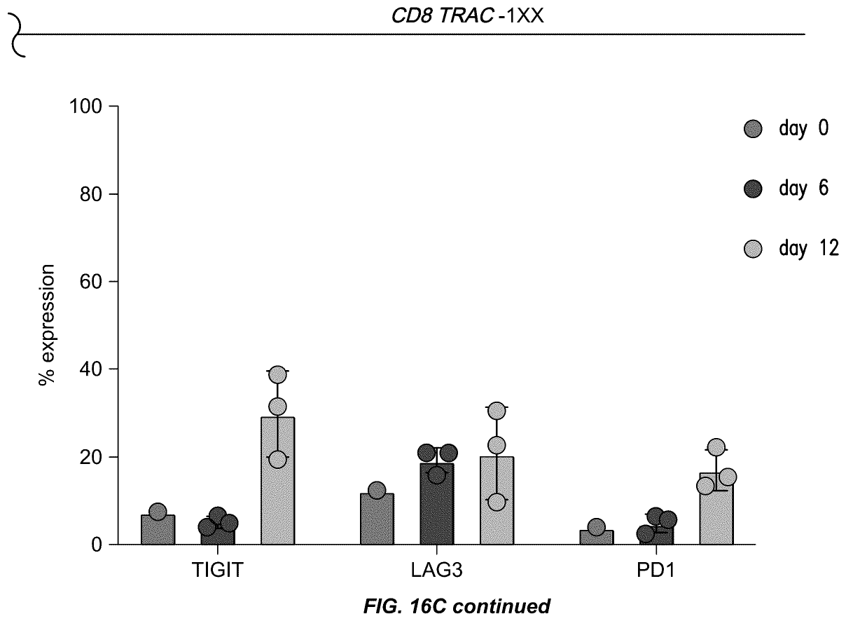

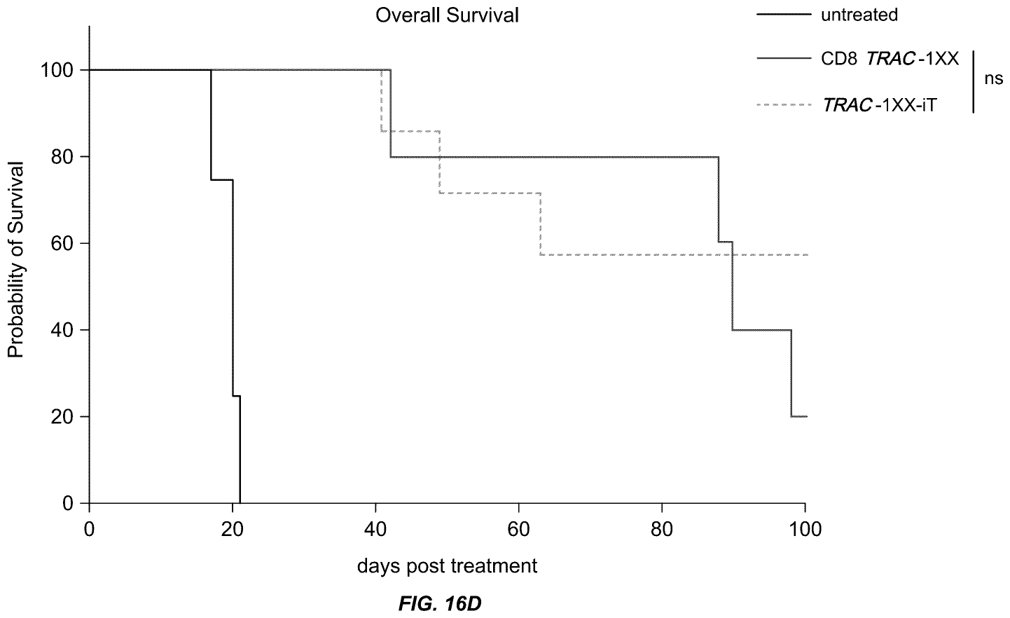

To provide a potency benchmark under these conditions, diminishing doses of CD8 TRAC-1XX T cells were administered and it was found that 2×10⁶ TRAC-1XX-iT cells provided a similar response to 4×10⁵ CD8 TRAC-1XX T cells (FIGS. 15J and 15H). 4×10⁶ cells TRAC-1XX-iT cells induced complete and durable responses (FIGS. 6E-6G). Enumeration of tumor cells in bone marrow, spleen and blood 6 and 12 days after infusion, showed complete absence of detectable tumor in the bone marrow for both treatment groups at either time point (FIGS. 6H and 16B). TRAC-1XX-iT cells showed similar persistence to CD8 TRAC-1XX T cells in bone marrow, spleen and blood by day 6 and in bone marrow on day 12, but were less abundant in spleen and blood by day 12 (FIGS. 6H and 16B). Phenotypically, both populations increased CD45RA expression in vivo, and diminished CD62L expression (FIGS. 6I and 16C). TRAC-1XX-iT cells down-regulated CD27 and CD28 but did not display increased exhaustion markers compared to peripheral blood CD8 TRAC-1XX T cells. Notably, the latter succumbed while presenting GvHD-like symptoms including weight loss, diarrhea and loss of fur, likely caused by the remaining small population of TCR⁺ cells (Qasim et al., Sci Transl Med 9 (2017)), which is not present in TRAC-1XX-iT cells (FIGS. 16A and 16D).

### DISCUSSION

The present example reports here on the generation of therapeutic CD8αβ CAR iT cells from TiPS. It was investigated how premature TCR or CAR expression interferes with adaptive T cell maturation and demonstrated that delayed expression and calibrated CAR signaling enable DP T cell development and terminal CD8αβ iT cell expansion in the absence of a TCR.

In vitro T cell development from TiPS that constitutively express a CAR yields T cells with an innate-like CD8αα T cell phenotype (Themeli et al., Nat Biotechnol 31, 928-933 (2013); Harada et al., Mol Ther (2021)) or NK-like features (Ueda et al., Cancer Sci 111, 1478-1490 (2020); Maluski et al., J Clin Invest 129, 5108-5122 (2019)). The present example shows that premature CAR expression at the DN stage interferes with Notch signaling, skewing differentiation away from DP differentiation and towards the acquisition of an innate-like phenotype. The Notch ligand DLL1 was sufficient to induce DP cell formation during T lineage development from precursor cells that bear TCR VDJ genes in germline configuration, but not from WT-TiPS, which encode a rearranged αβTCR and required DLL4 to progress to the DP stage (FIG. 1D). In the presence of constitutive CAR expression however, DLL4 was no longer sufficient to evoke DP differentiation (FIG. 1F). Constitutive CAR expression diminished NOTCH1 expression and deregulated downstream gene expression, including DTX1, TCF7 and PTCRA (FIG. 3B). Consistent with in vitro models of lymphopoiesis, stronger TCR signals or interference with Notch signaling impairs the DN to DP transition in αβTCR T cells (Ciofani et al., Immunity 25, 105-116 (2006); Hayes et al., Immunity 22, 583-593 (2005)) and defaults the cells towards an innate/γδTCR-like fate.

The combination of regulating CAR expression under the control of the TRAC locus (Eyquem et al., Nature 543, 113-117 (2017)) and inactivating the CAR's second and third ITAM (Feucht et al., Nat Med 25, 82-88 (2019)) brought down the potential for constitutive signaling (FIGS. 12A-12C) and rescued the induction of genes downstream of Notch and DP development (FIGS. 2D and 3B). The partially restored induction of PTCRA in differentiating TRAC-1XX-TiPS is noteworthy (FIGS. 3B-3D), given the crucial role the preTCR-complex plays in the development of αβTCR-T cells (but not γδTCR-T cells (Fehling et al., Nature 375, 795-798 (1995))). PreTCR expression is required for R chain selection and its absence diverts T cell differentiation towards a 76TCR-T cell phenotype (Terrence et al., J Exp Med 192, 537-548 (2000)). βTCRA expression is absent in the presence of a constitutively expressed CAR (FIG. 3B), consistent with Notch downregulation (Reizis & Leder, Genes Dev 16, 295-300 (2002)). Facilitating DP development through attenuation of CAR signaling is also consistent with the finding that attenuated 76TCR signaling can allow for DP maturation in absence of an αβTCR (Haks et al., Immunity 22, 595-606 (2005)).

TRAC-1XX-TiPS cannot assemble a functional αβTCR and therefore depend on the CAR to direct T cell maturation past the DP stage. Exposure to the CAR antigen resulted in the maturation of CD8αβ SP by day D42, but failed to support their expansion (FIGS. 4A and 4C). Provision of 4-1BB costimulation together with antigen enabled the emerging SP CAR T cells to expand. Up-regulation of 4-1BB has been observed in murine DP cells undergoing positive selection in vivo (Kim et al., Exp Mol Med 41, 896-911 (2009)). The benefit of providing costimulation is consistent with TCR/MHC interaction alone not sufficing to induce complete CD8 T cell maturation (Groves et al., J Immunol 158, 65-75 (1997); Anderson et al., Immunol Today 20, 463-468 (1999)). The emerging SP TRAC-1XX-iT cells exposed to 4-1BBL not only expanded but also acquired robust effector functions (FIGS. 4I-4M) and the ability to expand upon repeated antigen stimulation (FIG. 4K). However, these iT cells do not have a classical naïve phenotype, as they maintain CD5 and CD7 expression but do not homogeneously express CD45RA, CD62L and CCR7, as would be expected in naïve T cells and recent thymic emigrants (McFarland et al., Proc Natl Acad Sci USA 97, 4215-4220 (2000)). They rather express CD45RO, CD28, CD25 and CD56, hallmarks of recently activated T cells (FIG. 13C). This effector-like phenotype is commonly observed following extrathymic differentiation of T cells, irrespective of CAR expression or maturation protocol (Iriguchi et al., Nat Commun 12, 430 (2021); Ito et al., Commun Biol 4, 694 (2021)). Whist the TRAC-1XX-iT described here do not display a canonical naïve phenotype, it is noteworthy that they also do not express common exhaustion markers. Transcriptional studies confirmed that CAR-induced maturation produces CD8αβ TRAC-1XX-iT cells that are more similar to peripheral blood-derived TRAC-1XX CD8αβ T cells (CD8 TRAC-1XX) than CD4 αβTCR-T cells and are more distinct yet from 76TCR-T cells and NK cells (FIG. 5b, c).

When comparing TRAC-1XX-iT function to CAR-iT and peripheral blood-derived CD8 TRAC-1XX, it was found that TRAC-1XX-iT had improved cytolytic capacity and cytokine secretion compared to CAR-iT (FIGS. 6D and 6D), as well as improved anti-tumor activity in vivo (FIGS. 15E-15H). TRAC-1XX-iT cells still produced significantly lower levels of cytokines than CD8 TRAC-1XX cells, notably lacking IL-2 production (FIG. 6D). TRAC-1XX-iT cells nonetheless provide substantial anti-tumor activity in a systemic NALM6 model, which CAR-iT cannot achieve (FIGS. 6G and 15G), while requiring a higher dosage than CD8 TRAC-1XX T cells (FIG. 15K). Phenotypically, both TRAC-1XX-iT and CD8 TRAC-1XX cells differentiated towards an effector phenotype upon encounter with the tumor in the bone marrow. TRAC-1XX-iT cells downregulated CD62L, CD27 and CD28, but did not show accelerated acquisition of exhaustion markers (FIGS. 6I and 16C). TRAC-1XX-iT cells showed reduced persistence in spleen and blood over time (FIG. 16B). Despite those differences, TRAC-1XX-iT cells were able to induce long-term remission and survival following intravenous infusion of a single dose of 4×10⁶ iT cells (FIGS. 6F and 6G). Tumor control by CAR-iT cells has only been hitherto achieved in intraperitoneal models (Themeli et al., Nat Biotechnol 31, 928-933 (2013); Harada et al., Mol Ther (2021); Ueda et al., Cancer Sci 111, 1478-1490 (2020)).

TiPS are a highly attractive resource for allogeneic, “off-the-shelf” immunotherapy (Themeli et al., Cell Stem Cell 16, 357-366 (2015)). The self-renewing capacity of TiPS allows for the establishment of gene edited, clonally selected master cell banks (Valamehr et al., Stem Cell Reports 2, 366¬381 (2014); Nagano et al., Mol Ther Methods Clin Dev 16, 126-135 (2020)) which can be utilized to mass produce genetically homogeneous T cell populations, eliminating donor-dependent T cell variability and thereby standardizing treatment. Combined with genome editing, the TiPS platform allows for careful selection of a desired genotype, screening for insertional mutagenesis (Fraietta et al., Nature 558, 307-312 (2018)), off-target editing and translocations (Poirot et al., Cancer Res 75, 3853-3864 (2015); Stadtmauer et al., Science 367 (2020)), and facilitates the complete elimination of TCR expression to prevent GvHD (Qasim et al., Sci Transl Med 9 (2017)) or the accidental transduction of malignant cells in apheresis products (Ruella et al., Blood 135, 505-509 (2020)). Genotype selection, including detection of off-target editing events, renders the TiPS platform particularly suitable for multiplexed gene editing strategies, such as combining TRAC locus editing with the ablation of CD52 (Poirot et al., Cancer Res 75, 3853-3864 (2015)), CD70 (Mansilla-Soto et al., Nat Med 28, 345-352 (2022)), or PD1 (Stadtmauer et al., Science 367 (2020)). The use of TiPS-derived T cells is not limited to targeting CD19 as described here and is applicable to other target tumor associated antigens, barring that potential interaction between the CAR with an antigen expressed during T cell development does not interfere with T lineage commitment, as well as applications beyond cancer immunotherapy (Amor et al., Nature 583, 127-132 (2020)). In the presently disclosed protocol, 1 million TRAC-1XX-iT cells are generated from a single TiPS in 42 days (FIG. 4H). This engineering flexibility and expansion potential provide a platform that can be feasibly scaled to generate clinically relevant CAR iT cell numbers, which in principle may allow for off-the-shelf application from batches of uniform and consistent CAR iT cells produced from the same engineered master cell bank.

In summary, the present example demonstrate that synthetic receptors like CARs can substitute for the TCR in driving directed T cell differentiation and show that induction of TCR^{-/-}, αβTCR-T cell-like CD8αβ CAR T cells is feasible and holds great potential for large scale production of potent T cell-based immunotherapies.

### Methods

Cell Lines: OP9-mDLL1. OP9-mDLL1 cells were cultured as previously described (Themeli et al., Nat Biotechnol 31, 928-933 (2013)).

Cell Lines: OP9-DLL1, -DLL4, -JAG1, -JAG2. Parental OP9 cells were obtained from ATCC. Plasmids encoding the Moloney murine leukemia virus-based SFGγ retroviral vector (Riviere et al., Proc Natl Acad Sci USA 92, 6733-6737 (1995)) were used to clone bi-cistronic constructs to express one of the human Notch ligands (DLL1, DLL4, JAG1 or JAG2) and GFP. Plasmids containing sequences of hDLL1, hDLL4, hJAG1 and hJAG2 were obtained from GenScript (NM_005618, NM_019074, NM_000214 and NM_002226 respectively) and were cloned using standard molecular biology techniques by replacing the FFLuc element in the SFG-FFLuc-P2A-GFP retroviral vector with the desired Notch ligand. Vesicular stomatitis virus glycoprotein G (VSV-G) pseudotyped retroviral supernatants derived from transduced gpg29 fibroblasts (H29) was used to transduce OP9. Transduced cells were purified by flow cytometry based on Notch ligand and GFP expression. Antibodies used to detect Notch ligand expression were hDLL1-PE (MHD1-314; BioLegend), hDLL4-PE (MHD4-46; BioLegend), hJagged-1-PE (MHJ1-152; BD) and hJagged-2-PE (MHJ2-523; BioLegend) respectively. Purified OP9 cells were cultured in MEMa (Gibco) media with 20% Fetal Bovine Serum (FBS, Hyclone), 1×MEM Non-Essential Amino Acids (NEAA, Corning), 2 mM GlutaMAX (Gibco), 100 U/mL Penicillin (Pen) and 100 μg/mL Streptomycin (Strep, Corning), 55 tM 2-Mercaptoethanol (2-ME, Gibco) and 50 mg/mL ascorbic acid (Sigma) as previously described (Themeli et al., Nat Biotechnol 31, 928-933 (2013)).

Cell Lines: NALM6. NALM6 cells were obtained from ATCC. NALM6 CD19^{-/-} were generated as previously described (Hamieh et al., Nature 568, 112-116 (2019)). NALM6 were transduced to express GFP and firefly Luciferase (FFLuc) for in vitro and in vivo detection (Zhao et al., Cancer Cell 28, 415-428 (2015)). Cells were cultured in RPMI 1640 (Corning) with 10% FBS (Hyclone) 1×NEAA, 2 mM GlutaMAX, 100 U/mL Pen, 100 tg/mL Strep, 2 mM HEPES (Corning) and 55 tM 2-ME). For Incucyte-based analysis, NALM6 CD19⁺ and NALM6 CD19^{-/-} were transduced with Incucyte NucLight Red lentiviral reagent (NLR, Essen BioScience) and selected with puromycin according to manufacturer's instructions.

Cell Lines: 3T3-CD19. NIH 3T3 cells expressing CD19 were used as artificial antigen presenting cells as previously described (Zhao et al., Cancer Cell 28, 415-428 (2015)). 3T3-CD19-4-1BBL were generated utilizing a previously described SFGg retroviral vector encoding the 4-1BBL transgene (Riviere et al., Proc Natl Acad Sci USA 92, 6733-6737 (1995)). VSV-G pseudotyped retroviral supernatant derived from transduced H29 was used to transduce 3T3-CD19. Transduced cells were purified by flow cytometry based on 4-1BBL (4-1BBL-PE, 5F4; BioLegend) expression. Cells were cultured in DMEM media (Corning) with 10% Cosmic Calf Serum (Hyclone).

Cell Lines: K562-mbIL-21-4-1BBL. K562 cells were transduced to express membrane-bound IL-21 and 4-1BBL as previously described (Cichocki et al., Sci Transl Med 12 (2020)). Cells were cultured in RPMI 1640 (Corning) with 10% FBS (Hyclone), 2 mM GlutaMAX, 100 U/mL Pen 100 tg/mL Strep, 55 tM 2-ME.

Cell Lines: H1. The human embryonic stem cell line was cultured on MEF in hES media (DMEM-F12 (Corning) with 20% knock-out serum replacement (KSR), 1×NEAA, 2 mM GlutaMAX, 100 U/mL Pen 100 μg/mL Strep, 55 μM 2-ME) supplemented with 8 ng/mL hbFGF (R&D systems).

Cell Lines: Primary CLL cells. Apheresis product before CAR T cell infusion were obtained from patients that were consented and enrolled in phase I 1928z CAR T cell clinical trials.

Generation of iPS: FiPS. Fibroblast-derived iPS (FiPS) were generated as previously described (Valamehr et al., Stem Cell Reports 2, 366¬381 (2014)), transfected with a transient plasmid-based reprogramming system to initiate cellular reprogramming. In brief, cells were transfected with reprogramming vector backbone (pCEP4, Life Technologies) containing OCT4, NANOG, and SOX2 under regulation of the EF1a promoter. Transfected cells were plated on Matrigel and selected with hygromycin until FiPS colonies were established.

Generation of iPS: WT-TiPS. WT-TiPS were generated as previously described (Clone T-iPSC-1.10) (Themeli et al., Nat Biotechnol 31, 928-933 (2013)). In brief, healthy-donor peripheral blood mononuclear cells (PBMCs) were activated with phytohemagglutinin (PHA, 2 μg/mL) and transduced with two tri-cistronic SFGγ retroviral vectors, each vector encoding reprogramming factors and a different fluorescent marker (f-Citrine-P2A-cMYC-E2A-SOX2 and f-vexGFP-P2A-OCT4-T2A-KLF4). Transduced cells were seeded on MEF feeder cells and TiPS colonies were established.

Generation of iPS: CAR-TiPS. CAR-TIPS were generated as previously described (1928z-T-iPSC) (Themeli et al., Nat Biotechnol 31, 928-933 (2013)). In brief, clone T-iPSC-1.10 was stably transduced with a bi-cistronic lentiviral vector (mCherry-P2A-1928z) and purified for mCherry expression by flow cytometry.

Generation of iPS: TRAC^{-/-}-TiPS. TRAC^{-/-}-TiPS were generated through CRISPR/Cas9-targeted integration of a EF1a-GFP-P2A-Puromycin-bGHpA(G2AP) expression unit into the TRAC locus leading to knockout of the TCRα gene into the WT-TiPS (Clone T-iPSC-1.10). WT-TiPS were electroporated using Lonza Cell Line Nucleofector Kit V solution and Lonza Nucleofector-II. Five million cells were resuspended in 100 uL nucleofection solution, with 2.6 μg pBS-TRAC gRNA1, 2.6 μg pBS-TRAC-HR-G2AP and 2.6 tg hCas9 plasmid, and electroporated using program B-025. Transfected cells were plated on Matrigel-coated plates using TiPS complete medium containing 10 μM ROCK inhibitor. After 48 h cells were selected with 0.8 μg/ml Puromycin and sorted by flow cytometry for GFP expression. pBS-TRAC gRNA1 was generated by cloning the TRAC gRNA target sequence (5′-CAGGGTTCTGGATATCTGT) into pBS-gRNA MCS plasmid, which contains the human U6 promoter and the gRNA fold described in Mali et al., Science 2013. pBS-TRAC-HR-G2AP was generated by cloning the left (˜0.9 kb) and right (1 kb) TRAC homology arms (HAs) into pBluescript II SK (+), followed by the insertion of the EF1a-GFP-P2A-Puromycin-bGHpA expression unit in between of the HAs. hCas9 plasmid (Mali et al., Science 339, 823-826 (2013)) was obtained from Addgene (41815).

Generation of iPS: TRAC-1928z-TiPS. TRAC-1928z T cells were generated as previously described (Eyquem et al., Nature 543, 113-117 (2017); Mansilla-Soto et al., Nat Med 28, 345-352 (2022)). In brief, αβTCR-T cells were purified from PBMCs with the Pan T cell Isolation kit (Miltenyi Biotec) on the AutoMACS Pro according to manufacturer instructions. Purified cells were activated with CD3/CD28 Dynabeads (1:1 beads:cell) in X-Vivo 15 media (Lonza) supplemented with 5% human serum (HS) (Gemini Bioproducts) with 5 ng/mL rhIL-7 (R&D Systems) and 5 ng/mL rhIL-15 (R&D Systems). 48 h after αβTCR-T cell activation, CD3/CD28 beads were magnetically removed, and T cells were transfected by electrotransfer of TRAC ribonucleoprotein (RNP) using the Nucleofector II device (Lonza). Then 2×10⁶ T cells were resuspended in P3 buffer (Lonza) and mixed with 60pmol TRAC RNP in a total volume of 20p L. Following electroporation and considering 66.7% viability, cells were diluted into culture medium and 1×10⁶/mL and incubated at 37° C., 5% CO2. Recombinant AAV6 donor vector pAAV-TRAC-1928z (Eyquem et al., Nature 543, 113-117 (2017)) was added to the culture 30 min after electroporation at a multiplicity of infection of 3×10⁵ genome copies (GC). Twenty-four h after targeting, T cells were reprogrammed as described above (WT-TiPS) and TRAC-1928z-TiPS colonies were established and cloned on MEF feeder cells. PCRs were performed to determine biallelic, specific target transgene integration into the TRAC locus.

Generation of iPS: TRAC-1XX-TiPS. αβTCR-T cells were isolated, activated and transfected as described above (TRAC-1928z-TiPS). Following electroporation cells were transduced with the previously described recombinant AAV6 donor vector pAAV-TRAC-1XX containing the 1928z-1XX CAR construct. The 1928z-1XX CAR contains tyrosine-to-phenylalanine point mutations within ITAM2 and ITAM3 of the CD3z domain rendering only ITAM1 functional (Feucht et al., Nat Med 25, 82-88 (2019)) TRAC-1XX-T cells were reprogrammed as described above for the FiPS. Emerging iPSCs colonies were expanded and cloned by limiting dilution. PCRs were performed to determine biallelic, specific target transgene integration into the TRAC locus.

Generation of iPS: iPS culture. iPS lines were maintained on MEF prior to EB-based differentiation, in serum-free hES medium supplemented with 8 ng/mL hbFGF. Prior to monolayer-based iCD34 differentiation, iPS lines were cultured on Matrigel in hES media containing 0.4 μM PD032590, 1 μM CHIR99021, 5 μM Thiazovivin, 2 μM SB431542 (all Biovision), 10 μM ROCK-inhibitor (Ascent) and 10 ng/mL hbFGF (R&D Systems) as previously described (Valamehr et al., Sci Rep 2, 213 (2012)). Fresh media was provided every day and cells were passaged every 3-4 days as previously described (Themeli et al., Nat Biotechnol 31, 928-933 (2013); Valamehr et al., Stem Cell Reports 2, 366¬381 (2014)). iPS lines were tested for mycoplasma contamination every 2 months. iPSC surface marker expression. iPS lines were assessed for cell surface pluripotency marker expression including SSEA4-FITC (MC813-70; BD), TRA-1-81-af647 (TRA-1-81; BD) and CD30-PE (BerH8; BD).

Verification of transgene integration into the TRAC locus. Genomic DNA was isolated using QuickExtract DNA Extraction Solution (Lucigen) following manufacturer's protocols. PCRs were performed using KAPA 2× HiFi Hot Start Ready Mix following manufacturer's recommended conditions. PCR products were analyzed using ethidium bromide-stained agarose gel electrophoresis and imaged using the Bio-Rad ChemiDoc.

Verification of transgene integration into the TRAC locus: TRAC^{-/-}-TiPS. Successful disruption of the TRAC locus through insertion of the G2AP expression unit was verified through PCR of the region spanning between the left- and right homology arms. Primers: 5′-GATGATACGCGTCTCTTCTCCTTTCTCATTGAGC and 5′-TCGAGTAAACGGTAGTGCTG. Non-targeted allelles produce a 1603 bp PCR product, targeted alleles a 4434 bp product.

Verification of transgene integration into the TRAC locus: TRAC-1928z-TiPS and TRAC-1XX-TiPS. Successful integration of the 1928z and 1928z-1XX CAR construct respectively were assessed as previously described (Feucht et al., Nat Med 25, 82-88 (2019); Eyquem et al., Nature 543, 113-117 (2017)).

T cell differentiation from iPS and expansion of TRAC-1XX-iT cells. For the differentiation of iPS to hematopoietic precursors, optimized serum- and feeder-free in vitro differentiation protocols were used based on Embryoid Body (EB) formation (Themeli et al., Nat Biotechnol 31, 928-933 (2013)) or monolayer-based (iCD34) (Valamehr et al., Stem Cell Reports 2, 366¬381 (2014)).

Hematopoietic precursor differentiation. EB-based precursor differentiation was performed as previously described (Themeli et al., Nat Biotechnol 31, 928-933 (2013)). Undifferentiated TRAC^{-/-}-TiPS or WT-TiPS colonies were transferred to ultra-low attachment plates to allow for EB formation in serum-free differentiation medium (StemPro-34 (Invitrogen), with 2 mM GlutaMAX, 1×NEAA, 100 U/mL Pen, 100 μg/mL Strep, 55 μM 2-ME, and 50 mg/mL ascorbic acid). Mesoderm induction was facilitated through EB culture with 30 ng/mL hBMP-4 and 5 ng/mL hbFGF until day 4. Next, hematopoietic specification and expansion was achieved in the presence of 20 ng/mL hVEGF and a cocktail of hematopoietic cytokines (100 ng/mL rhSCF, 20 ng/mL rhFlt3L, 20 ng/mL rhIL-3 and 5 ng/mL hbFGF). Cells were transferred to fresh media with cytokines every 48 h. All cytokines were obtained from R&D systems. Day 10 EBs containing hematopoietic progenitor cells were dissociated with Accutase (StemCell Technologies) prior to culture on OP9 for T lymphoid commitment and expansion.

Monolayer-based iCD34 differentiation was performed as previously described (Cichocki et al., Sci Transl Med 12 (2020)). H1, FiPS or TiPS were differentiated to mesoderm and subsequently to CD34⁺ hematopoietic progenitors on matrigel in StemPro differentiation media supplemented with a combination of 5 ng/mL hBMP-4, 10 ng/mL hbFGF, 10 ng/mL rhVEGF, 50 ng/mL hSCF, 10 ng/mL hIL-6, 10 ng/mL hIL-11 for 10 days. Media with fresh cytokines was supplemented every 48 h. All cytokines were obtained from R&D systems. On day 10, CD34⁺ cells were enriched through positive selection with CD34 microbeads (Miltenyi Biotec) on the AutoMACS Pro according to manufacturer instructions, prior to T lymphoid differentiation on OP9.

T lymphoid commitment and expansion. Day 10 single cells were seeded on OP9 monolayers in OP9 medium (MEMa with 20% FBS), supplemented with 10 ng/mL rhTPO, 5 ng/mL rhIL-3, 30 ng/mL rhSCF, 10 ng/mL rhIL-7 and 10 ng/mL rhFlt3L to initiate lymphoid lineage commitment until day 20, and with 30 ng/mL rhSCF, 10 ng/mL rhIL-7 and 10 ng/mL rhFlt3L to complete T lineage commitment until day 35. Differentiating T lymphoid cells were passaged onto fresh OP9 monolayers every four days, fresh media with cytokines was supplemented 48 h after each passage. For the stimulation and expansion of TRAC-1XX-iT cells, differentiated cells were harvested from OP9 monolayers on day 35 and seeded on a monolayer of irradiated 3T3-CD19±4-1BBL at a 3:1 E:T ratio in T cell expansion media (CTS Optimizer Media (Gibco) with 1×CTS T cell maturation/expansion supplement and 1×CTS Immune cell serum replacement, 5 ng/mL rhIL-7 and 25 ng/mL rhIL-21). Cells were fed with fresh expansion media every 48 h. For maturation of iT cells on CD19 recombinant protein, flat-bottom tissue culture plates were coated with recombinant human CD19-Fc chimeric protein (R&D Systems), in 100 mM sodium-bicarbonate coating buffer, overnight at 4° C. Plate were blocked with PBS+5% FBS for 30 min at room temperature and washed twice with PBS. iT cells were resuspended at 0.25×10⁶ cells/mL in T cell expansion media with 5 ng/mL rhIL-7, 25 ng/mL rhIL-21 and 3 μg/mL Urelumab (Creative Biolabs). Cells were passaged after 48 h and fresh media supplemented with cytokines was added every 2 days.

PBMC derived cell isolation, activation, culture, and transduction. Buffy coats from healthy volunteer donors were obtained from the New York Blood Center. PBMCs were isolated by density gradient centrifugation.

αμTCR-T cells. αβTCR-T cells were purified and engineered as described above (TRAC-1928z-TiPS). After AAV6 transduction with pAAV-TRAC-1XX, cells were cultured in media supplemented with 5 ng/mL IL-7 and 5 ng/mL IL-15 for 3-5 days. After expansion, CD4 and CD8 cells were purified using EasySep™ CD4⁺ or CD8⁺ negative selection T cell isolation kit (Stem Cell Technologies) and purified for CAR expression by flow cytometry.

γδTCR-T cells. PBMCs were resuspended in lymphocyte media (RPMI 1640 media with 10% FBS) supplemented with 1p g/mL Zoledronic Acid (Stem Cell Technologies) 10 ng/mL rhIL-15 (R&D systems) and 100 U/mL IL-2 (Proleukin). After 72 h cells were fed with additional media and cytokines, after 6 days γδTCR-T cells were purified with the Miltenyi Biotec TCRγ/δ⁺ T cell isolation kit. Purified γδTCR-T cells were transduced with SFGg-1928z-1XX-P2A-LNGFR to induce CAR expression. Cells were expanded for 5-7 days in media supplemented with cytokines and purified for LNGFR expression using magnetic isolation with LNGFR-PE (C401457; BD) and anti-PE microbeads (Miltenyi Biotec).

NK cells. NK cells were purified from PBMCs with the NK Cell Isolation Kit (Miltenyi Biotec). NK cells were resuspended in lymphocyte medium and activated with K562-mbIL21-41BBL at a 1:1 E:T, supplemented with 1000 U/mL IL-2. 48 h after activation NK cells were transduced with SFGg-1928z-1XX-P2A-LNGFR to induce CAR expression. Cells were expanded for five days in media supplemented with cytokines and purified for LNGFR expression using magnetic isolation with LNGFR-PE (C401457; BD) and anti-PE microbeads (Miltenyi Biotec) on the AutoMACS Pro.

Retroviral vector constructs, retroviral production, and transduction. Plasmids encoding the SFGγ-retroviral vector (Riviere et al., Proc Natl Acad Sci USA 92, 6733-6737 (1995)) were prepared as previously described (Brentjens et al., Nat Med 9, 279¬286 (2003); Maher et al., Nat Biotechnol 20, 70-75 (2002)). VSV-G pseudotyped retroviral supernatants derived from transduced H29 were used to construct stable retroviral-producing cells lines as previously described (Gong et al., Neoplasia 1, 123-127 (1999)). T and NK cells were transduced by centrifugation on Retronectin (Takara)-coated plates.

iT transduction. Day 35 WT-TiPS iT cells were harvested and transduced by centrifugation on Retronectin-coated plates in the presence of 100 U/mL IL-2. Cells were fed every 48 h with fresh lymphocyte media and cytokines.

Flow cytometry: Cell surface proteins. The following conjugated antibodies were used to monitor T lymphocyte lineage development during differentiation. CD45-BV605 (2D1; BioLegend), CD3-BUV737 (UCHT1; BD), TCRab-PE-Cy7 (IP16; Invitrogen), CD4-BV785 (SK3; BioLegend), CD8α-BUV395 (HIT8a; BD), CD8b-PE (SIDI8BEE; Invitrogen), CD8ab-APC (2ST8.5H7; BD), CD7-APC-H7 (M-T701; BD), CD5-PerCP-Cy5.5 (UCHT2; BioLegend), CD56-BV421 (HCD56; BioLegend), CD1a-PE-Cy7 (HI149; BioLegend), CD2-BV711 (RPA-2.10; BD). The αβTCR-T, 76TCR-T and NK cell phenotypes were determined with CD45RA-BV605 (HI100; BioLegend), CD45RO-BV421 (UCHL1; BioLegend), CD62L-BV711 (DREG-56; BioLegend), CCR7-PE-Cy7 (G043H7; BioLegend), CD25-BB515 (2A3; BD), CD69-PerCP-Cy5.5 (FN50; BioLegend), CD27-BUV737 (M-T271; BD), CD28-PE-Cy7 (CD28.2; BioLegend), CD56-BV605 (HCD56; BioLegend), CD16-BUV737 (3G8; BD), NKG2C-PE (S19005E; BioLegend), KIR2D-FITC (NKVFS1; Miltenyi Biotec), NKp46-FITC (9E2; BioLegend), NKp44-PE-Cy7 (P44-8; BioLegend), NKp80-PE (5D12; BioLegend), NKp30-PerCP-Cy5.5 (P30-15; BioLegend), TCRgd-FITC (B1; BioLegend), TCRVd2-PerCP-Cy5.5 (B6; Biolegend) and CD161-BV421 (HP-3G10; BioLegend), CCR2-PE (K036C2; BioLegend) in addition to the aforementioned T lymphocyte lineage commitment markers. 4-1BB induction was measured with 4-1BB-BV605 (4B4-1; BioLegend). CAR expression was measured with biotin-conjugated goat anti-mouse F(ab′)2 antibody (GaM-biotin; Jackson ImmunoResearch), followed by a blocking incubation with 2% mouse serum (MP Biomedicals) and streptavidin-PE (BioLegend) or streptavidin-APC (BioLegend).

Flow cytometry: pTα stain. Cells were incubated with anti-pTα antibody (2F1; BD), biotin-labelled anti-mouse IgG1 (RMG1-1; BioLegend) and Streptavidin-PE (BD). Staining for additional cell surface proteins was performed after completion of pTα staining.

Flow cytometry: Intracellular phospho-protein analyses. SFGg-1928z, TRAC-1928z and TRAC-1XX T cells were fixed with Phosflow Fix Buffer I (BD) and stained for the CAR with GaM-af647 (Jackson ImmunoResearch), followed by 2% mouse serum, and subsequently permeabilized with Phosflow Perm Buffer III (BD) following the manufacturer's procedure. Permeabilized samples were stained with antibodies detecting phosphorylated CD3z ITAM1 (EP776(2)Y; Abcam) or phosphorylated CD3z ITAM3 (K25-407.69; BD).

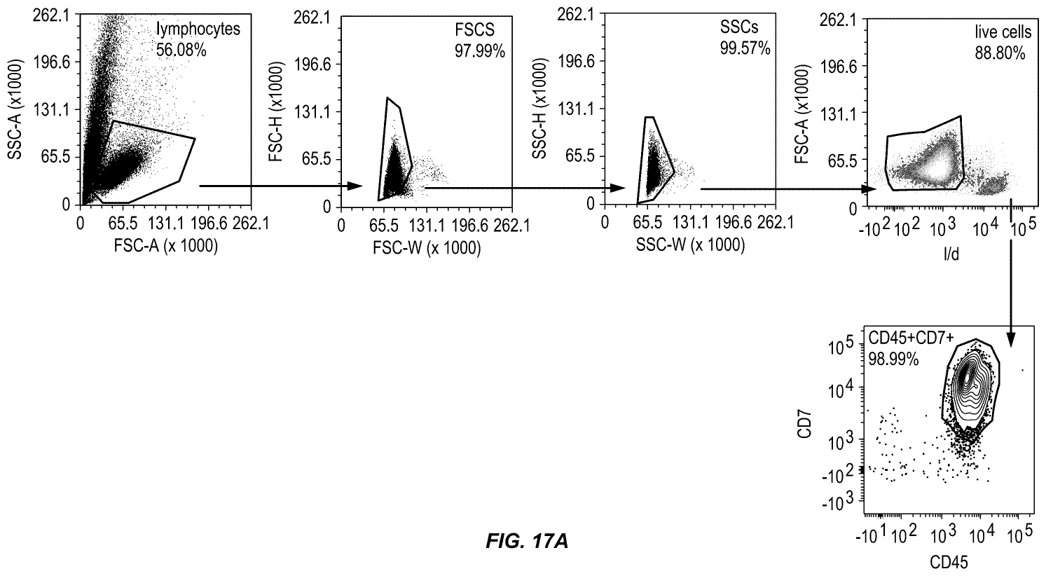

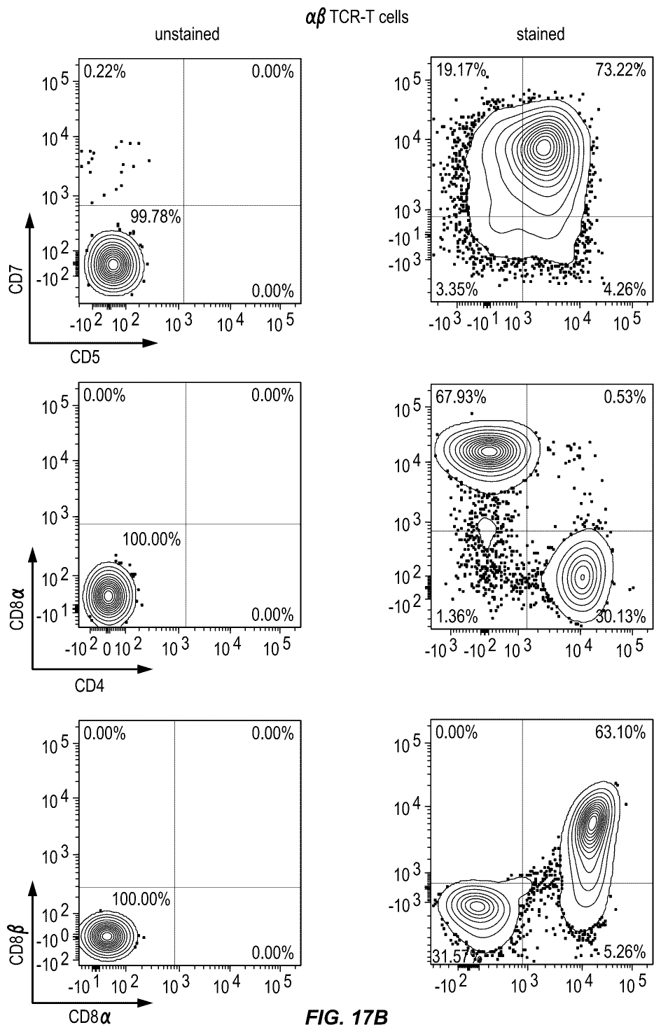

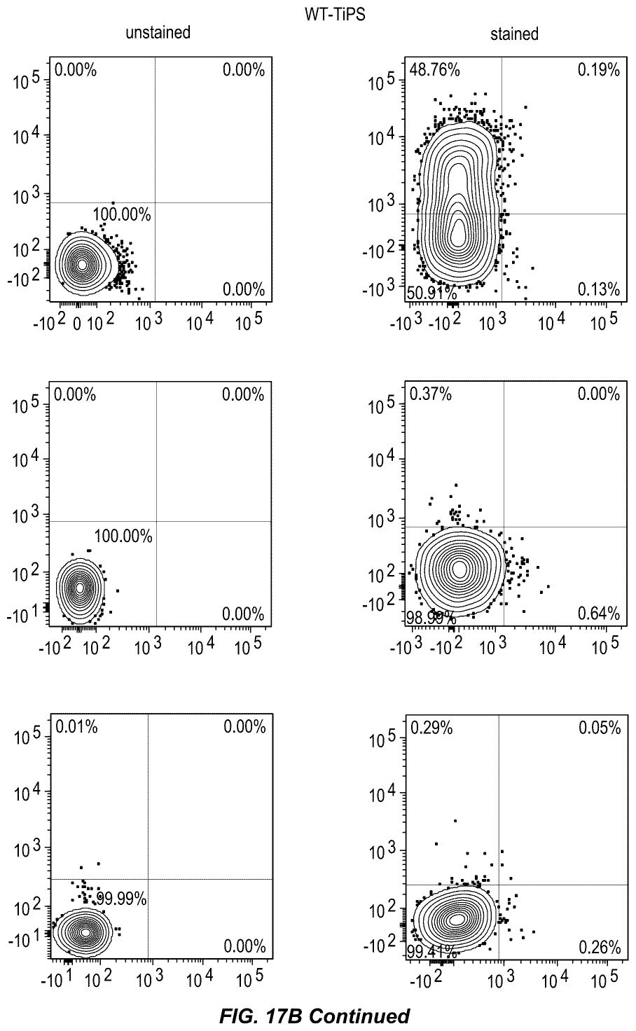

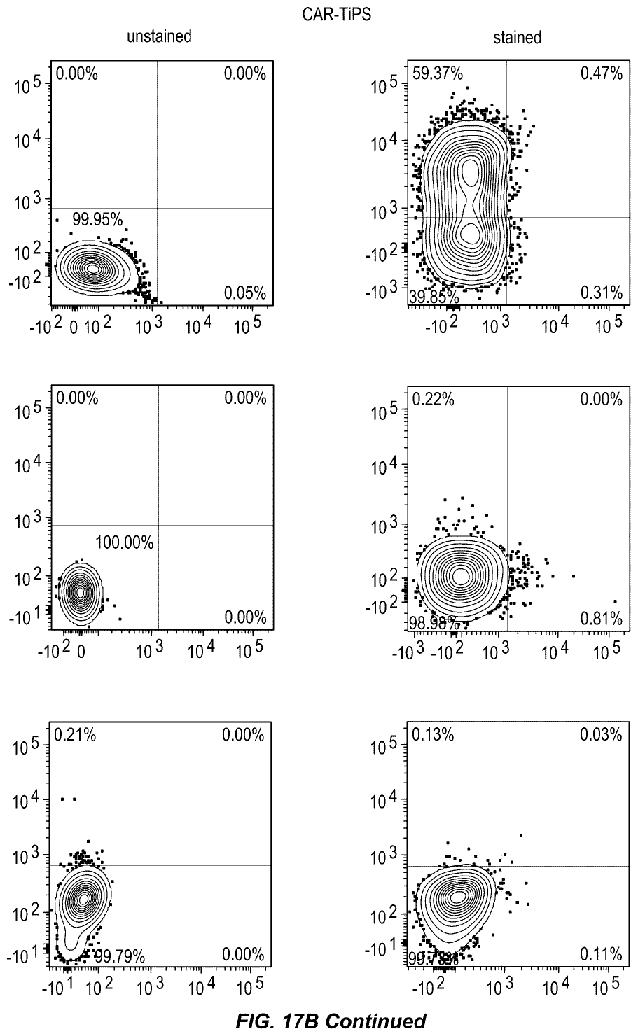

Flow cytometry. All antibodies were titrated prior to use. Flow cytometric data were acquired on Fortessa X-20 (BD) or 5-laser Aurora (Cytek Biosciences) Flow cytometer voltages were calibrated with Ultra Rainbow Calibration Kit (SpheroTech, URCP-38-2K) prior to every acquisition. Analysis was performed using FCS Express 7 (De Novo Software). Negative and positive gates were set based on (un)stained PBMC and TiPS controls (FIGS. 17A and 17B).

Apoptosis Analysis. WT-TiPS and CAR-TiPS cells were harvested daily between D27 and D35 of the differentiation and stained for viability and Annexin-V-PE-Cy7 (Invitrogen) according to manufacturer's instructions, followed by cell surface staining for CD45, CD7, CD4, CD8α and CD80 as described above. Percentage of apoptotic cells within populations (CD45⁺CD7+, DN, DP, CD4, CD8αα or CD8αβ) was calculated based on live Annexin-V⁺ stain.

Notch induction in differentiating TiPS cells. WT-TiPS D20 lymphoid progenitor cells were co-cultured with parental OP9, OP9-hDLL1, OP9-hDLL4, OP9-hJAG1 or OP9-hJAG2. At 0, 4, 8, 12, 24, 48 and 72 h of co-culture, cells were harvested, cell pellets were snap-frozen and stored at −80° C. for until RNA extraction. Gene induction was measured by ddPCR as described below. Relative level of DTX1 induction was normalized to 0 h.

Notch/TCR target gene induction during iT differentiation. TiPS (WT-TiPS, CAR-TiPS and TRAC-1XX-TiPS) were differentiated as described. During the T lymphoid commitment phase of the differentiation (D24, D27, D31 and D35) suspension cells were harvested, cell pellets were snap-frozen and stored at −80° C. until RNA extraction. Gene induction was measured by ddPCR as described below.

Digital droplet PCR. Digital droplet PCR (ddPCR) gene expression assays for Notch1 (dHsaCPE5050282), Notch 3 (dHsaCPE5046836), TCF7 (dHsaCPE5031804), DTX1 (dHsaCPE5192773), GATA3 (dHsaCPE5034292), ID3 (dHsaCPE5027720), βTCRA (dHsaCPE5031466) and RPL13A (dHsaCPE5037592) were obtained from Bio-Rad. ddPCR reactions were set up according to One-Step RT-ddPCR Advanced Kit for Probes protocol on a QX200 ddPCR system (Bio-Rad). Each sample was evaluated in technical triplicates. Reactions were partitioned into a median of ˜15,000 droplets per well using the QX200 droplet generator. Emulsified reactions were amplified on a 96-well thermal cycler. Plates were read and analyzed with the QuantaSoft software to assess the number of droplets positive for the target gene. The number of mRNA molecules per droplet relative to RPL13A was calculated assuming a Poisson distribution.

RNA extraction, library generation and sequencing. Total RNA was isolated from 0.30.5×10⁶ cells using the RNeasy 96 Kit (Qiagen, 74181) according to the manufacturer's protocol. RNA quality was measured by High Sensitivity RNA ScreenTape (Agilent) on the Agilent 42000 TapeStation System. RNA quantity was measured using the Thermo Scientific® Qubit® Flex Fluorometer (Invitrogen). 200 ng of total RNA was used per sample to generate mRNA library using NEBNext® Ultra® II Directional RNA Library Kit for Illumina® (New England BioLabs) per sample. Final libraries were quantified using the Qubit® 1×dsDNA HS Assay kit on the QubitŌ Flex Fluorometer. Library quality and size were measured using Agilent High Sensitivity D1000 ScreenTape. Libraries were calculated to nM, diluted to 4 nM, and pooled evenly for high throughput sequencing. Sequencing was performed on Illumina NextSeq 500 Instrument (Illumina) with 2×76 pair-end reads targeting a minimum of 16 million pair-end reads per sample.

RNA sequencing analysis. Sequencing data were trimmed using Trim Galore! 0.6.0 to remove Illumina adapters. Resulting reads were mapped to the human reference genome (assembly GRCh38.86) using Salmon v0.13.1 in quasi-mapping-based mode, with GC bias correction, selective alignment, and range factorization. The data was analyzed using the statistical software R. The aggregated read counts were normalized for sequencing depth and RNA composition with DESeq2. Pseudogenes identified by the GENCODE project and lowly expressed genes were filtered out prior to downstream analysis. Principal Component Analysis (PCA) was performed with normalized read counts in R. Hierarchical Clustering Analysis was carried out with UPGMA method on Euclidean distance matrix. Correlation matrix was generated using Pearson's statistics.

Cytotoxicity Assays. The in vitro toxicity of TRAC-1XX T cells was determined by a standard firefly luciferase (FFLuc)-based assay (Hamieh et al., Nature 568, 112-116 (2019)) or by NLR⁺ imaging on the Incucyte Live Cell Analysis System (Sartorius). For FFLuc based cytotoxicity, FFLuc-expressing NALM6 served as target cells. The effector (E) and tumor target (T) cells were co-cultured in triplicates at the indicated E:T ratio using black-walled 96-well plates with 1×10⁵ target cells in a total volume of 100.iL per well in T cell expansion medium. Four hours later, 50.iL luciferase substrate (Bright-Glo, Promega) was directly added to each well. Emitted light (RLU) was detected in a luminescence plate reader (Agilent BioTek SlL), and lysis was calculated using the formula 100×(1−(RLUsample)/(RLUtarget alone)). For the Incucyte cytotoxicity assay, flat-bottom 96-well plates were pre-coated with 5.ig/mL Fibronectin (Sigma) at 4° C. overnight. The E:T ratios were plated in triplicates with 3×10⁴ target cells in a total volume of 200.iL per well in T cell expansion medium. Hourly brightfield and fluorescence imaging was performed for a 72 h period. Cell survival was quantified based on NLR⁺ surface area by Incucyte S3 software (Essen BioScience) and normalized to the NLR⁺ surface area at 0 h. For the flow-cytometry based CTL, CD19+ cells were purified from apheresis product (EasySep CD19 Positive selection kit, StemCell Technologies) and cultured overnight in RPMI media with 10% human serum (HS), 1×NEAA, 2 mM GlutaMAX, 100 U/mL Pen, 100.ig/mL Strep. TRAC-1XX-iT cells were counted and plated in triplicate at the indicated E:T ratios with 1×10⁵ CD19⁺ CLL target cells in a total volume of 100 μL per well in T cell expansion media. Six hours later, cells were stained with CD19-PE-Cy7 (SJ25C1, BioLegend), CD45-BV605 (HI30, BioLegend), CD7-APC-H7 and Sytox Blue Dead Cell Stain (Invitrogen) and the number of remaining, target cells (live, CD7⁻CD19⁺ cells) were enumerated by flow using AccuCount beads (Spherotech). Percentage lysis was calculated using the formula (sample count×100)/(target alone count).

Antigen restimulation assay. Restimulation assays were performed as previously described (Zhao et al., Cancer Cell 28, 415-428 (2015)). In brief, 1×10⁶ T cells were co-cultured with 3×10⁵ 3T3-CD19 in 1 mL T cell expansion media. Fresh media was supplied every 48 h. Cells were counted after seven days and restimulated on fresh 3T3-CD19 monolayers.

In vitro NALM6 rechallenge assay. 3×10⁴ iT cells were co-cultured with 3×10⁴ NLR⁺ NALM6 CD19⁺ tumor cells in 200 μL T cell expansion media. Hourly brightfield and fluorescence imaging was performed for a 10-day period. At day 3 and day 6, plates were removed from the Incucyte, and 50 μL media was replaced with 50 μL media supplemented with 3×10⁴ fresh NLR⁺ NALM6 cells and 4× cytokines. Cell survival was quantified based on NLR⁺ surface area by Incucyte S3 software (Essen BioScience) and normalized to the NLR⁺ surface area at 0, 72 and 144 h respectively.

Cytokine analyses. To measure intracellular levels, iT cells were cultured for 4 h at 1×10⁶ cells/mL together with NALM6 at a 1:1 ratio in the presence of Brefeldin A (BD) monensin (BioLegend) and CD107a-BV421 (H4A3; BD). Cells were stained with ef506 Fixable Viability dye (ThermoFisher) prior to fixation and permeabilization using BD Cytofix/Cytoperm Plus kit as per manufacturer's instructions, followed by staining with anti-cytokine and cell-surface antibodies GranzymeB-APC (GB12; Invitrogen), IFN1-PE-Cy7 (4S.B3; Invitrogen), IL-2-BUV737 (MQ1-17H12; BD), TNFa-PE (Mab11; Invitrogen), IL-17-af488 (BL168, BioLegend), CD45-BV605 (2D1; BioLegend). Percentage of cytokine producing cells was determined by flow cytometry. To measure secreted cytokine levels, 0.5×10⁶ T cells were cultured together with NALM6 at a 1:1 ratio or without target cells for 24 h. Supernatants were collected and stored at −80° C. Secreted cytokines were quantified using BD Cytometric Bead Array kits (IL-2-558270, IFN1-560111, TNFa-560112) and flow cytometry.

ERK1/2 phosphorylation analysis. Phosphorylated-ERK1/2 was quantified in day 35 WT-TiPS and CAR-TiPS. Cells were lysed in 1× denaturation buffer supplemented with 10 μg/mL aprotonin, leupeptin and pepstatin at 1 mg/mL total protein content. Phosphorylated ERK1/2 was quantified using the BD Cell Signaling Master Buffer Kit (560005) and Phospho ERK1/2 (560012) according to manufacturer's instructions.

Mouse systemic tumor model. 8-12 week-old NOD/SCID/IL-2R1-null (NSG) mice were obtained from Jackson Laboratory. A dose of 0.1×10⁶ FFLuc-NALM6 was administered by tail vein injection and three days later a dose of 2×10⁶ or 4×10⁶ T cells were administered by tail vein injection per mouse. Mice received IL-2 (Proleukin 100 KU/mouse) and rhIL-15 (150 ng/mouse) in 200 μL PBS intraperitoneally twice per week for three weeks post T cell injection. Tumor burden was measured by bioluminescence imaging using the Xenogen IVIS Imaging System (Xenogen). Living Image software (Xenogen) was used to analyze the acquired bioluminescence data. No blinding method was used. All animal experiments were conducted in accordance with protocols approved by MSKCC Institutional Animal Care and Use Committee (IACUC) and following National Institutes of Health (NIH) guidelines for animal welfare.

Cell enumeration. Three mice per group were euthanized at day 6 or day 12 post T cell injection and cells were isolated from the blood, bone marrow and spleen as described³⁷. Cells were stained for viability (ef506), mCD45-BV421 (30-F11; BioLegend), hCD45-BV605 (HI30; BioLegend), CAR-GaM-af647 (Jackson ImmunoResearch), CD4-BV785 (SK3; BioLegend), CD8α-BUV395 (HIT8a; BD), CD8b-PE (SIDI8BEE; Invitrogen), CD45RA-BV605 (HI100; BioLegend), CD45RO-BV421 (UCHL1; BioLegend), CD27-BUV737 (M-T271; BD), CD28-PE-Cy7 (CD28.2; BioLegend), CD25-BB515 (2A3; BD), CD62L-BV711 (DREG-56; BioLegend), TIGIT-BV605 (A15153G; BioLegend), LAG3-PE-Cy7 (11C3c65; BioLegend), PD1-BV711 (EH12.2H7; BioLegend), CD56-BV421 (NCAM16.2; BD), CD19-PE-Cy7 (SJ25C1; BioLegend) and GFP (tumor cells) and analyzed by flow cytometry in the presence of counting beads (Countbright, Invitrogen).

Statistics. All experimental data are presented as mean±S.D. No statistical methods were used to predetermine sample size. Appropriate statistical tests were used to analyze data, as described in the figure legends. Statistical analysis was performed on GraphPad Prism 7 software and R. Significance was set at p<0.05.

### Example 2

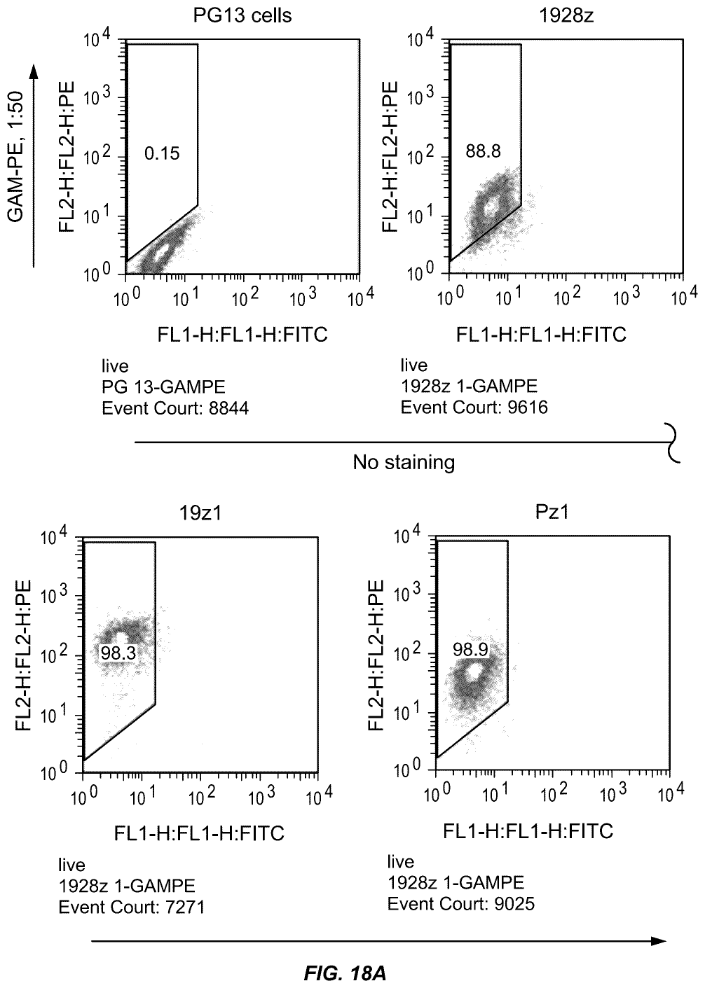

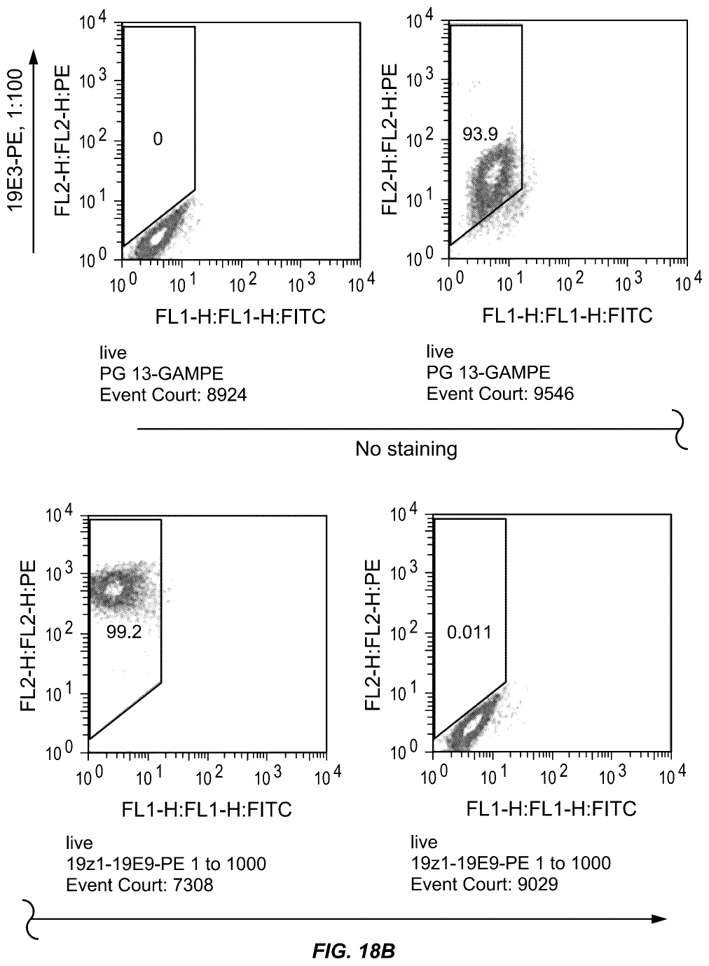

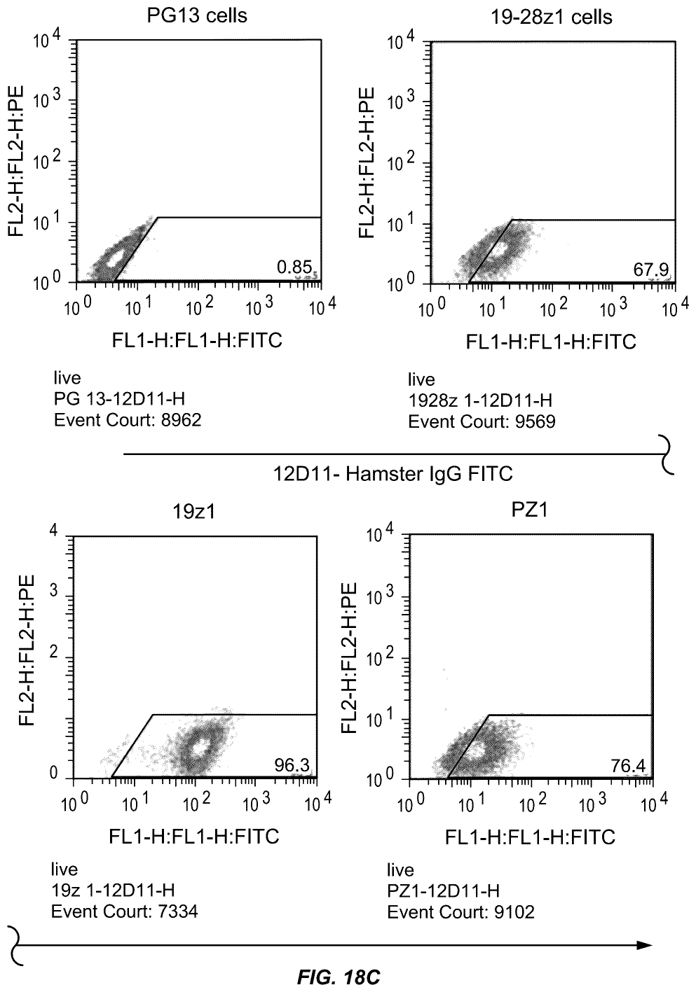

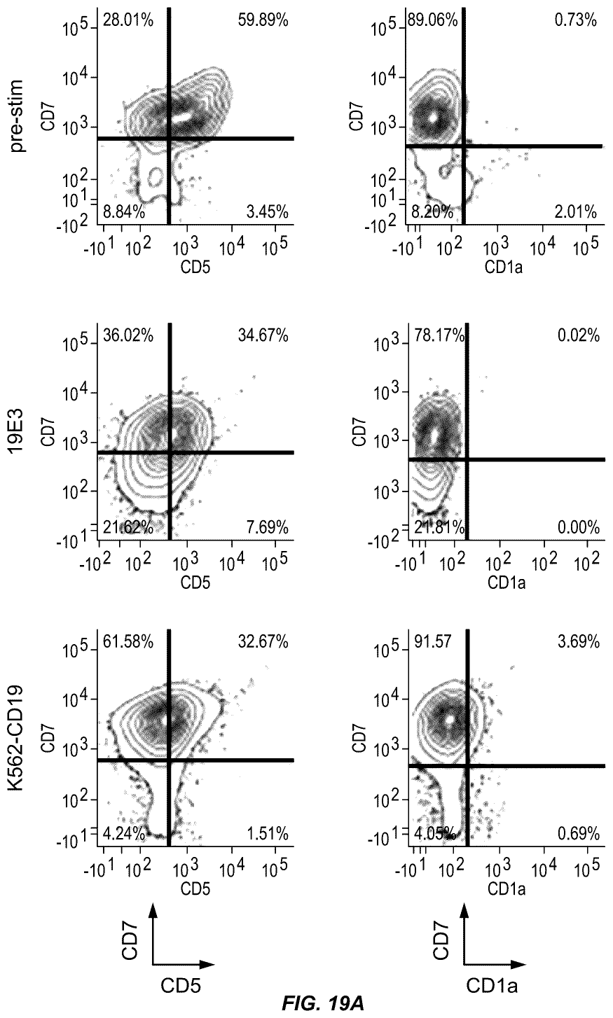

The presented example relates to the field of immunotherapy, specifically the development of allogeneic, point-of-care immunotherapy, by facilitating the maturation of induced pluripotent stem cell (iPSC)-derived T cells. Stimulation, through anti-idiotype antibodies against the extracellular antigen-binding domain of a chimeric antigen receptor (CAR), can initiate CAR-signaling (e.g., by engaging, stimulating, or activating a CAR), allowing for the maturation of iPSC-derived T cells, or differentiation of peripheral blood-derived T cells. The development of mature CAR T cells from T-iPSC was facilitated through cytokine- and feeder-cell (OP9-DL4) based induction of hematopoietic and T lymphoid lineage differentiation. At the final stage of T cell development, positive selection relies on carefully titrated TCR-signaling, which can be mimicked with the presently disclosed anti-idiotype antibodies 19E3 (“CAR1”) or 12D11 (“CAR2”). The 19E3 monoclonal antibody binds to the SJ25C1 scFv in the CD19-targeting CAR, whereas the 12D11 monoclonal antibody binds to both CD19 and PSMA-targeting CARs (FIGS. 18A-18C)

### Development of Anti-Idiotype Antibodies

Antibodies were developed at the Memorial Sloan Kettering Cancer Center's Antibody Core Facility. Armenian Hamsters were inoculated intra-peritoneally with 50 μg of the monoclonal (mouse anti-human) CD19 antibody SJ25C1. Animals were immunized four times and hybridomas were generated through the fusion with mouse myeloma P3×63 cells. The antibody-producing hybridomas were screened by ELISA and flow cytometry (FIG. 18).

Flow cytometric assessment of antibody specificity was determined utilizing the CAR+PG13 fibroblast. Expression of the CD19-targeting second-generation 1928z CAR and first generation 19z1 CAR, as well as the first-generation PSMA-targeting Pz1 CAR was confirmed with the polyclonal Goat-anti-Mouse F(ab)′ fragment (FIG. 18A). Staining with 19E3 (FIG. 18B) showed specific SJ25C1 recognition in the 1928z and 19z1 CARs. Notably, staining with 12D11 (FIG. 18C) showed scFv-independent CAR recognition, detecting both CD19- and PSMA-targeting CARs.

### Anti-Idiotype Antibody Stimulation Facilitates CAR T Cell Maturation

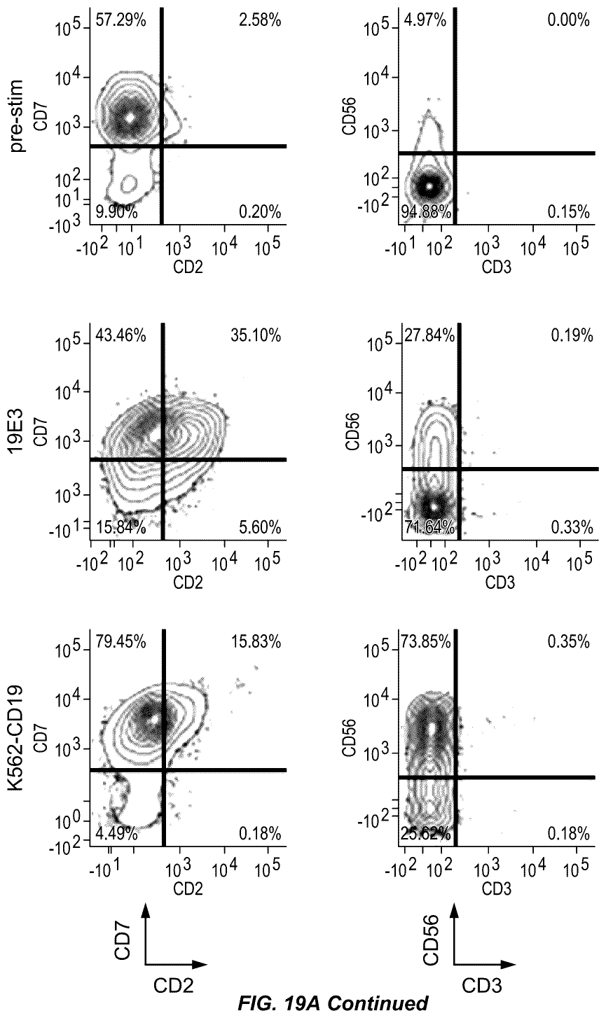

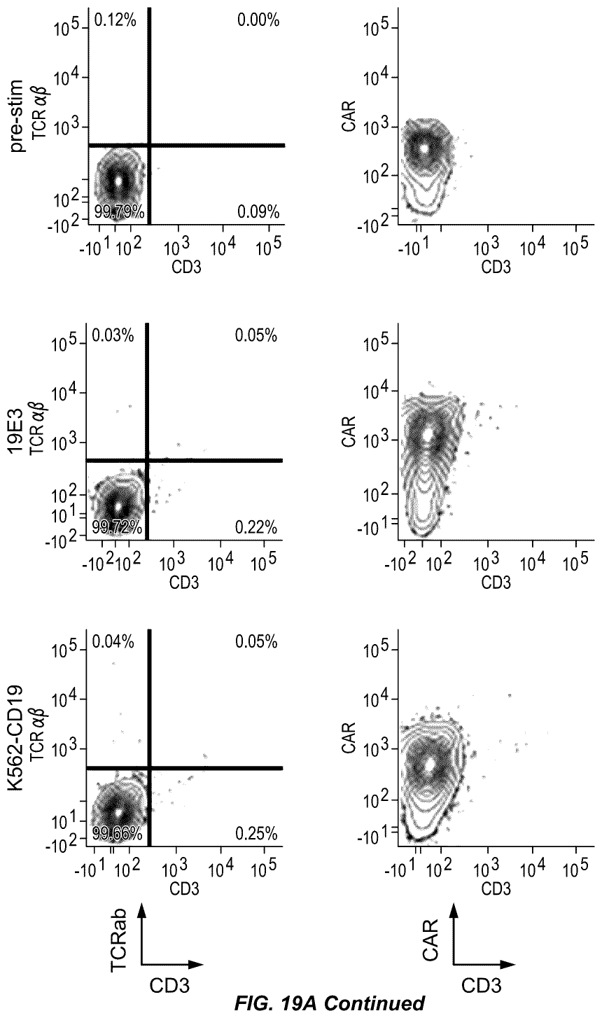

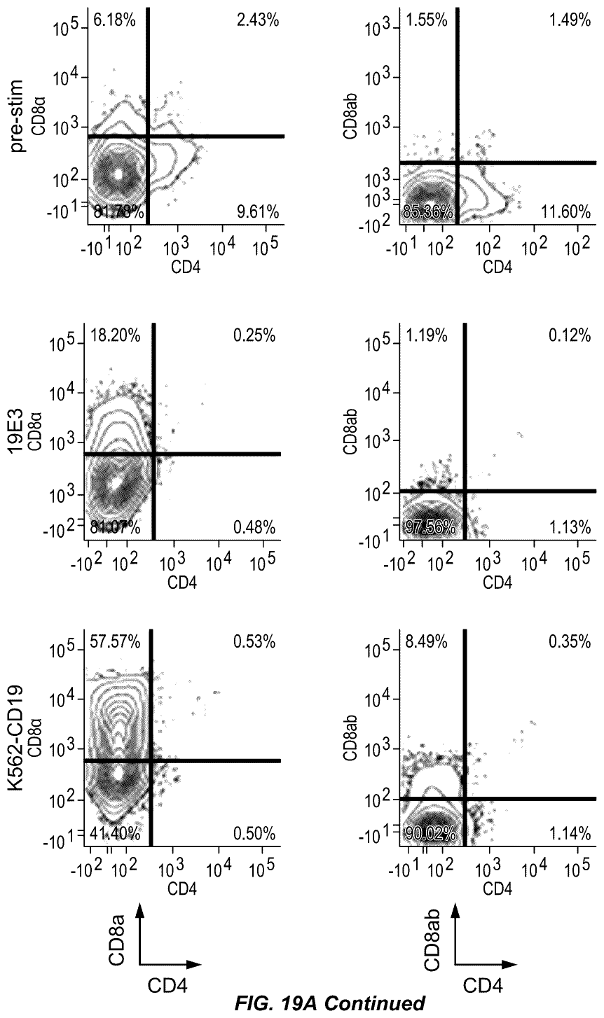

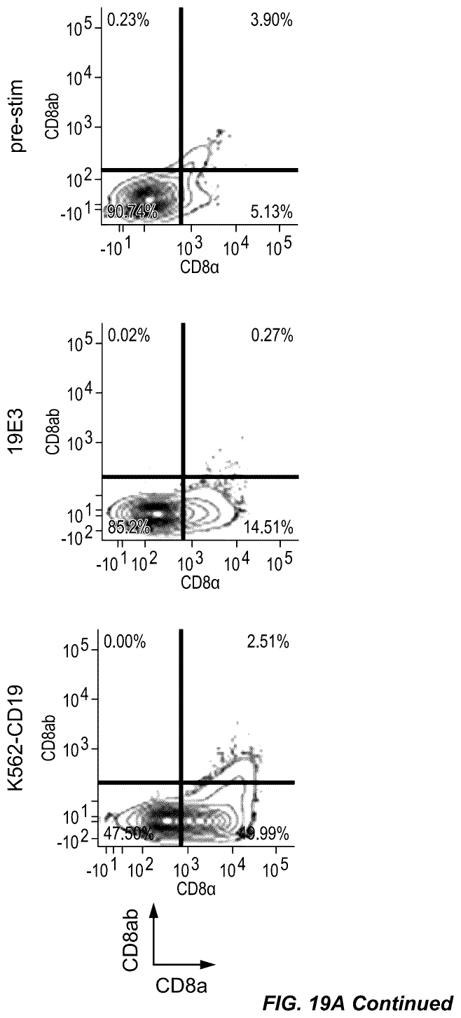

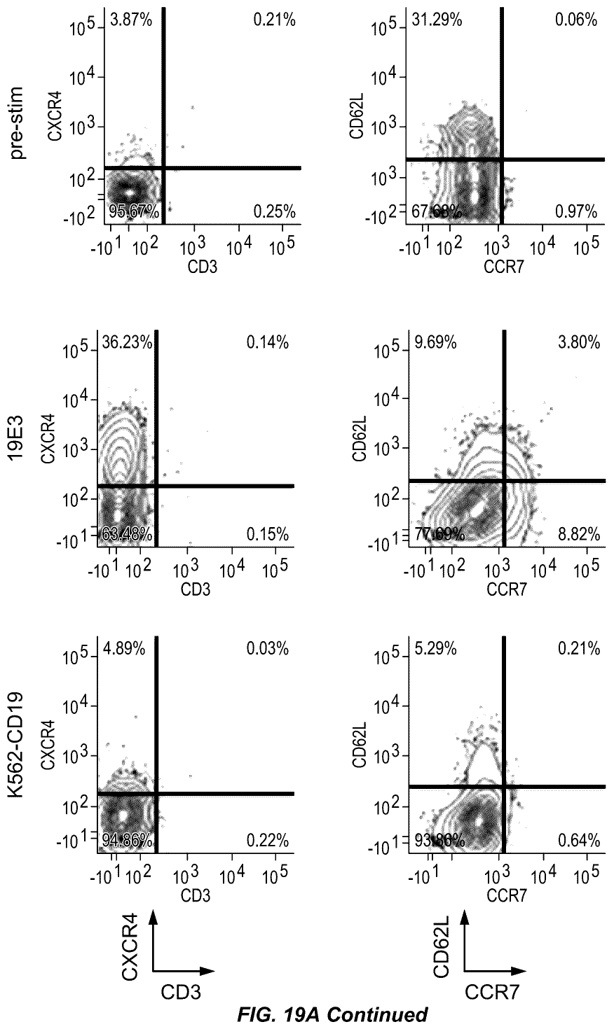

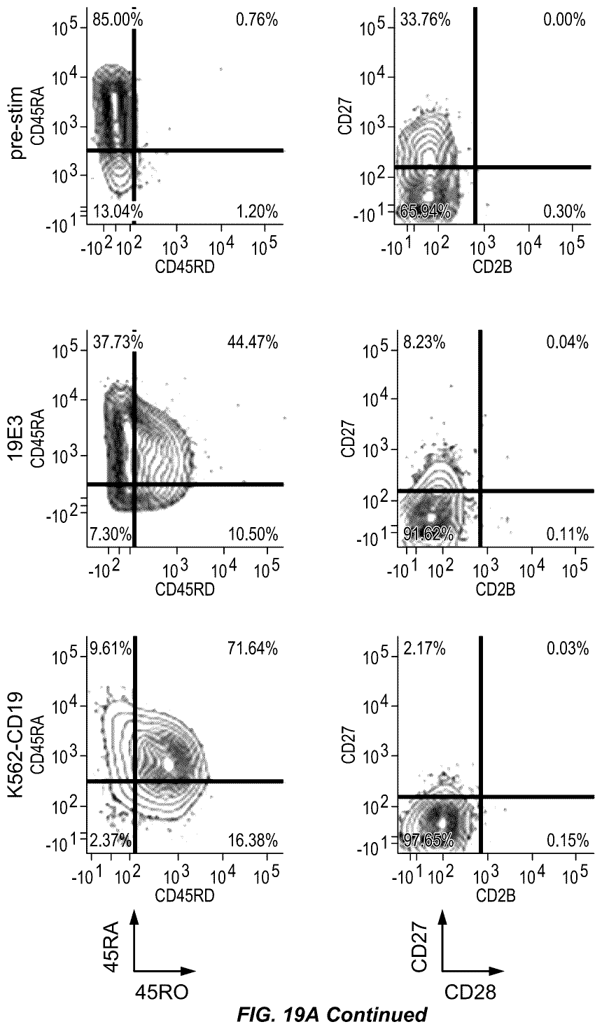

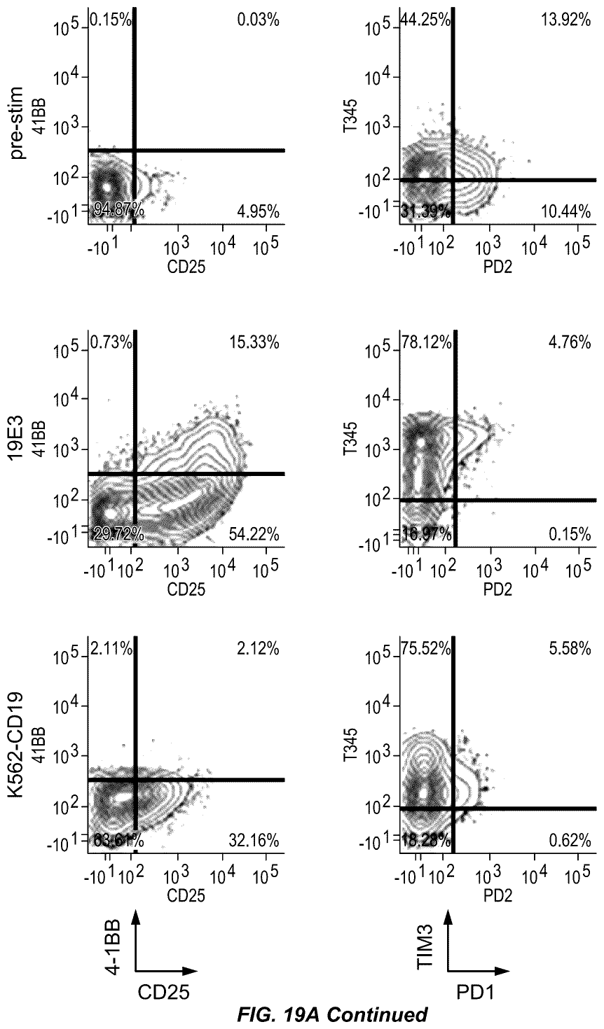

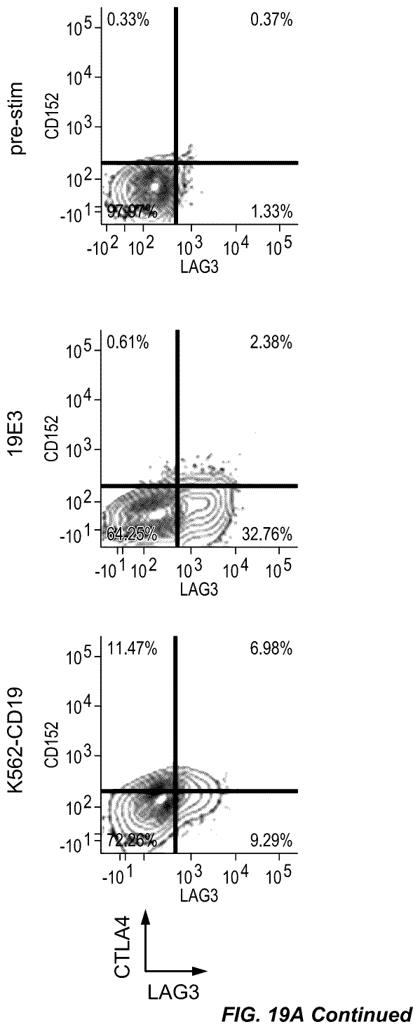

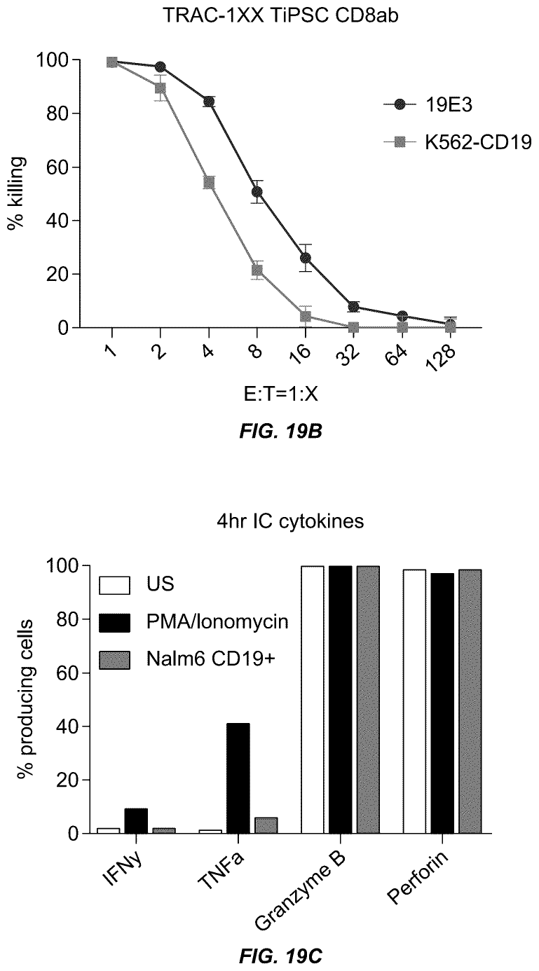

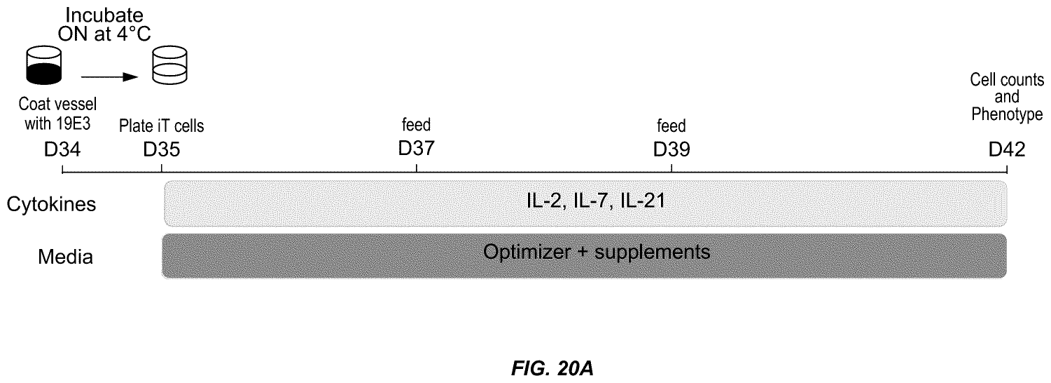

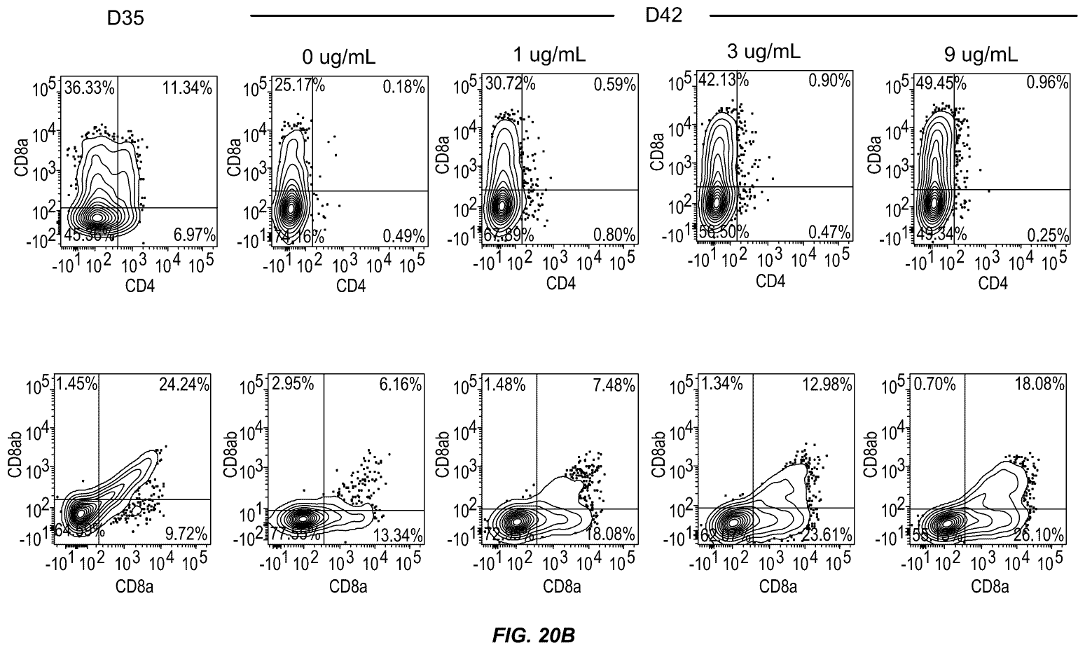

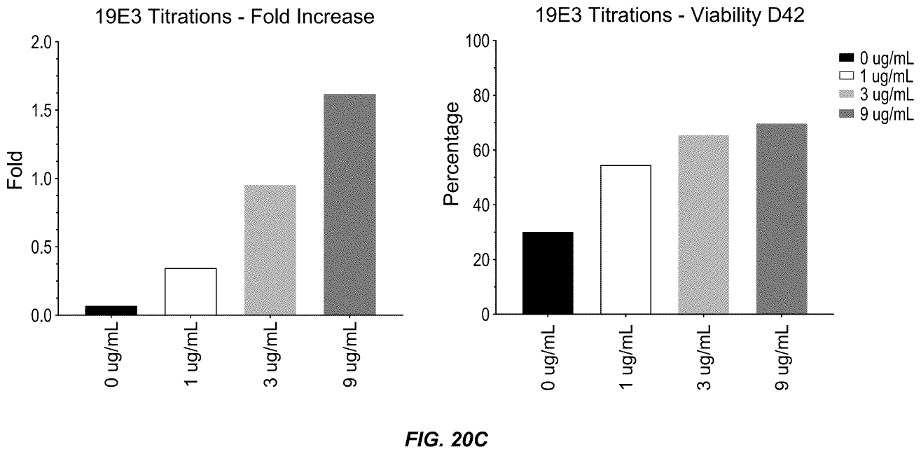

The presently disclosed anti-idiotype antibodies facilitated the maturation of iPSC-derived CAR⁺ T cells. Stimulation of TiPSC-derived T cells with 19E3 or 12D11 antibodies mimicked positive selection through the engagement of CAR signaling (FIGS. 19A-19C) and induced proliferation in control peripheral-blood-derived CD8⁺ CAR T cells. The use of 19E3 or 12D11 anti-idiotype antibodies resulted in the final maturation and the development of a favorable effector phenotype (CD2⁺, CD56^{lo}, CD45RA⁺, CD62L⁺, CCR7^{lo}, CXCR4⁺, CD25⁺), compared to stronger stimulation with CD19-protein expressing feeder cells (K562-CD19) (FIG. 19A). In vitro-assessed cytolytic capacity (FIG. 19B) over 18 hours showed that 19E3-maturated T cells had superior functional capacity and the ability to produce cytokines in a stimulation-dependent manner (FIG. 19C). Moreover, 19E3 promoted T-cell maturation and expansion in a dose-dependent manner (FIG. 20A-20C).

CAR T Cell Maturation Obtained by the Anti-Idiotype Antibodies was Enhanced by Co-Stimulation with 4-1BB Agonists

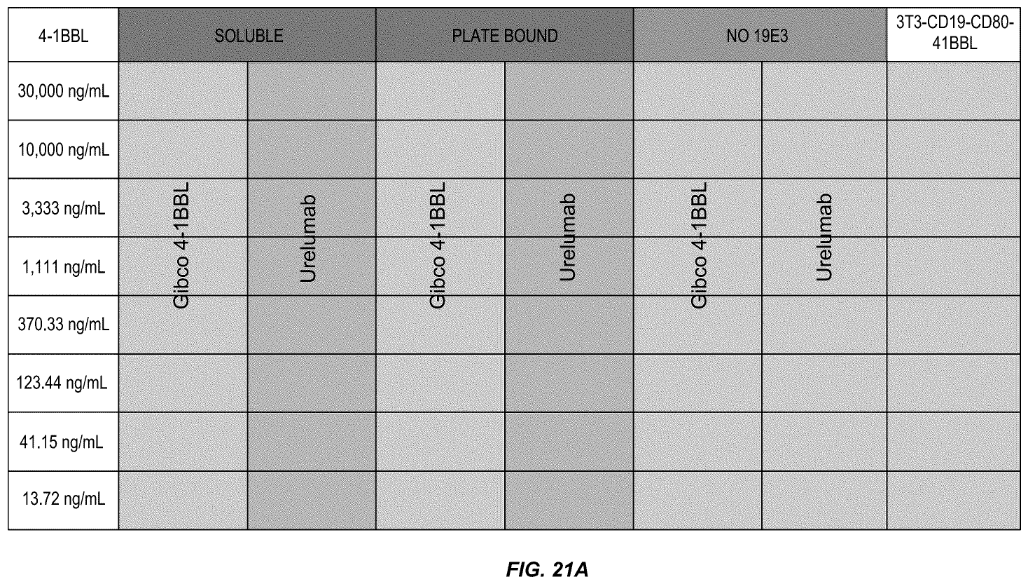

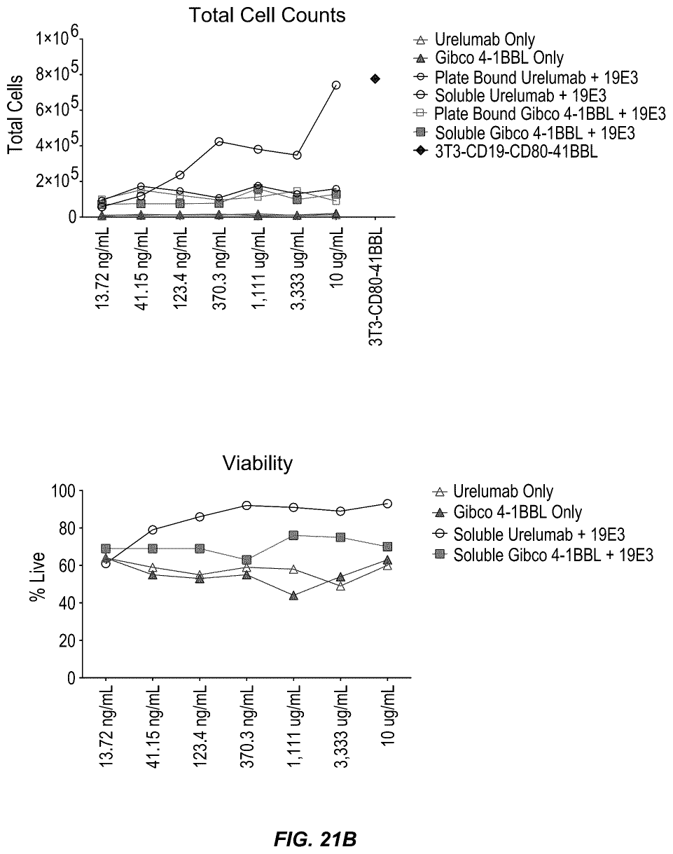

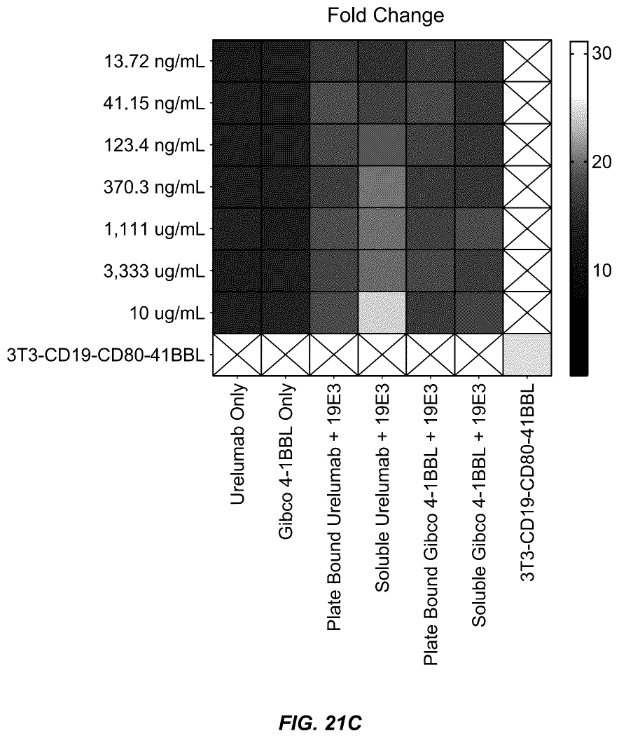

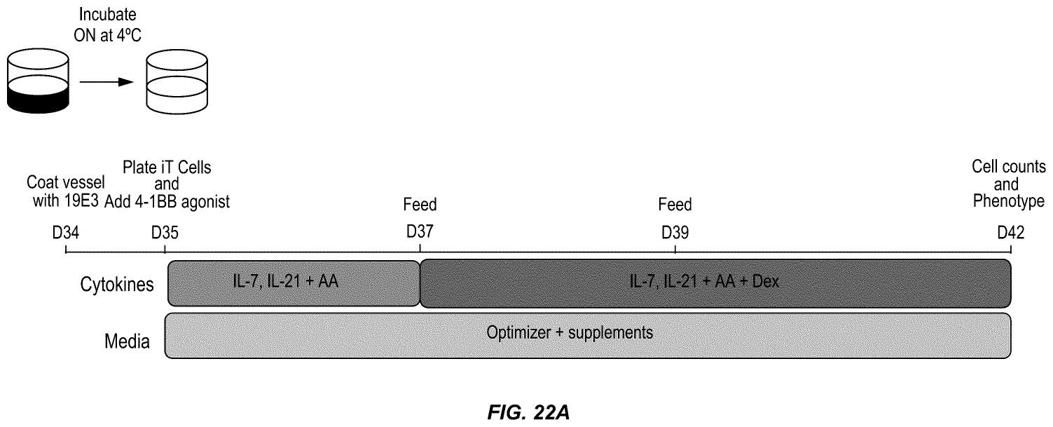

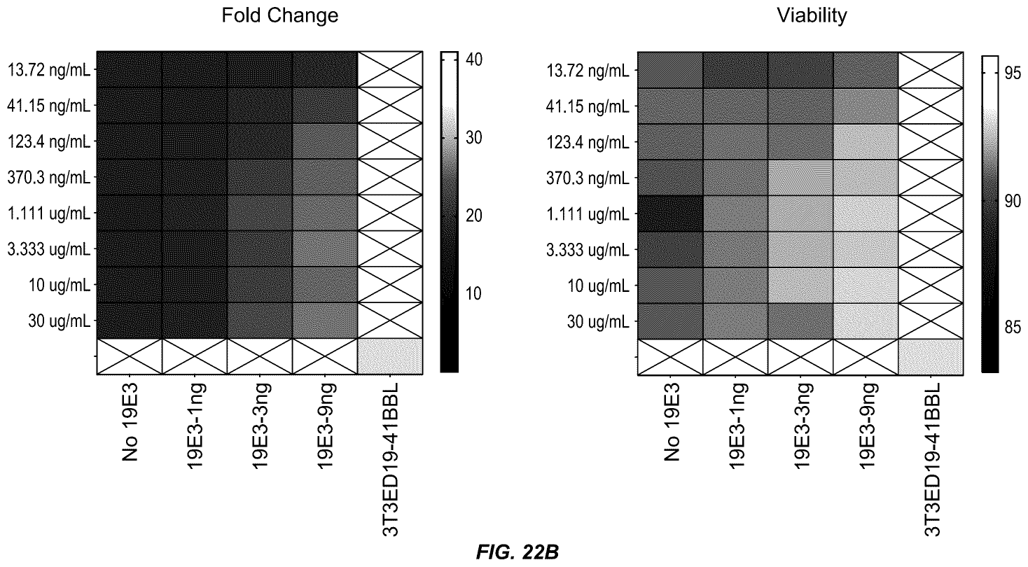

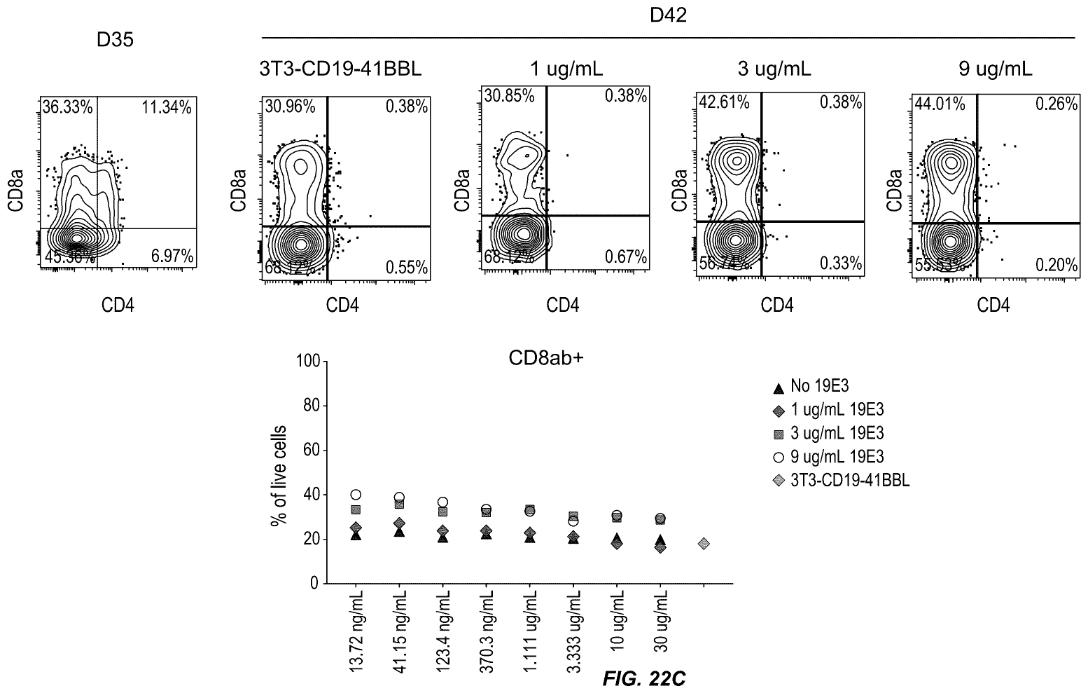

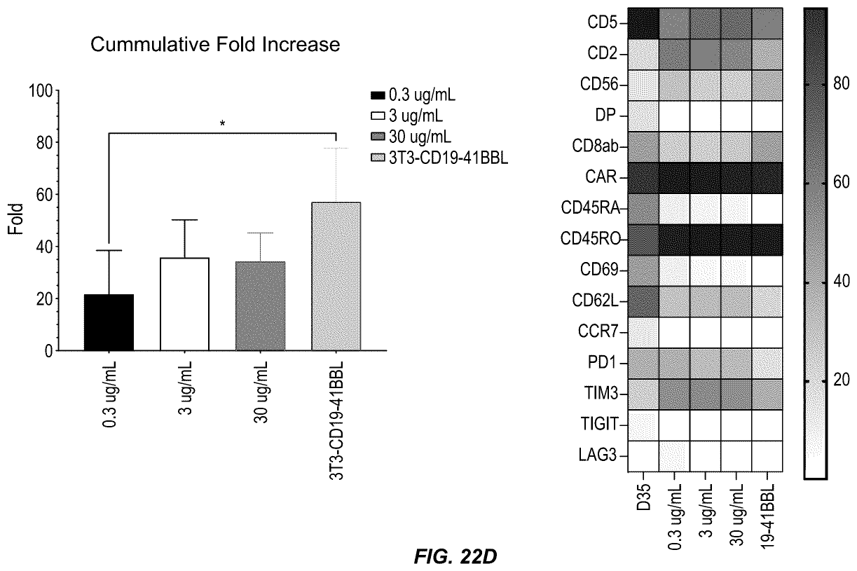

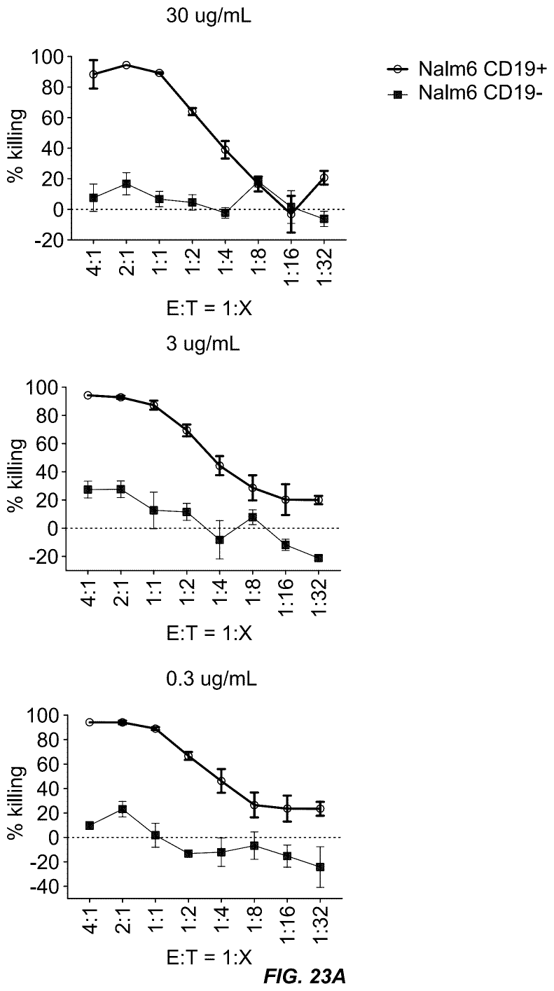

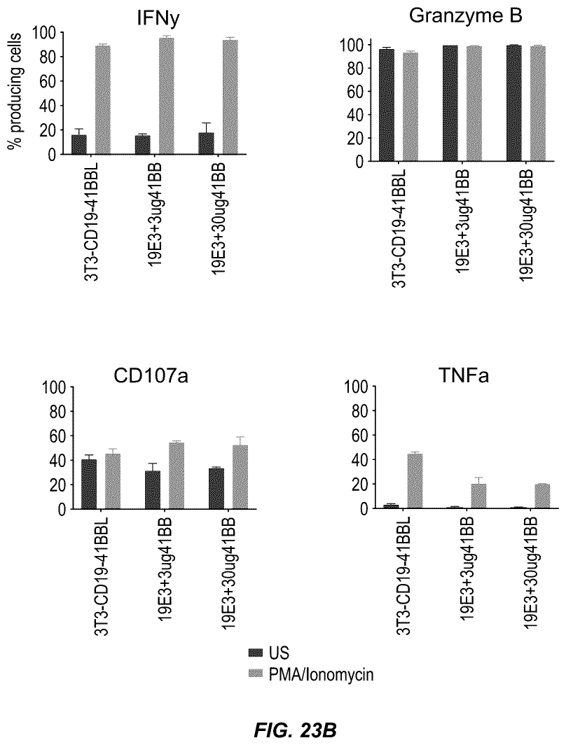

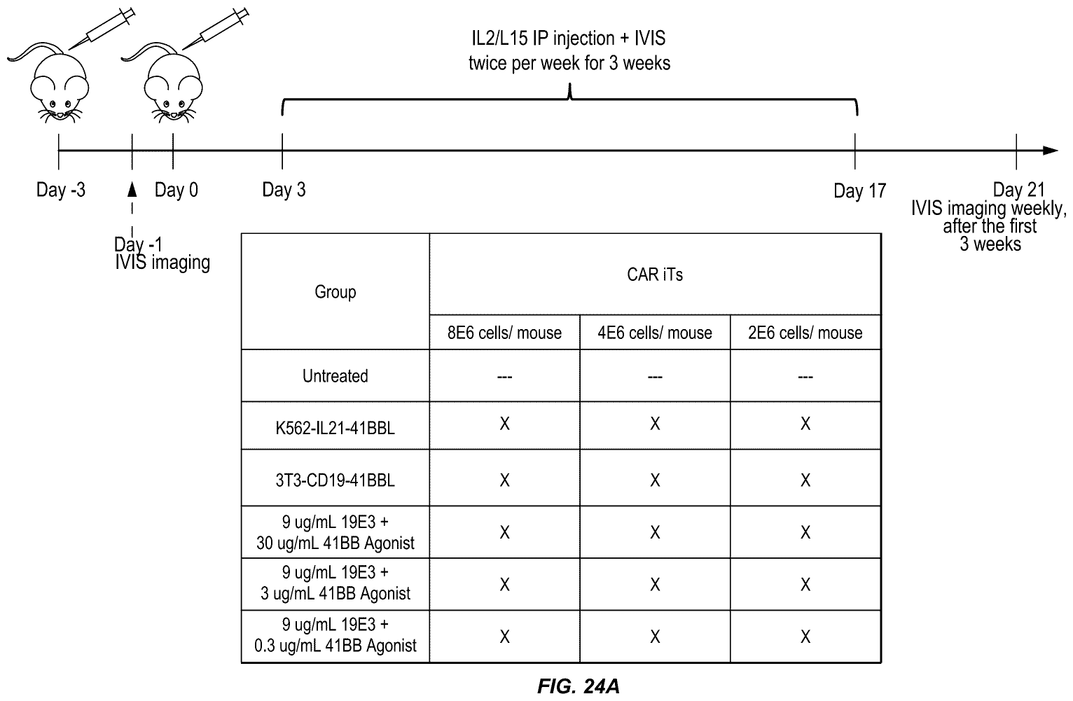

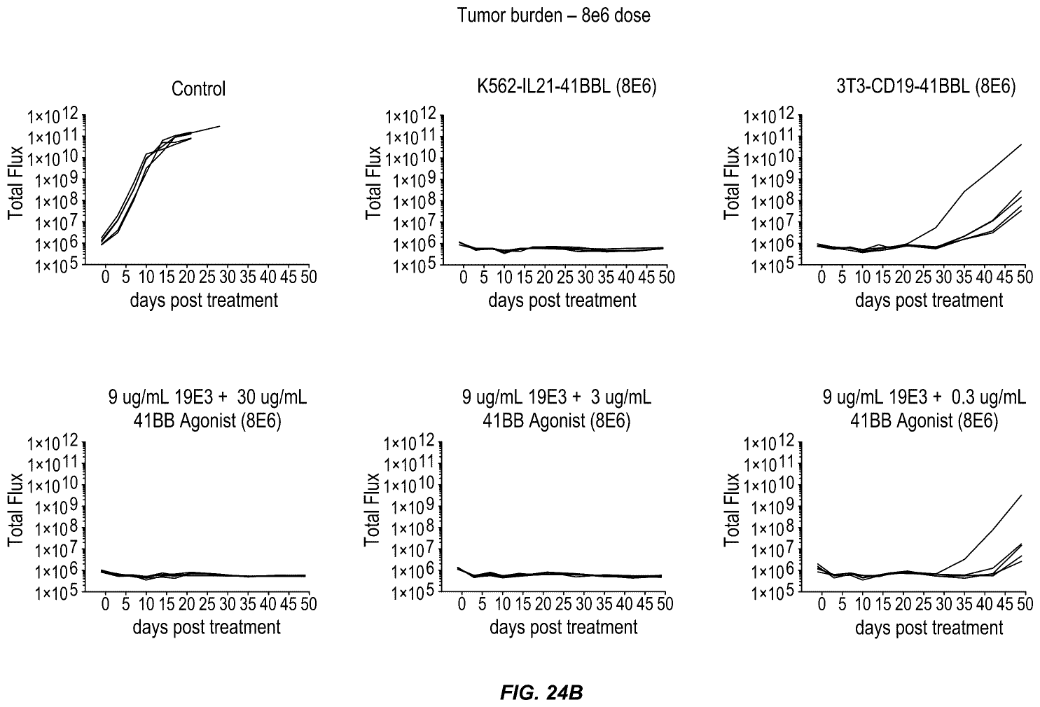

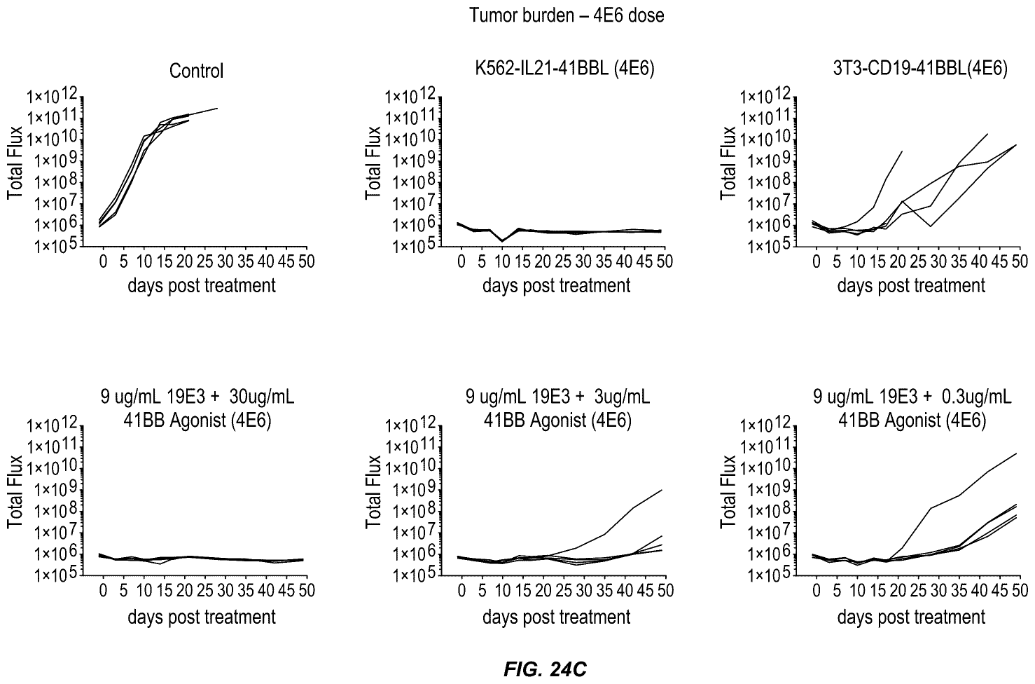

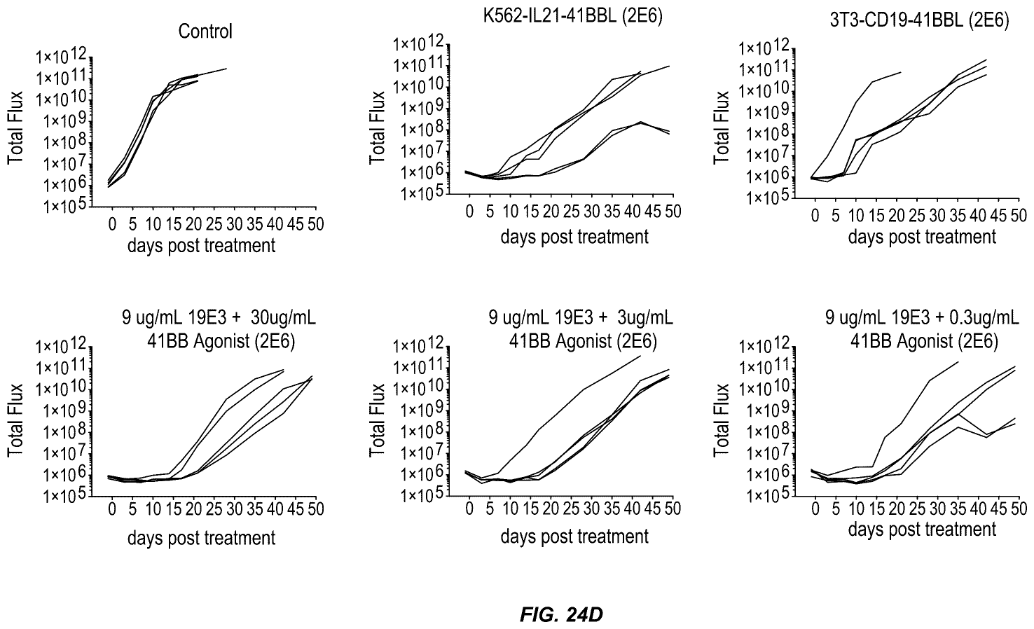

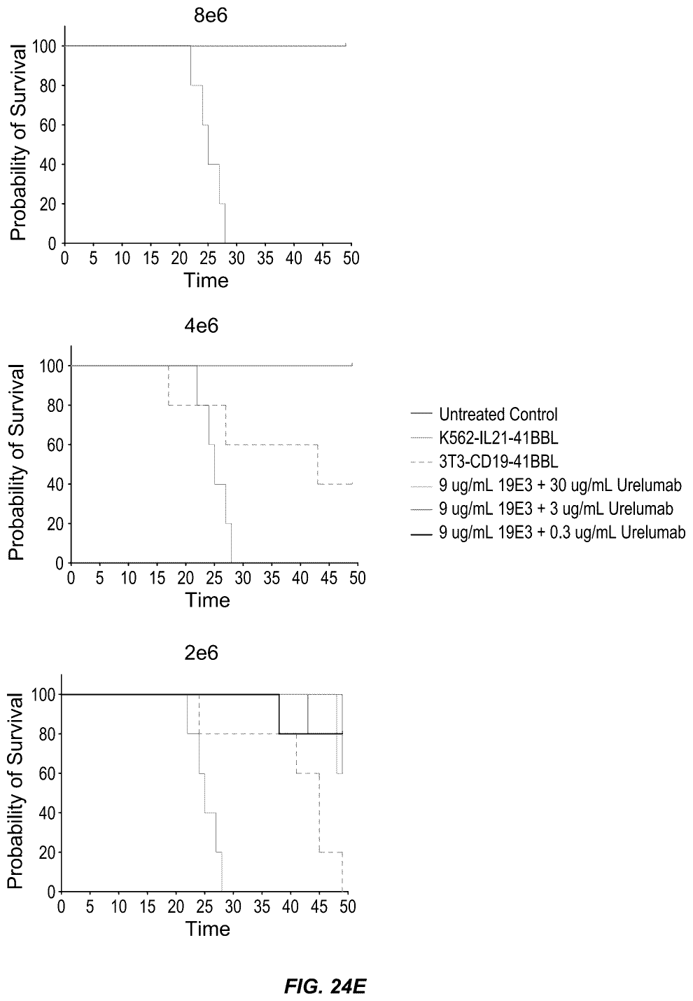

The effects of the presently disclosed 19E3 and 12D11 antibodies were enhanced by 4-1BB agonists. As outlined in FIG. 21A, premature T cells were treated with different doses of 4-1BB, either in soluble or plate-bound form. Alternately, cells were treated with urelumab, either in soluble or plate-bound form. It was observed that T cells developed in presence of 19E3 and a 4-1BB agonist (e.g., 4-1BB or urelumab) had improved proliferation and increased cell count and cell viability (FIGS. 21B and 21C). Importantly, it was observed a dose-response effect of 19E3 and 4-1BB agonist (e.g., urelumab) in the cell maturation process, cell viability, and cell proliferation (FIGS. 22A-22D). Moreover, use of both 19E3 and urelumab provided improved the polyfunctionality of iT cells (FIGS. 23A and 23B). Next, it was determined T cells obtained by using the 19E3 antibody and a 4-1BB agonist were capable of reducing tumor burden in vivo. As demonstrated in FIGS. 24A-24E, iT cells obtained using 19E3 and urelumab improved tumor control even at the lowest tested dose (e.g., 4×10⁶ cells).

### DISCUSSION

The present example reports here on the generation of mature and functional CAR T cells facilitated by the use of the presently disclosed antibodies. Specifically, it was investigated how the presently disclosed antibodies can engage chimeric receptors (e.g., CAR) and induce maturation of iPSC-derived T cells.

Exposure to the presently disclosed antibodies resulted in the maturation of CAR-iT which had cytolytic capacity, cytokine secretion, and anti-tumor activity in vivo. Importantly, the use of the presently disclosed antibodies allowed the maturation of CAR-iT cells able to induce long-term remission and survival following intravenous infusion of a single dose of 4×10⁶ iT cells.

The presently disclosed antibodies provide a tool that can be used to generate clinically relevant CAR iT cell numbers, which in principle may allow for off-the-shelf application from batches of uniform and consistent CAR iT cells produced from the same engineered master cell bank.

In summary, the present example demonstrates that the presently disclosed antibodies can engage synthetic receptors like CARs which substitute for the TCR in driving directed T cell differentiation allowing for large-scale production of potent T cell-based immunotherapies

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of expanding a population of induced T cells, the method comprising:
(a) contacting an induced T cell comprising an antigen-recognizing receptor with an antibody or antigen-binding fragment thereof or an antigen-containing polypeptide that engages the antigen-recognizing receptor and an agonist of 4-1BB, and
(b) culturing the induced T cell to thereby produce an expanded population of induced T cells;
wherein the antibody or antigen-binding fragment thereof binds to an antigen-binding domain or to an idiotypic variable domain of the antigen-recognizing receptor, and
wherein the antigen-recognizing receptor is a chimeric antigen receptor (CAR) or a TCR like fusion protein (HIT).

2. The method of claim 1, wherein
(a) the antibody or antigen-binding fragment thereof binds to a scFv of the CAR;
(b) the antibody or antigen-binding fragment thereof binds to an idiotypic variable domain of a scFv of the CAR;
(c) the antibody or antigen-binding fragment thereof binds to an antigen-binding chain of the HIT; or
(d) the antibody or antigen-binding fragment thereof binds to an idiotypic variable domain of an antigen-binding chain of the HIT.

3. The method of claim 1, wherein the antibody or antigen-binding fragment thereof comprises:
(a) a heavy variable region comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62; and a light variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; or
(b) a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

4. The method of claim 1, wherein
(a) the antigen-recognizing receptor binds to CD19 and the antibody or antigen-binding fragment thereof comprises a heavy variable region comprising a CDR1 comprising the amino acid sequence set forth in SEQ ID NO: 60, a CDR2 comprising the amino acid sequence set forth in SEQ ID NO: 61, and a CDR3 comprising the amino acid sequence set forth in SEQ ID NO: 62; and a light variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65;
(b) the antigen-recognizing receptor binds to CD19 and the antibody or antigen-binding fragment thereof comprises a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75;
(c) the antigen-recognizing receptor binds to PSMA and the antibody or antigen-binding fragment thereof comprises a heavy variable region comprising a CDR1 comprising the amino acid sequence set forth in SEQ ID NO: 70, a CDR2 comprising the amino acid sequence set forth in SEQ ID NO: 71, and a CDR3 comprising the amino acid sequence set forth in SEQ ID NO: 72; and a light variable region comprising a CDR1 comprising the amino acid sequence set forth in SEQ ID NO: 73, a CDR2 comprising the amino acid sequence set forth in SEQ ID NO: 74, and a CDR3 comprising the amino acid sequence set forth in SEQ ID NO: 75.

5. The method of claim 1, wherein the antigen-containing polypeptide is an antigen or a fragment thereof or an Fc-fusion protein.

6. The method of claim 1, wherein the agonist of 4-1BB is an antibody or antigen-binding fragment thereof that binds 4-1BB.

7. The method of claim 6, wherein the antibody or antigen-binding fragment thereof that binds 4-1BB is urelumab.

8. The method of claim 1, wherein the antigen-recognizing receptor binds to a first antigen that is a tumor antigen or a pathogen antigen.

9. The method of claim 8, wherein the first antigen is selected from the group consisting of CD19, CD70, IL1RAP, ABCG2, AChR, ACKR6, ADAMTS13, ADGRE2 (EMR2), ADORA3, ADRA1D, AGER, ALS2, an antigen of a cytomegalovirus (CMV) infected cell, ANO9, AQP2, ASIC3, ASPRV1, ATP6VOA4, B3GNT4, B7-H3, BCMA, BEST4, C3orf35, CADM3, CAIX, CAPN3, CCDCl55, CCR1, CD10, CD117, CD123, CD133, CD135 (FLT3), CD138, CD20, CD22, CD244 (2B4), CD25, CD26, CD30, CD300LF, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBPlB, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

10. The method of claim 1, wherein the CAR comprises an extracellular antigen-binding domain that binds to the first antigen and an intracellular signaling domain that is capable of delivering an activation signal to the cell, wherein the intracellular signaling domain comprises a native CD3ζ polypeptide or a modified CD3ζ polypeptide.

11. The method of claim 1, wherein the antigen-recognizing receptor is encoded by a polynucleotide integrated at a locus within the genome of the induced T cell selected from the group consisting of a TRAC locus, a TRBC locus, a TRDC locus, and a TRGC locus.

12. The method of claim 1, wherein culturing comprises contacting the induced T cell comprising a chimeric receptor with IL-7, IL-21, or a combination thereof.

13. The method of claim 1, wherein the induced T cell is a cytotoxic T lymphocyte (CTL), a regulatory T cell, or a Natural Killer T (NKT) cell.

14. The method of claim 1, wherein the induced T cell is
(a) CD3⁺, TCR⁻;
(b) CD4⁺, CD3⁺, and TCR⁻; or
(c) CD8⁺, CD3⁺, and TCR⁻.

15. The method of claim 14, wherein the induced T cell is:
(a) CD3⁺, TCR⁻, CD25+, CD28+, CD69+, CD56⁺, CD45RA⁺;
(b) CD3⁺, TCR⁻, CD4⁻, CD8αα⁻;
(c) CD3⁺, TCR⁻, CD4⁻, CD8αβ⁻;
(d) CD3⁺, TCR⁻, CD4⁻, CD8αα⁺;
(e) CD3⁺, TCR⁻, CD4⁻, CD8αβ⁺;
(f) CD3⁺, TCR⁻, CD4⁺, CD8αα⁻;
(h) CD3⁺, TCR⁻, CD4⁺, CD8αβ⁻;
(i) CD3⁺, TCR⁻, CD4⁺, CD8αα⁺; or
(j) CD3⁺, TCR⁻, CD4⁺, CD8αβ⁺.

16. The method of claim 1, wherein the induced T cell further comprises (a) a gene disruption at a second locus selected from the group consisting of a CD52 locus, a CD70 locus, a PD1 locus, a CD38 locus, a PLZF locus, a SOX13 locus, and a combination thereof; and/or (b) a second antigen-recognizing receptor that targets a second antigen.

17. A method of obtaining and expanding a population of induced T cells, the method comprising:
(a) introducing into a pluripotent stem cell a polynucleotide encoding an antigen-recognizing receptor, wherein the antigen-recognizing receptor is a chimeric antigen receptor (CAR) or a TCR like fusion protein (HIT);
(b) contacting the pluripotent stem cell with a first cell culture medium comprising an activator of the bone morphogenic protein pathway to differentiate the pluripotent stem cell into a hematopoietic precursor;
(c) contacting the pluripotent stem cell with a second cell culture medium comprising a Notch ligand to differentiate the hematopoietic precursor into induced T cell; and
(d) expanding the induced T cell with the method of claim 1.

18. The method of claim 17, wherein the pluripotent stem cell is (a) an induced pluripotent stem cell; or (b) a T cell-derived induced pluripotent stem cell.

19. The method of claim 17, wherein the activator of the bone morphogenic protein pathway is a BMP-4 polypeptide (BMP-4).

20. The method of claim 17, wherein the first cell culture medium further comprises a fibroblast growth factor, VEGF, SCF, FLT3L, IL3, IL-6, IL-11, TPO, IGF-1, EPO, SHH, angiotensin II, AGTR1, or a combination thereof.

21. The method of claim 20, wherein the fibroblast growth factor is a basic fibroblast growth factor (bFGF).

22. The method of claim 17, wherein the pluripotent stem cell is in contact with the first cell culture medium for up to about 4 days or up to about 10 days.

23. The method of claim 17, wherein the Notch ligand is a DLL-1 polypeptide, a DLL-4 polypeptide, a JAG-1 polypeptide, a JAG-2 polypeptide, or a combination thereof.

24. The method of claim 23, wherein the Notch ligand is expressed by a feeder cell.

25. The method of claim 17, wherein the second cell culture medium further comprises SCF, FLT3L, IL-3, IL-7, IL-6, IL-11, TPO, IGF-1, EPO, SHH, angiotensin II, AGTR1, or a combination thereof.

26. The method of claim 17, wherein the hematopoietic precursor is in contact with the second cell culture medium for up to about 25 days.

27. An induced T cell obtained by the method of claim 1.

28. A composition comprising the induced T cell obtained by the method of claim 1.

29. A method of preventing and/or treating a neoplasm or a tumor in the subject, a pathogen infection in a subject, an autoimmune disease in a subject, and/or an infectious disease in a subject, administering to the subject an effective amount of the induced T cell produced by the method of claim 1.

30. A kit comprising the induced T cell produced by the method of claim 1.
